SEQUENCE LISTING (28 sequences)

SEQ ID NO: 1 - protein, 9 aa, Unknown
  Description of Unknown “LAGLIDADG” family peptide motif sequence
LAGLIDADG

SEQ ID NO: 2 - DNA, 90 bp, Artificial Sequence
  Description of Artificial Sequence Synthetic oligonucleotide
gttgatcagaccgatgttcggaccttccaaggtttcgatcggacatacgcgaccgtagtgggtcgggtgtacgtctcgaacttcaaagcc

SEQ ID NO: 3 - DNA, 1536 bp, Artificial Sequence
  Description of Artificial Sequence Synthetic polynucleotide
ttgaagagtttgatcatggctcagattgaacgctggcggcaggcctaacacatgcaagtcgaacggtagcacagagagcttgctctcgggtgacgagtggcggacgggtgagtaatgtctgggaaactgcctgatggagggggataactactggaaacggtagctaataccgcataacgtcgcaagaccaaagagggggaccttcgggcctcttgccatcagatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtgtgaagaaggccttcgggttgtaaagcactttcagcggggaggaagggagtaaggttaataaccttattcattgacgttacccgcagaagaagcaccggctaactccgtgccagcagccgcggtaatacggagggtgcaagcgttaatcggaattactgggcgtaaagcgcacgcaggcggtctgtcaagtcggatgtgaaatccccgggctcaacctgggaactgcatccgaaactggcaggcttgagtctcgtagagggaggtagaattccaggtgtagcggtgaaatgcgtagagatctggaggaataccggtggcgaaggcggcctcctggacgaagactgacgctcaggtgcgaaagcgtggggagcaaacaggattagataccctggtagtccacgccgtaaacgatgtctatttggaggttgtgcccttgaggcgtggcttccggagctaacgcgttaaatagaccgcctggggagtacggccgcaaggttaaaactcaaatgaattgacgggggcccgcacaagcggtggagcatgtggtttaattcgatgcaacgcgaagaaccttacctggtcttgacatccacagaactttccagagatggattggtgccttcgggaactgtgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtggggatgacgtcaagtcatcatggcccttacgaccagggctacacacgtgctacaatggcgcatacaaagagaagcgacctcgcgagagtaagcggacctcataaagtgcgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctt

SEQ ID NO: 4 - DNA, 1537 bp, Artificial Sequence
  Description of Artificial Sequence Synthetic polynucleotide
  modified_base (450)..(450): a, c, t, g, unknown or other
  modified_base (452)..(452): a, c, t, g, unknown or other
  modified_base (455)..(455): a, c, t, g, unknown or other
  modified_base (473)..(473): a, c, t, g, unknown or other
ttgaagagtttgatcatggctcagattgaacgctggcggcaggcctaacacatgcaagtcgaacggtagcacagagagcttgctctcgggtgacgagtggcggacgggtgagtaatgtctgggaaactgcctgatggagggggataactactggaaacggtagctaataccgcataacgtcgcaagaccaaagagggggaccttcgggcctcttgccatcagatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtgtgaagaaggccttcgggttgtaaagcactttcagcggggaggaaggnantanggttaataacctgtgttnattgacgttacccgcagaagaagcaccggctaactccgtgccagcagccgcggtaatacggagggtgcaagcgttaatcggaattactgggcgtaaagcgcacgcaggcggtctgtcaagtcggatgtgaaatccccgggctcaacctgggaactgcatccgaaactggcaggcttgagtctcgtagagggaggtagaattccaggtgtagcggtgaaatgcgtagagatctggaggaataccggtggcgaaggcggcctcctggacgaagactgacgctcaggtgcgaaagcgtggggagcaaacaggattagataccctggtagtccacgccgtaaacgatgtctatttggaggttgtgcccttgaggcgtggcttccggagctaacgcgttaaatagaccgcctggggagtacggccgcaaggttaaaactcaaatgaattgacgggggcccgcacaagcggtggagcatgtggtttaattcgatgcaacgcgaagaaccttacctggtcttgacatccacagaacttagcagagatgctttggtgccttcgggaactgtgagacaggtgctgcatggctgtcgtcagctcgtgttgtgaaatgttgggttaagtcccgcaacgagcgcaacccttatcctttgttgccagcggttaggccgggaactcaaaggagactgccagtgataaactggaggaaggtggggatgacgtcaagtcatcatggcccttacgaccagggctacacacgtgctacaatggcgcatacaaagagaagcgacctcgcgagagtaagcggacctcataaagtgcgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctt

SEQ ID NO: 5 - DNA, 20 bp, Artificial Sequence
  Description of Artificial Sequence Synthetic primer
agagtttgatcmtggctcag

SEQ ID NO: 6 - DNA, 19 bp, Artificial Sequence
  Description of Artificial Sequence Synthetic primer
ggttaccttgttacgactt

SEQ ID NO: 7 - DNA, 449 bp, Artificial Sequence
  Description of Artificial Sequence Synthetic polynucleotide
tcaccacggcgataaccataggttttcggcgtggccacatccatggtgaatcccactttttccagcacgcgcgccacttcatcgggtcttaaatacatagattttcctcgtcatctttccaaagcctcgccaccttacatgactgagcatggaccgtgactcagaaaattccacaaacgaacctgaaaggcgtgattgccgtctggccttaaaaattatggtctaaactaaaatttacatcgaaaacgagggaggatcctatgcataacaatgaacccaaaacgcagtcagaccctactctgggctatacattccaaaatgattttctggcgttaagtaaggcattttcattgcctgaaattgattacgccgatatttcccagcgagaacagttggcggcggcatttaagcgctggccgctgctggcagaattagcccgtcaacaataa

SEQ ID NO: 8 - DNA, 1425 bp, Artificial Sequence
  Description of Artificial Sequence Synthetic polynucleotide
cgtcctgtaataataaccggacaattcggactgattaaaaaagcgcccttgtggcgctttttttatattcccgcctccatttaaaataaaaaatccaatcggatttcactatttaaactggccattatctaagatgaatccgatggaagctcgctgttttaacacgcgttttttaaccttttattgaaagtcggtgcttctttgagcgaacgatcaaatttaagtggattcccatcaaaaaaatattctcaacctaaaaaagtttgtgtaatacttgtaacgctacatggagattaactcaatctagagggtattaataatgaatcgtactaaactggtactgggcatgcctttattcttttctgtgcaagatgataacaacatgaaaaaaatcatcggaattattttcacgctgatgctgctgttattatcgctgctggtagtgattactcccatgagcagcgataaacaatacctctttggccttagcgtgatggcagcggtatttatcctgggaagagtgaaaagcaaaaaagccgtactggcaatgctttccctctccgtattgatgtcgacacgttatatttggtggcgcgccacgacaacgctgcattttgattcgacggtagagatgctgctcggcagtttactttttgctgcggaaatttactcgtggacgattttactgctgggttatattcaaatggcgtggccattagaacgcccgattgcgcctttaccggcagataaatcaacctggccgaccgtcgatatttatgtcccctcttataatgaaagccttgaggttgttcgcgatacggttctggcggcgcagtgtatcgaatatccgcaagataaagtgaaggtttatattcttgacgacggaaagcgcgatgaattccgcgatttcgccgctgaagcgggcgttgggtatattacccgcccggataacagccacgccaaagccggtaatctcaaccacgcgatgaccatcacccatggtgagcttatctgcgtgttcgactgcgaccacgtggcaacgcgcgtgttcctgcaagccacgattggcgagtttttccgcgacgataaactggcgctgatccagacgccgcaccacttctattcgctcgatccatttgagcgcaacctgacagcagcaaagcgcgttccccacgaaggggcgctgttctacggcccggtgcagcaaggcaatgacaactggaacgccacgttcttctgcggctcctgcgcggtgatccgtcgtagcgcgctcaatgaagtcggcggctttgccgtggaaaccgtgaccgaagatgcgcataccgcgctgaaattacagcgtcgcggctggaacaccgcgtttctggatattccgctggcggcggggctggcaaccgagcgtctggcgctgcatgttaaccagcg

SEQ ID NO: 9 - DNA, 1339 bp, Artificial Sequence
  Description of Artificial Sequence Synthetic polynucleotide
tcaccacggcgataaccataggttttcggcgtggccacatccatggtgaatcccactttttccagcacgcgcgccacttcatcgggtcttaaatacatagattttcctcgtcatctttccaaagcctcgccaccttacatgactgagcatggaccgtgactcagaaaattccacaaacgaacctgaaaggcgtgattgccgtctggccttaaaaattatggtctaaactaaaatttacatcgaaaacgagggaggatcctatgcctttattcttttctgtgcaagatgataacaacatgaaaaaaatcatcggaattattttcacgctgatgctgctgttattatcgctgctggtagtgattactcccatgagcagcgataaacaatacctctttggccttagcgtgatggcagcggtatttatcctgggaagagtgaaaagcaaaaaagccgtactggcaatgctttccctctccgtattgatgtcgacacgttatatttggtggcgcgccacgacaacgctgcattttgattcgacggtagagatgctgctcggcagtttactttttgctgcggaaatttactcgtggacgattttactgctgggttatattcaaatggcgtggccattagaacgcccgattgcgcctttaccggcagataaatcaacctggccgaccgtcgatatttatgtcccctcttataatgaaagccttgaggttgttcgcgatacggttctggcggcgcagtgtatcgaatatccgcaagataaagtgaaggtttatattcttgacgacggaaagcgcgatgaattccgcgatttcgccgctgaagcgggcgttgggtatattacccgcccggataacagccacgccaaagccggtaatctcaaccacgcgatgaccatcacccatggtgagcttatctgcgtgttcgactgcgaccacgtggcaacgcgcgtgttcctgcaagccacgattggcgagtttttccgcgacgataaactggcgctgatccagacgccgcaccacttctattcgctcgatccatttgagcgcaacctgacagcagcaaagcgcgttccccacgaaggggcgctgttctacggcccggtgcagcaaggcaatgacaactggaacgccacgttcttctgcggctcctgcgcggtgatccgtcgtagcgcgctcaatgaagtcggcggctttgccgtggaaaccgtgaccgaagatgcgcataccgcgctgaaattacagcgtcgcggctggaacaccgcgtttctggatattccgctggcggcggggctggcaaccgagcgtctggcgctgcatgttaaccagcg

SEQ ID NO: 10 - DNA, 1408 bp, Artificial Sequence
  Description of Artificial Sequence Synthetic polynucleotide
atattgacaccatgacgcgcgtaatgctgattggttctgtgacgctggtaatgattgtcgaaattctgaacagtgccatcgaagccgtagtagaccgtattggtgcagaattccatgaactttccgggcgggcgaaggatatggggtcggcggcggtgctgatgtccatcctgctggcgatgtttacctggatcgcattactctggtcacattttcgataacgcttccagaattcgataacgccctggttttttgcttaaatttggttccaaaatcgcctttagctgtatatactcacagcataactgtatatacacccagggggcgggatgcctttattcttttctgtgcaagatgataacaacatgaaaaaaatcatcggaattattttcacgctgatgctgctgttattatcgctgctggtagtgattactcccatgagcagcgataaacaatacctctttggccttagcgtgatggcagcggtatttatcctgggaagagtgaaaagcaaaaaagccgtactggcaatgctttccctctccgtattgatgtcgacacgttatatttggtggcgcgccacgacaacgctgcattttgattcgacggtagagatgctgctcggcagtttactttttgctgcggaaatttactcgtggacgattttactgctgggttatattcaaatggcgtggccattagaacgcccgattgcgcctttaccggcagataaatcaacctggccgaccgtcgatatttatgtcccctcttataatgaaagccttgaggttgttcgcgatacggttctggcggcgcagtgtatcgaatatccgcaagataaagtgaaggtttatattcttgacgacggaaagcgcgatgaattccgcgatttcgccgctgaagcgggcgttgggtatattacccgcccggataacagccacgccaaagccggtaatctcaaccacgcgatgaccatcacccatggtgagcttatctgcgtgttcgactgcgaccacgtggcaacgcgcgtgttcctgcaagccacgattggcgagtttttccgcgacgataaactggcgctgatccagacgccgcaccacttctattcgctcgatccatttgagcgcaacctgacagcagcaaagcgcgttccccacgaaggggcgctgttctacggcccggtgcagcaaggcaatgacaactggaacgccacgttcttctgcggctcctgcgcggtgatccgtcgtagcgcgctcaatgaagtcggcggctttgccgtggaaaccgtgaccgaagatgcgcataccgcgctgaaattacagcgtcgcggctggaacaccgcgtttctggatattccgctggcggcggggctggcaaccgagcgtctggcgctgcatgttaaccagcg

SEQ ID NO: 11 - DNA, 773 bp, Artificial Sequence
  Description of Artificial Sequence Synthetic polynucleotide
tcaccacggcgataaccataggttttcggcgtggccacatccatggtgaatcccactttttccagcacgcgcgccacttcatcgggtcttaaatacatagattttcctcgtcatctttccaaagcctcgccaccttacatgactgagcatggaccgtgactcagaaaattccacaaacgaacctgaaaggcgtgattgccgtctggccttaaaaattatggtctaaactaaaatttacatcgaaaacgagggaggatcctatgggtcattacgatgatttgcagaggtttaaggacaagactcgcaatcagaagcacgattttaaggatctctccgcacagaacctcgcacaagaccagagcaattgggcaatcattaaccagctttctcctgccacggatgaatcaatgctggcaatgggcggccatgtgtcactgcctgtcccgcaatccgtcgatcctgcgctttttgccgcgcaagagccggctgcgttggtggatgatattgcacctgcgcctgcgtcaacaacttctctgctaaaagacgtcgccagccagcttgatgcagtggctccggcattcaacgccgcagcctacgcgcctgcgcctgcaagcgttgcaccggttatgcagatgccatcgcctgcaaatccttcttcggctcctgtcagctatgcgcgcctgttcgcagcgaaagcgacggaggagaagccccgcgcggaaaaaaatcagcccttacactcgttgttagaaaggatcgcttcatgccgttga

SEQ ID NO: 12 - DNA, 1108 bp, Artificial Sequence
  Description of Artificial Sequence Synthetic polynucleotide
cgtcctgtaataataaccggacaattcggactgattaaaaaagcgcccttgtggcgctttttttatattcccgcctccatttaaaataaaaaatccaatcggatttcactatttaaactggccattatctaagatgaatccgatggaagctcgctgttttaacacgcgttttttaaccttttattgaaagtcggtgcttctttgagcgaacgatcaaatttaagtggattcccatcaaaaaaatattctcaacctaaaaaagtttgtgtaatacttgtaacgctacatggagattaactcaatctagagggtattaataatgaatcgtactaaactggtactgggcatggtttcatcatccccgtctgttgctcgcagcggcatgttttacttttcccagggctggaaactggtcacactgccaggcattcgtcgctttgtgatcttgccgttaatggtgaatattttgctgatgggcggcgcgttctggtggctgtttacccgcctggatagttggatcccttcgctgatgagccatgtgccggactggctgcaatggctgagctatttactgtggccaattgccgttgtctcaattctgttggtcttcggctacttcttctcaacccttgcgaactggattgccgctccctttaatggcttgctggcagaacagctggaagcacgccttaccggcgcgacaccacccgacgtcggcgtttttgggctgctcaaagatgtaccgcgcatcatgaagcgcgaatggcaaaaactggcctggtatctgccccgcgccttgctgctgctggtgctttattttattcccggtattgggcaaaccgtcgccccggtgctgtggttcctgttcagtgcgtggatgttggctatccagtattgcgactatccgtttgataaccataaggtgccgtttaaagcgatgcgcgtttccttgcgcgaacgcaaggttattaatatgcaattcggtgcgctcaccagcctgtttacgatgattccggtgttgaatttggtcgtgatgcccgtagcggtgtgtggcgcaacggcgatgtgggtcgattgctaccgggaaaaacacgcggtctggcgataa

SEQ ID NO: 13 - DNA, 4222 bp, Artificial Sequence
  Description of Artificial Sequence Synthetic polynucleotide
cgtcctgtaataataaccggacaattcggactgattaaaaaagcgcccttgtggcgctttttttatattcccgcctccatttaaaataaaaaatccaatcggatttcactatttaaactggccattatctaagatgaatccgatggaagctcgctgttttaacacgcgttttttaaccttttattgaaagtcggtgcttctttgagcgaacgatcaaatttaagtggattcccatcaaaaaaatattctcaacctaaaaaagtttgtgtaatacttgtaacgctacatggagattaactcaatctagagggtattaataatgaatcgtactaaactggtactgggcgtgattaattcggaaaagaaagtccaacgccttgtaagaactcagctggccccgttgacggtaacaatgatattcgcattacctgctgttgcgcattccgctggtttaagtatgaaaaatggcacgctatataatgcgaatggcgtgccggtcgtggatattaataaacctaacagcaatggcttgtcacataacgtctgggacaacctgaacgtcgataaaaacggtgttatttttaataacagcggctcagcatccgatacggtattgggcggcaccatccagggtaacagcaatctgacatccgggtcggcaaaagttattctcaacgaagttacctccaaaaatgcgtcgacgatcaacggtatgatggaagttgcgggtgataaagccgcgttgattatcgccaaccccaatggcattgcggtaaatggtggcggtacgatcaataccgataaactcactctgaccactggcacaccagatattcagaacgacaaactcgctggttattccgtcaacggcggtaccatcacgatcagcaaactggataacgccagcccaactgaaattttgtcacgcaacgtagtgctcagcgggaaagttaccgctgacgaactgaatattgttgcgggtaataactacgttaatacagacggccaggtgaccggaactgtcactgcgtcaggctcgcgtaacggctatagcgttgacgttgcgcaagttggcggcatgtacgcgaataaaattaatcttatcagtaccgaaagcggcgtgggcgtgcgtaatcagggcgtaattgccggtggcactggtgggatcagcattgatgctaatggccagctactgaacagtaatgcgcagattcaatcgaccggttcgacaaagattaataccaacggcaaattggacaatacaacgggtcttattacttctgctggtgctatttatctcgatactaataagaacgctattgttaatgcccgttcaggtacgatttcgacgacggcagatatctacgttaatagcggcgcagttgataataccaacggcaaaattgccgccagcggcgtgctggctgtcgataccaataacaatacttttaccaacgccggtaaagggaacgatgtcggtatcgaagcgggtattgtcaccttaaaaaccggtacgctaaataatagtaacggccagatccacggcggctatctggggcttgaatccggcgcgattaacaataacagcggtattctggaaacgtcaggcgacatggacattgtcagccttggaaatgtcgacaataacaaaggcctgatccgatctgccagcggccatatcaacatcctgtctggcggcactatcaacaacggcagtaccaaaacagcggataccgtcagttccgactccttaggcatcgcagccgataccggtgtagtaatcaacgcaaacagcatcaataatagcggtggtcagatcgcatcgaacggcgatgtgtcgctaaacagtaagggcctggtcaataacgattccggtaatcttctttccaatagcaaagtgattgtgaagggcggttcttatcgtaatgactacggtggcatcggcggcaagaagggcgtggaggtgtcggttaacggcgacctgagcaactatatcggcgttgtgagtgcggaagaaggtgatatctcactgagtgcaaatgcagtggatacccacggcggtttcatgatggggcaaaatatctccatcgacgcgaaagccggcgtaaataccacccgatcgctggtattagccagtgaaaagctggctatccatgcaggcggaaatgttgataaccgtgatggtaatagctttggcaatgacttcggcctctatttcggcatgccgcaacagaccggtggcttgatcggtaaaggtggcgttgagatttccgggcagaatatttacaacaacaacagccgcatcattgcagaaaatgggccaatgagcctgcaagcgaaagggaacattgataacacgcgtgcgctgatggtcagcggggccgataccgcaatcaaaaccggcggggctttctacaataactacgcaacgatttccagcggaggcaacctcacactcgataccgtttccctggaaaactccagcagcggcacgcttatcgataataacgccacaggtttcatcaactcagataaagatatcacgctgaatgtggacaacaatttcacgaactacggctggatcagtggtaaaggcgcagtggcgctgaatgtcgtgaaaggggcgttttataaccgtaataccattgcggctgataagtcacttgcgatcagtgccctgaacggtatcgagaactttaaagatatctctgcaggcggcagcctgacgctggatacgcaacgtcatgtgacaaataacagcaacagcaatatggtcggtaacgggattactattaacgcggtaaacgacattaataaccgcggcaatattgtcagtgatggcgatatgagtgttgtgaccaaaggtaacctgtataactacctctatatgctgggcgctggtgatgtcgcgattaccgcaaacaacgtcaacaacaataattcacttattgaggctggtggtgatctgacgattacctctactggcaatgtgggtaataaccgtggcacgctgcgctccctgacgggcgatgtaaatgtggcggcaaaaggaacggttgataactatagcggtaagattacctctgctggcgatgtcggagtcagtggaaactggttgctcaatgattatggtcagattgctggcttaagcaatattaacctgattctgacgggcaacttcgacagttacaaaggttctgtgacctctcaaatgggcgatatcaagctggttgccaactcggtggataataacaacgggttaatcgcaggtgaaaatatctccattgattccaaatcaggtgtctataacaacacggcgctgattgccgcgaataaaacacttaccgtaaatgctgtcggcaatattgaaaacagagacggtaataacttcagttattacaacggcgagtattttggtgtcgcaggcgatgtgggtggcatggtgggtaacgcgggcatctcgctttccgcacagaatatctacagcacaaacagccgtattctggcagaaaatggcccacttaacgtgctgtcccggggcgtattcgataacacccgcagcatgctgatgagcggtgccaacgccattattaaagcggccggtgttttctataataactatgcgatgacatatagcgcaggtaacctcgacatcacttctgcttaccttgagaactacagcaacggtaagctggaagatggaacggcaacaggcgttatcgcttcggataagaacctgaacctgagcgtggataatagcaccaccaactacggctggatcagcggtaagggcgatgtgaatttcagcgtgttaaaaggtgtgctgtataaccgcaacaccatcgccgctaataacgcgctggcgattaatgccctgaacggtatcgagaacttcaaagatatcctggcgggaaccagccttacccttactacccaacggcacgttaccaacaacagcaacagtaatatgcttgggcaaagcattgggattaatgcggtaaacgatattaataaccgcggcaatattgtcagcgattatgcattaaccgtgaaaacagacgggaacgtgtataactacctcaatatgctgagttatggcaccgcaaaagtaaccgcgaataaagtcacgaacagcggcaaggacgcggtgctgggcggtttttacggccttgaccttgaatcgaatgcgatcaccaataccggcaccattgtaggtctgtaa

SEQ ID NO: 14 - DNA, 4136 bp, Artificial Sequence
  Description of Artificial Sequence Synthetic polynucleotide
tcaccacggcgataaccataggttttcggcgtggccacatccatggtgaatcccactttttccagcacgcgcgccacttcatcgggtcttaaatacatagattttcctcgtcatctttccaaagcctcgccaccttacatgactgagcatggaccgtgactcagaaaattccacaaacgaacctgaaaggcgtgattgccgtctggccttaaaaattatggtctaaactaaaatttacatcgaaaacgagggaggatcctgtgattaattcggaaaagaaagtccaacgccttgtaagaactcagctggccccgttgacggtaacaatgatattcgcattacctgctgttgcgcattccgctggtttaagtatgaaaaatggcacgctatataatgcgaatggcgtgccggtcgtggatattaataaacctaacagcaatggcttgtcacataacgtctgggacaacctgaacgtcgataaaaacggtgttatttttaataacagcggctcagcatccgatacggtattgggcggcaccatccagggtaacagcaatctgacatccgggtcggcaaaagttattctcaacgaagttacctccaaaaatgcgtcgacgatcaacggtatgatggaagttgcgggtgataaagccgcgttgattatcgccaaccccaatggcattgcggtaaatggtggcggtacgatcaataccgataaactcactctgaccactggcacaccagatattcagaacgacaaactcgctggttattccgtcaacggcggtaccatcacgatcagcaaactggataacgccagcccaactgaaattttgtcacgcaacgtagtgctcagcgggaaagttaccgctgacgaactgaatattgttgcgggtaataactacgttaatacagacggccaggtgaccggaactgtcactgcgtcaggctcgcgtaacggctatagcgttgacgttgcgcaagttggcggcatgtacgcgaataaaattaatcttatcagtaccgaaagcggcgtgggcgtgcgtaatcagggcgtaattgccggtggcactggtgggatcagcattgatgctaatggccagctactgaacagtaatgcgcagattcaatcgaccggttcgacaaagattaataccaacggcaaattggacaatacaacgggtcttattacttctgctggtgctatttatctcgatactaataagaacgctattgttaatgcccgttcaggtacgatttcgacgacggcagatatctacgttaatagcggcgcagttgataataccaacggcaaaattgccgccagcggcgtgctggctgtcgataccaataacaatacttttaccaacgccggtaaagggaacgatgtcggtatcgaagcgggtattgtcaccttaaaaaccggtacgctaaataatagtaacggccagatccacggcggctatctggggcttgaatccggcgcgattaacaataacagcggtattctggaaacgtcaggcgacatggacattgtcagccttggaaatgtcgacaataacaaaggcctgatccgatctgccagcggccatatcaacatcctgtctggcggcactatcaacaacggcagtaccaaaacagcggataccgtcagttccgactccttaggcatcgcagccgataccggtgtagtaatcaacgcaaacagcatcaataatagcggtggtcagatcgcatcgaacggcgatgtgtcgctaaacagtaagggcctggtcaataacgattccggtaatcttctttccaatagcaaagtgattgtgaagggcggttcttatcgtaatgactacggtggcatcggcggcaagaagggcgtggaggtgtcggttaacggcgacctgagcaactatatcggcgttgtgagtgcggaagaaggtgatatctcactgagtgcaaatgcagtggatacccacggcggtttcatgatggggcaaaatatctccatcgacgcgaaagccggcgtaaataccacccgatcgctggtattagccagtgaaaagctggctatccatgcaggcggaaatgttgataaccgtgatggtaatagctttggcaatgacttcggcctctatttcggcatgccgcaacagaccggtggcttgatcggtaaaggtggcgttgagatttccgggcagaatatttacaacaacaacagccgcatcattgcagaaaatgggccaatgagcctgcaagcgaaagggaacattgataacacgcgtgcgctgatggtcagcggggccgataccgcaatcaaaaccggcggggctttctacaataactacgcaacgatttccagcggaggcaacctcacactcgataccgtttccctggaaaactccagcagcggcacgcttatcgataataacgccacaggtttcatcaactcagataaagatatcacgctgaatgtggacaacaatttcacgaactacggctggatcagtggtaaaggcgcagtggcgctgaatgtcgtgaaaggggcgttttataaccgtaataccattgcggctgataagtcacttgcgatcagtgccctgaacggtatcgagaactttaaagatatctctgcaggcggcagcctgacgctggatacgcaacgtcatgtgacaaataacagcaacagcaatatggtcggtaacgggattactattaacgcggtaaacgacattaataaccgcggcaatattgtcagtgatggcgatatgagtgttgtgaccaaaggtaacctgtataactacctctatatgctgggcgctggtgatgtcgcgattaccgcaaacaacgtcaacaacaataattcacttattgaggctggtggtgatctgacgattacctctactggcaatgtgggtaataaccgtggcacgctgcgctccctgacgggcgatgtaaatgtggcggcaaaaggaacggttgataactatagcggtaagattacctctgctggcgatgtcggagtcagtggaaactggttgctcaatgattatggtcagattgctggcttaagcaatattaacctgattctgacgggcaacttcgacagttacaaaggttctgtgacctctcaaatgggcgatatcaagctggttgccaactcggtggataataacaacgggttaatcgcaggtgaaaatatctccattgattccaaatcaggtgtctataacaacacggcgctgattgccgcgaataaaacacttaccgtaaatgctgtcggcaatattgaaaacagagacggtaataacttcagttattacaacggcgagtattttggtgtcgcaggcgatgtgggtggcatggtgggtaacgcgggcatctcgctttccgcacagaatatctacagcacaaacagccgtattctggcagaaaatggcccacttaacgtgctgtcccggggcgtattcgataacacccgcagcatgctgatgagcggtgccaacgccattattaaagcggccggtgttttctataataactatgcgatgacatatagcgcaggtaacctcgacatcacttctgcttaccttgagaactacagcaacggtaagctggaagatggaacggcaacaggcgttatcgcttcggataagaacctgaacctgagcgtggataatagcaccaccaactacggctggatcagcggtaagggcgatgtgaatttcagcgtgttaaaaggtgtgctgtataaccgcaacaccatcgccgctaataacgcgctggcgattaatgccctgaacggtatcgagaacttcaaagatatcctggcgggaaccagccttacccttactacccaacggcacgttaccaacaacagcaacagtaatatgcttgggcaaagcattgggattaatgcggtaaacgatattaataaccgcggcaatattgtcagcgattatgcattaaccgtgaaaacagacgggaacgtgtataactacctcaatatgctgagttatggcaccgcaaaagtaaccgcgaataaagtcacgaacagcggcaaggacgcggtgctgggcggtttttacggccttgaccttgaatcgaatgcgatcaccaataccggcaccattgtaggtctgtaa

SEQ ID NO: 15 - DNA, 4205 bp, Artificial Sequence
  Description of Artificial Sequence Synthetic polynucleotide
atattgacaccatgacgcgcgtaatgctgattggttctgtgacgctggtaatgattgtcgaaattctgaacagtgccatcgaagccgtagtagaccgtattggtgcagaattccatgaactttccgggcgggcgaaggatatggggtcggcggcggtgctgatgtccatcctgctggcgatgtttacctggatcgcattactctggtcacattttcgataacgcttccagaattcgataacgccctggttttttgcttaaatttggttccaaaatcgcctttagctgtatatactcacagcataactgtatatacacccagggggcggggtgattaattcggaaaagaaagtccaacgccttgtaagaactcagctggccccgttgacggtaacaatgatattcgcattacctgctgttgcgcattccgctggtttaagtatgaaaaatggcacgctatataatgcgaatggcgtgccggtcgtggatattaataaacctaacagcaatggcttgtcacataacgtctgggacaacctgaacgtcgataaaaacggtgttatttttaataacagcggctcagcatccgatacggtattgggcggcaccatccagggtaacagcaatctgacatccgggtcggcaaaagttattctcaacgaagttacctccaaaaatgcgtcgacgatcaacggtatgatggaagttgcgggtgataaagccgcgttgattatcgccaaccccaatggcattgcggtaaatggtggcggtacgatcaataccgataaactcactctgaccactggcacaccagatattcagaacgacaaactcgctggttattccgtcaacggcggtaccatcacgatcagcaaactggataacgccagcccaactgaaattttgtcacgcaacgtagtgctcagcgggaaagttaccgctgacgaactgaatattgttgcgggtaataactacgttaatacagacggccaggtgaccggaactgtcactgcgtcaggctcgcgtaacggctatagcgttgacgttgcgcaagttggcggcatgtacgcgaataaaattaatcttatcagtaccgaaagcggcgtgggcgtgcgtaatcagggcgtaattgccggtggcactggtgggatcagcattgatgctaatggccagctactgaacagtaatgcgcagattcaatcgaccggttcgacaaagattaataccaacggcaaattggacaatacaacgggtcttattacttctgctggtgctatttatctcgatactaataagaacgctattgttaatgcccgttcaggtacgatttcgacgacggcagatatctacgttaatagcggcgcagttgataataccaacggcaaaattgccgccagcggcgtgctggctgtcgataccaataacaatacttttaccaacgccggtaaagggaacgatgtcggtatcgaagcgggtattgtcaccttaaaaaccggtacgctaaataatagtaacggccagatccacggcggctatctggggcttgaatccggcgcgattaacaataacagcggtattctggaaacgtcaggcgacatggacattgtcagccttggaaatgtcgacaataacaaaggcctgatccgatctgccagcggccatatcaacatcctgtctggcggcactatcaacaacggcagtaccaaaacagcggataccgtcagttccgactccttaggcatcgcagccgataccggtgtagtaatcaacgcaaacagcatcaataatagcggtggtcagatcgcatcgaacggcgatgtgtcgctaaacagtaagggcctggtcaataacgattccggtaatcttctttccaatagcaaagtgattgtgaagggcggttcttatcgtaatgactacggtggcatcggcggcaagaagggcgtggaggtgtcggttaacggcgacctgagcaactatatcggcgttgtgagtgcggaagaaggtgatatctcactgagtgcaaatgcagtggatacccacggcggtttcatgatggggcaaaatatctccatcgacgcgaaagccggcgtaaataccacccgatcgctggtattagccagtgaaaagctggctatccatgcaggcggaaatgttgataaccgtgatggtaatagctttggcaatgacttcggcctctatttcggcatgccgcaacagaccggtggcttgatcggtaaaggtggcgttgagatttccgggcagaatatttacaacaacaacagccgcatcattgcagaaaatgggccaatgagcctgcaagcgaaagggaacattgataacacgcgtgcgctgatggtcagcggggccgataccgcaatcaaaaccggcggggctttctacaataactacgcaacgatttccagcggaggcaacctcacactcgataccgtttccctggaaaactccagcagcggcacgcttatcgataataacgccacaggtttcatcaactcagataaagatatcacgctgaatgtggacaacaatttcacgaactacggctggatcagtggtaaaggcgcagtggcgctgaatgtcgtgaaaggggcgttttataaccgtaataccattgcggctgataagtcacttgcgatcagtgccctgaacggtatcgagaactttaaagatatctctgcaggcggcagcctgacgctggatacgcaacgtcatgtgacaaataacagcaacagcaatatggtcggtaacgggattactattaacgcggtaaacgacattaataaccgcggcaatattgtcagtgatggcgatatgagtgttgtgaccaaaggtaacctgtataactacctctatatgctgggcgctggtgatgtcgcgattaccgcaaacaacgtcaacaacaataattcacttattgaggctggtggtgatctgacgattacctctactggcaatgtgggtaataaccgtggcacgctgcgctccctgacgggcgatgtaaatgtggcggcaaaaggaacggttgataactatagcggtaagattacctctgctggcgatgtcggagtcagtggaaactggttgctcaatgattatggtcagattgctggcttaagcaatattaacctgattctgacgggcaacttcgacagttacaaaggttctgtgacctctcaaatgggcgatatcaagctggttgccaactcggtggataataacaacgggttaatcgcaggtgaaaatatctccattgattccaaatcaggtgtctataacaacacggcgctgattgccgcgaataaaacacttaccgtaaatgctgtcggcaatattgaaaacagagacggtaataacttcagttattacaacggcgagtattttggtgtcgcaggcgatgtgggtggcatggtgggtaacgcgggcatctcgctttccgcacagaatatctacagcacaaacagccgtattctggcagaaaatggcccacttaacgtgctgtcccggggcgtattcgataacacccgcagcatgctgatgagcggtgccaacgccattattaaagcggccggtgttttctataataactatgcgatgacatatagcgcaggtaacctcgacatcacttctgcttaccttgagaactacagcaacggtaagctggaagatggaacggcaacaggcgttatcgcttcggataagaacctgaacctgagcgtggataatagcaccaccaactacggctggatcagcggtaagggcgatgtgaatttcagcgtgttaaaaggtgtgctgtataaccgcaacaccatcgccgctaataacgcgctggcgattaatgccctgaacggtatcgagaacttcaaagatatcctggcgggaaccagccttacccttactacccaacggcacgttaccaacaacagcaacagtaatatgcttgggcaaagcattgggattaatgcggtaaacgatattaataaccgcggcaatattgtcagcgattatgcattaaccgtgaaaacagacgggaacgtgtataactacctcaatatgctgagttatggcaccgcaaaagtaaccgcgaataaagtcacgaacagcggcaaggacgcggtgctgggcggtttttacggccttgaccttgaatcgaatgcgatcaccaataccggcaccattgtaggtctgtaa

SEQ ID NO: 16 - DNA, 860 bp, Artificial Sequence
  Description of Artificial Sequence Synthetic polynucleotide
gaatttacttacattaaggcggcgaggggcgcctatacttgatagttctgataccagaagaaggaagaactatggataaccagatatctgtaccgcctgcgctaaccggaaactacgcttttttctttgacctcgacggtactcttgccgatatccaaccgcaccccgatcaggtggtgatacccgacaacacgttgcaggcgctcaacgcgctggcccagcagcagggtggggcggtggcattgatttcagggcgctcaatggctgaacttgacgcgctaacccatccctggcggctaccgctggccggcgttcacggggcagaacgccgtgatatcaatggtaaaacctatatcgtttctctgccgtcggcgctgcgcgatgaaatcgcgactgagctgacctcggcgctgcaggggctctccggctgcgagctggagagtaaggagatggccttcgcgctgcactaccgccaggctccgcagcagcagagcgcagtactggagctggctcagcgcatcgttcagcgctacccgctgctggcactgcagctgggtaaatgtgtggtggagattaaaccccgcggagtgaacaaaggggaggcgatcagcgccttcatgcaggaggcgccgtttgccggccgcgaaccggtttttgtcggcgatgatttgaccgatgaggcgggatttagcgtggttaatcaactgcagggaatgtcggtaaaagtcggcgccggggaaactcaggcgcactggcggttggcggatgccgccgctgtaaggacgtggttgcaacatctggcgtatgacgcgcaaaccgaaaggagggatgaccatgagtcgtttagtcgtagtctctaa

SEQ ID NO: 17 - DNA, 1088 bp, Artificial Sequence
  Description of Artificial Sequence Synthetic polynucleotide
cgccgtcctcgcagtaccattgcaaccgactttacagcaagaagtgattctggcacgcatggaacaaattcttgccagtcgggctttatccgatgacgaacgcgcacagcttttatatgagcgcggagtgttgtatgatagtctcggtctgagggcattagcgcgaaatgatttttcacaagcgctggcaatccgacccgatatgcctgaagtattcaattacttaggcatttacttaacgcaggcaggcaattttgatgctgcctatgaagcgtttgattctgtacttgagcttgatcatggataaccagatatctgtaccgcctgcgctaaccggaaactacgcttttttctttgacctcgacggtactcttgccgatatccaaccgcaccccgatcaggtggtgatacccgacaacacgttgcaggcgctcaacgcgctggcccagcagcagggtggggcggtggcattgatttcagggcgctcaatggctgaacttgacgcgctaacccatccctggcggctaccgctggccggcgttcacggggcagaacgccgtgatatcaatggtaaaacctatatcgtttctctgccgtcggcgctgcgcgatgaaatcgcgactgagctgacctcggcgctgcaggggctctccggctgcgagctggagagtaaggagatggccttcgcgctgcactaccgccaggctccgcagcagcagagcgcagtactggagctggctcagcgcatcgttcagcgctacccgctgctggcactgcagctgggtaaatgtgtggtggagattaaaccccgcggagtgaacaaaggggaggcgatcagcgccttcatgcaggaggcgccgtttgccggccgcgaaccggtttttgtcggcgatgatttgaccgatgaggcgggatttagcgtggttaatcaactgcagggaatgtcggtaaaagtcggcgccggggaaactcaggcgcactggcggttggcggatgccgccgctgtaaggacgtggttgcaacatctggcgtatgacgcgcaaaccgaaaggagggatgaccatgagtcgtttagtcgtagtctctaa

SEQ ID NO: 18 - DNA, 1153 bp, Artificial Sequence
  Description of Artificial Sequence Synthetic polynucleotide
cgtcctgtaataataaccggacaattcggactgattaaaaaagcgcccttgtggcgctttttttatattcccgcctccatttaaaataaaaaatccaatcggatttcactatttaaactggccattatctaagatgaatccgatggaagctcgctgttttaacacgcgttttttaaccttttattgaaagtcggtgcttctttgagcgaacgatcaaatttaagtggattcccatcaaaaaaatattctcaacctaaaaaagtttgtgtaatacttgtaacgctacatggagattaactcaatctagagggtattaataatgaatcgtactaaactggtactgggcgtggaagacgcactatctataccgcctctcatttccggaaattttgccttttttttcgatttggatggcacccttgcggacatcaaaccgcacccggatgatgtttttatccctgaagatgtgctgaccagactttcattgcttgctgagctaaatgacagggcgttggcattgatttcagggcgttcaattgctgagctggaacagctcgccaaaccgtatcgtttcccgctggccggggtacatggggcggagcgccgcgatatcaatggtcgaaccgaacgaatgaccttgccggaagcggttttacaaccgctggaacgtgaactccggcatggcattgcgccgctgaccggcgttgaacttgaaaccaaagggatggcatttgcactgcattatcgccaggcgccgcagcatgaagaggcggtctttgccctggcaaaaacactcatcgcgcactacccacaactggcaatgcagccgggaaaatgtgtggtcgagctgaaaccctctggcattcataaaggcgccgctattggcgcgtttatgcaaacgccgccctttctcggcaagaaaccggtgtttcttggcgacgatctgactgacgaacatggatttaaaaccgttaataaacttggcggcatatcgataaaagttggccctggcgcaacacaggcaatgtggcgtctggctggcgtaaatgatgtctatcaatggcttgaaaaacttgtcgaccatcaacaacaagaacaacgggcgctaacaaacaggagagatggctatgagtcgcttagtcgtagtatctaa

SEQ ID NO: 19 - DNA, 1067 bp, Artificial Sequence
  Description of Artificial Sequence Synthetic polynucleotide
tcaccacggcgataaccataggttttcggcgtggccacatccatggtgaatcccactttttccagcacgcgcgccacttcatcgggtcttaaatacatagattttcctcgtcatctttccaaagcctcgccaccttacatgactgagcatggaccgtgactcagaaaattccacaaacgaacctgaaaggcgtgattgccgtctggccttaaaaattatggtctaaactaaaatttacatcgaaaacgagggaggatcctgtggaagacgcactatctataccgcctctcatttccggaaattttgccttttttttcgatttggatggcacccttgcggacatcaaaccgcacccggatgatgtttttatccctgaagatgtgctgaccagactttcattgcttgctgagctaaatgacagggcgttggcattgatttcagggcgttcaattgctgagctggaacagctcgccaaaccgtatcgtttcccgctggccggggtacatggggcggagcgccgcgatatcaatggtcgaaccgaacgaatgaccttgccggaagcggttttacaaccgctggaacgtgaactccggcatggcattgcgccgctgaccggcgttgaacttgaaaccaaagggatggcatttgcactgcattatcgccaggcgccgcagcatgaagaggcggtctttgccctggcaaaaacactcatcgcgcactacccacaactggcaatgcagccgggaaaatgtgtggtcgagctgaaaccctctggcattcataaaggcgccgctattggcgcgtttatgcaaacgccgccctttctcggcaagaaaccggtgtttcttggcgacgatctgactgacgaacatggatttaaaaccgttaataaacttggcggcatatcgataaaagttggccctggcgcaacacaggcaatgtggcgtctggctggcgtaaatgatgtctatcaatggcttgaaaaacttgtcgaccatcaacaacaagaacaacgggcgctaacaaacaggagagatggctatgagtcgcttagtcgtagtatctaa

SEQ ID NO: 20 - DNA, 730 bp, Artificial Sequence
  Description of Artificial Sequence Synthetic polynucleotide
cgtcctgtaataataaccggacaattcggactgattaaaaaagcgcccttgtggcgctttttttatattcccgcctccatttaaaataaaaaatccaatcggatttcactatttaaactggccattatctaagatgaatccgatggaagctcgctgttttaacacgcgttttttaaccttttattgaaagtcggtgcttctttgagcgaacgatcaaatttaagtggattcccatcaaaaaaatattctcaacctaaaaaagtttgtgtaatacttgtaacgctacatggagattaactcaatctagagggtattaataatgaatcgtactaaactggtactgggcatgggatatgacgcgtgtaattttattaactcgatcgttaaaaattttaaagaatccattggaataatgaacgtgttgttactggaacctgatgagtccgacgctttgcgcttattgtatctaaaactggatgttatcagggctggcattgagcaggatgtccgcatgttaaccaaagcccgtctggacagcaacgccgcactttgcctttaccaggtacataatgcgttaaatgctttgaactattgcattgggattgtcagaaatacgccgttaaatcagcaggtttattacgatttcgaagatctaaagcgtattgtgaacactcttgatgcaacatccggacgtgtttttaataaactggatggtaatgagtacacataa

SEQ ID NO: 21 - DNA, 644 bp, Artificial Sequence
  Description of Artificial Sequence Synthetic polynucleotide
tcaccacggcgataaccataggttttcggcgtggccacatccatggtgaatcccactttttccagcacgcgcgccacttcatcgggtcttaaatacatagattttcctcgtcatctttccaaagcctcgccaccttacatgactgagcatggaccgtgactcagaaaattccacaaacgaacctgaaaggcgtgattgccgtctggccttaaaaattatggtctaaactaaaatttacatcgaaaacgagggaggatcctatgggatatgacgcgtgtaattttattaactcgatcgttaaaaattttaaagaatccattggaataatgaacgtgttgttactggaacctgatgagtccgacgctttgcgcttattgtatctaaaactggatgttatcagggctggcattgagcaggatgtccgcatgttaaccaaagcccgtctggacagcaacgccgcactttgcctttaccaggtacataatgcgttaaatgctttgaactattgcattgggattgtcagaaatacgccgttaaatcagcaggtttattacgatttcgaagatctaaagcgtattgtgaacactcttgatgcaacatccggacgtgtttttaataaactggatggtaatgagtacacataa

SEQ ID NO: 22 - DNA, 713 bp, Artificial Sequence
  Description of Artificial Sequence Synthetic polynucleotide
atattgacaccatgacgcgcgtaatgctgattggttctgtgacgctggtaatgattgtcgaaattctgaacagtgccatcgaagccgtagtagaccgtattggtgcagaattccatgaactttccgggcgggcgaaggatatggggtcggcggcggtgctgatgtccatcctgctggcgatgtttacctggatcgcattactctggtcacattttcgataacgcttccagaattcgataacgccctggttttttgcttaaatttggttccaaaatcgcctttagctgtatatactcacagcataactgtatatacacccagggggcgggatgggatatgacgcgtgtaattttattaactcgatcgttaaaaattttaaagaatccattggaataatgaacgtgttgttactggaacctgatgagtccgacgctttgcgcttattgtatctaaaactggatgttatcagggctggcattgagcaggatgtccgcatgttaaccaaagcccgtctggacagcaacgccgcactttgcctttaccaggtacataatgcgttaaatgctttgaactattgcattgggattgtcagaaatacgccgttaaatcagcaggtttattacgatttcgaagatctaaagcgtattgtgaacactcttgatgcaacatccggacgtgtttttaataaactggatggtaatgagtacacataa

SEQ ID NO: 23 - DNA, 2048 bp, Artificial Sequence
  Description of Artificial Sequence Synthetic polynucleotide
tcaccacggcgataaccataggttttcggcgtggccacatccatggtgaatcccactttttccagcacgcgcgccacttcatcgggtcttaaatacatagattttcctcgtcatctttccaaagcctcgccaccttacatgactgagcatggaccgtgactcagaaaattccacaaacgaacctgaaaggcgtgattgccgtctggccttaaaaattatggtctaaactaaaatttacatcgaaaacgagggaggatcctatggcgtcgaaatcgttttgtaaaagttggggagccgaatacctcgcgaatggtcgcgtgcgttttcgcttatgggccaccgggcagcagcaggtttcactgcgtttaccggctggcgaattgccaatgcaacgccttgatgacggctggtttgaactgaccgtcgataatatcgccgcaggtacggcgtaccagtttgtcctgagcaacggtttggcggttcccgatcccgcctcgcgcgcgcaggccgccgatgttaacggcccttcgctggttgtcgacccggatcattacgtctggcaacacccgcagtggcagggtcgcccgtggcaagaatccgtggtctatgaactgcatattgggacatttaccccgcggggcacttttcgcgcggcaatggaaaaactgccgtacctcgcgcaactcggcatcaccatgatagaggtgatgcccgtctcccagtttggcggcaatcgcggctggggctatgacggcgtgctgctctatgcgccgcactcggcgtatggcacgccggatgattttaaagcctttatcgacgccgctcacggctacggcctttcagtggtgctggatattgtgctcaaccactttggcccggaaggtaactacttacccgcgctgtcaccggattttttccacgccgaacggcagacgccgtgggggccgggcatcgcgtatgacgttgacgcggtgcggcgttacatcgttgaagcaccgctctactggctcaacgaatacaacctggacgggctgcgatttgatgccatcgatcagattgaagacagcgccgaaccgccggtgctggtggagattgcccgccgtattcgcgaggagatcaccgaccggccggtgcatctgaccaccgaagattgccgcaatattattgcgctgcatccgcgcgagccggatgggcgtgcgccgctgtttaccgccgagtggaatgacgatctgcacaatgccgcgcatgtgtttgccaccggtgaaacccatgcctattaccaggattttgcggataagcccgaagtgtggctggcgcgcacgttaactgaaggattcgcgtttcagggggagattgccccgtcaaccggtgaaccacgcggagtggacagccgcaaccagccgccggtagcgtttgtcgattttatccagaaccacgatcagaccggcaaccgcgcccagggcgagcggcttatcacgctggcgggcgaggagagaacgcgcgtgctgctcggcgcgctgctgttttccccgcatattccgctgctgtttatgggcgaagagtatggcgaaaccaatcctttcctgttctttaccgattttcacggcgatctggcgcgcgccgtgcgtgaagggcgcgcgcgggaattccagggccatgccggacatgaaggggaagcggttcccgatccgaatgcgcctgacacttttacccgatcacaactggactgggcaaaaacgcagcaggagagcggccagcgctggctgacgcaaacccgcgactggctgacgctgcgccagaaatatgtggttccgctgctgacaaatgcgcagcggcaaaatggggaagtgatcgccaccgcgccagggtttgtcgcggtccgctggcgcttcccgcaagggacattatcgctggcgctcaatattggcgaggcgacgcaaccgttgccggatcttcccggcaaaacgctggcagcctggccgcgcggacaagatgacttgccgccgaatgcgttcattgttcgtctggctttgggagagggaatgtga

SEQ ID NO: 24 - DNA, 589 bp, Artificial Sequence
  Description of Artificial Sequence Synthetic polynucleotide
cgtcctgtaataataaccggacaattcggactgattaaaaaagcgcccttgtggcgctttttttatattcccgcctccatttaaaataaaaaatccaatcggatttcactatttaaactggccattatctaagatgaatccgatggaagctcgctgttttaacacgcgttttttaaccttttattgaaagtcggtgcttctttgagcgaacgatcaaatttaagtggattcccatcaaaaaaatattctcaacctaaaaaagtttgtgtaatacttgtaacgctacatggagattaactcaatctagagggtattaataatgaatcgtactaaactggtactgggcatgaaaaaagtcctgtatggcatttttgccatatccgcgcttgcggcgacttctgcttttgctgcaccggtgcaaattggtgaagcggcagggtcggcagcgacctctgtttctgcgggtagctccgcagcaacgagcgttagcaccgtagggtccgcggtgggtgtcgcgctggcggcaaccggtggcggtgatggctccaacacgggaaccacgaccaccacgaccacaagtacccagtaa

SEQ ID NO: 25 - DNA, 503 bp, Artificial Sequence
  Description of Artificial Sequence Synthetic polynucleotide
tcaccacggcgataaccataggttttcggcgtggccacatccatggtgaatcccactttttccagcacgcgcgccacttcatcgggtcttaaatacatagattttcctcgtcatctttccaaagcctcgccaccttacatgactgagcatggaccgtgactcagaaaattccacaaacgaacctgaaaggcgtgattgccgtctggccttaaaaattatggtctaaactaaaatttacatcgaaaacgagggaggatcctatgaaaaaagtcctgtatggcatttttgccatatccgcgcttgcggcgacttctgcttttgctgcaccggtgcaaattggtgaagcggcagggtcggcagcgacctctgtttctgcgggtagctccgcagcaacgagcgttagcaccgtagggtccgcggtgggtgtcgcgctggcggcaaccggtggcggtgatggctccaacacgggaaccacgaccaccacgaccacaagtacccagtaa

SEQ ID NO: 26 - DNA, 572 bp, Artificial Sequence
  Description of Artificial Sequence Synthetic polynucleotide
atattgacaccatgacgcgcgtaatgctgattggttctgtgacgctggtaatgattgtcgaaattctgaacagtgccatcgaagccgtagtagaccgtattggtgcagaattccatgaactttccgggcgggcgaaggatatggggtcggcggcggtgctgatgtccatcctgctggcgatgtttacctggatcgcattactctggtcacattttcgataacgcttccagaattcgataacgccctggttttttgcttaaatttggttccaaaatcgcctttagctgtatatactcacagcataactgtatatacacccagggggcgggatgaaaaaagtcctgtatggcatttttgccatatccgcgcttgcggcgacttctgcttttgctgcaccggtgcaaattggtgaagcggcagggtcggcagcgacctctgtttctgcgggtagctccgcagcaacgagcgttagcaccgtagggtccgcggtgggtgtcgcgctggcggcaaccggtggcggtgatggctccaacacgggaaccacgaccaccacgaccacaagtacccagtaa

SEQ ID NO: 27 - DNA, 600 bp, Artificial Sequence
  Description of Artificial Sequence Synthetic polynucleotide
gataccgagtgagagcaaggcgttagaaagggtgcgaccaatcagaaactccatcgataagtagtaaacctggcgcacttcctgagagagctgtgcacgggtagaacgcagccagcgctccaccagccgatcgcgcacggcaaacagcgtggcgttcagccattcgtggcgattggcgacagccggatctttaccgatggtgaacatcagtttgtaggcaatcgaatgttttaacgcctcaatacttaacgtaggtgaagcataagtaaaaggtgcattcatataatgattcctggaaaatatcgctcctgttcaaaacccgttggccttgatgccagaaatggggaaacagcgcgcggaccccttccgcgcaaggttataatcagatgctttcagcttttaatttgcgcagcatttcacgtgtgaccaacacaatgccttcttcggaacggtaaaagcgacgcgcatcttcttccgcattttcaccaatcaccgttccttccggtatcacgcagcctctgtcgataatgcagcgatggaggcgacaggagcgccccacccacacttccggcagcaagaccgaagagtcgatattgcaga

SEQ ID NO: 28 - DNA, 600 bp, Artificial Sequence
  Description of Artificial Sequence Synthetic polynucleotide
cggcggctgcattatctccgggtcggtggtggtgcagtcggtgctgttcccgcgcgtgcgggtgaactcattctgtaacattgattcggcagtcttgttgccagatgtctgggtggggcgctcatgccgcctgcgtcgctgcgtgattgaccgcgcctgcgtcatcccggaaggtatggtgattggtgagaacgcggaagaggatgcacgccgcttctatcgctcagaggaaggtatcgtgctggtgacccgcgacatgctgcggaaattggggcacaaacaggagcgctaatgcaggtcttgatgtaaccactggagtcattgttatgaatgtaccctttagctatgcatcaccgacgctgagcgttgaggcgttaaagcactctattgcctataagctgatgtttatcatcggtaaagatccggccatcgccaacaagcatgaatggcttaacgccacgctgtttgccgtccgcgatcgcatggtggagcgctggctgcgctccaaccgcgcgcagctctcgcaggaagtgcgccaggtatactatctgtcgatggagttcctgattggccgtaccctctctaacgcgctgctgtcgc

(19) United States
(12) Patent Application Publication
(10) Pub. No.: US 2021/0163374 A1
(43) Pub. Date: Jun. 3, 2021

(54) METHODS AND COMPOSITIONS FOR IMPROVING ENGINEERED MICROBES

(71) Applicant: Pivot Bio, Inc., Berkeley, CA (US)

(72) Inventors: Sarah Bloch, Emeryville, CA (US); Karsten Temme, Oakland, CA (US); Alvin Tamsir, San Francisco, CA (US)

(21) Appl. No.: 16/637,565

(22) PCT Filed: Aug. 9, 2018
(86) PCT No.: PCT/US2018/046148

§ 371 Date: Feb. 7, 2020

Related U.S. Application Data
(60) Provisional application No. 62/543,288, filed on Aug. 9, 2017.

(51) Int. Cl. C05F 11/08; C07K 14/235; C12N 15/74; C07K 14/195; C12N 9/24; C12N 9/16; A01C 21/00

(52) U.S. Cl. CPC C05F 11/08; C07K 14/235; C12N 15/74; C07K 14/195; A01C 21/00; C12Y 302/01015; C12N 9/16; C12Y 301/03012; C12Y 302/01141; C12N 9/2402

(57) ABSTRACT

The present disclosure provides a bacterial composition, comprising: at least one genetically engineered bacterial strain that fixes atmospheric nitrogen in an agricultural system, wherein the bacterial strain comprises a modification in or one or more genes selected from the group consisting of bcsll, bcslll, yjbE, fhaB, pehA, glgA, otsB, treZ, and cysZ. The present disclosure further provides a bacterial composition and method for increasing the colonization of a plant growth promoting bacterial strain on a plant, wherein the plant growth promoting bacterial strain has been remodeled to increase colonization of said plant, In a further aspect, the present disclosure provides methods of increasing nitrogen or nitrogen fixation available to a plant.

### CROSS-REFERENCE

This application is a National Stage application under 35 U.S.C. § 371 of International Application No. PCT/US2018/046148 having an International Filing Date of Aug. 9, 2018, which claims the benefit of U.S. Provisional Application No. 62/543,288, filed Aug. 9, 2017, which applications are incorporated herein by reference.

The Sequence Listing associated with this application is provided in text form in lieu of a paper copy, and is hereby incorporated by reference into the specification. The name of the text file containing the Sequence Listing is sequencelisting.txt. The text file is 48.4 KB, and was created and submitted electronically via EFS-Web on Dec. 10, 2020.

### BACKGROUND OF THE INVENTION

By 2050 the United Nations' Food and Agriculture Organization projects that total food production must increase by 70% to meet the needs of the growing population, a challenge that is exacerbated by numerous factors, including diminishing freshwater resources, increasing competition for arable land, rising energy prices, increasing input costs, and the likely need for crops to adapt to the pressures of a drier, hotter, and more extreme global climate.

One area of interest is in the improvement of nitrogen fixation. Nitrogen gas (N₂) is a major component of the atmosphere of Earth. In addition, elemental nitrogen (N) is an important component of many chemical compounds which make up living organisms. However, many organisms cannot use N₂ directly to synthesize the chemicals used in physiological processes, such as growth and reproduction. In order to utilize the N₂, the N₂ must be combined with hydrogen. The combining of hydrogen with N₂ is referred to as nitrogen fixation. Nitrogen fixation, whether accomplished chemically or biologically, requires an investment of large amounts of energy. In biological systems, an enzyme known as nitrogenase catalyzes the reaction which results in nitrogen fixation. An important goal of nitrogen fixation research is the extension of this phenotype to non-leguminous plants, particularly to important agronomic grasses such as wheat, rice, and maize. Despite enormous progress in understanding the development of the nitrogen-fixing symbiosis between rhizobia and legumes, the path to use that knowledge to induce nitrogen-fixing nodules on non-leguminous crops is still not clear. Meanwhile, the challenge of providing sufficient supplemental sources of nitrogen, such as in fertilizer, will continue to increase with the growing need for increased food production.

### SUMMARY OF THE INVENTION

In one aspect, the present disclosure provides a bacterial composition, comprising: at least one genetically engineered bacterial strain that fixes atmospheric nitrogen in an agricultural system, wherein the bacterial strain comprises a modification in or one or more genes selected from the group consisting of bcsll, bcsIII, yjbE, fhaB, pehA, glgA, otsB, treZ, and cysZ.

In another aspect, the present disclosure provides a bacterial composition, comprising: a plant growth promoting bacterial strain, wherein said strain has been remodeled to increase colonization of said plant growth promoting bacterial strain on a plant. In some cases, said colonization of said plant growth promoting bacterial strain occurs on a root of said plant. In some cases, said bacterial strain comprises a genetic modification in an enzyme or pathway involved in production of exopolysaccharides. In some cases, said genetic modification is in a gene selected from the group consisting of bcsII, bcsIII, and yjbE. In some cases, said bacterial strain comprises a genetic modification in an enzyme or pathway involved in production of a filamentous hemagglutinin. In some cases, said genetic modification is in a fhaB gene. In some cases, said bacterial strain comprises a genetic modification in an enzyme or pathway involved in production of an endo-polygalaturonase. In some cases, said genetic modification is in a pehA gene. In some cases, said bacterial strain comprises a genetic modification in an enzyme or pathway involved in production of trehalose. In some cases, said genetic modification is in a gene selected from the group consisting of: otsB and treZ. In some cases, said bacterial composition is formulated for application to a field. In some cases, wherein said plant growth promoting bacteria provides a nutrient to said plant. In some cases, said plant growth promoting bacteria provides fixed nitrogen to said plant. In some cases, said plant is selected from the group consisting of corn, barley, wheat, sorghum, soy, and rice.

In a further aspect, the present disclosure provides a method of increasing the colonization of a plant growth promoting bacterial strain on a plant, said method comprising: introducing into said plant growth promoting bacterial strain a genetic modification in a gene involved in a pathway selected from the group consisting of: exopolysaccharide production, endo-polygalaturonase production, and trehalose production.

In a further aspect, the present disclosure provides a method of increasing nitrogen available to a plant, said method comprising: applying a plurality of remodeled bacteria onto a plant, said plurality of remodeled bacteria having decreased expression of glgA.

In some cases, said remodeled bacteria have lower degree assimilation of nitrogen within said remodeled bacteria as compared with a degree of assimilation of a non-remodeled bacteria of a same species as said remodeled bacteria.

In a further aspect, the present disclosure provides a method of increasing nitrogen fixation available to a plant, said method comprising: applying a plurality of remodeled bacteria onto a plant, said plurality of remodeled bacteria having increase expression of at least one nitrogenase cofactor. In some cases, said increased nitrogenase cofactor is sulfur. In some cases, said remodeled bacteria have increased expression of cysZ. In some cases, cysZ is a sulfur transporter.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A depicts a soil texture map of various field soils tested for colonization. Soils in which a few microbes were originally source from are indicated as stars.

FIG. 1B depicts the colonization rate of Strain 1 and Strain 5 that are tested across four different soil types (circles). Both strains showed relatively robust colonization profile across diverse soil types.

FIG. 1C depicts colonization of Strain 1 as tested in a field trial over the span of a growing season. Strain 1 persists in the corn tissue up to week 12 after planting and starts to show decline in colonization after that time.

FIG. 2A depicts a schematic of microbe breeding, in accordance with embodiments.

FIG. 2B depicts an expanded view of the measurement of microbiome composition as shown in FIG. 2A.

FIG. 2C depicts sampling of roots utilized in Example 7. [may be discuss in words then take out]

FIGS. 3A-3C illustrate derivative microbes that fix and excrete nitrogen in vitro under conditions similar to high nitrate agricultural soils. FIG. 3A illustrates the regulatory network controlling nitrogen fixation and assimilation in PBC6.1 is shown, including the key nodes NifL, NifA, GS, GlnE depicted as the two-domain ATase-AR enzyme, and AmtB. FIG. 3B illustrates the genome of Kosakonia sacchari isolate PBC6.1 is shown. The three tracks circumscribing the genome convey transcription data from PBC6.1, PBC6.38, and the differential expression between the strains respectively. FIG. 3C illustrates the nitrogen fixation gene cluster and transcription data is expanded for finer detail.

FIG. 4 illustrates PBC6.1 colonization to nearly 21% abundance of the root-associated microbiota in corn roots. Abundance data is based on 16S amplicon sequencing of the rhizosphere and endosphere of corn plants inoculated with PBC6.1 and grown in greenhouse conditions.

FIG. 5 illustrates increased expression of three genes of interest in remodeled strains of K. sacchari, shown by qPCR. Strains were cultured in media containing 5 mM glutamine.

FIG. 6 illustrates nitrogen reduction activity of several remodeled strains in the presence or absence of glutamine.

FIG. 7 illustrates results of an in vitro attachment assay with several strains described herein.

FIG. 8A illustrates results of an in vitro attachment assay with a remodeled strain having increased expression of fhaB as described herein. This strain showed significantly increased fold recovery compared to the parent strain, 6-848 (2.3×, p=0.013).

FIG. 8B illustrates results of an in vitro attachment assay with several strains as described herein.

FIG. 9 illustrates increased attachment by strains with upregulated yjbE2 gene and bcsIII operon. Two remodeled strains showed a slight increase compared to 6-848: 6-112 (6-848+yjbE2-prm1): 1.9× (p=0.07), and 6-1126 (6-848+bcsIII-prm2): 1.7× (p=0.06). Strain 6-1127 (6-848+bcsIII-prm9) showed a significant 2.5× increase over 6-848 (p=0.0005)

FIG. 10 illustrates results of an in vitro attachment assay. 0 is no microbe added to the water, 462 is DH10B E. coli (a non plant associated control) added to the water, 6-848 and 6-881 are parent strains of other strains used in the assays herein (positive controls). The uninoculated and E. coli controls show some background, however the wild-type strain 6 and parent controls 6-848 and 6-881 show significant attachment over the negative controls (p<0.05).

FIG. 11 illustrates increased attachment of strains with modifications in the pehA gene, the yjbE2 gene and the bcsII operon.

FIG. 12 illustrates over expression of modified genes in remodeled strains in the presence and absence of glutamine.

FIG. 13A illustrates over expression of modified genes in remodeled strains in the presence and absence of glutamine.

FIG. 13B illustrates over expression of modified genes in remodeled strains in the presence and absence of glutamine.

FIG. 13C illustrates over expression of modified genes in remodeled strains in the presence and absence of glutamine.

FIG. 14 illustrates ammonium excretion under anaerobic conditions for the three strains described herein.

FIG. 15 illustrates ammonium excretion for several strains described herein.

FIG. 16 illustrates ammonium excretion for further strains described herein.

FIG. 17 illustrates ammonium excretion for further strains described herein.

FIG. 18 illustrates nitrogen reduction activity for several strains described herein.

FIG. 19 illustrates ammonium excretion for further strains described herein.

FIG. 20 illustrates nitrogen reduction activity for several strains described herein.

FIG. 21 illustrates ammonium excretion for further strains described herein.

FIG. 22A illustrates nitrogen reduction activity for several strains described herein in the presence or absence of nitrogen.

FIG. 22B illustrates nitrogen reduction activity for several strains described herein in the presence or absence of nitrogen.

FIG. 23A illustrates ammonium excretion for strains described herein.

FIG. 23B illustrates ammonium excretion for strains described herein.

FIG. 24 illustrates nitrogen reduction activity for several strains described herein in the presence or absence of nitrogen.

FIG. 25 illustrates ammonium excretion for strains described herein.

FIG. 26A illustrates nitrogen reduction activity for several strains described herein in the presence or absence of nitrogen.

FIG. 26B illustrates nitrogen reduction activity for several strains described herein in the presence or absence of nitrogen.

FIG. 27A illustrates ammonium excretion for strains described herein.

FIG. 27B illustrates ammonium excretion for strains described herein.

FIG. 28 illustrates colonization of strains described herein in a greenhouse assay. The greenhouse assay for colonization lacked statistical power to distinguish differences with p<0.05.

FIG. 29 illustrates colonization of further strains described herein in a greenhouse assay.

FIG. 30 illustrates colonization of strains described herein in a greenhouse assay.

FIG. 31 illustrates colonization of strains described herein in a greenhouse assay.

FIG. 32 illustrates colonization of strains described herein in a greenhouse assay.

FIG. 33 illustrates ammonium excretion for strains described herein.

FIG. 34 illustrates nitrogen reduction activity for several strains described herein in the presence or absence of nitrogen.

FIG. 35 illustrates ammonium excretion for strains described herein.

FIG. 36 illustrates nitrogen reduction activity for several strains described herein in the presence or absence of nitrogen.

FIG. 37 illustrates colonization of strains described herein.

FIG. 38 illustrates colonization of strains described herein.

### DETAILED DESCRIPTION OF THE INVENTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Increased fertilizer utilization brings with it environmental concerns and is also likely not possible for many economically stressed regions of the globe. Furthermore, many industry players in the microbial arena are focused on creating intergeneric microbes. However, there is a heavy regulatory burden placed on engineered microbes that are characterized/classified as intergeneric. These intergeneric microbes face not only a higher regulatory burden, which makes widespread adoption and implementation difficult, but they also face a great deal of public perception scrutiny.

Currently, there are no engineered microbes on the market that are non-intergeneric and that are capable of increasing nitrogen fixation in non-leguminous crops. This dearth of such a microbe is a missing element in helping to usher in a truly environmentally friendly and more sustainable 21^{st} century agricultural system.

The present disclosure solves the aforementioned problems and provides a non-intergeneric microbe that has been engineered to readily fix nitrogen in crops. These microbes are not characterized/classified as intergeneric microbes and thus will not face the steep regulatory burdens of such. Further, the taught non-intergeneric microbes will serve to help 21^{st} century farmers become less dependent upon utilizing ever increasing amounts of exogenous nitrogen fertilizer.

Guided microbial remodeling, is a state-of-the-art process that identifies, characterizes and fine-tunes microbes to realize their full potential to colonize crop roots and increase nutrient uptake. In some cases guided microbial remodeling may be use genetic material naturally present in each microbe to increase the nitrogen uptake by the crop. Better nutrient availability improves crop quality and yield potential.

There is a complex of enzymes called nitrogenases in microbes that are responsible for the reduction of atmospheric nitrogen to ammonia, and both the microbes and plants convert ammonia to glutamine by glutamine synthase (GS) and hence to other amino acids that are essential for growth and development of both the microbes and plants. The nif genes are genes encoding the complex of nitrogenase enzymes. Besides the nitrogenase enzymes, the nif genes also encode a number of regulatory proteins involved in nitrogen fixation. The expression of the nif genes is induced as a response to low levels of fixed nitrogen and oxygen concentrations in the rhizosphere. In most nitrogen-fixing bacteria, regulation of nif genes transcription is by the nitrogen sensitive NifA protein. When there isn't enough fixed nitrogen available for the organism's use, NifA expression is activated, and NifA activates the rest of the nif genes. If there is a sufficient amount of reduced nitrogen or oxygen present in the rhizosphere, another protein is activated, NifL. NifL inhibits NifA activity resulting in the inhibition of nitrogenase formation.

Examples of aspects of microbial nitrogen fixation which may be altered to increase nitrogen availability to the cereal crop include:

a) Enhance the interaction between the microbe and the crop roots in the rhizosphere.
b) Enhance microbial nitrogen fixation by removing negative regulation of the NifA protein. For example, deletion of the nifL gene abolishes inhibition of NifA activity and improves nif expression in the presence of both oxygen and exogenous fixed nitrogen. Furthermore, expressing nifA under the control of a nitrogen-independent promoter can decouple nitrogenase biosynthesis from sensing environmental nitrogen and oxygen.
c) Enhance ammonia availability to the plant by inhibiting ammonia assimilation to glutamine in the microbe and increasing ammonia excretion from the microbe. For example, the rapid assimilation of fixed nitrogen by the microbe to glutamine by GS is reversibly regulated the by the two-domain adenylyltransferase (ATase) enzyme GlnE through the adenylylation and deadenylylation of GS to attenuate and restore activity, respectively. Truncation of the GlnE protein to delete its adenyiyi4emoving (AR) domain leads to constitutively adenylylated glutamine synthetase, limiting ammonia assimilation by the microbe and increasing intra- and extracellular ammonia. Finally, deleting the amtB gene, which encodes the transporter responsible for uptake of ammonia by the microbe, leads to greater excretion of ammonia.

In some cases, the final microbes produced by the methods described herein contain no foreign (transgenic) genetic material, and may be suitable for release into a field. The genetic changes in the microbe may consist of either sequence deletions or small sequence rearrangements within the organism's own genome, such as creating a copy of a promoter sequence within the microbe's genome and inserting the copy in front of a different gene. Therefore, any genetic elements introduced in the genome of the microbe may be derived from the same parental microbe. Furthermore, genetic elements introduced in the microbial genome may be non-coding genetic elements. The resulting mutations may be “markerless”, meaning they contain no introduced antibiotic resistance or other selectable marker genes that are often used to generate mutations in bacteria. In some cases, the process of guided microbial remodeling may involve introducing some helper DNA molecules into the bacterial cells to facilitate the generation of the non-transgenic mutations. However, all helper DNA can be thoroughly removed from the strains. For example, in some cases all foreign DNA may be removed from a strain before said strain is considered for field release. Removal of all helper DNA can be confirmed, for example through illumina sequencing, a sensitive method that can detect even single molecules of helper DNA sequences. Thus, microbes generated through guided microbial remodeling may contain no transgenes or foreign DNA, and the genetic changes in the strain consist of only sequence deletions and promoter rearrangements, which have been shown to occur in nature.

### Definitions

The terms “polynucleotide”, “nucleotide”, “nucleotide sequence”, “nucleic acid” and “oligonucleotide” are used interchangeably. They refer to a polymeric form of nucleotides of any length, either deoxyribonucleotides or ribonucleotides, or analogs thereof. Polynucleotides may have any three dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

As used herein, “expression” refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

The terms “polypeptide”, “peptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” includes natural and/or unnatural or synthetic amino acids, including glycine and both the D or L optical isomers, and amino acid analogs and peptidomimetics.

As used herein, the term “about” is used synonymously with the term “approximately.” Illustratively, the use of the term “about” with regard to an amount indicates that values slightly outside the cited values, e.g., plus or minus 0.1% to 10%.

Microbes in and around food crops can influence the traits of those crops. Plant traits that may be influenced by microbes include: yield (e.g., grain production, biomass generation, fruit development, flower set); nutrition (e.g., nitrogen, phosphorus, potassium, iron, micronutrient acquisition); abiotic stress management (e.g., drought tolerance, salt tolerance, heat tolerance); and biotic stress management (e.g., pest, weeds, insects, fungi, and bacteria). Strategies for altering crop traits include: increasing key metabolite concentrations; changing temporal dynamics of microbe influence on key metabolites; linking microbial metabolite production/degradation to new environmental cues; reducing negative metabolites; and improving the balance of metabolites or underlying proteins.

In some embodiments, native or endogenous control sequences of genes of the present disclosure are replaced with one or more intrageneric control sequences.

As used herein, “introduced” refers to the introduction by means of modern biotechnology, and not a naturally occurring introduction.

In some embodiments, the bacteria of the present disclosure have been modified such that they are not naturally occurring bacteria.

In some embodiments, the bacteria of the present disclosure are present on the plant in an amount of at least 10³ cfu, 10⁴ cfu, 10⁵ cfu, 10⁶ cfu, 10⁷ cfu, 10⁸ cfu, 10⁹ cfu, 10¹⁰ cfu, 10¹¹ cfu, or 10¹² cfu per gram of fresh or dry weight of the plant. In some embodiments, the bacteria of the present disclosure are present on the plant in an amount of at least about 10³ cfu, about 10⁴ cfu, about 10⁵ cfu, about 10⁶ cfu, about 10⁷ cfu, about 10⁸ cfu, about 10⁹ cfu, about 10¹⁰ cfu, about 10¹¹ cfu, or about 10¹² cfu per gram of fresh or dry weight of the plant. In some embodiments, the bacteria of the present disclosure are present on the plant in an amount of at least 10³ to 10⁹, 10³ to 10⁷, 10³ to 10⁵, 10⁵ to 10⁹, 10⁵ to 10⁷, 10⁶ to 10¹⁰, 10⁶ to 10⁷ cfu per gram of fresh or dry weight of the plant.

Fertilizers and exogenous nitrogen of the present disclosure may comprise the following nitrogen-containing molecules: ammonium, nitrate, nitrite, ammonia, glutamine, etc. Nitrogen sources of the present disclosure may include anhydrous ammonia, ammonia sulfate, urea, diammonium phosphate, urea-form, monoammonium phosphate, ammonium nitrate, nitrogen solutions, calcium nitrate, potassium nitrate, sodium nitrate, etc.

As used herein, “exogenous nitrogen” refers to non-atmospheric nitrogen readily available in the soil, field, or growth medium that is present under non-nitrogen limiting conditions, including ammonia, ammonium, nitrate, nitrite, urea, uric acid, ammonium acids, etc.

As used herein, “non-nitrogen limiting conditions” refers to non-atmospheric nitrogen available in the soil, field, media at concentrations greater than about 4 mM nitrogen, as disclosed by Kant et al. (2010. J. Exp. Biol. 62(4):1499-1509), which is incorporated herein by reference.

As used herein, an “intergeneric microorganism” is a microorganism that is formed by the deliberate combination of genetic material originally isolated from organisms of different taxonomic genera. An “intergeneric mutant” can be used interchangeably with “intergeneric microorganism”. An exemplary “intergeneric microorganism” includes a microorganism containing a mobile genetic element which was first identified in a microorganism in a genus different from the recipient microorganism.

As used herein, an “intrageneric microorganism” is a microorganism that is formed by the deliberate combination of genetic material originally isolated from organisms of the same taxonomic genera. An “intrageneric mutant” can be used interchangeably with “intrageneric microorganism”.

As used herein, “introduced genetic material” means genetic material that is added to, and remains as a component of, the genome of the recipient.

In some embodiments, the nitrogen fixation and assimilation genetic regulatory network comprises polynucleotides encoding genes and non-coding sequences that direct, modulate, and/or regulate microbial nitrogen fixation and/or assimilation and can comprise polynucleotide sequences of the nif cluster (e.g., nifA, nifB, nifC, . . . , nifZ), polynucleotides encoding nitrogen regulatory protein C, polynucleotides encoding nitrogen regulatory protein B, polynucleotide sequences of the gin cluster (e.g. glnA and glnD), draT, and ammonia transporters/permeases. In some cases, the Nif cluster may comprise NifB, NifH, NifD, NifK, NifE, NifN, NifX, hesa, and NifV. In some cases, the Nif cluster may comprise a subset of NifB, NifH, NifD, NifK, NifE, NifN, NifX, hesa, and NifV.

In some embodiments, fertilizer of the present disclosure comprises at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% nitrogen by weight.

In some embodiments, fertilizer of the present disclosure comprises at least about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% nitrogen by weight.

In some embodiments, fertilizer of the present disclosure comprises about 5% to 50%, about 5% to 75%, about 10% to 50%, about 10% to 75%, about 15% to 50%, about 15% to 75%, about 20% to 50%, about 20% to 75%, about 25% to 50%, about 25% to 75%, about 30% to 50%, about 30% to 75%, about 35% to 50%, about 35% to 75%, about 40% to 50%, about 40% to 75%, about 45% to 50%, about 45% to 75%, or about 50% to 75% nitrogen by weight.

In some embodiments, the increase of nitrogen fixation and/or the production of nitrogen in the plant are measured relative to control plants, which have not been exposed to the bacteria of the present disclosure. All increases or decreases in bacteria are measured relative to control bacteria. All increases or decreases in plants are measured relative to control plants.

As used herein, a “constitutive promoter” is a promoter, which is active under most conditions and/or during most development stages. There are several advantages to using constitutive promoters in expression vectors used in biotechnology, such as: high level of production of proteins used to select transgenic cells or organisms; high level of expression of reporter proteins or scorable markers, allowing easy detection and quantification; high level of production of a transcription factor that is part of a regulatory transcription system; production of compounds that requires ubiquitous activity in the organism; and production of compounds that are required during all stages of development. Non-limiting exemplary constitutive promoters include, CaMV 35S promoter, opine promoters, ubiquitin promoter, alcohol dehydrogenase promoter, etc.

As used herein, a “non-constitutive promoter” is a promoter which is active under certain conditions, in certain types of cells, and/or during certain development stages. For example, tissue specific, tissue preferred, cell type specific, cell type preferred, inducible promoters, and promoters under development control are non-constitutive promoters. Examples of promoters under developmental control include promoters that preferentially initiate transcription in certain tissues.

As used herein, “inducible” or “repressible” promoter is a promoter which is under chemical or environmental factors control. Examples of environmental conditions that may affect transcription by inducible promoters include anaerobic conditions, certain chemicals, the presence of light, acidic or basic conditions, etc.

As used herein, a “tissue specific” promoter is a promoter that initiates transcription only in certain tissues. Unlike constitutive expression of genes, tissue-specific expression is the result of several interacting levels of gene regulation. As such, in the art sometimes it is preferable to use promoters from homologous or closely related species to achieve efficient and reliable expression of transgenes in particular tissues, is one of the main reasons for the large amount of tissue-specific promoters isolated from particular tissues found in both scientific and patent literature.

As used herein, the term “operably linked” refers to the association of nucleic acid sequences on a single nucleic acid fragment so that the function of one is regulated by the other. For example, a promoter is operably linked with a coding sequence when it is capable of regulating the expression of that coding sequence (i.e., that the coding sequence is under the transcriptional control of the promoter). Coding sequences can be operably linked to regulatory sequences in a sense or antisense orientation. In another example, the complementary RNA regions of the disclosure can be operably linked, either directly or indirectly, 5′ to the target mRNA, or 3′ to the target mRNA, or within the target mRNA, or a first complementary region is 5′ and its complement is 3′ to the target mRNA.

In aspects, “applying to the plant a plurality of non-intergeneric bacteria,” includes any means by which the plant (including plant parts such as a seed, root, stem, tissue, etc.) is made to come into contact (i.e. exposed) with said bacteria at any stage of the plant's life cycle. Consequently, “applying to the plant a plurality of non-intergeneric bacteria,” includes any of the following means of exposing the plant (including plant parts such as a seed, root, stem, tissue, etc.) to said bacteria spraying onto plant, dripping onto plant, applying as a seed coat, applying to a field that will then be planted with seed, applying to a field already planted with seed, applying to a field with adult plants, etc.

The term mmol is an abbreviation for millimole, which is a thousandth (10⁻³) of a mole, abbreviated herein as mol.

As used herein the terms “microorganism” or “microbe” should be taken broadly. These terms, used interchangeably, include but are not limited to, the two prokaryotic domains, Bacteria and Archaea. The term may also encompass eukaryotic fungi and protists.

The term “microbial consortia” or “microbial consortium” refers to a subset of a microbial community of individual microbial species, or strains of a species, which can be described as carrying out a common function, or can be described as participating in, or leading to, or correlating with, a recognizable parameter, such as a phenotypic trait of interest.

The term “microbial community” means a group of microbes comprising two or more species or strains. Unlike microbial consortia, a microbial community does not have to be carrying out a common function, or does not have to be participating in, or leading to, or correlating with, a recognizable parameter, such as a phenotypic trait of interest.

As used herein, “isolate,” “isolated,” “isolated microbe,” and like terms, are intended to mean that the one or more microorganisms has been separated from at least one of the materials with which it is associated in a particular environment (for example soil, water, plant tissue, etc.). Thus, an “isolated microbe” does not exist in its naturally occurring environment; rather, it is through the various techniques described herein that the microbe has been removed from its natural setting and placed into a non-naturally occurring state of existence. Thus, the isolated strain or isolated microbe may exist as, for example, a biologically pure culture, or as spores (or other forms of the strain). In aspects, the isolated microbe may be in association with an acceptable carrier, which may be an agriculturally acceptable carrier.

In certain aspects of the disclosure, the isolated microbes exist as “isolated and biologically pure cultures.” It will be appreciated by one of skill in the art, that an isolated and biologically pure culture of a particular microbe, denotes that said culture is substantially free of other living organisms and contains only the individual microbe in question. The culture can contain varying concentrations of said microbe. The present disclosure notes that isolated and biologically pure microbes often “necessarily differ from less pure or impure materials.” See, e.g. In re Bergstrom, 427 F.2d 1394, (CCPA 1970) (discussing purified prostaglandins), see also, In re Bergy, 596 F.2d 952 (CCPA 1979) (discussing purified microbes), see also, Parke-Davis & Co. v. H. K. Mulford & Co., 189 F. 95 (S.D.N.Y. 1911) (Learned Hand discussing purified adrenaline), aff'd in part, rev'd in part, 196 F. 496 (2d Cir. 1912), each of which are incorporated herein by reference. Furthermore, in some aspects, the disclosure provides for certain quantitative measures of the concentration, or purity limitations, that must be found within an isolated and biologically pure microbial culture. The presence of these purity values, in certain embodiments, is a further attribute that distinguishes the presently disclosed microbes from those microbes existing in a natural state. See, e.g., Merck & Co. v. Olin Mathieson Chemical Corp., 253 F.2d 156 (4th Cir. 1958) (discussing purity limitations for vitamin B12 produced by microbes), incorporated herein by reference.

As used herein, “individual isolates” should be taken to mean a composition, or culture, comprising a predominance of a single genera, species, or strain, of microorganism, following separation from one or more other microorganisms.

Microbes of the present disclosure may include spores and/or vegetative cells. In some embodiments, microbes of the present disclosure include microbes in a viable but non-culturable (VBNC) state. As used herein, “spore” or “spores” refer to structures produced by bacteria and fungi that are adapted for survival and dispersal. Spores are generally characterized as dormant structures; however, spores are capable of differentiation through the process of germination. Germination is the differentiation of spores into vegetative cells that are capable of metabolic activity, growth, and reproduction. The germination of a single spore results in a single fungal or bacterial vegetative cell. Fungal spores are units of asexual reproduction, and in some cases are necessary structures in fungal life cycles. Bacterial spores are structures for surviving conditions that may ordinarily be nonconducive to the survival or growth of vegetative cells.

As used herein, “microbial composition” refers to a composition comprising one or more microbes of the present disclosure. In some embodiments, a microbial composition is administered to plants (including various plant parts) and/or in agricultural fields.

As used herein, “carrier,” “acceptable carrier,” or “agriculturally acceptable carrier” refers to a diluent, adjuvant, excipient, or vehicle with which the microbe can be administered, which does not detrimentally effect the microbe.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. For example, if the range 10-15 is disclosed, then 11, 12, 13, and 14 are also disclosed. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

### Bacterial Colonization

In some embodiments microbes may be modified to increase delivery of nitrogen to plants. For example, microbes may be modified to increase colonization of plant roots or to increase cofactors required for nitrogen fixation.

In some cases, exopolysaccharides may be involved in bacterial colonization of plants. In some cases, plant colonizing microbes may produce a biofilm. In some cases, plant colonizing microbes secrete molecules which may assist in adhesion to the plant, or in evading a plant immune response. In some cases, plant colonizing microbes may excrete signaling molecules which alter the plants response to the microbes. In some cases, plant colonizing microbes may secrete molecules which alter the local microenvironment. In some cases, a plant colonizing microbe may alter expression of genes to adapt to a plant said microbe is in proximity to. In some cases, a plant colonizing microbe may detect the presence of a plant in the local environment and may change expression of genes in response.

### Regulation of Nitrogen Fixation

In some cases, nitrogen fixation pathway may act as a target for genetic engineering and optimization. One trait that may be targeted for regulation by the methods described herein is nitrogen fixation. Nitrogen fertilizer is the largest operational expense on a farm and the biggest driver of higher yields in row crops like corn and wheat. Described herein are microbial products that can deliver renewable forms of nitrogen in non-leguminous crops. While some endophytes have the genetics necessary for fixing nitrogen in pure culture, the fundamental technical challenge is that wild-type endophytes of cereals and grasses stop fixing nitrogen in fertilized fields. The application of chemical fertilizers and residual nitrogen levels in field soils signal the microbe to shut down the biochemical pathway for nitrogen fixation.

Changes to the transcriptional and post-translational levels of components of the nitrogen fixation regulatory network may be beneficial to the development of a microbe capable of fixing and transferring nitrogen to corn in the presence of fertilizer. To that end, described herein is Host-Microbe Evolution (HoME) technology to precisely evolve regulatory networks and elicit novel phenotypes. Also described herein are unique, proprietary libraries ofnitrogen-fixing endophytes isolated from corn, paired with extensive omics data surrounding the interaction of microbes and host plant under different environmental conditions like nitrogen stress and excess. In some embodiments, this technology enables precision evolution of the genetic regulatory network of endophytes to produce microbes that actively fix nitrogen even in the presence of fertilizer in the field. Also described herein are evaluations of the technical potential of evolving microbes that colonize corn root tissues and produce nitrogen for fertilized plants and evaluations of the compatibility of endophytes with standard formulation practices and diverse soils to determine feasibility of integrating the microbes into modern nitrogen management strategies.

In order to utilize elemental nitrogen (N) for chemical synthesis, life forms combine nitrogen gas (N₂) available in the atmosphere with hydrogen in a process known as nitrogen fixation. Because of the energy-intensive nature of biological nitrogen fixation, diazotrophs (bacteria and archaea that fix atmospheric nitrogen gas) have evolved sophisticated and tight regulation of the nifgene cluster in response to environmental oxygen and available nitrogen. Nifgenes encode enzymes involved in nitrogen fixation (such as the nitrogenase complex) and proteins that regulate nitrogen fixation. Shamseldin (2013. Global J. Biotechnol. Biochem. 8(4):84-94) discloses detailed descriptions of nif genes and their products, and is incorporated herein by reference. Described herein are methods of producing a plant with an improved trait comprising isolating bacteria from a first plant, introducing a genetic variation into a gene of the isolated bacteria to increase nitrogen fixation, exposing a second plant to the variant bacteria, isolating bacteria from the second plant having an improved trait relative to the first plant, and repeating the steps with bacteria isolated from the second plant.

In Proteobacteria, regulation of nitrogen fixation centers around the α₅₄-dependent enhancer-binding protein NifA, the positive transcriptional regulator of the nif cluster. Intracellular levels of active NifA are controlled by two key factors: transcription of the nifLA operon, and inhibition of NifA activity by protein-protein interaction with NifL. Both of these processes are responsive to intracelullar glutamine levels via the PII protein signaling cascade. This cascade is mediated by GlnD, which directly senses glutamine and catalyzes the uridylylation or deuridylylation of two PII regulatory proteins—GlnB and GlnK—in response the absence or presence, respectively, of bound glutamine. Under conditions of nitrogen excess, unmodified GlnB signals the deactivation of the nifLA promoter. However, under conditions of nitrogen limitation, GlnB is post-translationally modified, which inhibits its activity and leads to transcription of the nifLA operon. In this way, nifLA transcription is tightly controlled in response to environmental nitrogen via the PII protein signaling cascade. On the post-translational level of NifA regulation, GlnK inhibits the NifL/NifA interaction in a matter dependent on the overall level of free GlnK within the cell.

NifA is transcribed from the nifLA operon, whose promoter is activated by phosphorylated NtrC, another σ₅₄-dependent regulator. The phosphorylation state of NtrC is mediated by the histidine kinase NtrB, which interacts with deuridylylated GlnB but not uridylylated GlnB. Under conditions of nitrogen excess, a high intracellular level of glutamine leads to deuridylylation of GlnB, which then interacts with NtrB to deactivate its phosphorylation activity and activate its phosphatase activity, resulting in dephosphorylation of NtrC and the deactivation of the nifLA promoter. However, under conditions of nitrogen limitation, a low level of intracellular glutamine results in uridylylation of GlnB, which inhibits its interaction with NtrB and allows the phosphorylation of NtrC and transcription of the nifLA operon. In this way, nifLA expression is tightly controlled in response to environmental nitrogen via the PII protein signaling cascade, nifA, ntrB, ntrC, and glnB, are all genes that can be mutated in the methods described herein. These processes may also be responsive to intracellular or extracellular levels of ammonia, urea or nitrates.

The activity of NifA is also regulated post-translationally in response to environmental nitrogen, most typically through NifL-mediated inhibition of NifA activity. In general, the interaction of NifL and NifA is influenced by the PII protein signaling cascade via GlnK, although the nature of the interactions between GlnK and NifL/NifA varies significantly between diazotrophs. In Klebsiella pneumoniae, both forms of GlnK inhibit the NifL/NifA interaction, and the interaction between GlnK and NifL/NifA is determined by the overall level of free GlnK within the cell. Under nitrogen-excess conditions, deuridylylated GlnK interacts with the ammonium transporter AmtB, which serves to both block ammonium uptake by AmtB and sequester GlnK to the membrane, allowing inhibition of NifA by NifL. On the other hand, in Azotobacter vinelandii, interaction with deuridylylated GlnK is required for the NifL/NifA interaction and NifA inhibition, while uridylylation of GlnK inhibits its interaction with NifL. In diazotrophs lacking the nifL gene, there is evidence that NifA activity is inhibited directly by interaction with the deuridylylated forms of both GlnK and GlnB under nitrogen-excess conditions. In some bacteria the Nif cluster may be regulated by glnR, and further in some cases this may comprise negative regulation. Regardless of the mechanism, post-translational inhibition of NifA is an important regulator of the nif cluster in most known diazotrophs. Additionally, nifL, amtB, glnK, and glnR are genes that can be mutated in the methods described herein.

In addition to regulating the transcription of the nif gene cluster, many diazotrophs have evolved a mechanism for the direct post-translational modification and inhibition of the nitrogenase enzyme itself, known as nitrogenase shutoff. This is mediated by ADP-ribosylation of the Fe protein (NifH) under nitrogen-excess conditions, which disrupts its interaction with the MoFe protein complex (NifDK) and abolishes nitrogenase activity. DraT catalyzes the ADP-ribosylation of the Fe protein and shutoff of nitrogenase, while DraG catalyzes the removal of ADP-ribose and reactivation of nitrogenase. As with nifLA transcription and NifA inhibition, nitrogenase shutoff is also regulated via the PII protein signaling cascade. Under nitrogen-excess conditions, deuridylylated GlnB interacts with and activates DraT, while deuridylylated GlnK interacts with both DraG and AmtB to form a complex, sequestering DraG to the membrane. Under nitrogen-limiting conditions, the uridylylated forms of GlnB and GlnK do not interact with DraT and DraG, respectively, leading to the inactivation of DraT and the diffusion of DraG to the Fe protein, where it removes the ADP-ribose and activates nitrogenase. The methods described herein also contemplate introducing genetic variation into the nifH, nifD, nifK and draT genes.

Although some endophytes have the ability to fix nitrogen in vitro, often the genetics are silenced in the field by high levels of exogenous chemical fertilizers. One can decouple the sensing of exogenous nitrogen from expression of the nitrogenase enzyme to facilitate field-based nitrogen fixation. Improving the integral of nitrogenase activity across time further serves to augment the production of nitrogen for utilization by the crop. Specific targets for genetic variation to facilitate field-based nitrogen fixation using the methods described herein include one or more genes selected from the group consisting of nifA, nifL, ntrB, ntrC, glnA, glnB, glnK, draT, amtB, glnD, glnE, nifJ, nifH, nifD, nifK, nifY, nifE, nifN, nifU, nifS, nifV, nifW, nifZ, nifM, nifF, nifB, and nifQ.

An additional target for genetic variation to facilitate field-based nitrogen fixation using the methods described herein is the NifA protein. The NifA protein is typically the activator for expression of nitrogen fixation genes. Increasing the production of NifA (either constitutively or during high ammonia condition) circumvents the native ammonia-sensing pathway. In addition, reducing the production of NifL proteins, a known inhibitor of NifA, also leads to an increased level of freely active NifA. In addition, increasing the transcription level of the nifAL operon (either constitutively or during high ammonia condition) also leads to an overall higher level of NifA proteins. Elevated level of nifAL expression is achieved by altering the promoter itself or by reducing the expression of NtrB (part of ntrB and ntrC signaling cascade that originally would result in the shutoff of nifAL operon during high nitrogen condition). High level of NifA achieved by these or any other methods described herein increases the nitrogen fixation activity of the endophytes.

Another target for genetic variation to facilitate field-based nitrogen fixation using the methods described herein is the GlnD/GlnB/GlnK PII signaling cascade. The intracellular glutamine level is sensed through the GlnD/GlnB/GlnK PII signaling cascade. Active site mutations in GlnD that abolish the uridylyl-removing activity of GlnD disrupt the nitrogen-sensing cascade. In addition, reduction of the GlnB concentration short circuits the glutamine-sensing cascade. These mutations “trick” the cells into perceiving a nitrogen-limited state, thereby increasing the nitrogen fixation level activity. These processes may also be responsive to intracellular or extracellular levels of ammonia, urea or nitrates.

The amtB protein is also a target for genetic variation to facilitate field-based nitrogen fixation using the methods described herein. Ammonia uptake from the environment can be reduced by decreasing the expression level of amtB protein. Without intracellular ammonia, the endophyte is not able to sense the high level of ammonia, preventing the down-regulation of nitrogen fixation genes. Any ammonia that manages to get into the intracellular compartment is converted into glutamine. Intracellular glutamine level is the major currency of nitrogen sensing. Decreasing the intracellular glutamine level prevents the cells from sensing high ammonium levels in the environment. This effect can be achieved by increasing the expression level of glutaminase, an enzyme that converts glutamine into glutamate. In addition, intracellular glutamine can also be reduced by decreasing glutamine synthase (an enzyme that converts ammonia into glutamine). In diazotrophs, fixed ammonia is quickly assimilated into glutamine and glutamate to be used for cellular processes. Disruptions to ammonia assimilation may enable diversion of fixed nitrogen to be exported from the cell as ammonia. The fixed ammonia is predominantly assimilated into glutamine by glutamine synthetase (GS), encoded by glnA, and subsequently into glutamine by glutamine oxoglutarate aminotransferase (GOGAT). In some examples, glnS encodes a glutamine synthetase. GS is regulated post-translationally by GS adenylyl transferase (GlnE), a bi-functional enzyme encoded by glnE that catalyzes both the adenylylation and de-adenylylation of GS through activity of its adenylyl-transferase (AT) and adenylyl-removing (AR) domains, respectively. Under nitrogen limiting conditions, glnA is expressed, and GlnE's AR domain de-adynylylates GS, allowing it to be active. Under conditions of nitrogen excess, glnA expression is turned off, and GlnE's AT domain is activated allosterically by glutamine, causing the adenylylation and deactivation of GS.

Furthermore, the draT gene may also be a target for genetic variation to facilitate field-based nitrogen fixation using the methods described herein. Once nitrogen fixing enzymes are produced by the cell, nitrogenase shut-off represents another level in which cell downregulates fixation activity in high nitrogen condition. This shut-off could be removed by decreasing the expression level of DraT.

Methods for imparting new microbial phenotypes can be performed at the transcriptional, translational, and post-translational levels. The transcriptional level includes changes at the promoter (such as changing sigma factor affinity or binding sites for transcription factors, including deletion of all or a portion of the promoter) or changing transcription terminators and attenuators. The translational level includes changes at the ribosome binding sites and changing mRNA degradation signals. The post-translational level includes mutating an enzyme's active site and changing protein-protein interactions. These changes can be achieved in a multitude of ways. Reduction of expression level (or complete abolishment) can be achieved by swapping the native ribosome binding site (RBS) or promoter with another with lower strength/efficiency. ATG start sites can be swapped to a GTG, TTG, or CTG start codon, which results in reduction in translational activity of the coding region. Complete abolishment of expression can be done by knocking out (deleting) the coding region of a gene. Frameshifting the open reading frame (ORF) likely will result in a premature stop codon along the ORF, thereby creating a non-functional truncated product. Insertion of in-frame stop codons will also similarly create a non-functional truncated product. Addition of a degradation tag at the N or C terminal can also be done to reduce the effective concentration of a particular gene.

Conversely, expression level of the genes described herein can be achieved by using a stronger promoter. To ensure high promoter activity during high nitrogen level condition (or any other condition), a transcription profile of the whole genome in a high nitrogen level condition could be obtained and active promoters with a desired transcription level can be chosen from that dataset to replace the weak promoter. Weak start codons can be swapped out with an ATG start codon for better translation initiation efficiency. Weak ribosomal binding sites (RBS) can also be swapped out with a different RBS with higher translation initiation efficiency. In addition, site specific mutagenesis can also be performed to alter the activity of an enzyme.

In addition to regulation of nitrogenase genes and nitrogen assimilation genes levels of excreted nitrogen may also be affected by the availability of cofactors involved in fixation and/or assimilation. For example, sulfur is a component of some nitrogenases. Sulfur must, generally, be transferred across the cell membranes. In L. japonicus the sulfate transporter LjSST1 is essential for nitrogen fixation; knockout mutants are unable to develop functional nodules. In some embodiments, nitrogenase activity may be increased by upregulating transporters which increase the availability of cofactors such as sulfur. For example, expression or activity of the sulfur transporter, cysZ, may be increased.

Increasing the level of nitrogen fixation that occurs in a plant can lead to a reduction in the amount of chemical fertilizer needed for crop production and reduce greenhouse gas emissions (e.g., nitrous oxide).

### Generation of Bacterial Populations

### Isolation of Bacteria

Microbes useful in methods and compositions disclosed herein can be obtained by extracting microbes from surfaces or tissues of native plants. Microbes can be obtained by grinding seeds to isolate microbes. Microbes can be obtained by planting seeds in diverse soil samples and recovering microbes from tissues. Additionally, microbes can be obtained by inoculating plants with exogenous microbes and determining which microbes appear in plant tissues. Non-limiting examples of plant tissues may include a seed, seedling, leaf, cutting, plant, bulb, or tuber.

A method of obtaining microbes may be through the isolation of bacteria from soils. Bacteria may be collected from various soil types. In some example, the soil can be characterized by traits such as high or low fertility, levels of moisture, levels of minerals, and various cropping practices. For example, the soil may be involved in a crop rotation where different crops are planted in the same soil in successive planting seasons. The sequential growth of different crops on the same soil may prevent disproportionate depletion of certain minerals. The bacteria can be isolated from the plants growing in the selected soils. The seedling plants can be harvested at 2-6 weeks of growth. For example, at least 400 isolates can be collected in a round of harvest. Soil and plant types reveal the plant phenotype as well as the conditions, which allow for the downstream enrichment of certain phenotypes.

Microbes can be isolated from plant tissues to assess microbial traits. The parameters for processing tissue samples may be varied to isolate different types of associative microbes, such as rhizopheric bacteria, epiphytes, or endophytes. The isolates can be cultured in nitrogen-free media to enrich for bacteria that perform nitrogen fixation. Alternatively, microbes can be obtained from global strain banks.

In planta analytics are performed to assess microbial traits. In some embodiments, the plant tissue can be processed for screening by high throughput processing for DNA and RNA. Additionally, non-invasive measurements can be used to assess plant characteristics, such as colonization. Measurements on wild microbes can be obtained on a plant-by-plant basis. Measurements on wild microbes can also be obtained in the field using medium throughput methods. Measurements can be done successively over time. Model plant system can be used including, but not limited to, Setaria.

Microbes in a plant system can be screened via transcriptional profiling of a microbe in a plant system. Examples of screening through transcriptional profiling are using methods of quantitative polymerase chain reaction (qPCR), molecular barcodes for transcript detection, Next Generation Sequencing, and microbe tagging with fluorescent markers. Impact factors can be measured to assess colonization in the greenhouse including, but not limited to, microbiome, abiotic factors, soil conditions, oxygen, moisture, temperature, inoculum conditions, and root localization. Nitrogen fixation can be assessed in bacteria by measuring 15N gas/fertilizer (dilution) with IRMS or NanoSIMS as described herein NanoSIMS is high-resolution secondary ion mass spectrometry. The NanoSIMS technique is a way to investigate chemical activity from biological samples. The catalysis of reduction of oxidation reactions that drive the metabolism of microorganisms can be investigated at the cellular, subcellular, molecular and elemental level. NanoSIMS can provide high spatial resolution of greater than 0.1 μm. NanoSIMS can detect the use of isotope tracers such as ¹³C, ¹⁵N, and ¹⁸O. Therefore, NanoSIMS can be used to the chemical activity nitrogen in the cell.

Automated greenhouses can be used for planta analytics. Plant metrics in response to microbial exposure include, but are not limited to, biomass, chloroplast analysis, CCD camera, volumetric tomography measurements.

One way of enriching a microbe population is according to genotype. For example, a polymerase chain reaction (PCR) assay with a targeted primer or specific primer. Primers designed for the nifH gene can be used to identity diazotrophs because diazotrophs express the nifH gene in the process of nitrogen fixation. A microbial population can also be enriched via single-cell culture-independent approaches and chemotaxis-guided isolation approaches. Alternatively, targeted isolation of microbes can be performed by culturing the microbes on selection media. Premeditated approaches to enriching microbial populations for desired traits can be guided by bioinformatics data and are described herein.

### Domestication of Microbes

Microbes isolated from nature can undergo a domestication process wherein the microbes are converted to a form that is genetically trackable and identifiable. One way to domesticate a microbe is to engineer it with antibiotic resistance. The process of engineering antibiotic resistance can begin by determining the antibiotic sensitivity in the wild type microbial strain. If the bacteria are sensitive to the antibiotic, then the antibiotic can be a good candidate for antibiotic resistance engineering. Subsequently, an antibiotic resistant gene or a counterselectable suicide vector can be incorporated into the genome of a microbe using recombineering methods. A counterselectable suicide vector may consist of a deletion of the gene of interest, a selectable marker, and the counterselectable marker sacB. Counterselection can be used to exchange native microbial DNA sequences with antibiotic resistant genes. A medium throughput method can be used to evaluate multiple microbes simultaneously allowing for parallel domestication. Alternative methods of domestication include the use of homing nucleases to prevent the suicide vector sequences from looping out or from obtaining intervening vector sequences.

DNA vectors can be introduced into bacteria via several methods including electroporation and chemical transformations. A standard library of vectors can be used for transformations. An example of a method of gene editing is CRISPR preceded by Cas9 testing to ensure activity of Cas9 in the microbes.

The remodeling process may include transiently transfecting certain plasmids into the microbes. These plasmids may then be cured from the microbes to remove them. Various methods involving chemical and physical agents have been developed to eliminate plasmids. Protocols for curing plasmids consist frequently of exposure of a culture to sub-inhibitory concentrations of some chemical agents, e.g. acridine orange, acriflavine, and sodium dodecyl sulfate or to a super-optimal temperature followed by selection of cured derivatives.

In the instances where the plasmid is stable or the loss of property difficult to determine, the bacteria can be treated with curing agents. These include chemical and physical agents, some of which can mutate DNA, interfere specifically with its replication, or affect particular structural components or enzymes of the bacterial cell. Protocols for curing plasmids consist frequently of exposure of a culture to sub-inhibitory concentrations of some chemical agents, e.g. acridine orange, acriflavine, and sodium dodecyl sulfate or to a super-optimal temperature followed by selection of cured derivatives. The DNA intercalating agents such as Acridine orange and ethidium bromide are the most commonly used because they are found to be effective against plasmids in a wide variety of genera. Although all of these agents have been used to enhance the recovery of plasmid less derivatives of various bacteria, they are individually effective only against some plasmids and their likely response is unpredictable. The efficiency of curing can also vary widely depending on the plasmid and the particular bacterial host carrying it. In some cases, a difficult to cure plasmid may be removed by introducing an additional, easier to cure plasmid which encodes a protein or system able to target and degrade the difficult to cure plasmid. For example a difficult to cure plasmid may be cured by introducing a plasmid encoding for a CRIPSR system targeting the difficult to cure plasmid. Once a bacterial strain has been cured samples may be sequenced to ensure complete removal of the plasmid sequence.

### Non-Transgenic Engineering of Microbes

A microbial population with favorable traits can be obtained via directed evolution. Direct evolution is an approach wherein the process of natural selection is mimicked to evolve proteins or nucleic acids towards a user-defined goal. An example of direct evolution is when random mutations are introduced into a microbial population, the microbes with the most favorable traits are selected, and the growth of the selected microbes is continued. The most favorable traits in growth promoting rhizobacteria (PGPRs) may be in nitrogen fixation. The method of directed evolution may be iterative and adaptive based on the selection process after each iteration.

Plant growth promoting rhizobacteria (PGPRs) with high capability of nitrogen fixation can be generated. The evolution of PGPRs can be carried out via the introduction of genetic variation. Genetic variation can be introduced via polymerase chain reaction mutagenesis, oligonucleotide-directed mutagenesis, saturation mutagenesis, fragment shuffling mutagenesis, homologous recombination, CRISPR/Cas9 systems, chemical mutagenesis, and combinations thereof. These approaches can introduce random mutations into the microbial population. For example, mutants can be generated using synthetic DNA or RNA via oligonucleotide-directed mutagenesis. Mutants can be generated using tools contained on plasmids, which are later cured. Genes of interest can be identified using libraries from other species with improved traits including, but not limited to, improved PGPR properties, improved colonization of cereals, increased oxygen sensitivity, increased nitrogen fixation, and increased ammonia excretion. Intrageneric genes can be designed based on these libraries using software such as Geneious or Platypus design software. Mutations can be designed with the aid of machine learning. Mutations can be designed with the aid of a metabolic model. Automated design of the mutation can be done using a la Platypus and will guide RNAs for Cas-directed mutagenesis.

The intra-generic genes can be transferred into the host microbe. Additionally, reporter systems can also be transferred to the microbe. The reporter systems characterize promoters, determine the transformation success, screen mutants, and act as negative screening tools.

The microbes carrying the mutation can be cultured via serial passaging. A microbial colony contains a single variant of the microbe. Microbial colonies are screened with the aid of an automated colony picker and liquid handler. Mutants with gene duplication and increased copy number express a higher genotype of the desired trait.

### Selection of Plant Growth Promoting Microbes Based on Nitrogen Fixation

The microbial colonies can be screened using various assays to assess nitrogen fixation. One way to measure nitrogen fixation is via a single fermentative assay, which measures nitrogen excretion. An alternative method is the acetylene reduction assay (ARA) with in-line sampling over time. ARA can be performed in high throughput plates of microtube arrays. ARA can be performed with live plants and plant tissues. The media formulation and media oxygen concentration can be varied in ARA assays. Another method of screening microbial variants is by using biosensors. The use of NanoSIMS and Raman microspectroscopy can be used to investigate the activity of the microbes. In some cases, bacteria can also be cultured and expanded using methods of fermentation in bioreactors. The bioreactors are designed to improve robustness of bacteria growth and to decrease the sensitivity of bacteria to oxygen. Medium to high TP plate-based microfermentors are used to evaluate oxygen sensitivity, nutritional needs, nitrogen fixation, and nitrogen excretion. The bacteria can also be co-cultured with competitive or beneficial microbes to elucidate cryptic pathways. Flow cytometry can be used to screen for bacteria that produce high levels of nitrogen using chemical, colorimetric, or fluorescent indicators. The bacteria may be cultured in the presence or absence of a nitrogen source. For example, the bacteria may be cultured with glutamine, ammonia, urea or nitrates.

### Microbe Breeding

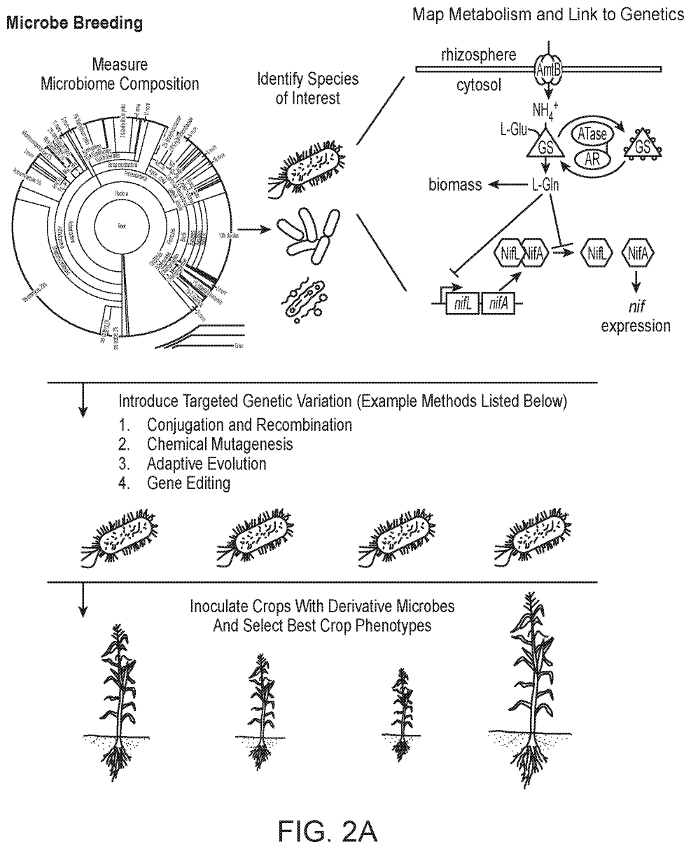

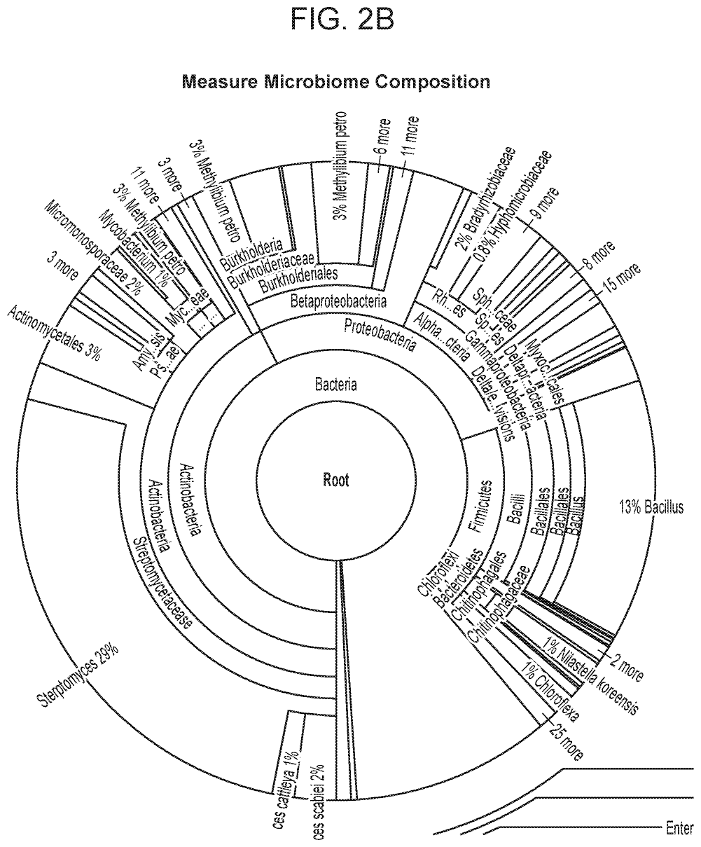

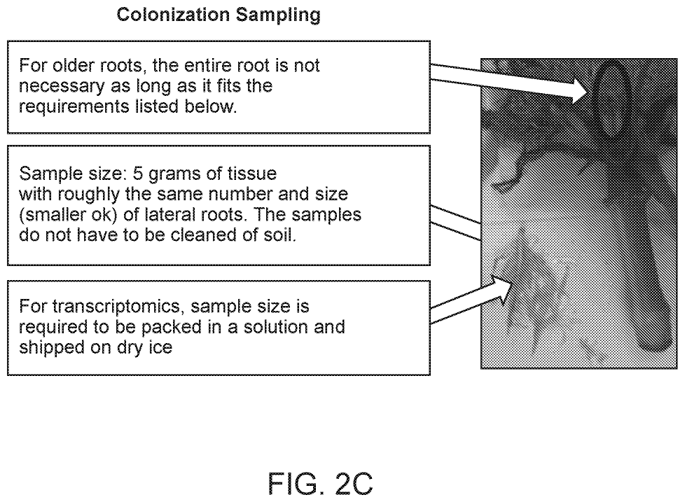

Microbe breeding is a method to systematically identify and improve the role of species within the crop microbiome. The method comprises three steps: 1) selection of candidate species by mapping plant-microbe interactions and predicting regulatory networks linked to a particular phenotype, 2) pragmatic and predictable improvement of microbial phenotypes through intra-species crossing of regulatory networks and gene clusters, and 3) screening and selection of new microbial genotypes that produce desired crop phenotypes. To systematically assess the improvement of strains, a model is created that links colonization dynamics of the microbial community to genetic activity by key species. The model is used to predict genetic targets breeding and improve the frequency of selecting improvements in microbiome-encoded traits of agronomic relevance. See, FIG. 2A for a graphical representation of an embodiment of the process. In particular, FIG. 2A depicts a schematic of microbe breeding, in accordance with embodiments. As illustrated in FIG. 2A, rational improvement of the crop microbiome may be used to increase soil biodiversity, tune impact of keystone species, and/or alter timing and expression of important metabolic pathways. To this end, the inventors have developed a microbe breeding pipeline to identify and improve the role of strains within the crop microbiome. The method comprises three steps: 1) selection of candidate species by mapping plant-microbe interactions and predicting regulatory networks linked to a particular phenotype, 2) pragmatic and predictable improvement of microbial phenotypes through intragenomic crossing of gene regulatory networks and gene clusters, and 3) screening and selection of new microbial genotypes that produce desired crop phenotypes. To systematically assess the improvement of strains, the inventors employ a model that links colonization dynamics of the microbial community to genetic activity by key species. This process represents a methodology for breeding and selecting improvements in microbiome-encoded traits of agronomic relevance.

Production of bacteria to improve plant traits (e.g., nitrogen fixation) can be achieved through serial passage. The production of this bacteria can be done by selecting plants, which have a particular improved trait that is influenced by the microbial flora, in addition to identifying bacteria and/or compositions that are capable of imparting one or more improved traits to one or more plants. One method of producing a bacteria to improve a plant trait includes the steps of: (a) isolating bacteria from tissue or soil of a first plant; (b) introducing a genetic variation into one or more of the bacteria to produce one or more variant bacteria; (c) exposing a plurality of plants to the variant bacteria; (d) isolating bacteria from tissue or soil of one of the plurality of plants, wherein the plant from which the bacteria is isolated has an improved trait relative to other plants in the plurality of plants; and (e) repeating steps (b) to (d) with bacteria isolated from the plant with an improved trait (step (d)). Steps (b) to (d) can be repeated any number of times (e.g., once, twice, three times, four times, five times, ten times, or more) until the improved trait in a plant reaches a desired level. Further, the plurality of plants can be more than two plants, such as 10 to 20 plants, or 20 or more, 50 or more, 100 or more, 300 or more, 500 or more, or 1000 or more plants.

In addition to obtaining a plant with an improved trait, a bacterial population comprising bacteria comprising one or more genetic variations introduced into one or more genes (e.g., genes regulating nitrogen fixation) is obtained. By repeating the steps described above, a population of bacteria can be obtained that include the most appropriate members of the population that correlate with a plant trait of interest. The bacteria in this population can be identified and their beneficial properties determined, such as by genetic and/or phenotypic analysis. Genetic analysis may occur of isolated bacteria in step (a). Phenotypic and/or genotypic information may be obtained using techniques including: high through-put screening of chemical components of plant origin, sequencing techniques including high throughput sequencing of genetic material, differential display techniques (including DDRT-PCR, and DD-PCR), nucleic acid microarray techniques, RNA-sequencing (Whole Transcriptome Shotgun Sequencing), and qRT-PCR (quantitative real time PCR). Information gained can be used to obtain community profiling information on the identity and activity of bacteria present, such as phylogenetic analysis or microarray-based screening of nucleic acids coding for components of rRNA operons or other taxonomically informative loci. Examples of taxonomically informative loci include 16S rRNA gene, 23S rRNA gene, 5S rRNA gene, 5.8S rRNA gene, 12S rRNA gene, 18S rRNA gene, 28S rRNA gene, gyrB gene, rpoB gene, fusA gene, recA gene, coxl gene, nifD gene. Example processes of taxonomic profiling to determine taxa present in a population are described in US20140155283. Bacterial identification may comprise characterizing activity of one or more genes or one or more signaling pathways, such as genes associated with the nitrogen fixation pathway. Synergistic interactions (where two components, by virtue of their combination, increase a desired effect by more than an additive amount) between different bacterial species may also be present in the bacterial populations.

### Genetic Variation—Locations and Sources of Genomic Alteration

The genetic variation may be a gene selected from the group consisting of: nifA, nifL, ntrB, ntrC, glnA, glnB, glnK, draT, amtB, glnD, glnE, nifJ, nifH, nifD, nifK, nifY, nifE, nifN, nifJ, nifS, nifV, nifW, nifZ, nifM, nifF, nifB, nifQ, bcsII, bcsIII, yjbE, fhaB, pehA, glgA, otsB, treZ, and cysZ. The genetic variation may be a variation in a gene encoding a protein with functionality selected from the group consisting of: glutamine synthetase, glutaminase, glutamine synthetase adenylyltransferase, transcriptional activator, anti-transcriptional activator, pyruvate flavodoxin oxidoreductase, flavodoxin, NAD+-dinitrogen-reductase aDP-D-ribosyltransferase, exopolysaccharide production, filamentous hemagglutinin, glycogen synthase, trehalose synthesis, or a sulfate transporter. The genetic variation may be a mutation that results in one or more of: increased expression or activity of NifA glutaminase, bcsII, bcsIII, yjbE, fhaB, pehA, otsB, treZ or CysZ; decreased expression or activity of NifL, NtrB, glutamine synthetase, GlnB, GlnK, DraT, AmtB, or glgA; decreased adenylyl-removing activity of GlnE; or decreased uridylyl-removing activity of GlnD. Introducing a genetic variation may comprise insertion and/or deletion of one or more nucleotides at a target site, such as 1, 2, 3, 4, 5, 10, 25, 50, 100, 250, 500, or more nucleotides. The genetic variation introduced into one or more bacteria of the methods disclosed herein may be a knock-out mutation (e.g. deletion of a promoter, insertion or deletion to produce a premature stop codon, deletion of an entire gene), or it may be elimination or abolishment of activity of a protein domain (e.g. point mutation affecting an active site, or deletion of a portion of a gene encoding the relevant portion of the protein product), or it may alter or abolish a regulatory sequence of a target gene. One or more regulatory sequences may also be inserted, including heterologous regulatory sequences and regulatory sequences found within a genome of a bacterial species or genus corresponding to the bacteria into which the genetic variation is introduced. Moreover, regulatory sequences may be selected based on the expression level of a gene in a bacterial culture or within a plant tissue. The genetic variation may be a pre-determined genetic variation that is specifically introduced to a target site. The genetic variation may be a random mutation within the target site. The genetic variation may be an insertion or deletion of one or more nucleotides. In some cases, a plurality of different genetic variations (e.g. 2, 3, 4, 5, 10, or more) are introduced into one or more of the isolated bacteria before exposing the bacteria to plants for assessing trait improvement. The plurality of genetic variations can be any of the above types, the same or different types, and in any combination. In some cases, a plurality of different genetic variations are introduced serially, introducing a first genetic variation after a first isolation step, a second genetic variation after a second isolation step, and so forth so as to accumulate a plurality of genetic variations in bacteria imparting progressively improved traits on the associated plants.

| Gene target | Genotype | Phenotype |
|---|---|---|
| bcsii, bcsiii yjbE | Encode an exopolysaccharide | Upregulation leads to increased |
| | production enzyme in the | attachment of microbes to corn |
| | microbes | roots, likely due to increased |
| | | bacterial agglutination and |
| | | biofilm formation on the root |
| | | surface. |
| fhaB | Encodes a filamentous hemagglutinin | Upregulation leads to increased |
| | | agglutination and enhanced |
| | | microbe attachment to corn roots. |
| pehA | Encodes an endo- | Upregulation leads to increased |
| | polygalaturonase | microbe colonization in corn |
| | precursor | roots, possibly due to breakdown |
| | | of root cell wall components and |
| | | increased endophytic entry. |
| gigA | Encodes glycogen synthase | Deletion of gigA leads to |
| | | increased excretion of ammonia, |
| | | likely due to increased carbon |
| | | flux away from glycogen |
| | | synthesis and into glycolysis and |
| | | oxidative phosphorylation, |
| | | leading to increased available |
| | | ATP for nitrogen fixation. |
| otsB and treZ | Trehalose synthesis genes | Upregulation of trehalose |
| | | synthesis genes otsB and treZ |
| | | leads to increased nitrogenase |
| | | expression in the presence of |
| | | extracellular nitrogen by an |
| | | unknown mechanism. |
| cysZ | Encodes a sulfate transporter | Upregulation of the cysZ gene, |
| | | leads to increased nitrogenase |
| | | activity, likely due to increased |
| | | sulfur uptake leading to elevated |
| | | synthesis of iron-sulfur clusters |
| | | in the nitrogenase protein. |
| nifA and nifL | Disruption of NifL expression or | Strains are able to fix nitrogen in |
| | elimination of the ammonia- | the presence of higher levels of |
| | sensing domain of NifA | exogenous ammonia than wild- |
| | deregulates repressions of nitrogen | type strains. |
| | fixation by ammonia. In some | |
| | strains, the nifL gene has been | |
| | replaced by well-characterized, | |
| | non-coding regulatory DNA to | |
| | improve expression of NifA. | |
| ginB | Disruption of GinB expression | Strains are able to fix nitrogen in |
| | contribute to deregulating | the presence of higher levels of |
| | nitrogenase gene expression from | exogenous ammonia than wild- |
| | environmental ammonia sensing. | type strains. |
| amtB | Disruption of AmtB expression | Strains are able to excrete |
| | leads to reduced uptake of | ammonia at higher levels than |
| | environmental ammonia by the | wild-type strains. |
| | strain. | |
| glnE | The glnE gene has been deleted I | Strains exhibited reduced |
| | some strains and in other strains | synthesis of glutamine and |
| | only the N-terminal region of the | increased excretion of ammonia |
| | glnE gene. | under nitrogen fixing conditions. |
| glnA | Point mutations in glnA. | Strains exhibited reduced |
| | | synthesis of glutamine and |
| | | increased excretion of ammonia |
| | | under nitrogen fixing conditions. |
| draT | The draT gene has been deleted. | Strains exhibit increased |
| | | nitrogenase activity in the |
| | | presence of environmental |
| | | ammonia and oxygen. |
| ginD | The ginD gene has been deleted. | Strains are able to fix nitrogen in |
| | Disruption of GinD expression | the presence of higher levels of |
| | contributes to deregulating | exogenous ammonia than wild- |
| | nitrogenase gene expression from | type strains. |
| | environmental ammonia sensing. | |

### Genetic Variation—Methods of Introducing Genomic Alteration

In general, the term “genetic variation” refers to any change introduced into a polynucleotide sequence relative to a reference polynucleotide, such as a reference genome or portion thereof, or reference gene or portion thereof. A genetic variation may be referred to as a “mutation,” and a sequence or organism comprising a genetic variation may be referred to as a “genetic variant” or “mutant”. Genetic variations can have any number of effects, such as the increase or decrease of some biological activity, including gene expression, metabolism, and cell signaling. Genetic variations can be specifically introduced to a target site, or introduced randomly. A variety of molecular tools and methods are available for introducing genetic variation. For example, genetic variation can be introduced via polymerase chain reaction mutagenesis, oligonucleotide-directed mutagenesis, saturation mutagenesis, fragment shuffling mutagenesis, homologous recombination, recombineering, lambda red mediated recombination, CRISPR/Cas9 systems, chemical mutagenesis, and combinations thereof. Chemical methods of introducing genetic variation include exposure of DNA to a chemical mutagen, e.g., ethyl methanesulfonate (EMS), methyl methanesulfonate (MMS), N-nitrosourea (EN U), N-methyl-N-nitro-N′-nitrosoguanidine, 4-nitroquinoline N-oxide, diethylsulfate, benzopyrene, cyclophosphamide, bleomycin, triethylmelamine, acrylamide monomer, nitrogen mustard, vincristine, diepoxyalkanes (for example, diepoxybutane), ICR-170, formaldehyde, procarbazine hydrochloride, ethylene oxide, dimethylnitrosamine, 7,12 dimethylbenz(a)anthracene, chlorambucil, hexamethylphosphoramide, bisulfan, and the like. Radiation mutation-inducing agents include ultraviolet radiation, γ-irradiation, X-rays, and fast neutron bombardment Genetic variation can also be introduced into a nucleic acid using, e.g., trimethylpsoralen with ultraviolet light. Random or targeted insertion of a mobile DNA element, e.g., a transposable element, is another suitable method for generating genetic variation. Genetic variations can be introduced into a nucleic acid during amplification in a cell-free in vitro system, e.g., using a polymerase chain reaction (PCR) technique such as error-prone PCR. Genetic variations can be introduced into a nucleic acid in vitro using DNA shuffling techniques (e.g., exon shuffling, domain swapping, and the like). Genetic variations can also be introduced into a nucleic acid as a result of a deficiency in a DNA repair enzyme in a cell, e.g., the presence in a cell of a mutant gene encoding a mutant DNA repair enzyme is expected to generate a high frequency of mutations (i.e., about 1 mutation/100 genes-1 mutation/10,000 genes) in the genome of the cell. Examples of genes encoding DNA repair enzymes include but are not limited to Mut H, Mut S, Mut L, and Mut U, and the homologs thereof in other species (e.g., MSH 1 6, PMS 1 2, MLH 1, GTBP, ERCC-1, and the like). Example descriptions of various methods for introducing genetic variations are provided in e.g., Stemple (2004) Nature 5:1-7; Chiang et al. (1993) PCR Methods Appl 2(3): 210-217; Stemmer (1994) Proc. Natl. Acad. Sci. USA 91:10747-10751; and U.S. Pat. Nos. 6,033,861, and 6,773,900.

Genetic variations introduced into microbes may be classified as transgenic, cisgenic, intragenomic, intrageneric, intergeneric, synthetic, evolved, rearranged, or SNPs.

Genetic variation may be introduced into numerous metabolic pathways within microbes to elicit improvements in the traits described above. Representative pathways include sulfur uptake pathways, glycogen biosynthesis, the glutamine regulation pathway, the molybdenum uptake pathway, the nitrogen fixation pathway, ammonia assimilation, ammonia excretion or secretion, nitrogen uptake, glutamine biosynthesis, annamox, phosphate solubilization, organic acid transport, organic acid production, agglutinins production, reactive oxygen radical scavenging genes, Indole Acetic Acid biosynthesis, trehalose biosynthesis, plant cell wall degrading enzymes or pathways, root attachment genes, exopolysaccharide secretion, glutamate synthase pathway, iron uptake pathways, siderophore pathway, chitinase pathway, ACC deaminase, glutathione biosynthesis, phosphorous signalig genes, quorum quenching pathway, cytochrome pathways, hemoglobin pathway, bacterial hemoglobin-like pathway, small RNA rsmZ, rhizobitoxine biosynthesis, lapA adhesion protein, AHL quorum sensing pathway, phenazine biosynthesis, cyclic lipopeptide biosynthesis, and antibiotic production.

CRISPR/Cas9 (Clustered regularly interspaced short palindromic repeats)/CRISPR-associated (Cas) systems can be used to introduce desired mutations. CRISPR/Cas9 provide bacteria and archaea with adaptive immunity against viruses and plasmids by using CRISPR RNAs (crRNAs) to guide the silencing of invading nucleic acids. The Cas9 protein (or functional equivalent and/or variant thereof, i.e., Cas9-like protein) naturally contains DNA endonuclease activity that depends on the association of the protein with two naturally occurring or synthetic RNA molecules called crRNA and tracrRNA (also called guide RNAs). In some cases, the two molecules are covalently link to form a single molecule (also called a single guide RNA (“sgRNA”). Tus, the Cas9 or Cas9-like protein associates with a DNA-targeting RNA (which term encompasses both the two-molecule guide RNA configuration and the single-molecule guide RNA configuration), which activates the Cas9 or Cas9-like protein and guides the protein to a target nucleic acid sequence. If the Cas9 or Cas9-like protein retains its natural enzymatic function, it will cleave target DNA to create a double-stranded break, which can lead to genome alteration (i.e., editing: deletion, insertion (when a donor polynucleotide is present), replacement, etc.), thereby altering gene expression. Some variants of Cas9 (which variants are encompassed by the term Cas9-like) have been altered such that they have a decreased DNA cleaving activity (in some cases, they cleave a single strand instead of both strands of the target DNA, while in other cases, they have severely reduced to no DNA cleavage activity). Further exemplary descriptions of CRISPR systems for introducing genetic variation can be found in, e.g. U.S. Pat. No. 8,795,965.

As a cyclic amplification technique, polymerase chain reaction (PCR) mutagenesis uses mutagenic primers to introduce desired mutations. PCR is performed by cycles of denaturation, annealing, and extension. After amplification by PCR, selection of mutated DNA and removal of parental plasmid DNA can be accomplished by: 1) replacement of dCTP by hydroxymethylated-dCTP during PCR, followed by digestion with restriction enzymes to remove non-hydroxymethylated parent DNA only; 2) simultaneous mutagenesis of both an antibiotic resistance gene and the studied gene changing the plasmid to a different antibiotic resistance, the new antibiotic resistance facilitating the selection of the desired mutation thereafter; 3) after introducing a desired mutation, digestion of the parent methylated template DNA by restriction enzyme Dpnl which cleaves only methylated DNA, by which the mutagenized unmethylated chains are recovered; or 4) circularization of the mutated PCR products in an additional ligation reaction to increase the transformation efficiency of mutated DNA. Further description of exemplary methods can be found in e.g. U.S. Pat. Nos. 7,132,265, 6,713,285, 6,673,610, 6,391,548, 5,789,166, 5,780,270, 5,354,670, 5,071,743, and US20100267147.

Oligonucleotide-directed mutagenesis, also called site-directed mutagenesis, typically utilizes a synthetic DNA primer. This synthetic primer contains the desired mutation and is complementary to the template DNA around the mutation site so that it can hybridize with the DNA in the gene of interest. The mutation may be a single base change (a point mutation), multiple base changes, deletion, or insertion, or a combination of these. The single-strand primer is then extended using a DNA polymerase, which copies the rest of the gene. The gene thus copied contains the mutated site, and may then be introduced into a host cell as a vector and cloned. Finally, mutants can be selected by DNA sequencing to check that they contain the desired mutation.

Genetic variations can be introduced using error-prone PCR. In this technique the gene of interest is amplified using a DNA polymerase under conditions that are deficient in the fidelity of replication of sequence. The result is that the amplification products contain at least one error in the sequence. When a gene is amplified and the resulting product(s) of the reaction contain one or more alterations in sequence when compared to the template molecule, the resulting products are mutagenized as compared to the template. Another means of introducing random mutations is exposing cells to a chemical mutagen, such as nitrosoguanidine or ethyl methanesulfonate (Nestmann, Mutat Res 1975 June; 28(3):323-30), and the vector containing the gene is then isolated from the host.

Saturation mutagenesis is another form of random mutagenesis, in which one tries to generate all or nearly all possible mutations at a specific site, or narrow region of a gene. In a general sense, saturation mutagenesis is comprised of mutagenizing a complete set of mutagenic cassettes (wherein each cassette is, for example, 1-500 bases in length) in defined polynucleotide sequence to be mutagenized (wherein the sequence to be mutagenized is, for example, from 15 to 100,000 bases in length). Therefore, a group of mutations (e.g. ranging from 1 to 100 mutations) is introduced into each cassette to be mutagenized. A grouping of mutations to be introduced into one cassette can be different or the same from a second grouping of mutations to be introduced into a second cassette during the application of one round of saturation mutagenesis. Such groupings are exemplified by deletions, additions, groupings of particular codons, and groupings of particular nucleotide cassettes.

Fragment shuffling mutagenesis, also called DNA shuffling, is a way to rapidly propagate beneficial mutations. In an example of a shuffling process, DNAse is used to fragment a set of parent genes into pieces of e.g. about 50-100 bp in length. This is then followed by a polymerase chain reaction (PCR) without primers—DNA fragments with sufficient overlapping homologous sequence will anneal to each other and are then be extended by DNA polymerase. Several rounds of this PCR extension are allowed to occur, after some of the DNA molecules reach the size of the parental genes. These genes can then be amplified with another PCR, this time with the addition of primers that are designed to complement the ends of the strands. The primers may have additional sequences added to their 5′ ends, such as sequences for restriction enzyme recognition sites needed for ligation into a cloning vector. Further examples of shuffling techniques are provided in US20050266541.

Homologous recombination mutagenesis involves recombination between an exogenous DNA fragment and the targeted polynucleotide sequence. After a double-stranded break occurs, sections of DNA around the 5′ ends of the break are cut away in a process called resection. In the strand invasion step that follows, an overhanging 3′ end of the broken DNA molecule then “invades” a similar or identical DNA molecule that is not broken. The method can be used to delete a gene, remove exons, add a gene, and introduce point mutations. Homologous recombination mutagenesis can be permanent or conditional. Typically, a recombination template is also provided. A recombination template may be a component of another vector, contained in a separate vector, or provided as a separate polynucleotide. In some embodiments, a recombination template is designed to serve as a template in homologous recombination, such as within or near a target sequence nicked or cleaved by a site-specific nuclease. A template polynucleotide may be of any suitable length, such as about or more than about 10, 15, 20, 25, 50, 75, 100, 150, 200, 500, 1000, or more nucleotides in length. In some embodiments, the template polynucleotide is complementary to a portion of a polynucleotide comprising the target sequence. When optimally aligned, a template polynucleotide might overlap with one or more nucleotides of a target sequences (e.g. about or more than about 1, 5, 10, 15, 20, 25, 30, 35, 40, 45, 50, 60, 70, 80, 90, 100 or more nucleotides). In some embodiments, when a template sequence and a polynucleotide comprising a target sequence are optimally aligned, the nearest nucleotide of the template polynucleotide is within about 1, 5, 10, 15, 20, 25, 50, 75, 100, 200, 300, 400, 500, 1000, 5000, 10000, or more nucleotides from the target sequence. Non-limiting examples of site-directed nucleases useful in methods of homologous recombination include zinc finger nucleases, CRISPR nucleases, TALE nucleases, and meganuclease. For a further description of the use of such nucleases, see e.g. U.S. Pat. No. 8,795,965 and US20140301990.

Mutagens that create primarily point mutations and short deletions, insertions, transversions, and/or transitions, including chemical mutagens or radiation, may be used to create genetic variations. Mutagens include, but are not limited to, ethyl methanesulfonate, methylmethane sulfonate, N-ethyl-N-nitrosurea, triethylmelamine, N-methyl-N-nitrosourea, procarbazine, chlorambucil, cyclophosphamide, diethyl sulfate, acrylamide monomer, melphalan, nitrogen mustard, vincristine, dimethylnitrosamine, N-methyl-N′-nitro-Nitrosoguanidine, nitrosoguanidine, 2-aminopurine, 7,12 dimethyl-benz(a)anthracene, ethylene oxide, hexamethylphosphoramide, bisulfan, diepoxyalkanes (diepoxyoctane, diepoxybutane, and the like), 2-methoxy-6-chloro-9[3-(ethyl-2-chloro-ethyl)aminopropylamino]acridine dihydrochloride and formaldehyde.

Introducing genetic variation may be an incomplete process, such that some bacteria in a treated population of bacteria carry a desired mutation while others do not. In some cases, it is desirable to apply a selection pressure so as to enrich for bacteria carrying a desired genetic variation. Traditionally, selection for successful genetic variants involved selection for or against some functionality imparted or abolished by the genetic variation, such as in the case of inserting antibiotic resistance gene or abolishing a metabolic activity capable of converting a non-lethal compound into a lethal metabolite. It is also possible to apply a selection pressure based on a polynucleotide sequence itself, such that only a desired genetic variation need be introduced (e.g. without also requiring a selectable marker). In this case, the selection pressure can comprise cleaving genomes lacking the genetic variation introduced to a target site, such that selection is effectively directed against the reference sequence into which the genetic variation is sought to be introduced. Typically, cleavage occurs within 100 nucleotides of the target site (e.g. within 75, 50, 25, 10, or fewer nucleotides from the target site, including cleavage at or within the target site). Cleaving may be directed by a site-specific nuclease selected from the group consisting of a Zinc Finger nuclease, a CRISPR nuclease, a TALE nuclease (TALEN), or a meganuclease. Such a process is similar to processes for enhancing homologous recombination at a target site, except that no template for homologous recombination is provided. As a result, bacteria lacking the desired genetic variation are more likely to undergo cleavage that, left unrepaired, results in cell death. Bacteria surviving selection may then be isolated for use in exposing to plants for assessing conferral of an improved trait.

A CRISPR nuclease may be used as the site-specific nuclease to direct cleavage to a target site. An improved selection of mutated microbes can be obtained by using Cas9 to kill non-mutated cells. Plants are then inoculated with the mutated microbes to re-confirm symbiosis and create evolutionary pressure to select for efficient symbionts. Microbes can then be re-isolated from plant tissues. CRISPR nuclease systems employed for selection against non-variants can employ similar elements to those described above with respect to introducing genetic variation, except that no template for homologous recombination is provided. Cleavage directed to the target site thus enhances death of affected cells.

Other options for specifically inducing cleavage at a target site are available, such as zinc finger nucleases, TALE nuclease (TALEN) systems, and meganuclease. Zinc-finger nucleases (ZFNs) are artificial DNA endonucleases generated by fusing a zinc finger DNA binding domain to a DNA cleavage domain. ZFNs can be engineered to target desired DNA sequences and this enables zinc-finger nucleases to cleave unique target sequences. When introduced into a cell, ZFNs can be used to edit target DNA in the cell (e.g., the cell's genome) by inducing double stranded breaks. Transcription activator-like effector nucleases (TALENs) are artificial DNA endonucleases generated by fusing a TAL (Transcription activator-like) effector DNA binding domain to a DNA cleavage domain. TALENS can be quickly engineered to bind practically any desired DNA sequence and when introduced into a cell, TALENs can be used to edit target DNA in the cell (e.g., the cell's genome) by inducing double strand breaks. Meganucleases (homing endonuclease) are endodeoxyribonucleases characterized by a large recognition site (double-stranded DNA sequences of 12 to 40 base pairs. Meganucleases can be used to replace, eliminate or modify sequences in a highly targeted way. By modifying their recognition sequence through protein engineering, the targeted sequence can be changed. Meganucleases can be used to modify all genome types, whether bacterial, plant or animal and are commonly grouped into four families: the LAGLIDADG family (SEQ ID NO: 1), the GIY-YIG family, the His-Cyst box family and the HNH family. Exemplary homing endonucleases include I-SceI, I-CeuI, PI-PspI, PI-Sce, I-SceIV, I-CsmI, I-PanI, I-SceII, I-PpoI, I-SceIII, I-CreI, I-TevI, I-TevII and I-TevIII.

### Genetic Variation—Methods of Identification

The microbes of the present disclosure may be identified by one or more genetic modifications or alterations, which have been introduced into said microbe. One method by which said genetic modification or alteration can be identified is via reference to a SEQ ID NO that contains a portion of the microbe's genomic sequence that is sufficient to identify the genetic modification or alteration.

Further, in the case of microbes that have not had a genetic modification or alteration (e.g. a wild type, WT) introduced into their genomes, the disclosure can utilize 16S nucleic acid sequences to identify said microbes. A 16S nucleic acid sequence is an example of a “molecular marker” or “genetic marker,” which refers to an indicator that is used in methods for visualizing differences in characteristics of nucleic acid sequences. Examples of other such indicators are restriction fragment length polymorphism (RFLP) markers, amplified fragment length polymorphism (AFLP) markers, single nucleotide polymorphisms (SNPs), insertion mutations, microsatellite markers (SSRs), sequence-characterized amplified regions (SCARs), cleaved amplified polymorphic sequence (CAPS) markers or isozyme markers or combinations of the markers described herein which defines a specific genetic and chromosomal location. Markers further include polynucleotide sequences encoding 16S or 18S rRNA, and internal transcribed spacer (ITS) sequences, which are sequences found between small-subunit and large-subunit rRNA genes that have proven to be especially useful in elucidating relationships or distinctions when compared against one another. Furthermore, the disclosure utilizes unique sequences found in genes of interest (e.g. nifH,D,K,L,A, glnE, amtB, etc.) to identify microbes disclosed herein.

The primary structure of major rRNA subunit 16S comprise a particular combination of conserved, variable, and hypervariable regions that evolve at different rates and enable the resolution of both very ancient lineages such as domains, and more modern lineages such as genera. The secondary structure of the 16S subunit include approximately 50 helices which result in base pairing of about 67% of the residues. These highly conserved secondary structural features are of great functional importance and can be used to ensure positional homology in multiple sequence alignments and phylogenetic analysis. Over the previous few decades, the 16S rRNA gene has become the most sequenced taxonomic marker and is the cornerstone for the current systematic classification of bacteria and archaea (Yarza et al. 2014. Nature Rev. Micro. 12:635-45).

### Genetic Variation—Methods of Detection: Primers, Probes, and Assays

The present disclosure teaches primers, probes, and assays that are useful for detecting the microbes taught herein. In some aspects, the disclosure provides for methods of detecting the WT parental strains. In other aspects, the disclosure provides for methods of detecting the non-intergeneric engineered microbes derived from the WT strains. In aspects, the present disclosure provides methods of identifying non-intergeneric genetic alterations in a microbe.

In aspects, the genomic engineering methods of the present disclosure lead to the creation of non-natural nucleotide “junction” sequences in the derived non-intergeneric microbes. These non-naturally occurring nucleotide junctions can be used as a type of diagnostic that is indicative of the presence of a particular genetic alteration in a microbe taught herein.

The present techniques are able to detect these non-naturally occurring nucleotide junctions via the utilization of specialized quantitative PCR methods, including uniquely designed primers and probes. In some aspects, the probes of the disclosure bind to the non-naturally occurring nucleotide junction sequences. In some aspects, traditional PCR is utilized. In other aspects, real-time PCR is utilized. In some aspects, quantitative PCR (qPCR) is utilized.

Thus, the disclosure can cover the utilization of two common methods for the detection of PCR products in real-time: (1)non-specific fluorescent dyes that intercalate with any double-stranded DNA, and (2) sequence-specific DNA probes consisting of oligonucleotides that are labelled with a fluorescent reporter which permits detection only after hybridization of the probe with its complementary sequence. In some aspects, only the non-naturally occurring nucleotide junction will be amplified via the taught primers, and consequently can be detected via either a non-specific dye, or via the utilization of a specific hybridization probe. In other aspects, the primers of the disclosure are chosen such that the primers flank either side of a junction sequence, such that if an amplification reaction occurs, then said junction sequence is present.

Aspects of the disclosure involve non-naturally occurring nucleotide junction sequence molecules per se, along with other nucleotide molecules that are capable of binding to said non-naturally occurring nucleotide junction sequences under mild to stringent hybridization conditions. In some aspects, the nucleotide molecules that are capable of binding to said non-naturally occurring nucleotide junction sequences under mild to stringent hybridization conditions are termed “nucleotide probes.”

_In aspects, genomic DNA can be extracted from samples and used to quantify the presence of microbes of the disclosure by using qPCR. The primers utilized in the qPCR reaction can be primers designed by Primer Blast (https://www.ncbi.nlm.nih.gov/tools/primer-blast) to amplify unique regions of the wild-type genome or unique regions of the engineered non-intergeneric mutant strains. The qPCR reaction can be carried out using the SYBR GreenER qPCR SuperMix Universal (Thermo Fisher P/N 11762100) kit, using only forward and reverse amplification primers; alternatively, the Kapa Probe Force kit (Kapa Biosystems P/N KK4301) can be used with amplification primers and a TaqMan probe containing a FAM dye label at the 5′ end, an internal ZEN quencher, and a minor groove binder and fluorescent quencher at the 3′ end (Integrated DNA Technologies).

qPCR reaction efficiency can be measured using a standard curve generated from a known quantity of gDNA from the target genome. Data can be normalized to genome copies per g fresh weight using the tissue weight and extraction volume.

Quantitative polymerase chain reaction (qPCR) is a method of quantifying, in real time, the amplification of one or more nucleic acid sequences. The real time quantification of the PCR assay permits determination of the quantity of nucleic acids being generated by the PCR amplification steps by comparing the amplifying nucleic acids of interest and an appropriate control nucleic acid sequence, which may act as a calibration standard.

TaqMan probes are often utilized in qPCR assays that require an increased specificity for quantifying target nucleic acid sequences. TaqMan probes comprise a oligonucleotide probe with a fluorophore attached to the 5′ end and a quencher attached to the 3′ end of the probe. When the TaqMan probes remain as is with the 5′ and 3′ ends of the probe in close contact with each other, the quencher prevents fluorescent signal transmission from the fluorophore. TaqMan probes are designed to anneal within a nucleic acid region amplified by a specific set of primers. As the Taq polymerase extends the primer and synthesizes the nascent strand, the 5′ to 3′ exonuclease activity of the Taq polymerase degrades the probe that annealed to the template. This probe degradation releases the fluorophore, thus breaking the close proximity to the quencher and allowing fluorescence of the fluorophore. Fluorescence detected in the qPCR assay is directly proportional to the fluorophore released and the amount of DNA template present in the reaction.

The features of qPCR allow the practitioner to eliminate the labor-intensive post-amplification step of gel electrophoresis preparation, which is generally required for observation of the amplified products of traditional PCR assays. The benefits of qPCR over conventional PCR are considerable, and include increased speed, ease of use, reproducibility, and quantitative ability

### Improvement of Traits

Methods of the present disclosure may be employed to introduce or improve one or more of a variety of desirable traits. Traits to be improved may be traits of the bacterium, or the bacterium may be modified to improve a trait in an associated plant. Examples of traits that may introduced or improved include: root biomass, root length, height, shoot length, leaf number, water use efficiency, overall biomass, yield, fruit size, grain size, photosynthesis rate, tolerance to drought, heat tolerance, salt tolerance, resistance to nematode stress, resistance to a fungal pathogen, resistance to a bacterial pathogen, resistance to a viral pathogen, level of a metabolite, and proteome expression. The desirable traits, including height, overall biomass, root and/or shoot biomass, seed germination, seedling survival, photosynthetic efficiency, transpiration rate, seed/fruit number or mass, plant grain or fruit yield, leaf chlorophyll content, photosynthetic rate, root length, or any combination thereof, can be used to measure growth, and compared with the growth rate of reference agricultural plants (e.g., plants without the improved traits) grown under identical conditions.

Further examples of traits which may be improved include the ability of a bacterium to adhere to and colonize a plant. For example, a bacterium may be modified to better adhere to a root of a plant, or to produce or secrete a compound beneficial to colonization.

A preferred trait to be introduced or improved is nitrogen fixation, as described herein. In some cases, a plant resulting from the methods described herein exhibits a difference in the trait that is at least about 5% greater, for example at least about 5%, at least about 8%, at least about 10%, at least about 15%, at least about 20%, at least about 25%, at least about 30%, at least about 40%, at least about 50%, at least about 60%, at least about 75%, at least about 80%, at least about 80%, at least about 90%, or at least 100%, at least about 200%, at least about 300%, at least about 400% or greater than a reference agricultural plant grown under the same conditions in the soil. In additional examples, a plant resulting from the methods described herein exhibits a difference in the trait that is at least about 5% greater, for example at least about 5%, at least about 8%, at least about 10%, at least about 15%, at least about 20%, at least about 25%, at least about 30%, at least about 40%, at least about 50%, at least about 60%, at least about 75%, at least about 80%, at least about 80%, at least about 90%, or at least 100%, at least about 200%, at least about 300%, at least about 400% or greater than a reference agricultural plant grown under similar conditions in the soil.

The trait to be improved may be assessed under conditions including the application of one or more biotic or abiotic stressors. Examples of stressors include abiotic stresses (such as heat stress, salt stress, drought stress, cold stress, and low nutrient stress) and biotic stresses (such as nematode stress, insect herbivory stress, fungal pathogen stress, bacterial pathogen stress, and viral pathogen stress).

The trait improved by methods and compositions of the present disclosure may be nitrogen fixation, including in a plant not previously capable of nitrogen fixation. In some cases, bacteria isolated according to a method described herein produce 1% or more (e.g. 2%, 3%, 4%, 5%, 6%, 7%, 8%, 9%, 10%, 15%, 20%, or more) of a plant's nitrogen, which may represent an increase in nitrogen fixation capability of at least 2-fold (e.g. 3-fold, 4-fold, 5-fold, 6-fold, 7-fold, 8-fold, 9-fold, 10-fold, 20-fold, 50-fold, 100-fold, 1000-fold, or more) as compared to bacteria isolated from the first plant before introducing any genetic variation. In some cases, the bacteria produce 5% or more of a plant's nitrogen. The desired level of nitrogen fixation may be achieved after repeating the steps of introducing genetic variation, exposure to a plurality of plants, and isolating bacteria from plants with an improved trait one or more times (e.g. 1, 2, 3, 4, 5, 10, 15, 25, or more times). In some cases, enhanced levels of nitrogen fixation are achieved in the presence of fertilizer supplemented with glutamine, ammonia, or other chemical source of nitrogen. Methods for assessing degree of nitrogen fixation are known, examples of which are described herein.

Microbe breeding is a method to systematically identify and improve the role of species within the crop microbiome. The method comprises three steps: 1) selection of candidate species by mapping plant-microbe interactions and predicting regulatory networks linked to a particular phenotype, 2) pragmatic and predictable improvement of microbial phenotypes through intra-species crossing of regulatory networks and gene clusters, and 3) screening and selection of new microbial genotypes that produce desired crop phenotypes. To systematically assess the improvement of strains, a model is created that links colonization dynamics of the microbial community to genetic activity by key species. The model is used to predict genetic targets breeding and improve the frequency of selecting improvements in microbiome-encoded traits of agronomic relevance.

### Measuring Nitrogen Delivered in an Agriculturally Relevant Field Context

In the field, the amount of nitrogen delivered can be determined by the function of colonization multiplied by the activity.

$$Nitrogen delivered = {\int }_{Time \& Space}^{ } Colonization \times Activity$$

The above equation requires (1) the average colonization per unit of plant tissue, and (2) the activity as either the amount of nitrogen fixed or the amount of ammonia excreted by each microbial cell. To convert to pounds of nitrogen per acre, corn growth physiology is tracked over time, e.g., size of the plant and associated root system throughout the maturity stages.

The pounds of nitrogen delivered to a crop per acre-season can be calculated by the following equation:

Nitrogen delivered=∫Plant Tissue(t)×Colonization(t)×Activity(t)dt

The Plant Tissue(t) is the fresh weight of corn plant tissue over the growing time (t). Values for reasonably making the calculation are described in detail in the publication entitled Roots, Growth and Nutrient Uptake (Mengel. Dept. of Agronomy Pub. #AGRY-95-08 (Rev. May 1995. p. 1-8.).

The Colonization (t) is the amount of the microbes of interest found within the plant tissue, per gram fresh weight of plant tissue, at any particular time, t, during the growing season. In the instance of only a single timepoint available, the single timepoint is normalized as the peak colonization rate over the season, and the colonization rate of the remaining timepoints are adjusted accordingly.

Activity(t) is the rate of which N is fixed by the microbes of interest per unit time, at any particular time, t, during the growing season. In the embodiments disclosed herein, this activity rate is approximated by in vitro acetylene reduction assay (ARA) in ARA media in the presence of 5 mM glutamine or Ammonium excretion assay in ARA media in the presence of 5 mM ammonium ions.

The Nitrogen delivered amount is then calculated by numerically integrating the above function. In cases where the values of the variables described above are discretely measured at set timepoints, the values in between those timepoints are approximated by performing linear interpolation.

### Nitrogen Fixation

Described herein are methods of increasing nitrogen fixation in a plant, comprising exposing the plant to bacteria comprising one or more genetic variations introduced into one or more genes regulating nitrogen fixation, wherein the bacteria produce 1% or more of nitrogen in the plant (e.g. 2%, 5%, 10%, or more), which may represent a nitrogen-fixation capability of at least 2-fold as compared to the plant in the absence of the bacteria. The bacteria may produce the nitrogen in the presence of fertilizer supplemented with glutamine, urea, nitrates or ammonia. Genetic variations can be any genetic variation described herein, including examples provided above, in any number and any combination. The genetic variation may be introduced into a gene selected from the group consisting of nifA, nifL, ntrB, ntrC, glutamine synthetase, glnA, glnB, glnK, draT, amtB, glutaminase, glnD, glnE, nifJ, nifH, nifD, nifK, nifY, nifE, nifN, nifJ, nifS, nifV, nifW, nifZ, nifM, nifF, nifB, and nifQ. The genetic variation may be a mutation that results in one or more of: increased expression or activity of nifA or glutaminase; decreased expression or activity of nifL, ntrB, glutamine synthetase, glnB, glnK, draT, amtB; decreased adenylyl-removing activity of GlnE; or decreased uridylyl-removing activity of GlnD. The genetic variation introduced into one or more bacteria of the methods disclosed herein may be a knock-out mutation or it may abolish a regulatory sequence of a target gene, or it may comprise insertion of a heterologous regulatory sequence, for example, insertion of a regulatory sequence found within the genome of the same bacterial species or genus. The regulatory sequence can be chosen based on the expression level of a gene in a bacterial culture or within plant tissue. The genetic variation may be produced by chemical mutagenesis. The plants grown in step (c) may be exposed to biotic or abiotic stressors.

The amount of nitrogen fixation that occurs in the plants described herein may be measured in several ways, for example by an acetylene-reduction (AR) assay. An acetylene-reduction assay can be performed in vitro or in vivo. Evidence that a particular bacterium is providing fixed nitrogen to a plant can include: 1) total plant N significantly increases upon inoculation, preferably with a concomitant increase in N concentration in the plant; 2) nitrogen deficiency symptoms are relieved under N-limiting conditions upon inoculation (which should include an increase in dry matter); 3) N₂ fixation is documented through the use of an ¹⁵N approach (which can be isotope dilution experiments, ¹⁵N₂ reduction assays, or ¹⁵N natural abundance assays); 4) fixed N is incorporated into a plant protein or metabolite; and 5) all of these effects are not be seen in non-inoculated plants or in plants inoculated with a mutant of the inoculum strain.

The wild-type nitrogen fixation regulatory cascade can be represented as a digital logic circuit where the inputs O₂ and NH₄⁺ pass through a NOR gate, the output of which enters an AND gate in addition to ATP. In some embodiments, the methods disclosed herein disrupt the influence of NH₄⁺ on this circuit, at multiple points in the regulatory cascade, so that microbes can produce nitrogen even in fertilized fields. However, the methods disclosed herein also envision altering the impact of ATP or O₂ on the circuitry, or replacing the circuitry with other regulatory cascades in the cell, or altering genetic circuits other than nitrogen fixation. Gene clusters can be re-engineered to generate functional products under the control of a heterologous regulatory system. By eliminating native regulatory elements outside of, and within, coding sequences of gene clusters, and replacing them with alternative regulatory systems, the functional products of complex genetic operons and other gene clusters can be controlled and/or moved to heterologous cells, including cells of different species other than the species from which the native genes were derived. Once re-engineered, the synthetic gene clusters can be controlled by genetic circuits or other inducible regulatory systems, thereby controlling the products' expression as desired. The expression cassettes can be designed to act as logic gates, pulse generators, oscillators, switches, or memory devices. The controlling expression cassette can be linked to a promoter such that the expression cassette functions as an environmental sensor, such as an oxygen, temperature, touch, osmotic stress, membrane stress, or redox sensor.

As an example, the nifL, nifA, nifT, and nifX genes can be eliminated from the nif gene cluster. Synthetic genes can be designed by codon randomizing the DNA encoding each amino acid sequence. Codon selection is performed, specifying that codon usage be as divergent as possible from the codon usage in the native gene. Proposed sequences are scanned for any undesired features, such as restriction enzyme recognition sites, transposon recognition sites, repetitive sequences, sigma 54 and sigma 70 promoters, cryptic ribosome binding sites, and rho independent terminators. Synthetic ribosome binding sites are chosen to match the strength of each corresponding native ribosome binding site, such as by constructing a fluorescent reporter plasmid in which the 150 bp surrounding a gene's start codon (from −60 to +90) is fused to a fluorescent gene. This chimera can be expressed under control of the Ptac promoter, and fluorescence measured via flow cytometry. To generate synthetic ribosome binding sites, a library of reporter plasmids using 150 bp (−60 to +90) of a synthetic expression cassette is generated. Briefly, a synthetic expression cassette can consist of a random DNA spacer, a degenerate sequence encoding an RBS library, and the coding sequence for each synthetic gene. Multiple clones are screened to identify the synthetic ribosome binding site that best matched the native ribosome binding site. Synthetic operons that consist of the same genes as the native operons are thus constructed and tested for functional complementation. A further exemplary description of synthetic operons is provided in US20140329326.

### Bacterial Species

Microbes useful in the methods and compositions disclosed herein may be obtained from any source. In some cases, microbes may be bacteria, archaea, protozoa or fungi. The microbes of this disclosure may be nitrogen fixing microbes, for example a nitrogen fixing bacteria, nitrogen fixing archaea, nitrogen fixing fungi, nitrogen fixing yeast, or nitrogen fixing protozoa. Microbes useful in the methods and compositions disclosed herein may be spore forming microbes, for example spore forming bacteria. In some cases, bacteria useful in the methods and compositions disclosed herein may be Gram positive bacteria or Gram negative bacteria. In some cases, the bacteria may be an endospore forming bacteria of the Firmicute phylum. In some cases, the bacteria may be a diazatroph. In some cases, the bacteria may not be a diazotroph.

Klebsiella variicola

Klebsiella variicola is a free-living nitrogen fixing soil bacteria, and has been isolated from banana, rice, sugarcane and corn rhizospheres. The 137 strain was originally isolated from a soil sample collected from the rhizosphere of corn roots from a field in Missouri in St. Charles County. The same strain has also been found in fields in California and Puerto Rico, as confirmed by alignments between the 137 strain 16S rRNA and the 16S rRNA of naturally-existing K. variicola organisms in the soils from California and Puerto Rico. Klebsiella variicola is not known to exhibit any plant pest characteristics; although one research group reported that a strain of K. variicola can cause Banana Soft RotI in China. No virulence factors have been found in the genome of the 137 strain of K. variicola.

Kosakonia sacchari

Kosakonia sacchari is a new species within the new genus Kosakonia, which was included in the genus Enterobacter2. K. sacchari is a free-living nitrogen-fixing bacterium named for its association with sugarcane (Saccharum officinarum L.). K. sacchari bacteria are Gram-negative, aerobic, non-spore-forming, motile rods, and are able to colonize and fix nitrogen in association with sugarcane plants, thus promoting plant growth. Strain CI006 was isolated from a soil sample taken from San Joaqin County, California. Kosakonia sacchari is not known to exhibit any plant pest characteristics.

The identities of the two wild-type microbes described above (strains 137 and CI006) were-confirmed by sequence analysis of the 16S rRNA gene, an established method for prokaryotic phylogenetic studies. Biological deposit information for these two strains is included in this application.

The methods and compositions of this disclosure may be used with an archaea, such as, for example, Methanothermobacter thermoautotrophicus.

In some cases, bacteria which may be useful include, but are not limited to, Agrobacterium radiobacter, Bacillus acidocaldarius, Bacillus acidoterrestris, Bacillus agri, Bacillus aizawai, Bacillus albolactis, Bacillus alcalophilus, Bacillus alvei, Bacillus aminoglucosidicus, Bacillus aminovorans, Bacillus amylolyticus (also known as Paenibacillus amylolyticus) Bacillus amyloliquefaciens, Bacillus aneurinolyticus, Bacillus atrophaeus, Bacillus azotoformans, Bacillus badius, Bacillus cereus (synonyms: Bacillus endorhythmos, Bacillus medusa), Bacillus chitinosporus, Bacillus circulans, Bacillus coagulans, Bacillus endoparasiticus Bacillus fastidiosus, Bacillus firmus, Bacillus kurstaki, Bacillus lacticola, Bacillus lactimorbus, Bacillus lactis, Bacillus laterosporus (also known as Brevibacillus laterosporus). Bacillus lautus, Bacillus lentimorbus Bacillus lentus, Bacillus licheniformis, Bacillus maroccanus, Bacillus megaterium, Bacillus metiens, Bacillus mycoides, Bacillus natto, Bacillus nematocida, Bacillus nigrificans, Bacillus nigrum, Bacillus pantothenticus, Bacillus popillae, Bacillus psychrosaccharolyticus, Bacillus pumilus, Bacillus siamensis, Bacillus smithii, Bacillus sphaericus, Bacillus subtilus, Bacillus thuringiensis, Bacillus unflagellatus, Bradyrhizobium japonicum, Brevibacillus brevis Brevibacillus laterosporus (formerly Bacillus laterosporus). Chromobacterium subtsugae, Delftia acidovorans, Lactobacillus acidophilus, Lysobacter antibioticus, Lysobacter enymogenes, Paenibacillus alvei, Paenibacillus polymyxa, Paenibacillus popilliae (formerly Bacillus popilliae). Pantoea agglomerans, Pasteuria penetrans (formerly Bacillus penetrans), Pasteuria usgae, Pectobacterium carotovorum (formerly Erwinia carotovora), Pseudomonas aeruginosa, Pseudomonas aureofaciens, Pseudomonas cepacia (formerly known as Burkholderia cepacia), Pseudomonas chlororaphis, Pseudomonas fluorescens, Pseudomonas proradix, Pseudomonas putida, Pseudomonas syringae, Serratia entomophila, Serratia marcescens, Streptomyces colombiensis, Streptomyces galbus, Streptomyces goshikiensis, Streptomyces griseoviridis, Streptomyces lavendulae, Streptomyces prasinus, Streptomyces saraceticus, Streptomyces venezuelae, Xanthomonas campestris, Xenorhabdus luminescens, Xenorhabdus nematophila, Rhodococcus globerulus AQ719 (NRRL Accession No. B-21663), Bacillus sp. AQ175 (ATCC Accession No. 55608), Bacillus sp. AQ 177 (ATCC Accession No. 55609), Bacillus sp. AQ178 (ATCC Accession No. 53522), and Streptomyces sp. strain NRRL Accession No. B-30145. In some cases the bacterium may be Azotobacter chroococcum, Methanosarcina barkeri Klesiella pneumoniae Azotobacter vinelandii Rhodobacter spharoides Rhodobacter capsulatus, Rhodobcter palustris, Rhodosporillum rubrum, Rhizobium leguminosarum or Rhizobium etli.

In some cases the bacterium may be a species of Clostridium, for example Clostridium pasteurianum, Clostridium beijerinckii, Clostridium perfringens Clostridium tetani, Clostridium acetobutylicum.

In some cases, bacteria used with the methods and compositions of the present disclosure may be cyanobacteria. Examples of cyanobacterial genuses include Anabaena (for example Anagaena sp. PCC7120), Nostoc (for example Nostoc punctiforme), or Synechocystis (for example Synechocystis sp. PCC6803).

In some cases, bacteria used with the methods and compositions of the present disclosure may belong to the phylum Chlorobi, for example Chlorobium tepidum.

In some cases, microbes used with the methods and compositions of the present disclosure may comprise a gene homologous to a known NifH gene. Sequences of known NifH genes may be found in, for example, the Zehr lab NifH database, (https://www.zehr.pmc.ucsc.edu/nifH_Database_Public/, Apr. 4, 2014), or the Buckley lab NifH database (http://www.css.cornell.edu/faculty/buckley/nifh.htm, and Gaby, John Christian, and Daniel H. Buckley. “A comprehensive aligned nifH gene database: a multipurpose tool for studies of nitrogen-fixing bacteria.” Database 2014 (2014): bau001.). In some cases, microbes used with the methods and compositions of the present disclosure may comprise a sequence which encodes a polypeptide with at least 60%, 70%, 80%, 85%, 90%, 95%, 96%, 96%, 98%, 99% or more than 99% sequence identity to a sequence from the Zehr lab NifH database, (https://www.zehr.pmc.ucsc.edu/nifH_Database_Public/, Apr. 4, 2014). In some cases, microbes used with the methods and compositions of the present disclosure may comprise a sequence which encodes a polypeptide with at least 60%, 70%, 80%, 85%, 90%, 95%, 96%, 96%, 98%, 99% or more than 99% sequence identity to a sequence from the Buckley lab NifH database, (Gaby, John Christian, and Daniel H. Buckley. “A comprehensive aligned nifH gene database: a multipurpose tool for studies of nitrogen-fixing bacteria.” Database 2014 (2014): bau001.).

Microbes useful in the methods and compositions disclosed herein can be obtained by extracting microbes from surfaces or tissues of native plants; grinding seeds to isolate microbes; planting seeds in diverse soil samples and recovering microbes from tissues; or inoculating plants with exogenous microbes and determining which microbes appear in plant tissues. Non-limiting examples of plant tissues include a seed, seedling, leaf, cutting, plant, bulb or tuber. In some cases, bacteria are isolated from a seed. The parameters for processing samples may be varied to isolate different types of associative microbes, such as rhizospheric, epiphytes, or endophytes. Bacteria may also be sourced from a repository, such as environmental strain collections, instead of initially isolating from a first plant. The microbes can be genotyped and phenotyped, via sequencing the genomes of isolated microbes; profiling the composition of communities in planta; characterizing the transcriptomic functionality of communities or isolated microbes; or screening microbial features using selective or phenotypic media (e.g., nitrogen fixation or phosphate solubilization phenotypes). Selected candidate strains or populations can be obtained via sequence data; phenotype data; plant data (e.g., genome, phenotype, and/or yield data); soil data (e.g., pH, N/P/K content, and/or bulk soil biotic communities); or any combination of these.

The bacteria and methods of producing bacteria described herein may apply to bacteria able to self-propagate efficiently on the leaf surface, root surface, or inside plant tissues without inducing a damaging plant defense reaction, or bacteria that are resistant to plant defense responses. The bacteria described herein may be isolated by culturing a plant tissue extract or leaf surface wash in a medium with no added nitrogen. However, the bacteria may be unculturable, that is, not known to be culturable or difficult to culture using standard methods known in the art. The bacteria described herein may be an endophyte or an epiphyte or a bacterium inhabiting the plant rhizosphere (rhizospheric bacteria). The bacteria obtained after repeating the steps of introducing genetic variation, exposure to a plurality of plants, and isolating bacteria from plants with an improved trait one or more times (e.g. 1, 2, 3, 4, 5, 10, 15, 25, or more times) may be endophytic, epiphytic, or rhizospheric. Endophytes are organisms that enter the interior of plants without causing disease symptoms or eliciting the formation of symbiotic structures, and are of agronomic interest because they can enhance plant growth and improve the nutrition of plants (e.g., through nitrogen fixation). The bacteria can be a seed-borne endophyte. Seed-borne endophytes include bacteria associated with or derived from the seed of a grass or plant, such as a seed-borne bacterial endophyte found in mature, dry, undamaged (e.g., no cracks, visible fungal infection, or prematurely germinated) seeds. The seed-borne bacterial endophyte can be associated with or derived from the surface of the seed; alternatively, or in addition, it can be associated with or derived from the interior seed compartment (e.g., of a surface-sterilized seed). In some cases, a seed-borne bacterial endophyte is capable of replicating within the plant tissue, for example, the interior of the seed. Also, in some cases, the seed-borne bacterial endophyte is capable of surviving desiccation.

The bacterial isolated according to methods of the disclosure, or used in methods or compositions of the disclosure, can comprise a plurality of different bacterial taxa in combination. By way of example, the bacteria may include Proteobacteria (such as Pseudomonas, Enterobacter, Stenotrophomonas, Burkholderia, Rhizobium, Herbaspirillum, Pantoea, Serratia, Rahnella, Azospirillum, Azorhizobium, Azotobacter, Duganella, Delftia, Bradyrhizobium, Sinorhizobium and Halomonas), Firmicutes (such as Bacillus, Paenibacillus, Lactobacillus, Mycoplasma, and Acetabacterium),and Actinobacteria (such as Streptomyces, Rhodacoccus, Microbacterium, and Curtobacterium). The bacteria used in methods and compositions of this disclosure may include nitrogen fixing bacterial consortia of two or more species. In some cases, one or more bacterial species of the bacterial consortia may be capable of fixing nitrogen. In some cases, one or more species of the bacterial consortia may facilitate or enhance the ability of other bacteria to fix nitrogen. The bacteria which fix nitrogen and the bacteria which enhance the ability of other bacteria to fix nitrogen may be the same or different. In some examples, a bacterial strain may be able to fix nitrogen when in combination with a different bacterial strain, or in a certain bacterial consortia, but may be unable to fix nitrogen in a monoculture. Examples of bacterial genuses which may be found in a nitrogen fixing bacterial consortia include, but are not limited to, Herbaspirillum, Azospirillum, Enterobacter, and Bacillus.

Bacteria that can be produced by the methods disclosed herein include Azotobacter sp. Bradyrhizobium sp. Klebsiella sp., and Sinorhizobium sp. In some cases, the bacteria may be selected from the group consisting of: Azotobacter vinelandii, Bradyrhizobium japonicum, Klebsiella pneumoniae, and Sinorhizobium meliloti. In some cases, the bacteria may be of the genus Enterobacter or Rahnella. In some cases, the bacteria may be of the genus Frankia, or Clostridium. Examples of bacteria of the genus Clostridium include, but are not limited to, Clostridium acetobutilicum, Clostridium pasteurianum, Clostridium beyerinckii, Clostridium perfringens, and Clostridium tetani. In some cases, the bacteria may be of the genus Paenibacillus, for example Paenibacillus azotofixans, Paembacillus borealis, Paenibacillus durus, Paenibacillus macerans, Paenibacillus polymyxa, Paenibacillus alvei, Paenibacillus amylolyticus, Paenibacillus campinasensis, Paenibacillus chibensis, Paenibacillus glucanolyticus, Paenibacillus illinoisensis, Paenibacillus larvae subsp. Larvae, Paenibacillus larvae subsp. Pulvifaciens, Paenibacillus lautus, Paenibacillus macerans, Paenibacillus macquariensis, Paenibacillus macquariensis, Paenibacillus pabuli, Paenibacillus peoriae, or Paenibacillus polymyxa.

In some examples, bacteria isolated according to methods of the disclosure can be a member of one or more of the following taxa: Achromobacter Acidithiobacillus, Acidovorax, Acidovraz, Acinetobacter, Actinoplanes, Adlercreutzia, Aerococcus, Aeromonas, Afipia, Agromyces, Ancylobacter, Arthrobacter, Atopostipes, Azospirillum, Bacillus, Bdellovibrio Beiyerinckia, Bosea, Bradyrhizobium, Brevibacillus, Brevundimonas, Burkholderia, Candidatus Haloredivivus, Caulobacter, Cellulomonas, Cellvibrio, Chryseobacterium, Citrobacter, Clostridium, Coraliomargarita, Corynebacterium, Cupriavidus, Curtobacterium, Curvibacter, Deinococcus, Delftia, Desemzia, Devosia, Dokdonella, Dyella, Enhydrobacter, Enterobacter, Enterococcus, Erwinia, Escherichia, Escherichia/Shigella, Exiguobacterium, Ferroglobus, Filimonas, Finegoldia, Flavisolibacter Flavobacterium, Frigoribacterium, Gluconacetobacter, Hafnia, Halobaculum, Halomonas, Halosimplex Herbaspirillum, Hymenobacter, Klebsiella Kocuria, Kosakonia, Lactobacillus, Leclercia, Lentzea, Luteibacter, Luteimonas, Massilia Mesorhizobium Methylobacterium Microbacterium, Micrococcus, Microvirga, Mycobacterium, Neisseria, Nocardia, Oceanibaculum, Ochrobactrum, Okibacterium, Oligotropha, Oryzihumus, Oxalophagus, Paenibacillus, Panteoa, Pantoea, Pelomonas, Perlucidibaca, Plantibacter, Polynucleobacter, Propionibacterium, Propioniciclava, Pseudoclavibacter, Pseudomonas, Pseudonocardia, Pseudoxanthomonas, Psychrobacter, Rahnella, Ralstonia, Rheinheimera, Rhizobium, Rhodococcus, Rhodopseudomonas, Roseateles, Ruminococcus, Sebaldella, Sediminibacillus, Sediminibacterium, Serratia, Shigella, Shinella, Sinorhizobium, Sinosporangium, Sphingobacterium, Sphingomonas, Sphingopyxis, Sphingosinicella, Staphylococcus, 25 Stenotrophomonas, Strenotrophomonas, Streptococcus, Streptomyces, Stygiolobus, Sulfurisphaera, Tatumella, Tepidimonas, Thermomonas, Thiobacillus, Variovorax, WPS-2 genera Incertae sedis, Xanthomonas, and Zimmermannella.

In some cases, a bacterial species selected from at least one of the following genera are utilized: Enterobacter, Klebsiella, Kosakonia, and Rahnella. In some cases, a combination of bacterial species from the following genera are utilized: Enterobacter, Klebsiella, Kosakonia, and Rahnella. In some cases, the species utilized can be one or more of: Enterobacter sacchari, Klebsiella variicola, Kosakonia sacchari, and Rahnella aquatilis.

In some cases, a Gram positive microbe may have a Molybdenum-Iron nitrogenase system comprising: nifH, nifD, nifK nifB, nifE, nifN, nifX, hesA, nifV, nifW, nifU, nifS, nifI1, and nifI2. In some cases, a Gram positive microbe may have a vanadium nitrogenase system comprising: vnfDG, vnfK, vnfE, vnfN, vupC, vupB, vupA, vnfV, vfnRf1, vnfH, vnfR2, vnfA (transcriptional regulator). In some cases, a Gram positive microbe may have an iron-only nitrogenase system comprising: anfK, anfG, anfD, anfH, anfA (transcriptional regulator). In some cases a Gram positive microbe may have a nitrogenase system comprising glnB, and glnK (nitrogen signaling proteins). Some examples of enzymes involved in nitrogen metabolism in Gram positive microbes include glnA (glutamine synthetase), gdh (glutamate dehydrogenase), bdh (3-hydroxybutyrate dehydrogenase), glutaminase, gltAB/gltB/gltS (glutamate synthase), asnA/asnB (aspartate-ammonia ligase/asparagine synthetase), and ansA/ansZ (asparaginase). Some examples of proteins involved in nitrogen transport in Gram positive microbes include amtB (ammonium transporter), glnK (regulator of ammonium transport), glnPHQ/glnQHMP (ATP-dependent glutamine/glutamate transporters), glnT/alsT/yrbD/yflA (glutamine-like proton symport transporters), and gltP/gltT/yhcl/nqt (glutamate-like proton symport transporters).

Examples of Gram positive microbes which may be of particular interest include Paenibacillus polymixa, Paenibacillus riograndensis, Paenibacillus sp., Frankia sp., Heliobacterium sp., Heliobacterium chlorum, Heliobacillus sp., Heliophilum sp., Heliorestis sp., Clostridium acetobutylicum, Clostridium sp., Mycobacterium flaum, Mycobacterium sp., Arthrobacter sp., Agromyces sp., Corynebacterium autitrophicum, Corynebacterium sp., Micromonspora sp., Propionibacteria sp., Streptomyces sp., and Microbacterium sp.

Some examples of genetic alterations which may be make in Gram positive microbes include: deleting glnR to remove negative regulation of BNF in the presence of environmental nitrogen, inserting different promoters directly upstream of the nif cluster to eliminate regulation by GlnR in response to environmental nitrogen, mutating glnA to reduce the rate of ammonium assimilation by the GS-GOGAT pathway, deleting amtB to reduce uptake of ammonium from the media, mutating glnA so it is constitutively in the feedback-inhibited (FBI-GS) state, to reduce ammonium assimilation by the GS-GOGAT pathway.

In some cases, glnR is the main regulator of N metabolism and fixation in Paenibacillus species. In some cases, the genome of a Paenibacillus species may not contain a gene to produce glnR. In some cases, the genome of a Paenibacillus species may not contain a gene to produce glnE or glnD. In some cases, the genome of a Paenibacillus species may contain a gene to produce glnB or glnK. For example Paenibacillus sp. WLY78 doesn't contain a gene for glnB, or its homologs found in the archaeon Methanococcus maripaludis, nifI1 and nifI2. In some cases, the genomes of Paenibacillus species may be variable. For example, Paenibacillus polymixa E681 lacks glnK and gdh, has several nitrogen compound transporters, but only amtB appears to be controlled by GlnR. In another example, Paenibacillus sp. JDR2 has glnK, gdh and most other central nitrogen metabolism genes, has many fewer nitrogen compound transporters, but does have glnPHQ controlled by GlnR. Paenibacillus riograndensis SBR5 contains a standard glnRA operon, an fdx gene, a main nif operon, a secondary nif operon, and an anf operon (encoding iron-only nitrogenase). Putative glnR/tnrA sites were found upstream of each of these operons. GlnR may regulate all of the above operons, except the anf operon. GlnR may bind to each of these regulatory sequences as a dimer.

Paenibacillus N-fixing strains may fall into two subgroups: Subgroup I, which contains only a minimal nif gene cluster and subgroup II, which contains a minimal cluster, plus an uncharacterized gene between nifX and hesA, and often other clusters duplicating some of the nif genes, such as mJH, nifHDK, nmBEN or clusters encoding vanadaium nitrogenase (vnf) or iron-only nitrogenase (anf) genes.

In some cases, the genome of a Paenibacillus species may not contain a gene to produce glnB or glnK. In some cases, the genome of a Paenibacillus species may contain a minimal nif cluster with 9 genes transcribed from a sigma-70 promoter. In some cases a Paenibacillus nif cluster may be negatively regulated by nitrogen or oxygen. In some cases, the genome of a Paenibacillus species may not contain a gene to produce sigma-54. For example, Paenibacillus sp. WLY78 does not contain a gene for sigma-54. In some cases, a nif cluster may be regulated by glnR, and/or TnrA. In some cases, activity of a nif cluster may be altered by altering activity of glnR, and/or TnrA.

In Bacilli, glutamine synthetase (GS) is feedback-inhibited by high concentrations of intracellular glutamine, causing a shift in confirmation (referred to as FBI-GS). Nif clusters contain distinct binding sites for the regulators GlnR and TnrA in several Bacilli species. GlnR binds and represses gene expression in the presence of excess intracellular glutamine and AMP. A role of GlnR may be to prevent the influx and intracellular production of glutamine and ammonium under conditions of high nitrogen availability. TnrA may bind and/or activate (or repress) gene expression in the presence of limiting intracellular glutamine, and/or in the presence of FBI-GS. In some cases the activity of a Bacilli nif cluster may be altered by altering the activity of GlnR.

Feedback-inhibited glutamine synthetase (FBI-GS) may bind GlnR and stabilize binding of GlnR to recognition sequences. Several bacterial species have a GlnR/TnrA binding site upstream of the nif cluster. Altering the binding of FBI-GS and GlnR may alter the activity of the nif pathway.

### Sources of Microbes

The bacteria (or any microbe according to the disclosure) may be obtained from any general terrestrial environment, including its soils, plants, fungi, animals (including invertebrates) and other biota, including the sediments, water and biota of lakes and rivers; from the marine environment, its biota and sediments (for example, sea water, marine muds, marine plants, marine invertebrates (for example, sponges), marine vertebrates (for example, fish)); the terrestrial and marine geosphere (regolith and rock, for example, crushed subterranean rocks, sand and clays); the cryosphere and its meltwater; the atmosphere (for example, filtered aerial dusts, cloud and rain droplets); urban, industrial and other man-made environments (for example, accumulated organic and mineral matter on concrete, roadside gutters, roof surfaces, and road surfaces).

The plants from which the bacteria (or any microbe according to the disclosure) are obtained may be a plant having one or more desirable traits, for example a plant which naturally grows in a particular environment or under certain conditions of interest. By way of example, a certain plant may naturally grow in sandy soil or sand of high salinity, or under extreme temperatures, or with little water, or it may be resistant to certain pests or disease present in the environment, and it may be desirable for a commercial crop to be grown in such conditions, particularly if they are, for example, the only conditions available in a particular geographic location. By way of further example, the bacteria may be collected from commercial crops grown in such environments, or more specifically from individual crop plants best displaying a trait of interest amongst a crop grown in any specific environment: for example the fastest-growing plants amongst a crop grown in saline-limiting soils, or the least damaged plants in crops exposed to severe insect damage or disease epidemic, or plants having desired quantities of certain metabolites and other compounds, including fiber content, oil content, and the like, or plants displaying desirable colors, taste or smell. The bacteria may be collected from a plant of interest or any material occurring in the environment of interest, including fungi and other animal and plant biota, soil, water, sediments, and other elements of the environment as referred to previously.

The bacteria (or any microbe according to the disclosure) may be isolated from plant tissue, his isolation can occur from any appropriate tissue in the plant, including for example root, stem and leaves, and plant reproductive tissues. By way of example, conventional methods for isolation from plants typically include the sterile excision of the plant material of interest (e.g. root or stem lengths, leaves), surface sterilization with an appropriate solution (e.g. 2% sodium hypochlorite), after which the plant material is placed on nutrient medium for microbial growth. Alternatively, the surface-sterilized plant material can be crushed in a sterile liquid (usually water) and the liquid suspension, including small pieces of the crushed plant material spread over the surface of a suitable solid agar medium, or media, which may or may not be selective (e.g. contain only phytic acid as a source of phosphorus). This approach is especially useful for bacteria which form isolated colonies and can be picked off individually to separate plates of nutrient medium, and further purified to a single species by well-known methods. Alternatively, the plant root or foliage samples may not be surface sterilized but only washed gently thus including surface-dwelling epiphytic microorganisms in the isolation process, or the epiphytic microbes can be isolated separately, by imprinting and lifting off pieces of plant roots, stem or leaves onto the surface of an agar medium and then isolating individual colonies as above. This approach is especially useful for bacteria, for example. Alternatively, the roots may be processed without washing off small quantities of soil attached to the roots, thus including microbes that colonize the plant rhizosphere. Otherwise, soil adhering to the roots can be removed, diluted and spread out onto agar of suitable selective and non-selective media to isolate individual colonies of rhizospheric bacteria.

### Budapest Treaty on the International Recognition of the Deposit of Microorganisms for the Purpose of Patent Procedures

The microbial deposits of the present disclosure were made under the provisions of the Budapest Treaty on the International Recognition of the Deposit of Microorganisms for the Purpose of Patent Procedure (Budapest Treaty).

Applicants state that pursuant to 37 C.F.R. § 1.808(a)(2) “all restrictions imposed by the depositor on the availability to the public of the deposited material will be irrevocably removed upon the granting of the patent.” This statement is subject to paragraph (b) of this section (i.e. 37 C.F.R. § 1.808(b)).

A biologically pure culture of Kosakonia sacchari (WT) was deposited on Jan. 6, 2017 with the Bigelow National Center for Marine Algae and Microbiota (NCMA), located at 60 Bigelow Drive, East Boothbay, Me. 04544, USA, and assigned NCMA Patent Deposit Designation number 201701001. The applicable deposit information is found below in Table 1.

A biologically pure culture of Klebsiella variicola (WT) was deposited on Aug. 11, 2017 with the Bigelow National Center for Marine Algae and Microbiota (NCMA), located at 60 Bigelow Drive, East Boothbay, Me. 04544, USA, and assigned NCMA Patent Deposit Designation number 201708001. The applicable deposit information is found below in Table 1.

TABLE 1
Microorganisms Deposited under the Budapest Treaty

| | Pivot Strain | | | |
|---|---|---|---|---|
| | Designation | | | |
| | (some strains | | | |
| De- | have multiple | | Accession | Date of |
| pository | designations) | Taxonomy | Number | Deposit |
| NCMA | C1006, PBC6.1, | Kosakonia | 201701001 | Jan. 06, 2017 |
| | 6 | sacchari (WT) | | |
| NCMA | C1137, 137, | Klebsiella | 201708001 | Aug. 11, 2017 |
| | PB137 | variicola (WT) | | |

### Isolated and Biologically Pure Microorganisms

The present disclosure, in certain embodiments, provides isolated and biologically pure microorganisms that have applications, inter alia, in agriculture. The disclosed microorganisms can be utilized in their isolated and biologically pure states, as well as being formulated into compositions (see below section for exemplary composition descriptions). Furthermore, the disclosure provides microbial compositions containing at least two members of the disclosed isolated and biologically pure microorganisms, as well as methods of utilizing said microbial compositions. Furthermore, the disclosure provides for methods of modulating nitrogen fixation in plants via the utilization of the disclosed isolated and biologically pure microbes.

In some aspects, the isolated and biologically pure microorganisms of the disclosure are those from Table 1. In other aspects, the isolated and biologically pure microorganisms of the disclosure are derived from a microorganism of Table 1. For example, a strain, child, mutant, or derivative, of a microorganism from Table 1 are provided herein. The disclosure contemplates all possible combinations of microbes listed in Table 1, said combinations sometimes forming a microbial consortia. The microbes from Table 1, either individually or in any combination, can be combined with any plant, active (synthetic, organic, etc.), adjuvant, carrier, supplement, or biological, mentioned in the disclosure.

### Compositions

Compositions comprising bacteria or bacterial populations produced according to methods described herein and/or having characteristics as described herein can be in the form of a liquid, a foam, or a dry product. Compositions comprising bacteria or bacterial populations produced according to methods described herein and/or having characteristics as described herein may also be used to improve plant traits. In some examples, a composition comprising bacterial populations may be in the form of a dry powder, a slurry of powder and water, or a flowable seed treatment. The compositions comprising bacterial populations may be coated on a surface of a seed, and may be in liquid form.

The composition can be fabricated in bioreactors such as continuous stirred tank reactors, batch reactors, and on the farm. In some examples, compositions can be stored in a container, such as a jug or in mini bulk. In some examples, compositions may be stored within an object selected from the group consisting of a bottle, jar, ampule, package, vessel, bag, box, bin, envelope, carton, container, silo, shipping container, truck bed, and/or case.

Compositions may also be used to improve plant traits. In some examples, one or more compositions may be coated onto a seed. In some examples, one or more compositions may be coated onto a seedling. In some examples, one or more compositions may be coated onto a surface of a seed. In some examples, one or more compositions may be coated as a layer above a surface of a seed. In some examples, a composition that is coated onto a seed may be in liquid form, in dry product form, in foam form, in a form of a slurry of powder and water, or in a flowable seed treatment. In some examples, one or more compositions may be applied to a seed and/or seedling by spraying, immersing, coating, encapsulating, and/or dusting the seed and/or seedling with the one or more compositions. In some examples, multiple bacteria or bacterial populations can be coated onto a seed and/or a seedling of the plant. In some examples, at least two, at least three, at least four, at least five, at least six, at least seven, at least eight, at least nine, at least ten, or more than ten bacteria of a bacterial combination can be selected from one of the following genera: Acidovorax Agrobacterium, Bacillus, Burkholderia, Chryseobacterium, Curtobacterium, Enterobacter, Escherichia, Methylobacterium, Paenibacillus, Pantoea, Pseudomonas, Ralstonia, Saccharibacillus, Sphingomonas, and Stenotrophomonas.

In some examples, at least two, at least three, at least four, at least five, at least six, at least seven, at least eight, at least nine, at least ten, or more than ten bacteria and bacterial populations of an endophytic combination are selected from one of the following families: Bacillaceae, Burkholderiaceae, Comamonadaceae, Enterobacteriaceae, Flavobacteriaceae Methylobacteriaceae, Microbacteriaceae, Paenibacillileae, Pseudomonnaceae, Rhizobiaceae, Sphingomonadaceae, Xanthomonadaceae, Cladosporiaceae, Gnomoniaceae, Incertae sedis. Lasiosphaeriaceae, Netriaceae, and Pleosporaceae.

In some examples, at least two, at least three, at least four, at least five, at least six, at least seven, at least eight, at least night, at least ten, or more than ten bacteria and bacterial populations of an endophytic combination are selected from one of the following families: Bacillaceae, Burkholderiaceae, Comamonadaceae, Enterobacteriaceae, Flavobacteriaceae Methylobacteriaceae, Microbacteriaceae, Paenibacillileae, Pseudomonnaceae, Rhizobiaceae, Sphingomonadaceae, Xanthomonadaceae, Cladosporiaceae, Gnomoniaceae, Incertae sedis. Lasiosphaeriaceae, Netriaceae, Pleosporaceae.

Examples of compositions may include seed coatings for commercially important agricultural crops, for example, sorghum, canola, tomato, strawberry, barley, rice, maize, and wheat. Examples of compositions can also include seed coatings for corn, soybean, canola, sorghum, potato, rice, vegetables, cereals, and oilseeds. Seeds as provided herein can be genetically modified organisms (GMO), non-GMO, organic, or conventional. In some examples, compositions may be sprayed on the plant aerial parts, or applied to the roots by inserting into furrows in which the plant seeds are planted, watering to the soil, or dipping the roots in a suspension of the composition. In some examples, compositions may be dehydrated in a suitable manner that maintains cell viability and the ability to artificially inoculate and colonize host plants. The bacterial species may be present in compositions at a concentration of between 10⁸ to 10¹¹ CFU/ml. In some examples, compositions may be supplemented with trace metal ions, such as molybdenum ions, iron ions, manganese ions, or combinations of these ions. The concentration of ions in examples of compositions as described herein may between about 0.1 mM and about 50 mM. Some examples of compositions may also be formulated with a carrier, such as beta-glucan, carboxylmethyl cellulose (CMC), bacterial extracellular polymeric substance (EPS), sugar, animal milk, or other suitable carriers. In some examples, peat or planting materials can be used as a carrier, or biopolymers in which a composition is entrapped in the biopolymer can be used as a carrier. The compositions comprising the bacterial populations described herein can improve plant traits, such as promoting plant growth, maintaining high chlorophyll content in leaves, increasing fruit or seed numbers, and increasing fruit or seed unit weight.

The compositions comprising the bacterial populations described herein may be coated onto the surface of a seed. As such, compositions comprising a seed coated with one or more bacteria described herein are also contemplated. The seed coating can be formed by mixing the bacterial population with a porous, chemically inert granular carrier. Alternatively, the compositions may be inserted directly into the furrows into which the seed is planted or sprayed onto the plant leaves or applied by dipping the roots into a suspension of the composition. An effective amount of the composition can be used to populate the sub-soil region adjacent to the roots of the plant with viable bacterial growth, or populate the leaves of the plant with viable bacterial growth. In general, an effective amount is an amount sufficient to result in plants with improved traits (e.g. a desired level of nitrogen fixation).

Bacterial compositions described herein can be formulated using an agriculturally acceptable carrier. The formulation useful for these embodiments may include at least one member selected from the group consisting of a tackifier, a microbial stabilizer, a fungicide, an antibacterial agent, a preservative, a stabilizer, a surfactant, an anti-complex agent, an herbicide, a nematicide, an insecticide, a plant growth regulator, a fertilizer, a rodenticide, a dessicant, a bactericide, a nutrient, or any combination thereof. In some examples, compositions may be shelf-stable. For example, any of the compositions described herein can include an agriculturally acceptable carrier (e.g., one or more of a fertilizer such as a non-naturally occurring fertilizer, an adhesion agent such as a non-naturally occurring adhesion agent, and a pesticide such as a non-naturally occurring pesticide). A non-naturally occurring adhesion agent can be, for example, a polymer, copolymer, or synthetic wax. For example, any of the coated seeds, seedlings, or plants described herein can contain such an agriculturally acceptable carrier in the seed coating. In any of the compositions or methods described herein, an agriculturally acceptable carrier can be or can include a non-naturally occurring compound (e.g., a non-naturally occurring fertilizer, a non-naturally occurring adhesion agent such as a polymer, copolymer, or synthetic wax, or a non-naturally occurring pesticide). Non-limiting examples of agriculturally acceptable carriers are described below. Additional examples of agriculturally acceptable carriers are known in the art.

In some cases, bacteria are mixed with an agriculturally acceptable carrier. The carrier can be a solid carrier or liquid carrier, and in various forms including microspheres, powders, emulsions and the like. The carrier may be any one or more of a number of carriers that confer a variety of properties, such as increased stability, wettability, or dispersability. Wetting agents such as natural or synthetic surfactants, which can be nonionic or ionic surfactants, or a combination thereof can be included in the composition. Water-in-oil emulsions can also be used to formulate a composition that includes the isolated bacteria (see, for example, U.S. Pat. No. 7,485,451). Suitable formulations that may be prepared include wettable powders, granules, gels, agar strips or pellets, thickeners, and the like, microencapsulated particles, and the like, liquids such as aqueous flowables, aqueous suspensions, water-in-oil emulsions, etc. The formulation may include grain or legume products, for example, ground grain or beans, broth or flour derived from grain or beans, starch, sugar, or oil.

In some embodiments, the agricultural carrier may be soil or a plant growth medium. Other agricultural carriers that may be used include water, fertilizers, plant-based oils, humectants, or combinations thereof. Alternatively, the agricultural carrier may be a solid, such as diatomaceous earth, loam, silica, alginate, clay, bentonite, vermiculite, seed cases, other plant and animal products, or combinations, including granules, pellets, or suspensions. Mixtures of any of the aforementioned ingredients are also contemplated as carriers, such as but not limited to, pesta (flour and kaolin clay), agar or flour-based pellets in loam, sand, or clay, etc. Formulations may include food sources for the bacteria, such as barley, rice, or other biological materials such as seed, plant parts, sugar cane bagasse, hulls or stalks from grain processing, ground plant material or wood from building site refuse, sawdust or small fibers from recycling of paper, fabric, or wood.

For example, a fertilizer can be used to help promote the growth or provide nutrients to a seed, seedling, or plant. Non-limiting examples of fertilizers include nitrogen, phosphorous, potassium, calcium, sulfur, magnesium, boron, chloride, manganese, iron, zinc, copper, molybdenum, and selenium (or a salt thereof). Additional examples of fertilizers include one or more amino acids, salts, carbohydrates, vitamins, glucose, NaCl, yeast extract, NH₄H₂PO₄, (NH₄)₂SO₄, glycerol, valine, L-leucine, lactic acid, propionic acid, succinic acid, malic acid, citric acid, KH tartrate, xylose, lyxose, and lecithin. In one embodiment, the formulation can include a tackifier or adherent (referred to as an adhesive agent) to help bind other active agents to a substance (e.g., a surface of a seed). Such agents are useful for combining bacteria with carriers that can contain other compounds (e.g., control agents that are not biologic), to yield a coating composition. Such compositions help create coatings around the plant or seed to maintain contact between the microbe and other agents with the plant or plant part. In one embodiment, adhesives are selected from the group consisting of: alginate, gums, starches, lecithins, formononetin, polyvinyl alcohol, alkali formononetinate, hesperetin, polyvinyl acetate, cephalins, Gum Arabic, Xanthan Gum, Mineral Oil, Polyethylene Glycol (PEG), Polyvinyl pyrrolidone (PVP), Arabino-galactan, Methyl Cellulose, PEG 400, Chitosan, Polyacrylamide, Polyacrylate, Polyacrylonitrile, Glycerol, Triethylene glycol, Vinyl Acetate, Gellan Gum, Polystyrene, Polyvinyl, Carboxymethyl cellulose, Gum Ghatti, and polyoxyethylene-polyoxybutylene block copolymers.

In some embodiments, the adhesives can be, e.g. a wax such as carnauba wax, beeswax, Chinese wax, shellac wax, spermaceti wax, candelilla wax, castor wax, ouricury wax, and rice bran wax, a polysaccharide (e.g., starch, dextrins, maltodextrins, alginate, and chitosans), a fat, oil, a protein (e.g., gelatin and zeins), gum arables, and shellacs. Adhesive agents can be non-naturally occurring compounds, e.g., polymers, copolymers, and waxes. For example, non-limiting examples of polymers that can be used as an adhesive agent include: polyvinyl acetates, polyvinyl acetate copolymers, ethylene vinyl acetate (EVA) copolymers, polyvinyl alcohols, polyvinyl alcohol copolymers, celluloses (e.g., ethylcelluloses, methylcelluloses, hydroxymethylcelluloses, hydroxypropylcelluloses, and carboxymethylcelluloses), polyvinylpyrolidones, vinyl chloride, vinylidene chloride copolymers, calcium lignosulfonates, acrylic copolymers, polyvinylacrylates, polyethylene oxide, acylamide polymers and copolymers, polyhydroxyethyl acrylate, methylacrylamide monomers, and polychloroprene.

In some examples, one or more of the adhesion agents, anti-fungal agents, growth regulation agents, and pesticides (e.g., insecticide) are non-naturally occurring compounds (e.g., in any combination). Additional examples of agriculturally acceptable carriers include dispersants (e.g., polyvinylpyrrolidone/vinyl acetate PVPIVA S-630), surfactants, binders, and filler agents.

The formulation can also contain a surfactant. Non-limiting examples of surfactants include nitrogen-surfactant blends such as Prefer 28 (Cenex), Surf-N(US), Inhance (Brandt), P-28 (Wilfarm) and Patrol (Helena); esterified seed oils include Sun-It II (AmCy), MSO (UAP), Scoil (Agsco), Hasten (Wilfarm) and Mes-100 (Drexel); and organo-silicone surfactants include Silwet L77 (UAP), Silikin (Terra), Dyne-Amic (Helena), Kinetic (Helena), Sylgard 309 (Wilbur-Ellis) and Century (Precision). In one embodiment, the surfactant is present at a concentration of between 0.01% v/v to 10% v/v. In another embodiment, the surfactant is present at a concentration of between 0.1% v/v to 1% v/v.

In certain cases, the formulation includes a microbial stabilizer. Such an agent can include a desiccant, which can include any compound or mixture of compounds that can be classified as a desiccant regardless of whether the compound or compounds are used in such concentrations that they in fact have a desiccating effect on a liquid inoculant. Such desiccants are ideally compatible with the bacterial population used, and should promote the ability of the microbial population to survive application on the seeds and to survive desiccation. Examples of suitable desiccants include one or more of trehalose, sucrose, glycerol, and Methylene glycol. Other suitable desiccants include, but are not limited to, non reducing sugars and sugar alcohols (e.g., mannitol or sorbitol). The amount of desiccant introduced into the formulation can range from about 5% to about 50% by weight/volume, for example, between about 10% to about 40%, between about 15% to about 35%, or between about 20% to about 30%. In some cases, it is advantageous for the formulation to contain agents such as a fungicide, an antibacterial agent, an herbicide, a nematicide, an insecticide, a plant growth regulator, a rodenticide, bactericide, or a nutrient. In some examples, agents may include protectants that provide protection against seed surface-borne pathogens. In some examples, protectants may provide some level of control of soil-borne pathogens. In some examples, protectants may be effective predominantly on a seed surface.

In some examples, a fungicide may include a compound or agent, whether chemical or biological, that can inhibit the growth of a fungus or kill a fungus. In some examples, a fungicide may include compounds that may be fungistatic or fungicidal. In some examples, fungicide can be a protectant, or agents that are effective predominantly on the seed surface, providing protection against seed surface-borne pathogens and providing some level of control of soil-borne pathogens. Non-limiting examples of protectant fungicides include captan, maneb, thiram, or fludioxonil.

In some examples, fungicide can be a systemic fungicide, which can be absorbed into the emerging seedling and inhibit or kill the fungus inside host plant tissues. Systemic fungicides used for seed treatment include, but are not limited to the following: azoxystrobin, carboxin, mefenoxam, metalaxyl, thiabendazole, trifloxystrobin, and various triazole fungicides, including difenoconazole, ipconazole, tebuconazole, and triticonazole. Mefenoxam and metalaxyl are primarily used to target the water mold fungi Pythium and Phytophthora. Some fungicides are preferred over others, depending on the plant species, either because of subtle differences in sensitivity of the pathogenic fungal species, or because of the differences in the fungicide distribution or sensitivity of the plants. In some examples, fungicide can be a biological control agent, such as a bacterium or fungus. Such organisms may be parasitic to the pathogenic fungi, or secrete toxins or other substances which can kill or otherwise prevent the growth of fungi. Any type of fungicide, particularly ones that are commonly used on plants, can be used as a control agent in a seed composition.

In some examples, the seed coating composition comprises a control agent which has antibacterial properties. In one embodiment, the control agent with antibacterial properties is selected from the compounds described herein elsewhere. In another embodiment, the compound is Streptomycin, oxytetracycline, oxolinic acid, or gentamicin. Other examples of antibacterial compounds which can be used as part of a seed coating composition include those based on dichlorophene and benzylalcohol hemi formal (Proxel® from ICI or Acticide® RS from Thor Chemie and Kathon® MK 25 from Rohm & Haas) and isothiazolinone derivatives such as alkylisothiazolinones and benzisothiazolinones (Acticide® MBS from Thor Chemie).

In some examples, growth regulator is selected from the group consisting of: Abscisic acid, amidochlor, ancymidol, 6-benzylaminopurine, brassinolide, butralin, chlormequat (chlormequat chloride), choline chloride, cyclanilide, daminozide, dikegulac, dimethipin, 2,6-dimethylpuridine, ethephon, flumetralin, flurprimidol, fluthiacet, forchlorfenuron, gibberellic acid, inabenfide, indole-3-acetic acid, maleic hydrazide, mefluidide, mepiquat (mepiquat chloride), naphthaleneacetic acid, N-6-benzyladenine, paclobutrazol, prohexadione phosphorotrithioate, 2,3,5-tri-iodobenzoic acid, trinexapac-ethyl and uniconazole. Additional non-limiting examples of growth regulators include brassinosteroids, cytokinines (e.g., kinetin and zeatin), auxins (e.g., indolylacetic acid and indolylacetyl aspartate), flavonoids and isoflavanoids (e.g., formononetin and diosmetin), phytoaixins (e.g., glyceolline), and phytoalexin-inducing oligosaccharides (e.g., pectin, chitin, chitosan, polygalacuronic acid, and oligogalacturonic acid), and gibellerins. Such agents are ideally compatible with the agricultural seed or seedling onto which the formulation is applied (e.g., it should not be deleterious to the growth or health of the plant). Furthermore, the agent is ideally one which does not cause safety concerns for human, animal or industrial use (e.g., no safety issues, or the compound is sufficiently labile that the commodity plant product derived from the plant contains negligible amounts of the compound).

Some examples of nematode-antagonistic biocontrol agents include ARF18; 30 Arthrobotrys spp.; Chaetomium spp.; Cylindrocarpon spp.; Exophilia spp.; Fusarium spp.; Gliocladium spp.; Hirsutella spp.; Lecanicillium spp.; Monacrosporium spp.; Myrothecium spp.; Neocosmospora spp.; Paecilomyces spp.; Pochonia spp.; Stagonospora spp.; vesicular-arbuscular mycorrhizal fungi, Burkholderia spp.; Pasteuria spp., Brevibacillus spp.; Pseudomonas spp.; and Rhizobacteria. Particularly preferred nematode-antagonistic biocontrol agents include ARF18, Arthrobotrys oligospora, Arthrobotrys dactyloides, Chaetomium globosum, Cylindrocarpon heteronema, Exophilia jeanselmei, Exophilia pisciphila, Fusarium aspergilus, Fusarium solani, Gliocladium catenulatum, Gliocladium roseum, Gliocladium vixens, Hirsutella rhossiliensis, Hirsutella minnesotensis, Lecanicillium lecanii, Monacrosporium drechsleri, Monacrosporium gephyropagum, Myrotehcium verrucaria, Neocosmospora vasinfecta, Paecilomyces lilacinus, Pochonia chlamydosporia, Stagonospora heteroderae, Stagonospora phaseoli, vesicular-arbuscular mycorrhizal fungi, Burkholderia cepacia, Pasteuria penetrans, Pasteuria thomei, Pasteuria nishizawae, Pasteuria ramosa, Pastrueia usage, Brevibacillus laterosporus strain G4, Pseudomonas fluorescens and Rhizobacteria.

Some examples of nutrients can be selected from the group consisting of a nitrogen fertilizer including, but not limited to Urea, Ammonium nitrate, Ammonium sulfate, Non-pressure nitrogen solutions, Aqua ammonia, Anhydrous ammonia, Ammonium thiosulfate, Sulfur-coated urea, Urea-formaldehydes, IBDU, Polymer-coated urea, Calcium nitrate, Ureaform, and Methylene urea, phosphorous fertilizers such as Diammonium phosphate, Monoammonium phosphate, Ammonium polyphosphate, Concentrated superphosphate and Triple superphosphate, and potassium fertilizers such as Potassium chloride, Potassium sulfate, Potassium-magnesium sulfate, Potassium nitrate. Such compositions can exist as free salts or ions within the seed coat composition. Alternatively, nutrients/fertilizers can be complexed or chelated to provide sustained release over time.

Some examples of rodenticides may include selected from the group of substances consisting of 2-isovalerylindan-1,3-dione, 4-(quinoxalin-2-ylamino) benzenesulfonamide, alpha-chlorohydrin, aluminum phosphide, antu, arsenous oxide, barium carbonate, bisthiosemi, brodifacoum, bromadiolone, bromethalin, calcium cyanide, chloralose, chlorophacinone, cholecalciferol, coumachlor, coumafuryl, coumatetralyl, crimidine, difenacoum, difethialone, diphacinone, ergocalciferol, flocoumafen, fluoroacetamide, flupropadine, flupropadine hydrochloride, hydrogen cyanide, iodomethane, lindane, magnesium phosphide, methyl bromide, norbormide, phosacetim, phosphine, phosphorus, pindone, potassium arsenite, pyrinuron, scilliroside, sodium arsenite, sodium cyanide, sodium fluoroacetate, strychnine, thallium sulfate, warfarin and zinc phosphide.

In the liquid form, for example, solutions or suspensions, bacterial populations can be mixed or suspended in water or in aqueous solutions. Suitable liquid diluents or carriers include water, aqueous solutions, petroleum distillates, or other liquid carriers.

Solid compositions can be prepared by dispersing the bacterial populations in and on an appropriately divided solid carrier, such as peat, wheat, bran, vermiculite, clay, talc, bentonite, diatomaceous earth, fuller's earth, pasteurized soil, and the like. When such formulations are used as wettable powders, biologically compatible dispersing agents such as non-ionic, anionic, amphoteric, or cationic dispersing and emulsifying agents can be used.

The solid carriers used upon formulation include, for example, mineral carriers such as kaolin clay, pyrophyllite, bentonite, montmorillonite, diatomaceous earth, acid white soil, vermiculite, and pearlite, and inorganic salts such as ammonium sulfate, ammonium phosphate, ammonium nitrate, urea, ammonium chloride, and calcium carbonate. Also, organic fine powders such as wheat flour, wheat bran, and rice bran may be used. The liquid carriers include vegetable oils such as soybean oil and cottonseed oil, glycerol, ethylene glycol, polyethylene glycol, propylene glycol, polypropylene glycol, etc.

### Application of Bacterial Populations on Crops

The composition of the bacteria or bacterial population described herein can be applied in furrow, in talc, or as seed treatment. The composition can be applied to a seed package in bulk, mini bulk, in a bag, or in talc.

The planter can plant the treated seed and grows the crop according to conventional ways, twin row, or ways that do not require tilling. The seeds can be distributed using a control hopper or an individual hopper. Seeds can also be distributed using pressurized air or manually. Seed placement can be performed using variable rate technologies. Additionally, application of the bacteria or bacterial population described herein may be applied using variable rate technologies. In some examples, the bacteria can be applied to seeds of corn, soybean, canola, sorghum, potato, rice, vegetables, cereals, pseudocereals, and oilseeds. Examples of cereals may include barley, fonio, oats, palmer's grass, rye, pearl millet, sorghum, spelt, teff, triticale, and wheat. Examples of pseudocereals may include breadnut, buckwheat, cattail, chia, flax, grain amaranth, hanza, quinoa, and sesame. In some examples, seeds can be genetically modified organisms (GMO), non-GMO, organic or conventional.

Additives such as micro-fertilizer, PGR, herbicide, insecticide, and fungicide can be used additionally to treat the crops. Examples of additives include crop protectants such as insecticides, nematicides, fungicide, enhancement agents such as colorants, polymers, pelleting, priming, and disinfectants, and other agents such as inoculant, PGR, softener, and micronutrients. PGRs can be natural or synthetic plant hormones that affect root growth, flowering, or stem elongation. PGRs can include auxins, gibberellins, cytokinins, ethylene, and abscisic acid (ABA).

The composition can be applied in furrow in combination with liquid fertilizer. In some examples, the liquid fertilizer may be held in tanks. NPK fertilizers contain macronutrients of sodium, phosphorous, and potassium.

The composition may improve plant traits, such as promoting plant growth, maintaining high chlorophyll content in leaves, increasing fruit or seed numbers, and increasing fruit or seed unit weight Methods of the present disclosure may be employed to introduce or improve one or more of a variety of desirable traits. Examples of traits that may introduced or improved include: root biomass, root length, height, shoot length, leaf number, water use efficiency, overall biomass, yield, fruit size, grain size, photosynthesis rate, tolerance to drought, heat tolerance, salt tolerance, tolerance to low nitrogen stress, nitrogen use efficiency, resistance to nematode stress, resistance to a fungal pathogen, resistance to a bacterial pathogen, resistance to a viral pathogen, level of a metabolite, modulation in level of a metabolite, proteome expression. The desirable traits, including height, overall biomass, root and/or shoot biomass, seed germination, seedling survival, photosynthetic efficiency, transpiration rate, seed/fruit number or mass, plant grain or fruit yield, leaf chlorophyll content, photosynthetic rate, root length, or any combination thereof, can be used to measure growth, and compared with the growth rate of reference agricultural plants (e.g., plants without the introduced and/or improved traits) grown under identical conditions. In some examples, the desirable traits, including height, overall biomass, root and/or shoot biomass, seed germination, seedling survival, photosynthetic efficiency, transpiration rate, seed/fruit number or mass, plant grain or fruit yield, leaf chlorophyll content, photosynthetic rate, root length, or any combination thereof, can be used to measure growth, and compared with the growth rate of reference agricultural plants (e.g., plants without the introduced and/or improved traits) grown under similar conditions.

An agronomic trait to a host plant may include, but is not limited to, the following: altered oil content, altered protein content, altered seed carbohydrate composition, altered seed oil composition, and altered seed protein composition, chemical tolerance, cold tolerance, delayed senescence, disease resistance, drought tolerance, ear weight, growth improvement, health e4nhancement, heat tolerance, herbicide tolerance, herbivore resistance improved nitrogen fixation, improved nitrogen utilization, improved root architecture, improved water use efficiency, increased biomass, increased root length, increased seed weight, increased shoot length, increased yield, increased yield under water-limited conditions, kernel mass, kernel moisture content, metal tolerance, number of ears, number of kernels per ear, number of pods, nutrition enhancement, pathogen resistance, pest resistance, photosynthetic capability improvement, salinity tolerance, stay-green, vigor improvement, increased dry weight of mature seeds, increased fresh weight of mature seeds, increased number of mature seeds per plant, increased chlorophyll content, increased number of pods per plant, increased length of pods per plant, reduced number of wilted leaves per plant, reduced number of severely wilted leaves per plant, and increased number of non-wilted leaves per plant, a detectable modulation in the level of a metabolite, a detectable modulation in the level of a transcript, and a detectable modulation in the proteome, compared to an isoline plant grown from a seed without said seed treatment formulation.

In some cases, plants are inoculated with bacteria or bacterial populations that are isolated from the same species of plant as the plant element of the inoculated plant. For example, a bacterium or bacterial population that is normally found in one variety of Zea mays (corn) is associated with a plant element of a plant of another variety of Zea mays that in its natural state lacks said bacteria and bacterial populations. In one embodiment, the bacteria and bacterial populations is derived from a plant of a related species of plant as the plant element of the inoculated plant. For example, an bacteria and bacterial populations that is normally found in Zea diploperennis Iltis et al., (diploperennial teosinte) is applied to a Zea mays (corn), or vice versa. In some cases, plants are inoculated with bacteria and bacterial populations that are heterologous to the plant element of the inoculated plant. In one embodiment, the bacteria and bacterial populations is derived from a plant of another species. For example, an bacteria and bacterial populations that is normally found in dicots is applied to a monocot plant (e.g., inoculating corn with a soybean-derived bacteria and bacterial populations), or vice versa. In other cases, the bacteria and bacterial populations to be inoculated onto a plant is derived from a related species of the plant that is being inoculated. In one embodiment, the bacteria and bacterial populations is derived from a related taxon, for example, from a related species. The plant of another species can be an agricultural plant. In another embodiment, the bacteria and bacterial populations is part of a designed composition inoculated into any host plant element.

In some examples, the bacteria or bacterial population is exogenous wherein the bacteria and bacterial population is isolated from a different plant than the inoculated plant. For example, in one embodiment, the bacteria or bacterial population can be isolated from a different plant of the same species as the inoculated plant. In some cases, the bacteria or bacterial population can be isolated from a species related to the inoculated plant.

In some examples, the bacteria and bacterial populations described herein are capable of moving from one tissue type to another. For example, the present invention's detection and isolation of bacteria and bacterial populations within the mature tissues of plants after coating on the exterior of a seed demonstrates their ability to move from seed exterior into the vegetative tissues of a maturing plant. Therefore, in one embodiment, the population of bacteria and bacterial populations is capable of moving from the seed exterior into the vegetative tissues of a plant. In one embodiment, the bacteria and bacterial populations that is coated onto the seed of a plant is capable, upon germination of the seed into a vegetative state, of localizing to a different tissue of the plant. For example, bacteria and bacterial populations can be capable of localizing to any one of the tissues in the plant, including: the root, adventitious root, seminal 5 root, root hair, shoot, leaf, flower, bud, tassel, meristem, pollen, pistil, ovaries, stamen, fruit, stolon, rhizome, nodule, tuber, trichome, guard cells, hydathode, petal, sepal, glume, rachis, vascular cambium, phloem, and xylem. In one embodiment, the bacteria and bacterial populations is capable of localizing to the root and/or the root hair of the plant. In another embodiment, the bacteria and bacterial populations is capable of localizing to the photosynthetic tissues, for example, leaves and shoots of the plant. In other cases, the bacteria and bacterial populations is localized to the vascular tissues of the plant, for example, in the xylem and phloem. In still another embodiment, the bacteria and bacterial populations is capable of localizing to the reproductive tissues (flower, pollen, pistil, ovaries, stamen, fruit) of the plant. In another embodiment, the bacteria and bacterial populations is capable of localizing to the root, shoots, leaves and reproductive tissues of the plant. In still another embodiment, the bacteria and bacterial populations colonizes a fruit or seed tissue of the plant. In still another embodiment, the bacteria and bacterial populations is able to colonize the plant such that it is present in the surface of the plant (i.e., its presence is detectably present on the plant exterior, or the episphere of the plant). In still other embodiments, the bacteria and bacterial populations is capable of localizing to substantially all, or all, tissues of the plant. In certain embodiments, the bacteria and bacterial populations is not localized to the root of a plant. In other cases, the bacteria and bacterial populations is not localized to the photosynthetic tissues of the plant.

The effectiveness of the compositions can also be assessed by measuring the relative maturity of the crop or the crop heating unit (CHU). For example, the bacterial population can be applied to corn, and corn growth can be assessed according to the relative maturity of the corn kernel or the time at which the corn kernel is at maximum weight. The crop heating unit (CHU) can also be used to predict the maturation of the corn crop. The CHU determines the amount of heat accumulation by measuring the daily maximum temperatures on crop growth.

In examples, bacterial may localize to any one of the tissues in the plant, including: the root, adventitious root, seminal root, root hair, shoot, leaf, flower, bud tassel, meristem, pollen, pistil, ovaries, stamen, fruit, stolon, rhizome, nodule, tuber, trichome, guard cells, hydathode, petal, sepal, glume, rachis, vascular cambium, phloem, and xylem. In another embodiment, the bacteria or bacterial population is capable of localizing to the photosynthetic tissues, for example, leaves and shoots of the plant. In other cases, the bacteria and bacterial populations is localized to the vascular tissues of the plant, for example, in the xylem and phloem. In another embodiment, the bacteria or bacterial population is capable of localizing to reproductive tissues (flower, pollen, pistil, ovaries, stamen, or fruit) of the plant. In another embodiment, the bacteria and bacterial populations is capable of localizing to the root, shoots, leaves and reproductive tissues of the plant. In another embodiment, the bacteria or bacterial population colonizes a fruit or seed tissue of the plant. In still another embodiment, the bacteria or bacterial population is able to colonize the plant such that it is present in the surface of the plant. In another embodiment, the bacteria or bacterial population is capable of localizing to substantially all, or all, tissues of the plant. In certain embodiments, the bacteria or bacterial population is not localized to the root of a plant. In other cases, the bacteria and bacterial populations is not localized to the photosynthetic tissues of the plant.

The effectiveness of the bacterial compositions applied to crops can be assessed by measuring various features of crop growth including, but not limited to, planting rate, seeding vigor, root strength, drought tolerance, plant height, dry down, and test weight.

### Plant Species

The methods and bacteria described herein are suitable for any of a variety of plants, such as plants in the genera Hordeum, Oryza, Zea, and Triticeae. Other non-limiting examples of suitable plants include mosses, lichens, and algae. In some cases, the plants have economic, social and/or environmental value, such as food crops, fiber crops, oil crops, plants in the forestry or pulp and paper industries, feedstock for biofuel production and/or ornamental plants. In some examples, plants may be used to produce economically valuable products such as a grain, a flour, a starch, a syrup, a meal, an oil, a film, a packaging, a nutraceutical product, a pulp, an animal feed, a fish fodder, a bulk material for industrial chemicals, a cereal product, a processed human-food product, a sugar, an alcohol, and/or a protein. Non-limiting examples of crop plants include maize, rice, wheat, barley, sorghum, millet, oats, rye triticale, buckwheat, sweet corn, sugar cane, onions, tomatoes, strawberries, and asparagus.

In some examples, plants that may be obtained or improved using the methods and composition disclosed herein may include plants that are important or interesting for agriculture, horticulture, biomass for the production of biofuel molecules and other chemicals, and/or forestry. Some examples of these plants may include pineapple, banana, coconut, lily, grasspeas and grass; and dicotyledonous plants, such as, for example, peas, alfalfa, tomatillo, melon, chickpea, chicory, clover, kale, lentil, soybean, tobacco, potato, sweet potato, radish, cabbage, rape, apple trees, grape, cotton, sunflower, thale cress, canola, citrus (including orange, mandarin, kumquat, lemon, lime, grapefruit, tangerine, tangelo, citron, and pomelo), pepper, bean, lettuce, Panicum virgatum (switch), Sorghum bicolor (sorghum, sudan), Miscanthus giganteus (miscanthus), Saccharum sp. (energycane), Populus balsamifera (poplar), Zea mays (corn), Glycine max (soybean), Brassica napus (canola), Triticum aestivum (wheat), Gossypium hirsutum (cotton), Oryza sativa (rice), Helianthus annuus (sunflower), Medicago sativa (alfalfa), Beta vulgaris (sugarbeet), Pennisetum glaucum (pearl millet), Panicum spp. Sorghum spp., Miscanthus spp., Saccharun spp., Erianthus spp., Populus spp., Secale cereale (rye), Salix spp. (willow), Eucalyptus spp. (eucalyptus), Triticosecale spp. (triticum-25 wheat X rye), Bamboo, Carthamus tinctorius (safflower), Jatropha curcas (Jatropha), Ricinus communis (castor), Elaeis guineensis (oil palm), Phoenix dactylifera (date palm), Archontophoenix cunninghamiana (king palm), Syagrus romanzoffiana (queen palm), Linum usitatissimum (flax), Brassica juncea, Manihot esculenta (cassava), Lycopersicon esculentum (tomato), Lactuca sativa (lettuce), Musa paradisiaca (banana), Solanum tuberosum (potato), Brassica oleracea (broccoli, cauliflower, brussel sprouts), Camellia sinensis (tea), Fragaria ananassa (strawberry), Theobroma cacao (cocoa), Coffea arabica (coffee), Vitis vinifera (grape), Ananas comosus (pineapple), Capsicum annum (hot & sweet pepper), Allium cepa (onion), Cucumis melo (melon), Cucumis sativus (cucumber), Cucurbita maxima (squash), Cucurbita moschata (squash), Spinacea oleracea (spinach), Citrullus lanatus (watermelon), Abelmoschus esculentus (okra), Solanum melongena (eggplant), Papaver somniferum (opium poppy), Papaver orientale, Taxus baccata, Taxus brevifolia, Artemisia annua, Cannabis sativa, Camptotheca acuminate, Catharanthus roseus, Vinca rosea, Cinchona officinalis, Coichicum autumnale, Veratrum californica, Digitalis lanata, Digitalis purpurea, Dioscorea 5 spp., Andrographis paniculata, Atropa belladonna, Datura stomonium, Berberis spp., Cephalotaxus spp., Ephedra sinica, Ephedra spp., Erythroxylum coca, Galanthus womorii, Scopolia spp., Lycopodium serratum (Huperzia serrata), Lycopodium spp., Rauwolfia serpentina, Rauwolfia spp., Sanguinaria canadensis, Hyoscyamus spp., Calendula officinalis, Chrysanthemum parthenium, Coleus forskohlii, Tanacetum parthenium, Parthenium argentatum (guayule), Hevea spp. (rubber), Mentha spicata (mint), Mentha piperita (mint), Bixa orellana, Alstroemeria spp., Rosa spp. (rose), Dianthus caryophyllus (carnation), Petunia spp. (petunia), Poinsettia pulcherrima (poinsettia), Nicotiana tabacum (tobacco), Lupinus albus (lupin), Uniola paniculata (oats), Hordeum vulgare (barley), and Lolium spp. (rye).

In some examples, a monocotyledonous plant may be used. Monocotyledonous plants belong to the orders of the Alismatales, Arales, Arecales, Bromeliales, Commelinales, Cyclanthales, Cyperales, Eriocaulales, Hydrocharitales, Juncales, Lilliales, Najadales, Orchidales, Pandanales, Poales, Restionales, Triuridales, Typhales, and Zingiberales. Plants belonging to the class of the Gymnospermae are Cycadales, Ginkgoales, Gnetales, and Pinales. In some examples, the monocotyledonous plant can be selected from the group consisting of a maize, rice, wheat, barley, and sugarcane.

In some examples, a dicotyledonous plant may be used, including those belonging to the orders of the Aristochiales, Asterales, Batales, Campanulales, Capparales, Caryophyllales, Casuarinales, Celastrales, Comales, Diapensales, Dilleniales, Dipsacales, Ebenales, Ericales, Eucomiales, Euphorbiales, Fabales, Fagales, Gentianales, Geraniales, Haloragales, Hamamelidales, Middles, Juglandales, Lamiales, Laurales, Lecythidales, Leitneriales, Magniolales, Malvales, Myricales, Myrtales, Nymphaeales, Papeverales, Piperales, Plantaginales, Plumb aginales, Podostemales, Polemoniales, Polygalales, Polygonales, Primulales, Proteales, Rafflesiales, Ranunculales, Rhamnales, Rosales, Rubiales, Salicales, Santales, Sapindales, Sarraceniaceae, Scrophulariales, Theales, Trochodendrales, Umbellales, Urticales, and Violates. In some examples, the dicotyledonous plant can be selected from the group consisting of cotton, soybean, pepper, and tomato.

In some cases, the plant to be improved is not readily amenable to experimental conditions. For example, a crop plant may take too long to grow enough to practically assess an improved trait serially over multiple iterations. Accordingly, a first plant from which bacteria are initially isolated, and/or the plurality of plants to which genetically manipulated bacteria are applied may be a model plant, such as a plant more amenable to evaluation under desired conditions. Non-limiting examples of model plants include Setaria, Brachypodium, and Arabidopsis. Ability of bacteria isolated according to a method of the disclosure using a model plant may then be applied to a plant of another type (e.g. a crop plant) to confirm conferral of the improved trait.

Traits that may be improved by the methods disclosed herein include any observable characteristic of the plant, including, for example, growth rate, height, weight, color, taste, smell, changes in the production of one or more compounds by the plant (including for example, metabolites, proteins, drugs, carbohydrates, oils, and any other compounds). Selecting plants based on genotypic information is also envisaged (for example, including the pattern of plant gene expression in response to the bacteria, or identifying the presence of genetic markers, such as those associated with increased nitrogen fixation). Plants may also be selected based on the absence, suppression or inhibition of a certain feature or trait (such as an undesirable feature or trait) as opposed to the presence of a certain feature or trait (such as a desirable feature or trait).

### Concentrations and Rates of Application of Agricultural Compositions

As aforementioned, the agricultural compositions of the present disclosure, which comprise a taught microbe, can be applied to plants in a multitude of ways. In two particular aspects, the disclosure contemplates an in-furrow treatment or a seed treatment

For seed treatment embodiments, the microbes of the disclosure can be present on the seed in a variety of concentrations. For example, the microbes can be found in a seed treatment at a cfu concentration, per seed of:1×10¹, 1×10², 1×10³, 1×10⁴, 1×10⁵, 1×10⁶, 1×10⁷, 1×10⁸, 1×10⁹, 1×10¹⁰, or more. In particular aspects, the seed treatment compositions comprise about 1×10⁴ to about 1×10⁸ cfu per seed. In other particular aspects, the seed treatment compositions comprise about 1×10⁵ to about 1×10⁷ cfu per seed. In other aspects, the seed treatment compositions comprise about 1×10⁶ cfu per seed.

In the United States, about 10% of corn acreage is planted at a seed density of above about 36,000 seeds per acre; ⅓ of the corn acreage is planted at a seed density of between about 33,000 to 36,000 seeds per acre; ⅓ of the corn acreage is planted at a seed density of between about 30,000 to 33,000 seeds per acre, and the remainder of the acreage is variable. See, “Corn Seeding Rate Considerations,” written by Steve Butzen, available at: https://www.pioneer.com/home/site/us/agronomy/library/com-seeding-rate-considerations/

Table 2 below utilizes various cfu concentrations per seed in a contemplated seed treatment embodiment (rows across) and various seed acreage planting densities (1^{st} column: 15K-41K) to calculate the total amount of cfu per acre, which would be utilized in various agricultural scenarios (i.e. seed treatment concentration per seed×seed density planted per acre). Thus, if one were to utilize a seed treatment with 1×10⁶ cfu per seed and plant 30,000 seeds per acre, then the total cfu content per acre would be 3×10¹⁰ (i.e. 30K*1×10⁶).

TABLE 2
Total CFU Per Acre Calculation for Seed Treatment Embodiments

| Corn | | | | | | | | |
|---|---|---|---|---|---|---|---|---|
| Population | | | | | | | | |
| (i.e. | | | | | | | | |
| seeds per | | | | | | | | |
| acre) | 1.00E+02 | 1.00E+03 | 1.00E+04 | 1.00E+05 | 1.00E+06 | 1.00E+07 | 1.00E+08 | 1.00E+09 |
| 15,000 | 1.50E+06 | 1.50E+07 | 1.50E+08 | 1.50E+09 | 1.50E+10 | 1.50E+11 | 1.50E+12 | 1.50E+13 |
| 16,000 | 1.60E+06 | 1.60E+07 | 1.60E+08 | 1.60E+09 | 1.60E+10 | 1.60E+11 | 1.60E+12 | 1.60E+13 |
| 17,000 | 1.70E+06 | 1.70E+07 | 1.70E+08 | 1.70E+09 | 1.70E+10 | 1.70E+11 | 1.70E+12 | 1.70E+13 |
| 18,000 | 1.80E+06 | 1.80E+07 | 1.80E+08 | 1.80E+09 | 1.80E+10 | 1.80E+11 | 1.80E+12 | 1.80E+13 |
| 19,000 | 1.90E+06 | 1.90E+07 | 1.90E+08 | 1.90E+09 | 1.90E+10 | 1.90E+11 | 1.90E+12 | 1.90E+13 |
| 20,000 | 2.00E+06 | 2.00E+07 | 2.00E+08 | 2.00E+09 | 2.00E+10 | 2.00E+11 | 2.00E+12 | 2.00E+13 |
| 21,000 | 2.10E+06 | 2.10E+07 | 2.10E+08 | 2.10E+09 | 2.10E+10 | 2.10E+11 | 2.10E+12 | 2.10E+13 |
| 22,000 | 2.20E+06 | 2.20E+07 | 2.20E+08 | 2.20E+09 | 2.20E+10 | 2.20E+11 | 2.20E+12 | 2.20E+13 |
| 23,000 | 2.30E+06 | 2.30E+07 | 2.30E+08 | 2.30E+09 | 2.30E+10 | 2.30E+11 | 2.30E+12 | 2.30E+13 |
| 24,000 | 2.40E+06 | 2.40E+07 | 2.40E+08 | 2.40E+09 | 2.40E+10 | 2.40E+11 | 2.40E+12 | 2.40E+13 |
| 25,000 | 2.50E+06 | 2.50E+07 | 2.50E+08 | 2.50E+09 | 2.50E+10 | 2.50E+11 | 2.50E+12 | 2.50E+13 |
| 26,000 | 2.60E+06 | 2.60E+07 | 2.60E+08 | 2.60E+09 | 2.60E+10 | 2.60E+11 | 2.60E+12 | 2.60E+13 |
| 27,000 | 2.70E+06 | 2.70E+07 | 2.70E+08 | 2.70E+09 | 2.70E+10 | 2.70E+11 | 2.70E+12 | 2.70E+13 |
| 28,000 | 2.80E+06 | 2.80E+07 | 2.80E+08 | 2.80E+09 | 2.80E+10 | 2.80E+11 | 2.80E+12 | 2.80E+13 |
| 29,000 | 2.90E+06 | 2.90E+07 | 2.90E+08 | 2.90E+09 | 2.90E+10 | 2.90E+11 | 2.90E+12 | 2.90E+13 |
| 30,000 | 3.00E+06 | 3.00E+07 | 3.00E+08 | 3.00E+09 | 3.00E+10 | 3.00E+11 | 3.00E+12 | 3.00E+13 |
| 31,000 | 3.10E+06 | 3.10E+07 | 3.10E+08 | 3.10E+09 | 3.10E+10 | 3.10E+11 | 3.10E+12 | 3.10E+13 |
| 32,000 | 3.20E+06 | 3.20E+07 | 3.20E+08 | 3.20E+09 | 3.20E+10 | 3.20E+11 | 3.20E+12 | 3.20E+13 |
| 33,000 | 3.30E+06 | 3.30E+07 | 3.30E+08 | 3.30E+09 | 3.30E+10 | 3.30E+11 | 3.30E+12 | 3.30E+13 |
| 34,000 | 3.40E+06 | 3.40E+07 | 3.40E+08 | 3.40E+09 | 3.40E+10 | 3.40E+11 | 3.40E+12 | 3.40E+13 |
| 35,000 | 3.50E+06 | 3.50E+07 | 3.50E+08 | 3.50E+09 | 3.50E+10 | 3.50E+11 | 3.50E+12 | 3.50E+13 |
| 36,000 | 3.60E+06 | 3.60E+07 | 3.60E+08 | 3.60E+09 | 3.60E+10 | 3.60E+11 | 3.60E+12 | 3.60E+13 |
| 37,000 | 3.70E+06 | 3.70E+07 | 3.70E+08 | 3.70E+09 | 3.70E+10 | 3.70E+11 | 3.70E+12 | 3.70E+13 |
| 38,000 | 3.80E+06 | 3.80E+07 | 3.80E+08 | 3.80E+09 | 3.80E+10 | 3.80E+11 | 3.80E+12 | 3.80E+13 |
| 39,000 | 3.90E+06 | 3.90E+07 | 3.90E+08 | 3.90E+09 | 3.90E+10 | 3.90E+11 | 3.90E+12 | 3.90E+13 |
| 40,000 | 4.00E+06 | 4.00E+07 | 4.00E+08 | 4.00E+09 | 4.00E+10 | 4.00E+11 | 4.00E+12 | 4.00E+13 |
| 41,000 | 4.10E+06 | 4.10E+07 | 4.10E+08 | 4.10E+09 | 4.10E+10 | 4.10E+11 | 4.10E+12 | 4.10E+13 |

For in-furrow embodiments, the microbes of the disclosure can be applied at a cfu concentration per acre of: 1×10⁶, 3.20×10¹⁰, 1.60×10¹,3.20×10¹¹, 8.0×10¹¹, 1.6×10¹², 3.20×10¹², or more. Therefore, in aspects, the liquid in-furrow compositions can be applied at a concentration of between about 1×10⁶ to about 3×10¹² cfu per acre.

In some aspects, the in-furrow compositions are contained in a liquid formulation. In the liquid in-furrow embodiments, the microbes can be present at a cfu concentration per milliliter of 1×10¹, 1×10², 1×10³, 1×10⁴, 1×1⁵, 1×10⁶, 1×10⁷, 1×10⁸, 1×10⁹, 1×10¹⁰, 1×10¹¹, 1×10¹², 1×10¹³, or more. In certain aspects, the liquid in-furrow compositions comprise microbes at a concentration of about 1×10⁶ to about 1×10¹¹ cfu per milliliter. In other aspects, the liquid in-furrow compositions comprise microbes at a concentration of about 1×10⁷ to about 1×10¹⁰ cfu per milliliter. In other aspects, the liquid in-furrow compositions comprise microbes at a concentration of about 1×⁸ to about 1×10⁹ cfu per milliliter. In other aspects, the liquid in-furrow compositions comprise microbes at a concentration of up to about 1×10¹³ cfu per milliliter.

### EXAMPLES

The examples provided herein describe methods of bacterial isolation, bacterial and plant analysis, and plant trait improvement. The examples are for illustrative purposes only and are not to be construed as limiting in any way.

### Example 1: Transcriptomic Profiling of Candidate Microbes

Transcriptomic profiling of strain CI010 was performed to identify promoters that are active in the presence of environmental nitrogen. Strain CI010 was cultured in a defined, nitrogen-free media supplemented with 10 mM glutamine. Total RNA was extracted from these cultures (QIAGEN RNeasy kit) and subjected to RNAseq sequencing via Illumina HiSeq (SeqMatic, Fremont Calif.). Sequencing reads were mapped to CI010 genome data using Geneious, and highly expressed genes under control of proximal transcriptional promoters were identified.

Tables 3 and 4 list genes and their relative expression level as measured through RNASeq sequencing of total RNA. Sequences of the proximal promoters were recorded for use in mutagenesis of nif pathways, nitrogen utilization related pathways, colonization related pathways, or other genes with a desired expression level.

TABLE 3

| Name | Minimum | Maximum | Length | Direction |
|---|---|---|---|---|
| murein lipoprotein CDS | 2,929,898 | 2,930,134 | 237 | forward |
| membrane protein CDS | 5,217,517 | 5,217,843 | 327 | forward |
| zinc/cadmium-binding | 3,479,979 | 3,480,626 | 648 | forward |
| protein CDS | | | | |
| acyl carrier protein | 4,563,344 | 4,563,580 | 237 | reverse |
| CDS | | | | |
| ompX CDS | 4,251,002 | 4,251,514 | 513 | forward |
| DNA-binding protein | 375,156 | 375,428 | 273 | forward |
| HU-beta CDS | | | | |
| sspA CDS | 629,998 | 630,636 | 639 | reverse |
| tatE CDS | 3,199,435 | 3,199,638 | 204 | reverse |
| LexA repressor CDS | 1,850,457 | 1,851,065 | 609 | forward |
| hisS CDS | <3999979 | 4,001,223 | >1245 | forward |

TABLE 4

| | | | RNASeq_ | RNASeq_ | RNASeq_ | RNASeq_ |
|---|---|---|---|---|---|---|
| | Differential | | nifL- | nifL- | WT- | WT- |
| | Expression | Differential | Raw | Raw | Raw | Raw |
| | Absolute | Expression | Read | Transcript | Read | Transcript |
| Name | Confidence | Ratio | Count | Count | Count | Count |
| murein | 1000 | −1.8 | 12950.5 | 10078.9 | 5151.5 | 4106.8 |
| lipoprotein | | | | | | |
| CDS | | | | | | |
| membrane | 1000 | −1.3 | 9522.5 | 5371.3 | 5400 | 3120 |
| protein | | | | | | |
| CDS | | | | | | |
| zinc/ | 3.3 | 1.1 | 6461 | 1839.1 | 5318 | 1550.6 |
| cadmium- | | | | | | |
| binding | | | | | | |
| protein | | | | | | |
| CDS | | | | | | |
| acyl | 25.6 | 1.6 | 1230.5 | 957.6 | 1473.5 | 1174.7 |
| carrier | | | | | | |
| protein | | | | | | |
| CDS | | | | | | |
| ompX | 1.7 | 1.1 | 2042 | 734.2 | 1687.5 | 621.5 |
| CDS | | | | | | |
| DNA- | 6.9 | −1.3 | 1305 | 881.7 | 725 | 501.8 |
| binding | | | | | | |
| protein | | | | | | |
| HU-beta | | | | | | |
| CDS | | | | | | |
| sspA | 0.2 | 1 | 654 | 188.8 | 504.5 | 149.2 |
| CDS | | | | | | |
| tatE CDS | 1.4 | 1.3 | 131 | 118.4 | 125 | 115.8 |
| LexA | 0.1 | −1.1 | 248 | 75.1 | 164 | 50.9 |
| repressor | | | | | | |
| CDS | | | | | | |
| hisS CDS | 0 | −1.1 | 467 | 69.2 | 325 | 49.3 |

### Example 2: Mutagenesis of Candidate Microbes

### Lambda-Red Mediated Knockouts

Several mutants of candidate microbes were generated using the plasmid pKD46 or a derivative containing a kanamycin resistance marker (Datsenko et al. 2000; PNAS 97(12): 6640-6645). Knockout cassettes were designed with 250 bp homology flanking the target gene and generated via overlap extension PCR. Candidate microbes were transformed with pKD46, cultured in the presence of arabinose to induce Lambda-Red machinery expression, prepped for electroporation, and transformed with the knockout cassettes to produce candidate mutant strains. Four candidate microbes and one laboratory strain, Klebsiella oxytoca M5A1, were used to generate thirteen candidate mutants of the nitrogen fixation regulatory genes nifL, glnB, and amtB, as shown in Table 5.

TABLE 5
List of single knockout mutants created
through Lambda-red mutagenesis

| Strain | nifL | glnB | amtB |
|---|---|---|---|
| M5A1 | X | X | X |
| Cl006 | X | X | X |
| Cl010 | X | X | X |
| Cl019 | X | X | |
| Cl028 | X | X | |

Oligo-Directed Mutagenesis with Cas9 Selection

Oligo-directed mutagenesis was used to target genomic changes to the rpoB gene in E. coli DH10b1946B, and mutants were selected with a CRISPR-Cas system. A mutagenic oligo (ss1283: “G*T*T*G*ATCAGACCGATGTTCGGACCTTCcaagGTITCGATCGGACATACGCGAC CGTAGTGGGTCGGGTGTACGTCTCGAACTTCAAAGCC” (SEQ ID NO: 2), where * denotes phosphorothioate bond) was designed to confer rifampicin resistance through a 4-bp mutation to the rpoB gene. Cells containing a plasmid encoding Cas9 were induced for Cas9 expression, prepped for electroporation, and then electroporated with both the mutagenic oligo and a plasmid encoding constitutive expression of a guide RNA (gRNA) that targets Cas9 cleavage of the WT rpoB sequence. Electroporated cells were recovered in nonselective media overnight to allow sufficient segregation of the resulting mutant chromosomes. After plating on selection for the gRNA-encoding plasmid, two out of ten colonies screened were shown to contain the desired mutation, while the rest were shown to be escape mutants generated through protospacer mutation in the gRNA plasmid or Cas9 plasmid loss.

Lambda-Red Mutagenesis with Cas9 Selection

Mutants of candidate microbes CI006 and CI010 were generated via lambda-red mutagenesis with selection by CRISPR-Cas. Knockout cassettes contained an endogenous promoter identified through transcriptional profiling (as depicted in Tables 3-4) and ˜250 bp homology regions flanking the deletion target. CI006 and CI010 were transformed with plasmids encoding the Lambda-red recombination system (exo, beta, gam genes) under control of an arabinose inducible promoter and Cas9 under control of an IPTG inducible promoter. The Red recombination and Cas9 systems were induced in resulting transformants, and strains were prepared for electroporation. Knockout cassettes and a plasmid-encoded selection gRNA were subsequently transformed into the competent cells. After plating on antibiotics selective for both the Cas9 plasmid and the gRNA plasmid, 7 of the 10 colonies screened showed the intended knockout mutation.

### Example 3: In Planta Phenotyping of Candidate Microbes

### Colonization of Plants by Candidate Microbes

Colonization of desired host plants by a candidate microbe was quantified through short-term plant growth experiments. Corn plants were inoculated with strains expressing RFP either from a plasmid or from a Tn5-integrated RFP expression cassette. Plants were grown in both sterilized sand and nonsterile peat medium, and inoculation was performed by pipetting 1 mL of cell culture directly over the emerging plant coleoptile three days post-germination. Plasmids were maintained by watering plants with a solution containing the appropriate antibiotic. After three weeks, plant roots were collected, rinsed three times in sterile water to remove visible soil, and split into two samples. One root sample was analyzed via fluorescence microscopy to identify localization patterns of candidate microbes. Microscopy was performed on 10 mm lengths of the finest intact plant roots.

A second quantitative method for assessing colonization was developed. A quantitative PCR assay was performed on whole DNA preparations from the roots of plants inoculated with the endophytes. Seeds of corn (Dekalb DKC-66-40) were germinated in previously autoclaved sand in a 2.5 inch by 2.5 inch by 10 inch pot. One day after planting, 1 ml of endophyte overnight culture (SOB media) was drenched right at the spot of where the seed was located. 1 mL of this overnight culture is roughly equivalent to about 10\hat{ }9 cfu, varying within 3-fold of each other, depending on which strain is being used. Each seedling was fertilized 3× weekly with 50 mL modified Hoagland's solution supplemented with either 2.5 mM or 0.25 mM ammonium nitrate. At four weeks after planting, root samples were collected for DNA extraction. Soil debris were washed away using pressurized water spray. These tissue samples were then homogenized using QIAGEN Tissuelyzer and the DNA was then extracted using QIAmp DNA Mini Kit (QIAGEN) according to the recommended protocol. qPCR assay was performed using Stratagene Mx3005P RT-PCR on these DNA extracts using primers that were designed (using NCBI's Primer BLAST) to be specific to a loci in each of the endophyte's genome. The presence of the genome copies of the endophytes was quantified. To further confirm the identity of the endophytes, the PCR amplification products were sequenced and are confirmed to have the correct sequence. The summary of the colonization profile of strain CI006 and CI008 from candidate microbes are presented in Table 6. Colonization rate as high as 10\hat{ }7×cfu/gfw of root was demonstrated in strain C008.

TABLE 6
Colonization of corn as measured by qPCR

| | Colonization Rate |
|---|---|
| Strain | (CFU/g fw) |
| CI006 | 1.45 × 10\hat{ }5 |
| CI008 | 1.24 × 10\hat{ }7 |

### In Planta RNA Profiling

Biosynthesis of nif pathway components in planta was estimated by measuring the transcription of nif genes. Total RNA was obtained from root plant tissue of CI006 inoculated plants (planting methods as described previously). RNA extraction was performed using RNEasy Mini Kit according to the recommended protocol (QIAGEN). Total RNA from these plant tissues was then assayed using Nanostring Elements kits (NanoString Technologies, Inc.) using probes that were specific to the nif genes in the genome of strain C006. The data of nif gene expression in planta is summarized in Table 7. Expression of nifH genes was detected in plants inoculated by CM013 strains whereas nifH expression was not detectable in CI006 inoculated plants. Strain CM013 is a derivative of strain CI006 in which the nifL gene has been knocked out.

Highly expressed genes of CM011, ranked by transcripts per kilobase million (PM), were measured in planta under fertilized condition. The promoters controlling expression of some of these highly expressed genes were used as templates for homologous recombination into targeted nitrogen fixation and assimilation loci. RNA samples from greenhouse grown CM011 inoculated plant were extracted, rRNA removed using Ribo-Zero kit, sequenced using Illumina's Truseq platform and mapped back to the genome of CM011. Highly expressed genes from CM011 are listed in Table 8.

TABLE 7
Expression of nifH in planta

| Strains | Relative Transcript Expression |
|---|---|
| CI006 | 9.4 |
| CM013 | 103.25 |

TABLE 8

| | | | | TPM |
|---|---|---|---|---|
| | | | | (Transcripts |
| | | | Raw | Per |
| | Gene | | Read | Kilobase |
| Gene Name | Location | Direction | Count | Million) |
| rpsH CDS | 18196-18588 | reverse | 4841.5 | 27206.4 |
| rplQ CDS | 11650-12039 | reverse | 4333 | 24536.2 |
| rpsJ CDS | 25013-25324 | reverse | 3423 | 24229 |
| rplV CDS | 21946-22278 | reverse | 3367.5 | 22333 |
| rpsN CDS | 18622-18927 | reverse | 2792 | 20150.1 |
| rplN CDS | 19820-20191 | reverse | 3317 | 19691.8 |
| rplF CDS | 17649-18182 | reverse | 4504.5 | 18628.9 |
| rpsD CDS | 13095-13715 | reverse | 5091.5 | 18106.6 |
| rpmF CDS | 8326-8493 | forward | 1363.5 | 17923.8 |
| rplW CDS | 23429-23731 | reverse | 2252 | 16413.8 |
| rpsM CDS | 14153-14509 | reverse | 2269 | 14036.2 |
| rplR CDS | 17286-17639 | reverse | 2243.5 | 13996.1 |
| rplC CDS | 24350-24979 | reverse | 3985 | 13969.2 |
| rplK CDS | 25526-25954 | reverse | 2648.5 | 13634.1 |
| rplP CDS | 20807-21217 | reverse | 2423 | 13019.5 |
| rplX CDS | 19495-19809 | reverse | 1824 | 12787.8 |
| rpsQ CDS | 20362-20616 | reverse | 1460.5 | 12648.7 |
| bhsA 3 CDS | 79720-79977 | reverse | 1464 | 12531.5 |
| rpmC CDS | 20616-20807 | reverse | 998.5 | 11485 |
| rpoA CDS | 12080-13069 | reverse | 4855 | 10830.2 |
| rplD CDS | 23728-24333 | reverse | 2916.5 | 10628.5 |
| bhsA 1 CDS | 78883-79140 | reverse | 1068 | 9141.9 |
| rpsS CDS | 22293-22571 | reverse | 1138.5 | 9011.8 |
| rpmA CDS | 2210-2467 | forward | 1028.5 | 8803.7 |
| rpmD CDS | 16585-16764 | reverse | 694.5 | 8520.8 |
| rplB CDS | 22586-23410 | reverse | 3132 | 8384 |
| rpsC CDS | 21230-21928 | reverse | 2574.5 | 8133.9 |
| rplE CDS | 18941-19480 | reverse | 1972.5 | 8066.9 |
| rplO CDS | 16147-16581 | reverse | 1551 | 7874.2 |
| preprotein translocase | 14808-16139 | reverse | 4657 | 7721.2 |
| subunit SecY CDS | | | | |
| rpsE CDS | 16771-17271 | reverse | 1671.5 | 7368 |
| rpsK CDS | 13746-14135 | reverse | 1223.5 | 6928.2 |
| tufA CDS | 27318-28229 | reverse | 2850 | 6901.3 |
| rpmI CDS | 38574-38771 | forward | 615 | 6859.5 |
| rplU CDS | 1880-2191 | forward | 935.5 | 6621.7 |
| rplT CDS | 38814-39170 | forward | 1045 | 6464.4 |
| bhsA 2 CDS | 79293-79550 | reverse | 754 | 6454.1 |
| rpmB CDS | 8391-8627 | reverse | 682 | 6355.1 |
| rpll CDS | 23983-24480 | reverse | 1408 | 6243.9 |
| fusA 2 CDS | 481-2595 | reverse | 5832 | 6089.6 |
| rpsA CDS | 25062-26771 | reverse | 4613 | 5957.6 |
| rpmJ CDS | 14658-14774 | reverse | 314 | 5926.9 |
| rpsR CDS | 52990-53217 | forward | 603 | 5840.7 |
| rpsG CDS | 2692-3162 | reverse | 1243 | 5828.2 |
| rpsI CDS | 11354-11746 | reverse | 980.5 | 5509.8 |
| cspC 1 CDS | 8091-8300 | reverse | 509 | 5352.8 |
| rpsF CDS | 52270-52662 | forward | 916 | 5147.4 |
| rpsT CDS | 55208-55471 | reverse | 602 | 5035.9 |
| infC CDS | 38128-38478 | forward | 755 | 4750.3 |
| cspG CDS | 30148-30360 | forward | 446 | 4624.2 |

### Example 4: Compatibility Across Geography

The ability of the improved microbes to colonize an inoculated plant is critical to the success of the plant under field conditions. While the described isolation methods are designed to select from soil microbes that may have a close relationship with crop plants such as corn, many strains may not colonize effectively across a range of plant genotypes, environments, soil types, or inoculation conditions. Since colonization is a complex process requiring a range of interactions between amicrobial strain and host plant, screening for colonization competence has become a central method for selecting priority strains for further development. Early efforts to assess colonization used fluorescent tagging of strains, which was effective but time-consuming and not scalable on a per-strain basis. As colonization activity is not amenable to straightforward improvement, it is imperative that potential product candidates are selected from strains that are natural colonizers.

An assay was designed to test for robust colonization of the wild-type strains in any given host plant using qPCR and primers designed to be strain-specific in a community sample. This assay is intended to rapidly measure the colonization rate of the microbes from corn tissue samples. Initial tests using strains assessed as probable colonizers using fluorescence microscopy and plate-based techniques indicated that a qPCR approach would be both quantitative and scalable.

A typical assay is performed as follows: Plants, mostly varieties of maize and wheat, are grown in a peat potting mix in the greenhouse in replicates of six per strain. At four or five days after planting, a 1 mL drench of early stationary phase cultures of bacteria diluted to an OD590 of 0.6-1.0 (approximately 5E+08 CFU/mL) is pipetted over the emerging coleoptile. The plants are watered with tap water only and allowed to grow for four weeks before sampling, at which time, the plants are uprooted and the roots washed thoroughly to remove most peat residues. Samples of clean root are excised and homogenized to create a slurry of plant cell debris and associated bacterial cells. We developed a high-throughput DNA extraction protocol that effectively produced a mixture of plant and bacterial DNA to use as template for qPCR. Based on bacterial cell spike-in experiments, this DNA extraction process provides a quantitative bacterial DNA sample relative to the fresh weight of the roots. Each strain is assessed using strain-specific primers designed using Primer BLAST (Ye 2012) and compared to background amplification from uninoculated plants. Since some primers exhibit off-target amplification in uninoculated plants, colonization is determined either by presence of amplification or elevated amplification of the correct product compared to the background level.

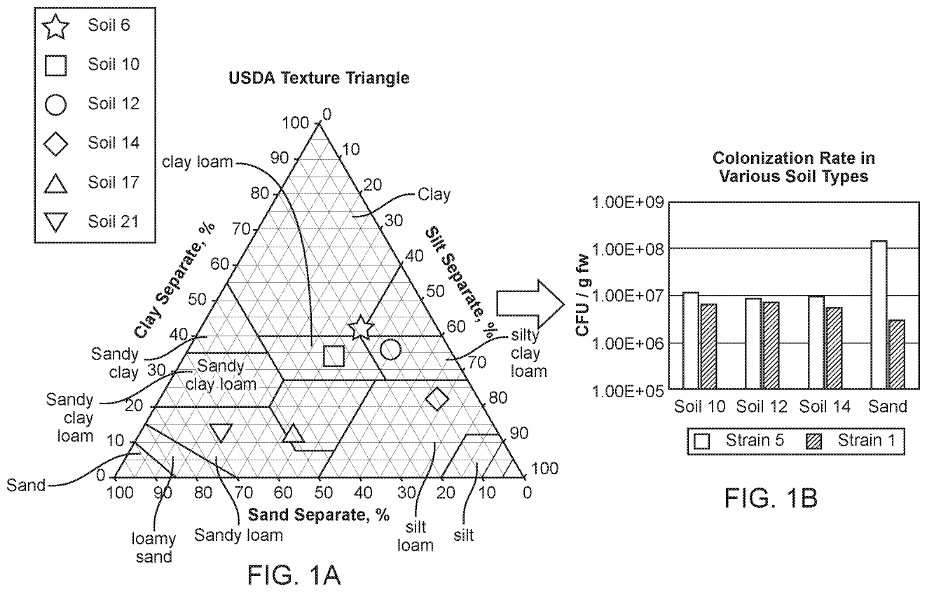

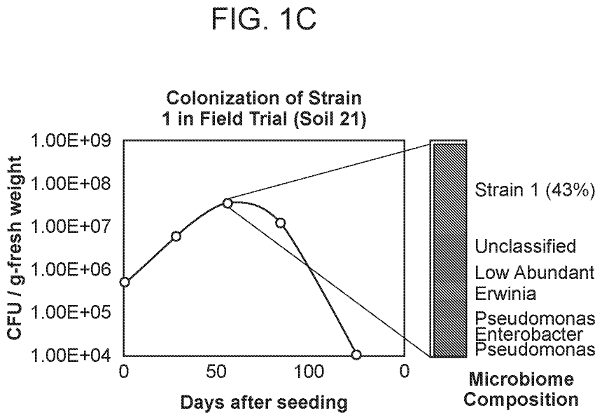

This assay was used to measure the compatibility of the microbial product across different soil geography. Field soil qualities and field conditions can have a huge influence on the effect of a microbial product. Soil pH, water retention capacity, and competitive microbes are only a few examples of factors in soil that can affect inoculum survival and colonization ability. A colonization assay was performed using three diverse soil types sampled from agricultural fields in California as the plant growth medium (FIG. 1A). An intermediate inoculation density was used to approximate realistic agricultural conditions. Within 3 weeks, Strain 5 colonized all plants at 1E+06 to 1E+07 CFU/g FW. After 7 weeks of plant growth, an evolved version of Strain 1 exhibited high colonization rates (1E+06 CFU/g FW) in all soil types. (FIG. 1B).

Additionally, to assess colonization in the complexity of field conditions, a 1-acre field trial in San Luis Obispo in June of 2015 was initiated to assess the impacts and colonization of seven of the wild-type strains in two varieties of field corn. Agronomic design and execution of the trial was performed by a contract field research organization, Pacific Ag Research. For inoculation, the same peat culture seed coating technique tested in the inoculation methods experiment was employed. During the course of the growing season, plant samples were collected to assess for colonization in the root and stem interior. Samples were collected from three replicate plots of each treatment at four and eight weeks after planting, and from all six reps of each treatment shortly before harvest at 16 weeks. Additional samples were collected from all six replicate plots of treatments inoculated with Strain 1 and Strain 2, as well as untreated controls, at 12 weeks. Numbers of cells per gram fresh weight of washed roots were assessed as with other colonization assays with qPCR and strain-specific primers. Two strains, Strain 1 and Strain 2, showed consistent and widespread root colonization that peaked at 12 weeks and then declined precipitously (FIG. 1C). While Strain 2 appeared to be present in numbers an order of magnitude lower than Strain 1, it was found in more consistent numbers from plant to plant. No strains appeared to effectively colonize the stem interior. In support of the qPCR colonization data, both strains were successfully re-isolated from the root samples using plating and 16S sequencing to identify isolates of matching sequence.

### Example 5: Microbe Breeding

Examples of microbe breeding can be summarized in the schematic of FIG. 2A. FIG. 2A depicts microbe breeding wherein the composition of the microbiome can be first measured and a species of interest is identified. The metabolism of the microbiome can be mapped and linked to genetics. Afterwards, a targeted genetic variation can be introduced using methods including, but not limited to, conjugation and recombination, chemical mutagenesis, adaptive evolution, and gene editing. Derivative microbes are used to inoculate crops. In some examples, the crops with the best phenotypes are selected.

As provided in FIG. 2A, the composition of the microbiome can be first measured and a species of interest is identified. FIG. 2B depicts an expanded view of the measurement of the microbiome step. The metabolism of the microbiome can be mapped and linked to genetics. The metabolism of nitrogen can involve the entrance of ammonia (NH₄⁺) from the rhizosphere into the cytosol of the bacteria via the AmtB transporter. Ammonia and L-glutamate (L-Glu) are catalyzed by glutamine synthetase and ATP into glutamine. Glutamine can lead to the formation of biomass (plant growth), and it can also inhibit expression of the nif operon. Afterwards, a targeted genetic variation can be introduced using methods including, but not limited to, conjugation and recombination, chemical mutagenesis, adaptive evolution, and gene editing. Derivative microbes are used to inoculate crops. The crops with the best phenotypes are selected.

### Example 6: Field Trials with Microbes of the Disclosure

A diversity of nitrogen fixing bacteria can be found in nature, including in agricultural soils. However, the potential of a microbe to provide sufficient nitrogen to crops to allow decreased fertilizer use may be limited by repression of nitrogenase genes in fertilized soils as well as low abundance in close association with crop roots. Identification, isolation and breeding of microbes that closely associate with key commercial crops might disrupt and improve the regulatory networks linking nitrogen sensing and nitrogen fixation and unlock significant nitrogen contributions by crop-associated microbes. To this end, nitrogen fixing microbes that associate with and colonize the root system of corn were identified.

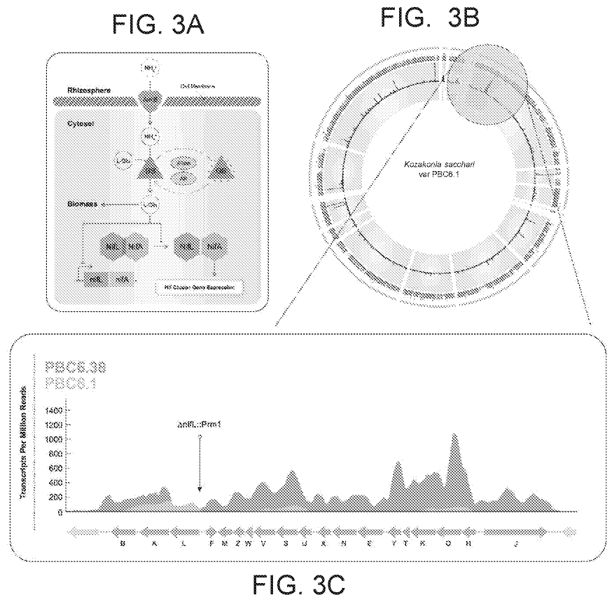

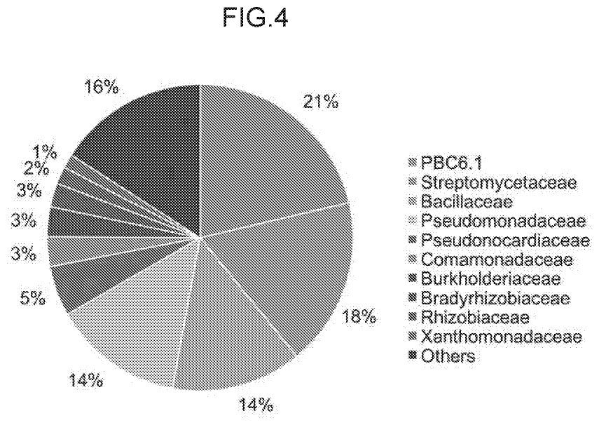

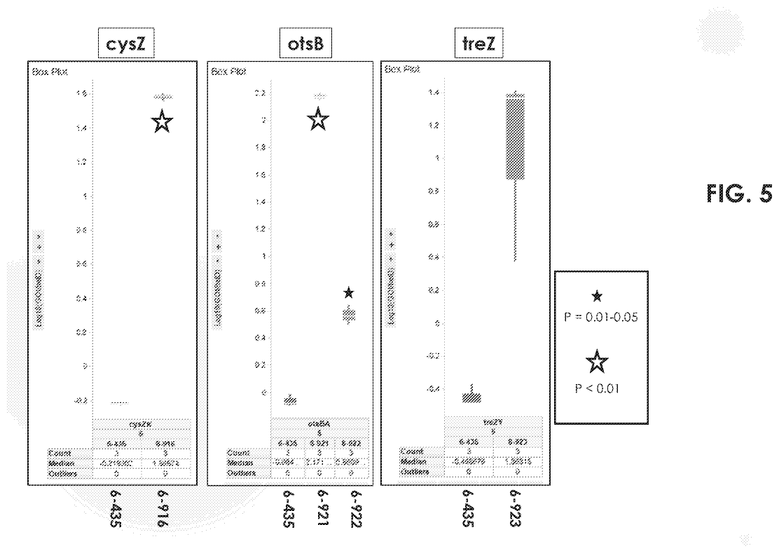

Root samples from corn plants grown in agronomically relevant soils were collected, and microbial populations extracted from the rhizosphere and endosphere. Genomic DNA from these samples was extracted, followed by 16S amplicon sequencing to profile the community composition. A Kosakonia sacchari microbe (strain PBC6.1) was isolated and classified through 16S rRNA and whole genome sequencing. This is a particularly interesting nitrogen fixer capable of colonizing to nearly 21% abundance of the root-associated microbiota (FIG. 4). To assess strain sensitivity to exogenous nitrogen, nitrogen fixation rates in pure culture were measured with the classical acetylene reduction assay (ARA) and varying levels of glutamine supplementation. The species exhibited a high level of nitrogen fixing activity in nitrogen-free media, yet exogenous fixed nitrogen repressed nif gene expression and nitrogenase activity (Strain PBC6.1, FIG. 3C). Additionally, when released ammonia was measured in the supernatant of PBC6.1 grown in nitrogen-fixing conditions, very little release of fixed nitrogen could be detected.

We hypothesized that PBC6.1 could be a significant contributor of fixed nitrogen in fertilized fields if regulatory networks controlling nitrogen metabolism were rewired to allow optimal nitrogenase expression and ammonia release in the presence of fixed nitrogen. Sufficient genetic diversity should exist within the PBC6.1 genome to enable broad phenotypic remodeling without the insertion of transgenes or synthetic regulatory elements. The isolated strain has a genome of at least 5.4 Mbp and a canonical nitrogen fixation gene cluster. Related nitrogen metabolism pathways in PBC6.1 are similar to those of the model organism for nitrogen fixation, Klebsiella oxytoca m5al.

Several gene regulatory network nodes were identified which may augment nitrogen fixation and subsequent transfer to a host plant, particularly in high exogenous concentrations of fixed nitrogen (FIG. 3A). The nifLA operon directly regulates the rest of the nif cluster through transcriptional activation by NifA and nitrogen- and oxygen-dependent repression of NifA by NifL. Disruption of nifL can abolish inhibition of NifA and improve nif expression in the presence of both oxygen and exogenous fixed nitrogen. Furthermore, expressing nifA under the control of a nitrogen-independent promoter may decouple nitrogenase biosynthesis from regulation by the NtrB/NtrC nitrogen sensing complex. The assimilation of fixed nitrogen by the microbe to glutamine by glutamine synthetase (GS) is reversibly regulated by the two-domain adenylyltransferase (ATase) enzyme GlnE through the adenylylation and deadenylylation of GS to attenuate and restore activity, respectively. Truncation of the GlnE protein to delete its adenylyl-removing (AR) domain may lead to constitutively adenylylated glutamine synthetase, limiting ammonia assimilation by the microbe and increasing intra- and extracellular ammonia. Finally, reducing expression of AmtB, the transporter responsible for uptake of ammonia, could lead to greater extracellular ammonia. To generate rationally designed microbial phenotypes without the use of transgenes, two approaches were employed: creating markerless deletions of genomic sequences encoding protein domains or whole genes, and rewiring regulatory networks by intragenomic promoter rearrangement Through an iterative mutagenesis process, several non-transgenic derivative strains of PBC6.1 were generated (Table 9).

TABLE 9
List of isolated and derivative strains used in this work.
Prm, promoter sequence derived from the PBC6.1 genome;
ΔglnE_{AR}1 and ΔglnE_{AR}2, different truncated versions of
glnE gene removing the adenylyl-removing domain sequence.

| Strain ID | Species | Genotype |
|---|---|---|
| C1006 | K. sacchari | wildtype |
| DH10B | E. coli | |
| PBC6.1 | K. sacchari | WT |
| PBC6.14 | K. sacchari | ΔnifL::Prm1 |
| PBC6.15 | K. sacchari | ΔnifL::Prm5 |
| PBC6.22 | K. sacchari | ΔnifL::Prm3 |
| PBC6.37 | K. sacchari | ΔnifL::Prm1 ΔglnE_{AR}2 |
| PBC6.38 | K. sacchari | ΔnifL::Prm1 ΔglnE_{AR}1 |
| PBC6.93 | K. sacchari | ΔnifL::Prm1 ΔglnE_{AR}2 ΔamtB |
| PBC6.94 | K. sacchari | ΔnifL::Prm1 ΔglnE_{AR}1 ΔamtB |

Several in vitro assays were performed to characterize specific phenotypes of the derivative strains. The ARA was used to assess strain sensitivity to exogenous nitrogen, in which PBC6.1 exhibited repression of nitrogenase activity at high glutamine concentrations. In contrast, most derivative strains showed a derepressed phenotype with varying levels of acetylene reduction observed at high glutamine concentrations. Transcriptional rates of nifA in samples analyzed by qPCR correlated well with acetylene reduction rates, supporting the hypothesis that nifL disruption and insertion of a nitrogen-independent promoter to drive nifA can lead to nif cluster derepression. Strains with altered GlnE or AmtB activity showed markedly increased ammonium excretion rates compared to wild type or derivative strains without these mutations, illustrating the effect of these genotypes on ammonia assimilation and reuptake.

Two experiments were performed to study the interaction of PBC6.1 derivatives with corn plants and quantify incorporation of fixed nitrogen into plant tissues. First, rates of microbial nitrogen fixation were quantified in a greenhouse study using isotopic tracers. Briefly, plants are grown with 15N labeled fertilizer, and diluted concentrations of 15N in plant tissues indicate contributions of fixed nitrogen from microbes. Corn seedlings were inoculated with selected microbial strains, and plants were grown to the V6 growth stage. Plants were subsequently deconstructed to enable measurement of microbial colonization and gene expression as well as measurement of 15N/14N ratios in plant tissues by isotope ratio mass spectrometry (IRMS). Analysis of the aerial tissue showed a small, nonsignificant contribution by PBC6.38 to plant nitrogen levels, and a significant contribution by PBC6.94 (p=0.011). Approximately 20% of the nitrogen found in above-ground corn leaves was produced by PBC6.94, with the remainder coming from the seed, potting mix, or “background” fixation by other soilborne microbes. This illustrates that our microbial breeding pipeline can generate strains capable of making significant nitrogen contributions to plants in the presence of nitrogen fertilizer. Microbial transcription within plant tissues was measured, and expression of the nif gene cluster was observed in derivative strains but not the wild type strain, showing the importance of nif derepression for contribution of BNF to crops in fertilized conditions. Root colonization measured by qPCR demonstrated that colonization density is different for each of the strains tested. A 50 fold difference in colonization was observed between PBC6.38 and PBC6.94. This difference could be an indication that PBC6.94 has reduced fitness in the rhizosphere relative to PBC6.38 as a result of high levels of fixation and excretion.

### Methods

### Media

Minimal medium contains (per liter) 25 g Na₂HPO₄, 0.1 g CaCL₂-2H₂O, 3 g KH₂PO₄, 0.25 g MgSO₄.7H₂O, 1 g NaCl, 2.9 mg FeCl₃, 0.25 mg Na₂MoO₄.2H₂O, and 20 g sucrose. Growth medium is defined as minimal medium supplemented with 50 ml of 200 mM glutamine per liter.

### Isolation of Diazotrophs

Corn seedlings were grown from seed (DKC 66-40, DeKalb, Ill.) for two weeks in a greenhouse environment controlled from 22° C. (night) to 26° C. (day) and exposed to 16 hour light cycles in soil collected from San Joaquin County, CA. Roots were harvested and washed with sterile deionized water to remove bulk soil. Root tissues were homogenized with 2 mm stainless steel beads in a tissue lyser (TissueLyser II, Qiagen P/N 85300) for three minutes at setting 30, and the samples were centrifuged for 1 minute at 13,000 rpm to separate tissue from root-associated bacteria. Supematants were split into two fractions, and one was used to characterize the microbiome through 16S rRNA amplicon sequencing and the remaining fraction was diluted and plated on Nitrogen-free Broth (NfB) media supplemented with 1.5% agar. Plates were incubated at 30° C. for 5-7 days. Colonies that emerged were tested for the presence of the nJH gene by colony PCR with primers Ueda9f and Ueda406r. Genomic DNA from strains with a positive nH colony PCR was isolated (QIAamp DNA Mini Kit, Cat No. 51306, QIAGEN, Germany) and sequenced (Illumina MiSeq v3, SeqMatic, Fremont, Calif.). Following sequence assembly and annotation, the isolates containing nitrogen fixation gene clusters were utilized in downstream research.

### Microbiome Profiling of Isolation Seedlings

Genomic DNA was isolated from root-associated bacteria using the ZR-96 Genomic DNA I Kit (Zymo Research P/N D3011), and 16S rRNA amplicons were generated using nextera-barcoded primers targeting 799f and 1114r. The amplicon libraries were purified and sequenced with the Illumina MiSeq v3 platform (SeqMatic, Fremont, Calif.). Reads were taxonomically classified using Kraken using the minikraken database.

### Acetylene Reduction Assay (ARA)

A modified version of the Acetylene Reduction Assay was used to measure nitrogenase activity in pure culture conditions. Strains were propagated from single colony in SOB (RPI, P/N S25040-1000) at 30° C. with shaking at 200 RPM for 24 hours and then subcultured 1:25 into growth medium and grown aerobically for 24 hours (30° C., 200 RPM). 1 ml of the minimal media culture was then added to 4 ml of minimal media supplemented with 0 to 10 mM glutamine in air-tight Hungate tubes and grown anaerobically for 4 hours (30° C., 200 RPM). 10% headspace was removed then replaced by an equal volume of acetylene by injection, and incubation continued for 1 hr. Subsequently, 2 ml of headspace was removed via gas tight syringe for quantification of ethylene production using an Agilent 6850 gas chromatograph equipped with a flame ionization detector (FID).

### Ammonium Excretion Assay

Excretion of fixed nitrogen in the form of ammonia was measured using batch fermentation in anaerobic bioreactors. Strains were propagated from single colony in 1 ml/well of SOB in a 96 well DeepWell plate. The plate was incubated at 30° C. with shaking at 200 RPM for 24 hours and then diluted 1:25 into a fresh plate containing 1 ml/well of growth medium. Cells were incubated for 24 hours (30° C., 200 RPM) and then diluted 1:10 into a fresh plate containing minimal medium. The plate was transferred to an anaerobic chamber with a gas mixture of >98.5% nitrogen, 1.2-1.5% hydrogen and <30 ppM oxygen and incubated at 1350 RPM, room temperature for 66-70 hrs. Initial culture biomass was compared to ending biomass by measuring optical density at 590 nm. Cells were then separated by centrifugation, and supernatant from the reactor broth was assayed for free ammonia using the Megazyme Ammonia Assay kit (P/N K-AMIAR) normalized to biomass at each timepoint.

### Extraction of Root-Associated Microbiome

Roots were shaken gently to remove loose particles, and root systems were separated and soaked in a RNA stabilization solution (Thermo Fisher P/N AM7021) for 30 minutes. The roots were then briefly rinsed with sterile deionized water. Samples were homogenized using bead beating with ½-inch stainless steel ball bearings in a tissue lyser (TissueLyser II, Qiagen P/N 85300) in 2 ml of lysis buffer (Qiagen P/N 79216). Genomic DNA extraction was performed with ZR-96 Quick-gDNA kit (Zymo Research P/N D3010), and RNA extraction using the RNeasy kit (Qiagen P/N 74104).

### Root Colonization Assay

Four days after planting, 1 ml of a bacterial overnight culture (approximately 10¹ cfu) was applied to the soil above the planted seed. Seedlings were fertilized three times weekly with 25 ml modified Hoagland's solution supplemented with 0.5 mM ammonium nitrate. Four weeks after planting, root samples were collected and the total genomic DNA (gDNA) was extracted. Root colonization was quantified using qPCR with primers designed to amplify unique regions of either the wild type or derivative strain genome. QPCR reaction efficiency was measured using a standard curve generated from a known quantity of gDNA from the target genome. Data was normalized to genome copies per g fresh weight using the tissue weight and extraction volume. For each experiment, the colonization numbers were compared to untreated control seedlings.

### In Planta Transcriptomics

Transcriptional profiling of root-associated microbes was measured in seedlings grown and processed as described in the Root Colonization Assay. Purified RNA was sequenced using the Illumina NextSeq platform (SeqMatic, Fremont, Calif.). Reads were mapped to the genome of the inoculated strain using bowtie2 using ‘—very-sensitive-local’ parameters and a minimum alignment score of 30. Coverage across the genome was calculated using samtools. Differential coverage was normalized to housekeeping gene expression and visualized across the genome using Circos and across the nif gene cluster using DNAplotlib. Additionally, the in planta transcriptional profile was quantified via targeted Nanostring analysis. Purified RNA was processed on an nCounter Sprint (Core Diagnostics, Hayward, Calif.).

### 15N Dilution Greenhouse Study

A 15N fertilizer dilution experiment was performed to assess optimized strain activity in planta. A planting medium containing minimal background N was prepared using a mixture of vermiculite and washed sand (5 rinses in DI H₂O). The sand mixture was autoclaved for 1 hour at 122° C. and approximately 600 g measured out into 40 cubic inch (656 mL) pots, which were saturated with sterile DI H₂O and allowed to drain 24 hours before planting. Corn seeds (DKC 66-40) were surface sterilized in 0.625% sodium hypochlorite for 10 minutes, then rinsed five times in sterile distilled water and planted 1 cm deep. The plants were maintained under fluorescent lamps for four weeks with 16-hour day length at room temperatures averaging 22° C. (night) to 26° C. (day).

Five days after planting, seedlings were inoculated with a 1 ml suspension of cells drenched directly over the emerging coleoptile. Inoculum was prepared from 5 ml overnight cultures in SOB, which were spun down and resuspended twice in 5 ml PBS to remove residual SOB before final dilution to OD of 1.0 (approximately 10⁹ CFU/ml). Control plants were treated with sterile PBS, and each treatment was applied to ten replicate plants.

Plants were fertilized with 25 ml fertilizer solution containing 2% 15N-enriched 2 mM KNO₃ on 5, 9, 14, and 19 days after planting, and the same solution without KNO₃ on 7, 12, 16, and 18 days after planting. The fertilizer solution contained (per liter) 3 mmol CaCl₂, 0.5 mmol KH₂PO₄, 2 mmol MgSO₄, 17.9 μmol FeSO₄, 2.86 mg H₃BO₃, 1.81 mg MnCl₂.4H₂O, 0.22 mg ZnSO₄.7H₂O, 51 μg CuSO₄/5H₂O, 0.12 mg Na₂MoO₄.2H₂O, and 0.14 nmol NiCl₂. All pots were watered with sterile DI H₂O as needed to maintain consistent soil moisture without runoff.

At four weeks, plants were harvested and separated at the lowest node into samples for root gDNA and RNA extraction and aerial tissue for IRMS. Aerial tissues were wiped as needed to remove sand, placed whole into paper bags and dried for at least 72 hours at 60° C. Once completely dry, total aerial tissue was homogenized by bead beating and 5-7 mg samples were analyzed by isotope ratio mass spectrometry (IRMS) for δ15N by the MBL Stable Isotope Laboratory (The Ecosystems Center, Woods Hole, Mass.). Percent NDFA was calculated using the following formula: % NDFA=(δ15N of UTC average−δ15N of sample)/(δ15N of UTC average)×100.

### Example 7: Methods and Assays for Detection of Non-Intrageneric Engineered Microbes

The present disclosure teaches primers, probes, and assays that are useful for detecting the microbes utilized in the various aforementioned Examples. The assays are able to detect the non-natural nucleotide “junction” sequences in the derived/mutant non-intergeneric microbes. These non-naturally occurring nucleotide junctions can be used as a type of diagnostic that is indicative of the presence of a particular genetic alteration in a microbe.

The present techniques are able to detect these non-naturally occurring nucleotide junctions via the utilization of specialized quantitative PCR methods, including uniquely designed primers and probes. The probes can bind to the non-naturally occurring nucleotide junction sequences. That is, sequence-specific DNA probes consisting of oligonucleotides that are labelled with a fluorescent reporter, which permits detection only after hybridization of the probe with its complementary sequence can be used. The quantitative methods can ensure that only the non-naturally occurring nucleotide junction will be amplified via the taught primers, and consequently can be detected via either a non-specific dye, or via the utilization of a specific hybridization probe. Another aspect of the method is to choose primers such that the primers flank either side of a junction sequence, such that if an amplification reaction occurs, then said junction sequence is present.

Consequently, genomic DNA can be extracted from samples and used to quantify the presence of microbes of the disclosure by using qPCR. The primers utilized in the qPCR reaction can be primers designed by Primer Blast (https://www.ncbi.nlm.nih.gov/tools/primer-blast/) to amplify unique regions of the wild-type genome or unique regions of the engineered non-intergeneric mutant strains. The qPCR reaction can be carried out using the SYBR GreenER qPCR SuperMix Universal (Thermo Fisher P/N 11762100) kit, using only forward and reverse amplification primers; alternatively, the Kapa Probe Force kit (Kapa Biosystems P/N KK4301) can be used with amplification primers and a TaqMan probe containing a FAM dye label at the 5′ end, an internal ZEN quencher, and a minor groove binder and fluorescent quencher at the 3′ end (Integrated DNA Technologies).

TABLE 10
Engineered Non-intergeneric Microbes

| | SEQ | | |
|---|---|---|---|
| Strain | ID | Geno- | |
| Name | NO. | type | SEQUENCE |
| CI006 | 3 | 16S | ttgaagagtt tgatcatggc tcagattgaa |
| | | rDNA - | cgctggcggc aggcctaaca catgcaagtc |
| | | contig | gaacggtagc acagagagct tgctctcggg |
| | | 5 | tgacgagtgg cggacgggtg agtaatgtct |
| | | | gggaaactgc ctgatggagg gggataacta |
| | | | ctggaaacgg tagctaatac cgcataacgt |
| | | | cgcaagacca aagaggggga ccttcgggcc |
| | | | tcttgccatc agatgtgccc agatgggatt |
| | | | agctagtagg tggggtaacg gctcacctag |
| | | | gcgacgatcc ctagctggtc tgagaggatg |
| | | | accagccaca ctggaactga gacacggtcc |
| | | | agactcctac gggaggcagc agtggggaat |
| | | | attgcacaat gggcgcaagc ctgatgcagc |
| | | | catgccgcgt gtgtgaagaa ggccttcggg |
| | | | ttgtaaagca ctttcagcgg ggaggaaggg |
| | | | agtaaggtta ataaccttat tcattgacgt |
| | | | tacccgcaga agaagcaccg gctaactccg |
| | | | tgccagcagc cgcggtaata cggagggtgc |
| | | | aagcgttaat cggaattact gggcgtaaag |
| | | | cgcacgcagg cggtctgtca agtcggatgt |
| | | | gaaatccccg ggctcaacct gggaactgca |
| | | | tccgaaactg gcaggcttga gtctcgtaga |
| | | | gggaggtaga attccaggtg tagcggtgaa |
| | | | atgcgtagag atctggagga ataccggtgg |
| | | | cgaaggcggc ctcctggacg aagactgacg |
| | | | ctcaggtgcg aaagcgtggg gagcaaacag |
| | | | gattagatac cctggtagtc cacgccgtaa |
| | | | acgatgtcta tttggaggtt gtgcccttga |
| | | | ggcgtggctt ccggagctaa cgcgttaaat |
| | | | agaccgcctg gggagtacgg ccgcaaggtt |
| | | | aaaactcaaa tgaattgacg ggggcccgca |
| | | | caagcggtgg agcatgtggt ttaattcgat |
| | | | gcaacgcgaa gaaccttacc tggtcttgac |
| | | | atccacagaa ctttccagag atggattggt |
| | | | gccttcggga actgtgagac aggtgctgca |
| | | | tggctgtcgt cagctcgtgt tgtgaaatgt |
| | | | tgggttaagt cccgcaacga gcgcaaccct |
| | | | tatcctttgt tgccagcggt ccggccggga |
| | | | actcaaagga gactgccagt gataaactgg |
| | | | aggaaggtgg ggatgacgtc aagtcatcat |
| | | | ggcccttacg accagggcta cacacgtgct |
| | | | acaatggcgc atacaaagag aagcgacctc |
| | | | gcgagagtaa gcggacctca taaagtgcgt |
| | | | cgtagtccgg attggagtct gcaactcgac |
| | | | tccatgaagt cggaatcgct agtaatcgtg |
| | | | gatcagaatg ccacggtgaa tacgttcccg |
| | | | ggccttgtac acaccgcccg tcacaccatg |
| | | | ggagtgggtt gcaaaagaag taggtagctt |
| | | | aaccttcggg agggcgctta ccactttgtg |
| | | | attcatgact ggggtgaagt cgtaacaagg |
| | | | taaccgtagg ggaacctgcg gttggatcac |
| | | | ctcctt |
| CI006 | 4 | 16S | ttgaagagtt tgatcatggc tcagattgaa |
| | | rDNA - | cgctggcggc aggcctaaca catgcaagtc |
| | | contig | gaacggtagc acagagagct tgctctcggg |
| | | 8 | tgacgagtgg cggacgggtg agtaatgtct |
| | | | gggaaactgc ctgatggagg gggataacta |
| | | | ctggaaacgg tagctaatac cgcataacgt |
| | | | cgcaagacca aagaggggga ccttcgggcc |
| | | | tcttgccatc agatgtgccc agatgggatt |
| | | | agctagtagg tggggtaacg gctcacctag |
| | | | gcgacgatcc ctagctggtc tgagaggatg |
| | | | accagccaca ctggaactga gacacggtcc |
| | | | agactcctac gggaggcagc agtggggaat |
| | | | attgcacaat gggcgcaagc ctgatgcagc |
| | | | catgccgcgt gtgtgaagaa ggccttcggg |
| | | | ttgtaaagca ctttcagcgg ggaggaaggn |
| | | | antanggtta ataacctgtg ttnattgacg |
| | | | ttacccgcag aagaagcacc ggctaactcc |
| | | | gtgccagcag ccgcggtaat acggagggtg |
| | | | caagcgttaa tcggaattac tgggcgtaaa |
| | | | gcgcacgcag gcggtctgtc aagtcggatg |
| | | | tgaaatcccc gggctcaacc tgggaactgc |
| | | | atccgaaact ggcaggcttg agtctcgtag |
| | | | agggaggtag aattccaggt gtagcggtga |
| | | | aatgcgtaga gatctggagg aataccggtg |
| | | | gcgaaggcgg cctcctggac gaagactgac |
| | | | gctcaggtgc gaaagcgtgg ggagcaaaca |
| | | | ggattagata ccctggtagt ccacgccgta |
| | | | aacgatgtct atttggaggt tgtgcccttg |
| | | | aggcgtggct tccggagcta acgcgttaaa |
| | | | tagaccgcct ggggagtacg gccgcaaggt |
| | | | taaaactcaa atgaattgac gggggcccgc |
| | | | acaagcggtg gagcatgtgg tttaattcga |
| | | | tgcaacgcga agaaccttac ctggtcttga |
| | | | catccacaga acttagcaga gatgctttgg |
| | | | tgccttcggg aactgtgaga caggtgctgc |
| | | | atggctgtcg tcagctcgtg ttgtgaaatg |
| | | | ttgggttaag tcccgcaacg agcgcaaccc |
| | | | ttatcctttg ttgccagcgg ttaggccggg |
| | | | aactcaaagg agactgccag tgataaactg |
| | | | gaggaaggtg gggatgacgt caagtcatca |
| | | | tggcccttac gaccagggct acacacgtgc |
| | | | tacaatggcg catacaaaga gaagcgacct |
| | | | cgcgagagta agcggacctc ataaagtgcg |
| | | | tcgtagtccg gattggagtc tgcaactcga |
| | | | ctccatgaag tcggaatcgc tagtaatcgt |
| | | | ggatcagaat gccacggtga atacgttccc |
| | | | gggccttgta cacaccgccc gtcacaccat |
| | | | gggagtgggt tgcaaaagaa gtaggtagct |
| | | | taaccttcgg gagggcgctt accactttgt |
| | | | gattcatgac tggggtgaag tcgtaacaag |
| | | | gtaaccgtag gggaacctgc ggttggatca |
| | | | cctcctt |

### Example 8: Identification of Klebsiella variicola and Kosakonia sacchari

The use of 16S rRNA gene sequences to study bacterial phylogeny and taxonomy have been by far the most common housekeeping genetic marker—used for a number of reasons. These reasons include (i) its presence in almost ali bacteria, often existing as a multigene family, or operons; (ii) the function of the 16S rRNA gene over time has not changed, suggesting that random sequence changes are a more accurate measure of time (evolution); and (iii) the 16S rRNA gene (1.500 bp) is large enough for informatics purposes.

The nucleotide sequence of the 16S rRNA gene of Klebsiella variicola and Kosakonia sacchari was determined by PCR analysis. The primers used are universal 16S primers, with the following nucleotide sequence:

Forward 16S primer 27f
(AGAGTTTGATCMTGGCTCAG SEQ ID NO: 5)
Reverse 165 primer 1492r.
(GGTTACCTTGTTACGACTT SEQ ID NO: 6)

The resulting PCR products were sequenced, and the resulting sequence was compared against the National Center for Biotechnology Information (NCBI) database for species identification during the original isolation. In any subsequent production of the microbe, the same PCR analysis was conducted to ensure a pure verified microbe that is free of contamination.

Two parental strains were identified by this method. The scientific name of the first is Klebsiella variicola. The species identity was determined by sequencing the organism's 16S rRNA multiple times and using BLAST in the NCBI database for genus and species verification. Furthermore, the entire genome of the organism was sequenced and BLASTed the extracted 16S rRNA (from the genome sequence). This sequence was aligned to the previously determined 16S rRNA sequences to confirm that the organism was isolated to a pure culture.

The scientific name of the second organism is Kosakonia sacchari. The species identity was determined by sequencing the organism's 16S rRNA multiple times and using BLAST in the NCBI database for genus and species verification. Furthermore, the entire genome of the organism was sequenced and the extracted 16S rRNA (from the genome sequence) was BLASTed. This sequence was aligned to the previously determined 16 rRNA sequences to confirm that the organism was isolated to a pure culture

### Example 9: Remodeled Strains with Increased Colonization or Nitrogen Fixation Activity

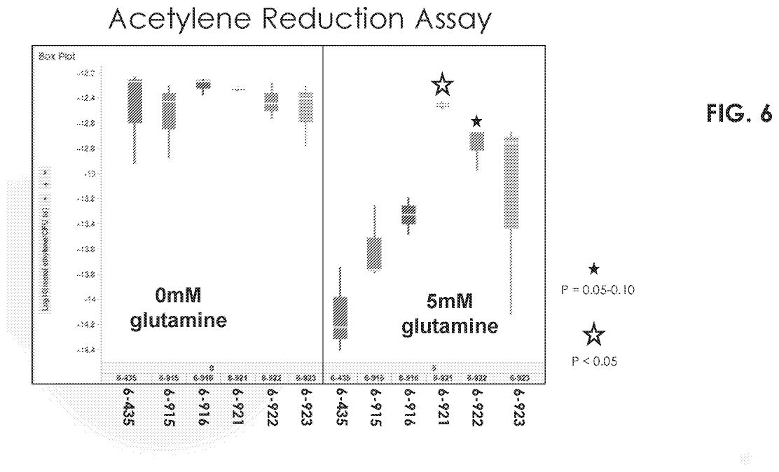

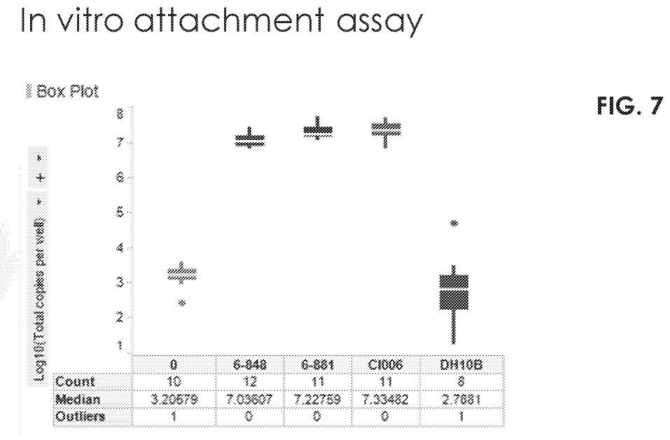

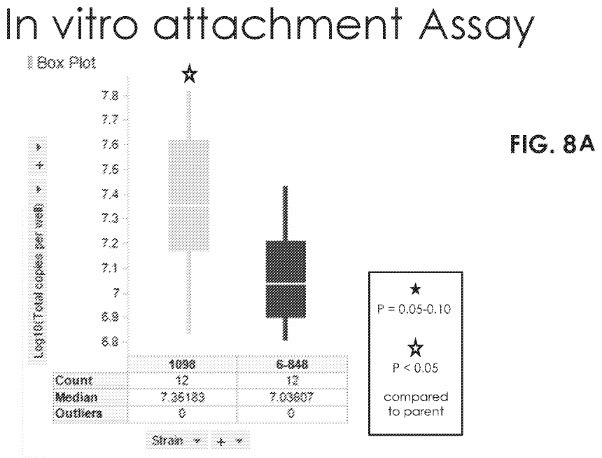

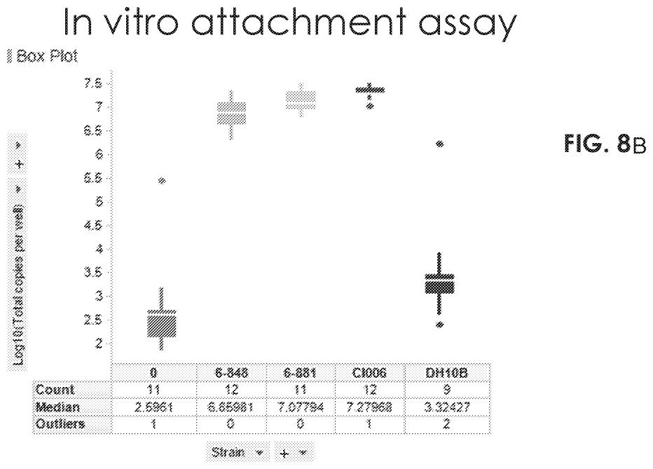

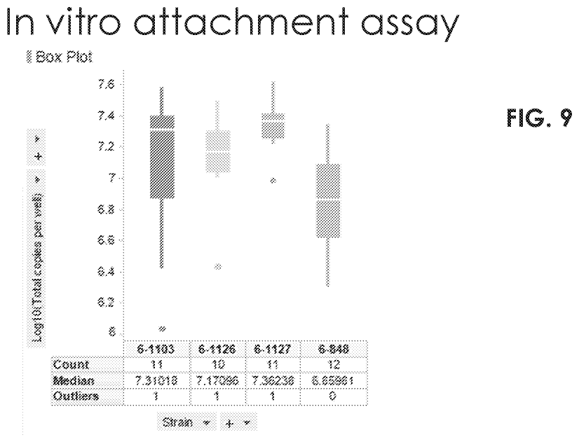

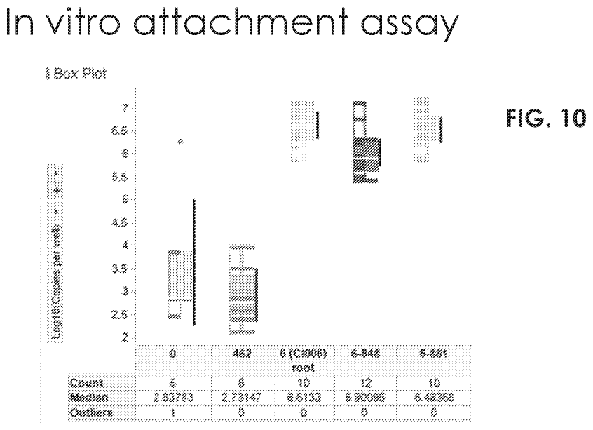

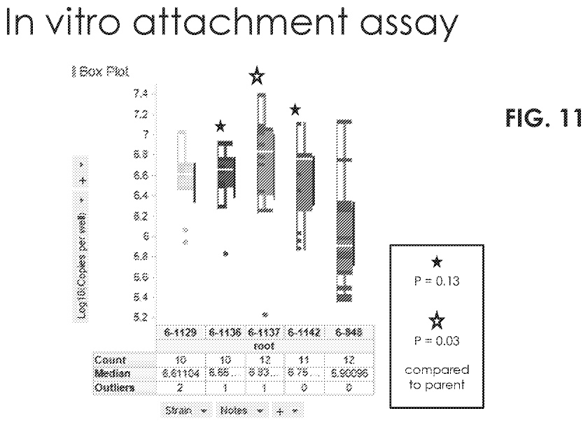

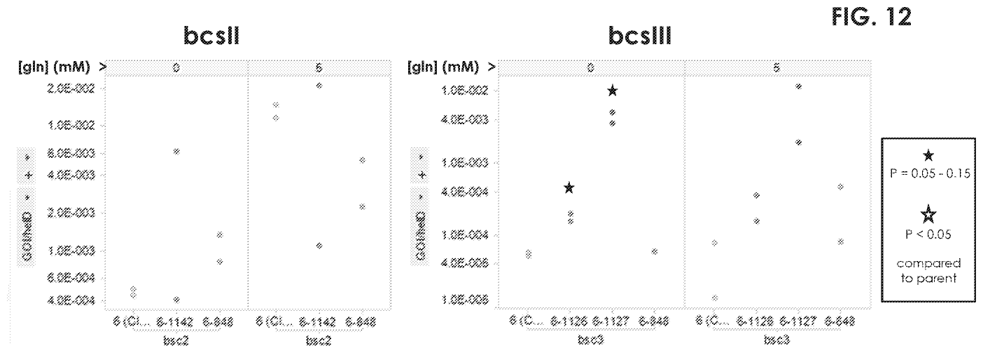

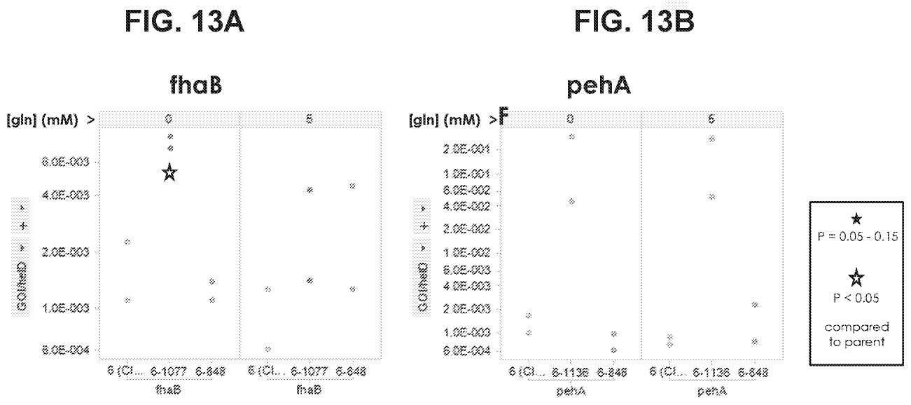

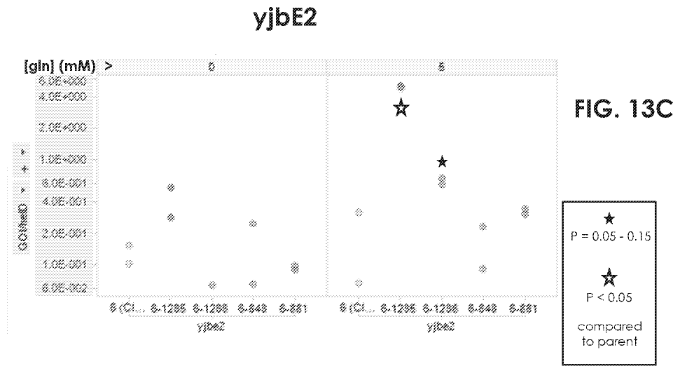

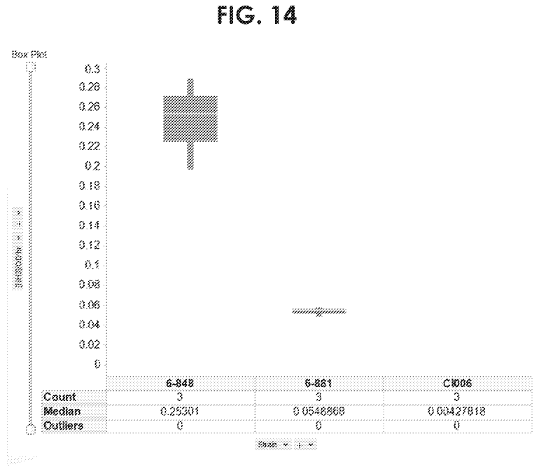

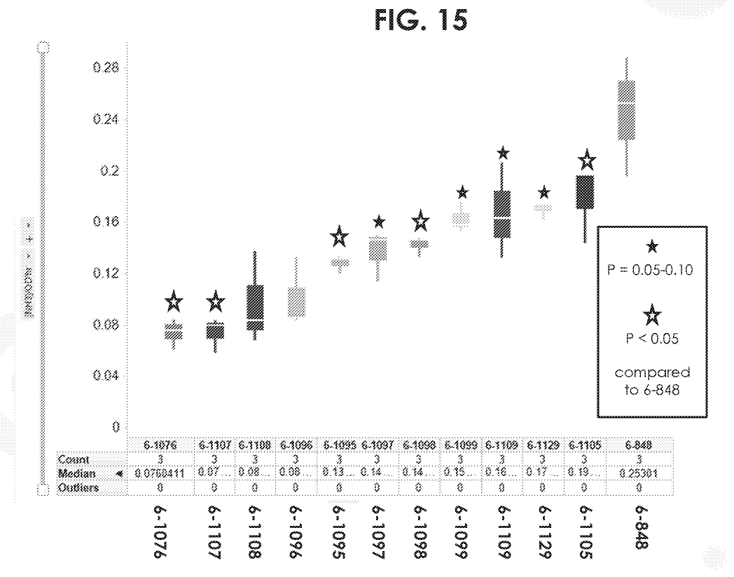

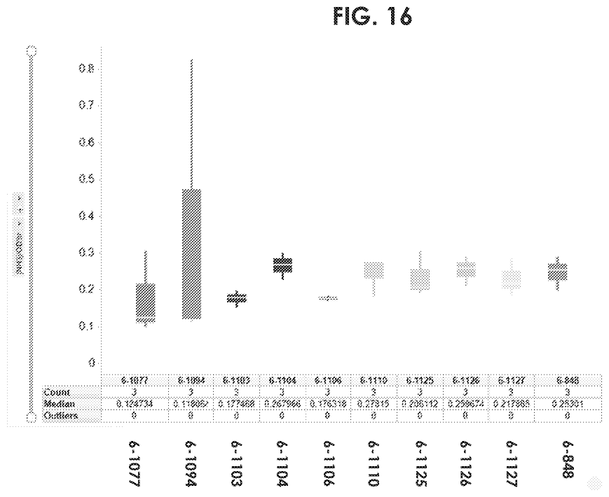

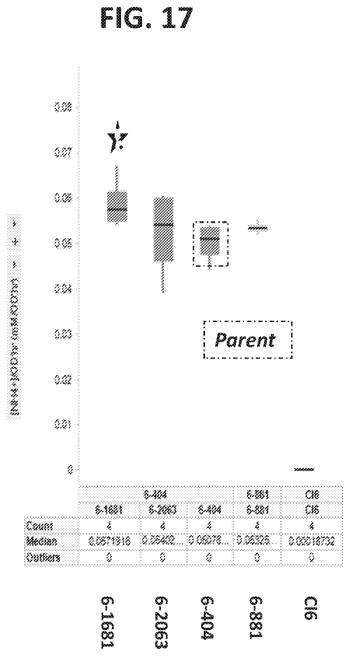

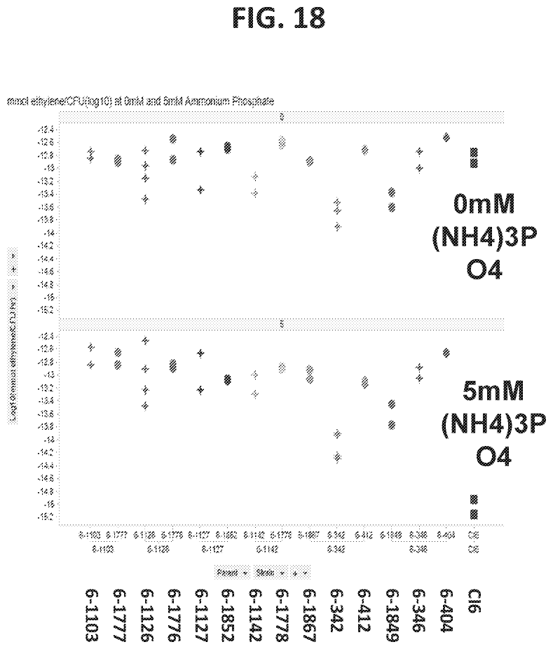

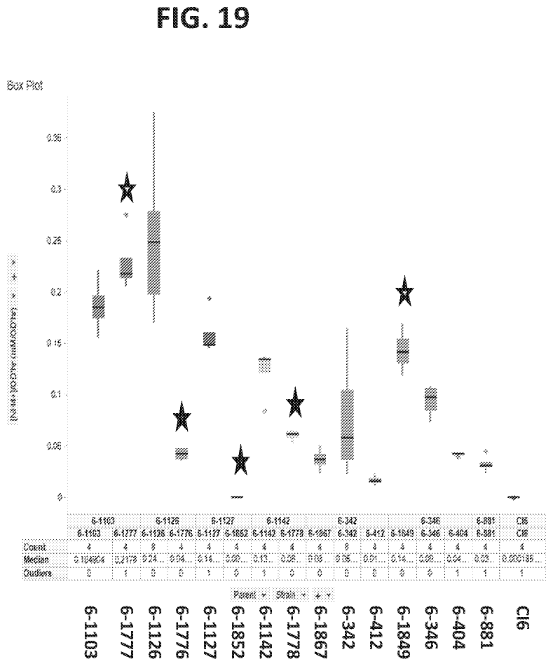

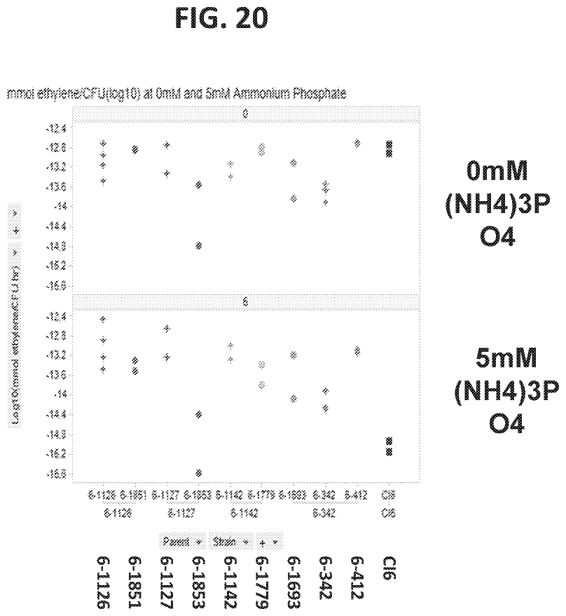

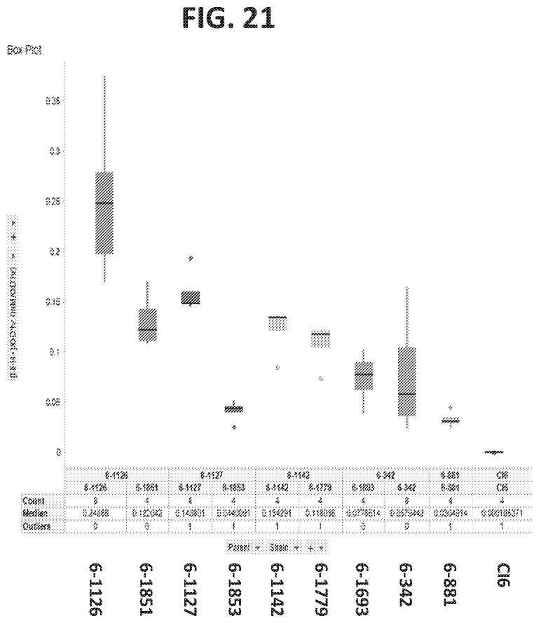

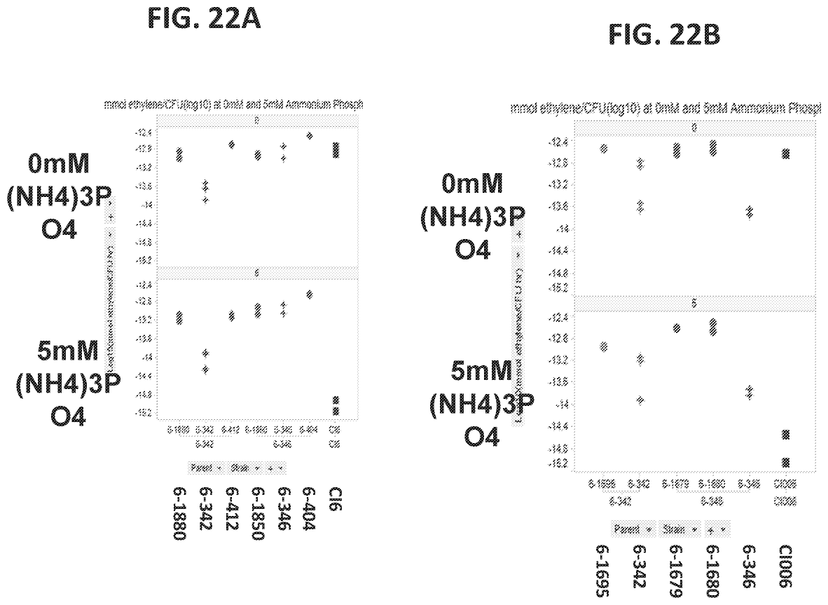

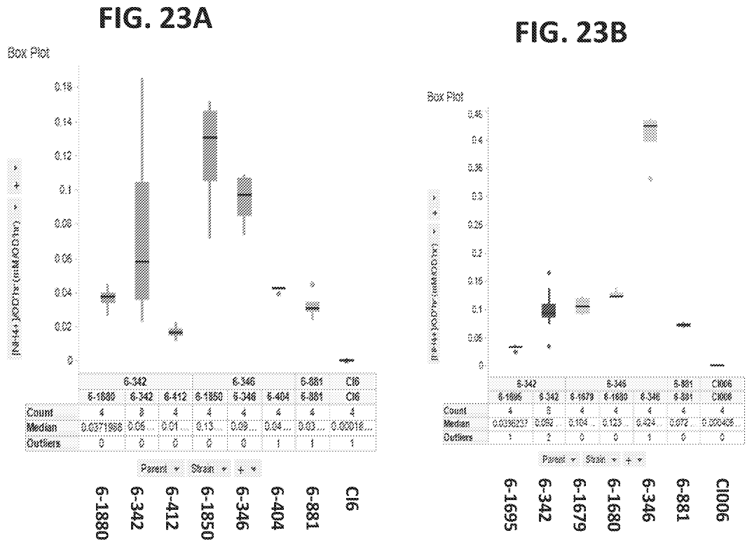

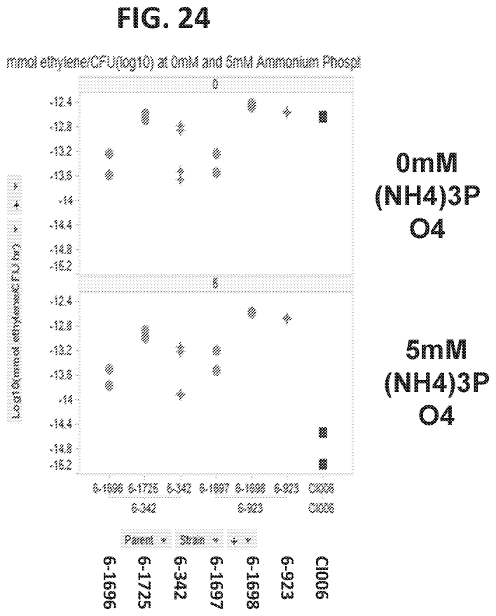

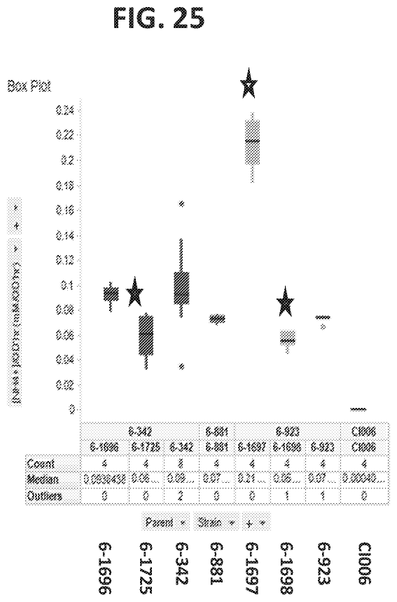

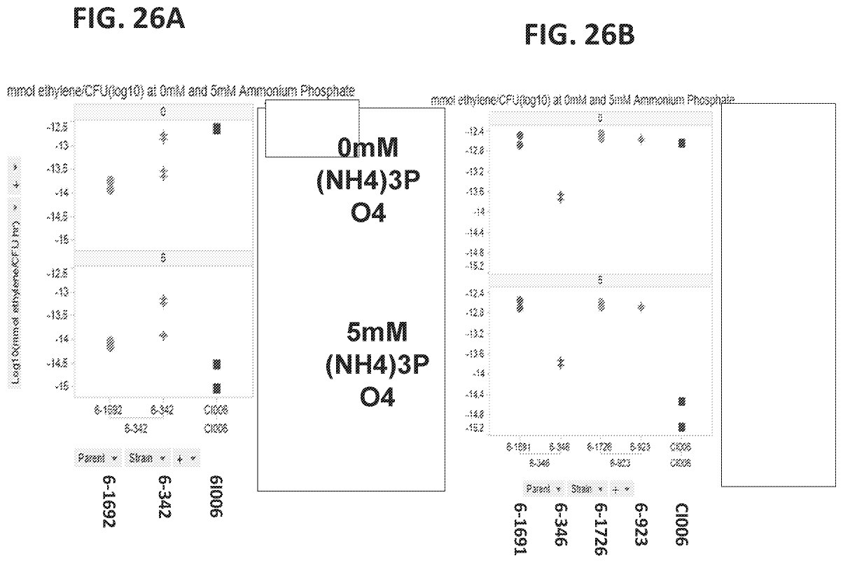

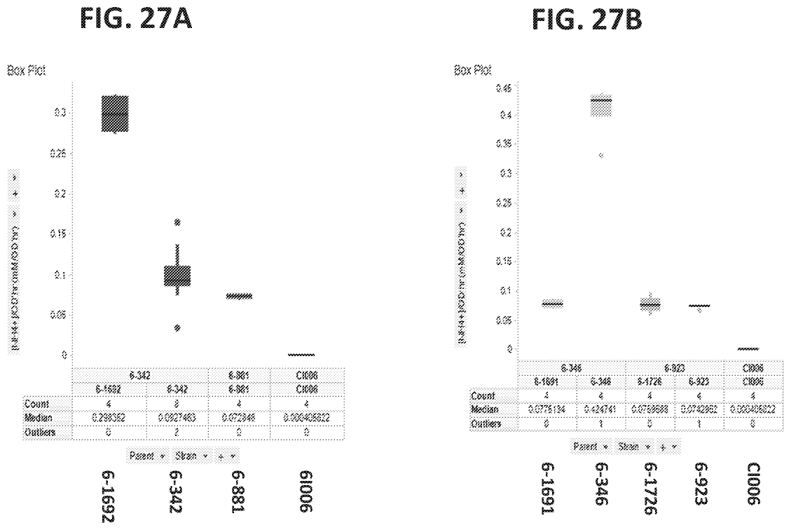

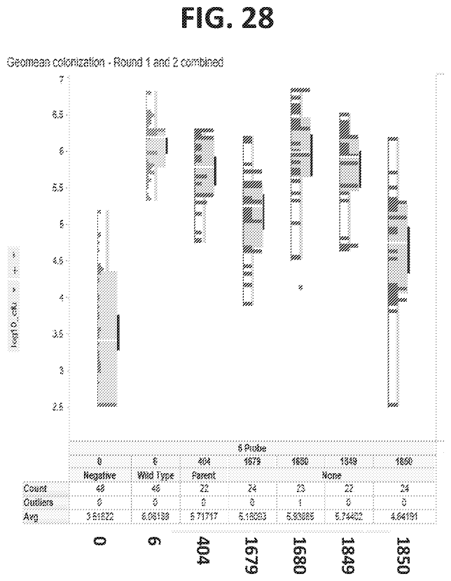

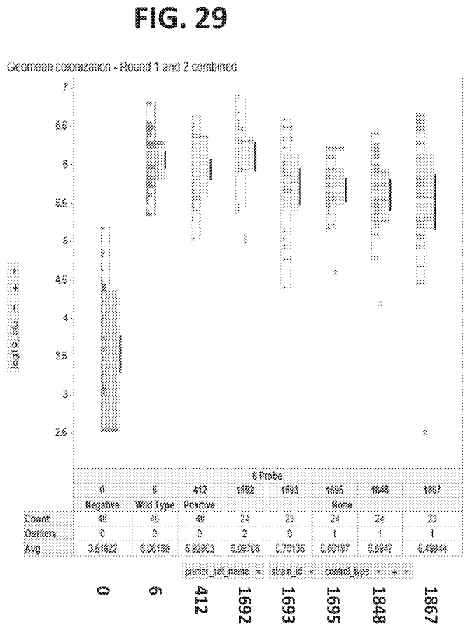

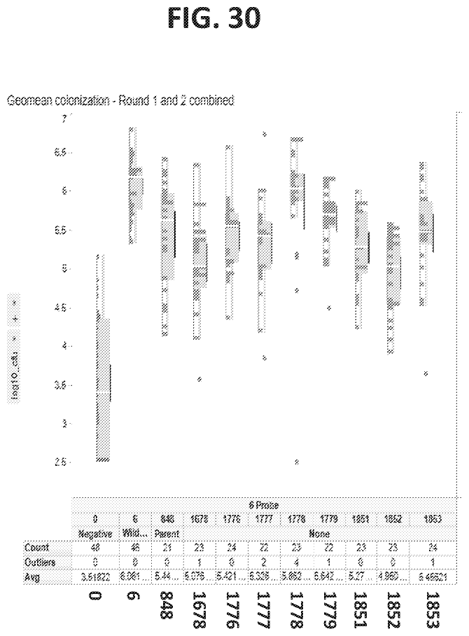

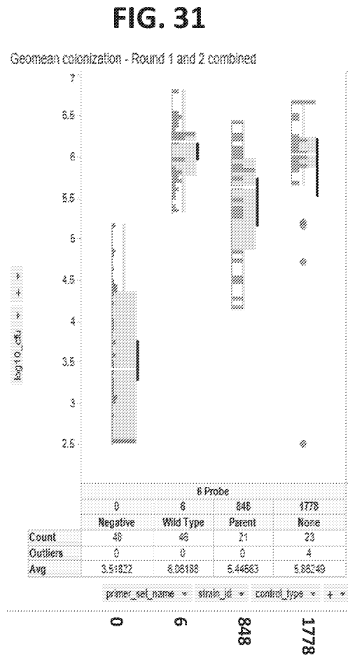

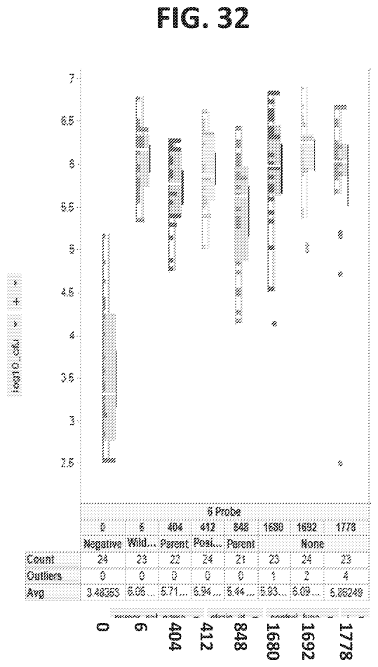

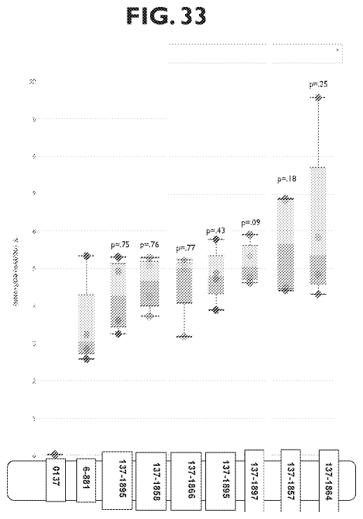

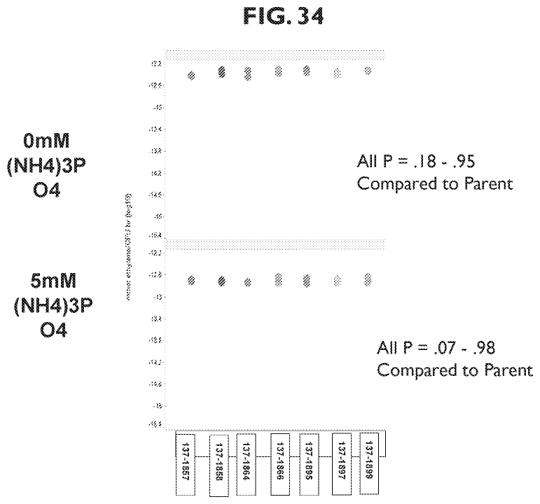

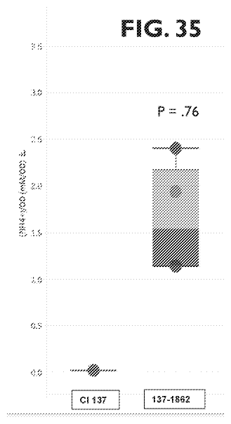

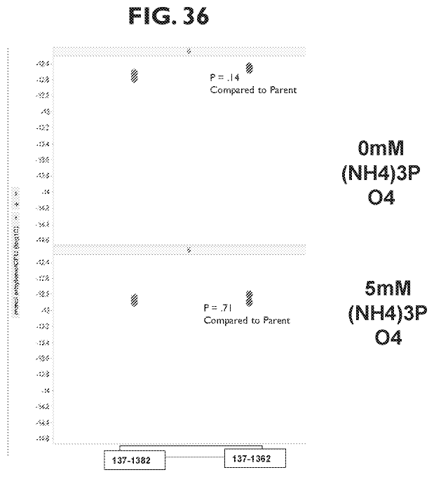

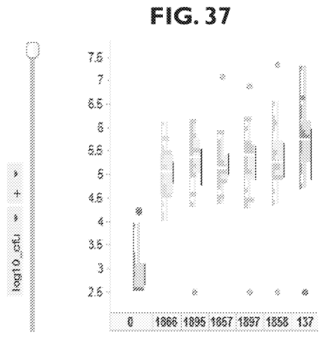

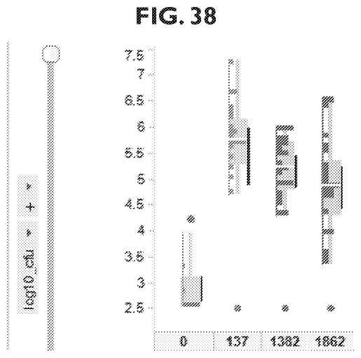

Several mutant strains were developed to investigate novel modifications which may increase colonization ability, nitrogen fixation activity, or nitrogen excretion. These strains are summarized in Table 11. Promoter and gene sequences are listed in Table 12. The remodeled strains were assessed relative to the wildtype, or to parental strains, to confirm altered expression of the modified gene (FIGS. 5, 12, and 13), for nitrogen reduction activity (FIGS. 6, 18, 20, 22A, 22B, 24, 26A, 26B, 34, and 36), nitrogen excretion (FIGS. 14, 15, 16,17, 19, 21, 23A, 23B, 25, 27A, 27B, 33, and 35), in vitro colonization (FIGS. 7, 8, 9,10, 11), and greenhouse colonization (FIGS. 28,29, 30, 31, 32, 37 and 38). The ammonium excretion assay was performed as described below. Some of the data is further summarized in Tables 13 and 14.

| Strain | | | | |
|---|---|---|---|---|
| ID | Species | Genotype (chromosome) | Plasmids | Cured? |
| 6-1098 | K. sacchari | ΔnifL::Prm1 ΔglnE ΔamtB Prm2-fahB | PB401, PB402, pUC- | no |
| | | | CI6-fhaB-prm-gRNA | |
| 462 | E. coli | F⁻ endA1 deoR⁺ recA1 galE15 galK16 nupG | none | N/A |
| | | rpsL Δ(lac)X74 φ80lacZΔM15 araD139 | | |
| | | Δ(ara, leu)7697 mcrA Δ(mrr-hsdRMS- | | |
| | | mcrBC) Str^{R} λ⁻ | | |
| 6-1076 | K. sacchari | ΔnifL::Prm1 ΔglnE::KO1ΔamtB fhaB_prm1 | PB401, PB402, pUC- | no |
| | | | CI6-fhaB-prm-gRNA | |
| 6-1077 | K. sacchari | ΔnifL::Prm1 ΔglnE::KO1ΔamtB fhaB_prm2 | PB401, PB402, pUC- | no |
| | | | CI6-fhaB-prm-gRNA | |
| 6-1094 | K. sacchari | ΔnifL::Prm1 ΔglnE::KO1 ΔamtB yjbE2_prm1 | PB401, PB402, pUC- | no |
| | | | CI6-yjbE 2-prm-gRNA | |
| 6-1095 | K. sacchari | ΔnifL::Prm1 ΔglnE::KO1 ΔamtB yjbE2_prm2 | PB401, PB402, pUC- | no |
| | | | CI6-yjbE 2-prm-gRNA | |
| 6-1096 | K. sacchari | ΔnifL::Prm1 ΔglnE::KO1 ΔamtB fhaB_prm9 | PB401, PB402, pUC- | no |
| | | | CI6-fhaB-prm-gRNA | |
| 6-1097 | K. sacchari | ΔnifL::Prm1 ΔglnE::KO2ΔamtB fhaB_prm1 | PB401, PB402, pUC- | no |
| | | | CI6-fhaB-prm-gRNA | |
| 6-1098 | K. sacchari | ΔnifL::Prm1 ΔglnE::KO2ΔamtB fhaB_prm2 | PB401, PB402, pUC- | no |
| | | | CI6-fhaB-prm-gRNA | |
| 6-1099 | K. sacchari | ΔnifL::Prm1 ΔglnE::KO2 ΔamtB fhaB_prm9 | PB401, PB402, pUC- | no |
| | | | CI6-fhaB-prm-gRNA | |
| 6-1103 | K. sacchari | ΔnifL::Prm1 ΔglnE::KO2 ΔamtB yjbE2_prm1 | PB401, PB402, pUC- | no |
| | | | CI6-yjbE_2-prm-gRNA | |
| 6-1104 | K. sacchari | ΔnifL::Prm1 ΔglnE::KO2 ΔamtB yjbE2_prm2 | PB401, PB402, pUC- | no |
| | | | CI6-yjbE_2-prm-gRNA | |
| 6-1105 | K. sacchari | ΔnifL::Prm1 ΔglnE::KO2 ΔamtB yjbE2_prm9 | PB401, PB402, pUC- | no |
| | | | CI6-yjbE_2-prm-gRNA | |
| 6-1106 | K. sacchari | ΔnifL::Prm1 ΔglnE::KO1 ΔamtB bcsII_prm2 | PB401, PB402, pUC- | no |
| | | | CI6-bcsII-prm-gRNA | |
| 6-1107 | K. sacchari | ΔnifL::Prm1 ΔglnE::KO1 ΔamtB bcsIII_prm1 | PB401, PB402, pUC- | no |
| | | | CI6-bcsIII-prm-gRNA | |
| 6-1108 | K. sacchari | ΔnifL::Prm1 ΔglnE::KO1 ΔamtB bcsIII_prm2 | PB401, PB402, pUC- | no |
| | | | CI6-bcsIII-prm-gRNA | |
| 6-1109 | K. sacchari | ΔnifL::Prm1 ΔglnE::KO1 ΔamtB bcsIII_prm9 | PB401, PB402, pUC- | no |
| | | | CI6-bcsIII-prm-gRNA | |
| 6-1110 | K. sacchari | ΔnifL::Prm1 ΔglnE::KO2 ΔamtB pehA-Prm2 | PB401, PB402, pUC- | no |
| | | | CI6-pehA-prm-gRNA | |
| 6-1125 | K. sacchari | ΔnifL::Prm1 ΔglnE::KO2 ΔamtB pehA-Prm1 | PB401, PB402, pUC- | no |
| | | | CI6-pehA-prm-gRNA | |
| 6-1126 | K. sacchari | ΔnifL::Prm1 ΔglnE::KO2 ΔamtB bcsIII_prm2 | PB401, PB402, pUC- | no |
| | | | CI6-bcsIII-prm-gRNA | |
| 6-1127 | K. sacchari | ΔnifL::Prm1 ΔglnE::KO2 ΔamtB bcsIII_prm9 | PB401, PB402, pUC- | no |
| | | | CI6-bcsIII-prm-gRNA | |
| 6-1129 | K. sacchari | ΔnifL::Prm1 ΔglnE::KO2 ΔamtB ΔglgA | PB401, PB402, pUC- | no |
| | | | CI6-glgA-del-gRNA | |
| 6-1136 | K. sacchari | ΔnifL::Prm1 ΔglnE::KO2 ΔamtB pehA-Prm9 | PB401, PB402, pUC- | no |
| | | | CI6-pehA-prm-gRNA | |
| 6-1137 | K. sacchari | ΔnifL::Prm1 ΔglnE::KO1 ΔamtB yjbE2_prm9 | PB401, PB402, pUC- | no |
| | | | CI6-yjbE_2-prm-gRNA | |
| 6-1142 | K. sacchari | ΔnifL::Prm1 ΔglnE::KO2 ΔamtB bcsll_prm2 | PB401, PB402, pUC- | no |
| | | | CI6-bcsII-prm-gRNA | |
| 6-1285 | K. sacchari | ΔnifL::Prm1 ΔglnE::KO1 ΔamtB yjbE2_prm1 | none | yes |
| 6-1286 | K. sacchari | ΔnifL::Prm1 ΔglnE::KO2 ΔamtB yjbE2_prm1 | none | yes |
| 6-1678 | K. sacchari | ΔnifL::Prm1 ΔglnE::KO2 ΔamtB bcsIII_prm9 | PB401, PB402 | no |
| 6-1679 | K. sacchari | ΔnifL::Prm1 ΔglnE::KO1 bcsIII_prm9 | PB401, PB402, pUC- | no |
| | | | CI6-bcsIII-prm-gRNA | |
| 6-1680 | K. sacchari | ΔnifL::Prm1 ΔglnE::KO1 bcsIII_prm2 | PB401, PB402, pUC- | no |
| | | | CI6-bcsIII-prm-gRNA | |
| 6-1681 | K. sacchari | ΔnifL::Prm1 ΔglnE::KO1 ΔglgA | PB401, PB402, pUC- | no |
| | | | CI6-glgA-del-gRNA | |
| 6-1691 | K. sacchari | ΔnifL::Prm1 ΔglnE::KO1 cysZ-Prm1 | PB401, PB402, pUC- | no |
| | | | CI6-cysZ-prm-gRNA | |
| 6-1692 | K. sacchari | ΔnifL::Prm5 ΔglnE::KO1 fhaB_prm2 | PB401, PB402, pUC- | no |
| | | | CI6-fhaB-prm-gRNA | |
| 6-1693 | K. sacchari | ΔnifL::Prm5 ΔglnE::KO1 yjbE2_prm1 | PB401, PB402, pUC- | no |
| | | | CI6-yjbE 2-prm-gRNA | |
| 6-1695 | K. sacchari | ΔnifL::Prm5 ΔglnE::KO1 bcsIII_prm9 | PB401, PB402, pUC- | no |
| | | | CI6-bcsIII-prm-gRNA | |
| 6-1696 | K. sacchari | AnifL::Prm5 ΔglnE::KO1 otsB-Prm1 | PB401, PB402, pUC- | no |
| | | | CI6-otsB-prm-gRNA | |
| 6-1697 | K. sacchari | ΔnifL::Prm1 ΔglnE::KO1 ΔamtB treZ-Prm2 | PB401, PB402, pUC- | no |
| | | otsB-Prm1 | CI6-otsB-prm-gRNA | |
| 6-1698 | K. sacchari | ΔnifL::Prm1 ΔglnE::KO1 ΔamtB treZ-Prm2 | PB401, PB402, pUC- | no |
| | | otsB-Prm2 | CI6-otsB-prm-gRNA | |
| 6-1725 | K. sacchari | AnifL::Prm5 ΔglnE::KO1 otsB-Prm2 | PB401, PB402, pUC- | no |
| | | | CI6-otsB-prm-gRNA | |
| 6-1726 | K. sacchari | ΔnifL::Prm1 ΔglnE::KO1 ΔamtB treZ-Prm2 | PB401, PB402, pUC- | no |
| | | cysZ-Prm1 | CI6-cysZ-prm-gRNA | |
| 6-1776 | K. sacchari | ΔnifL::Prm1 ΔglnE::KO2 ΔamtB bcsIII_prm2 | PB401, PB402, pUC- | no |
| | | pehA-Prm9 | CI6-pehA-prm-gRNA | |
| 6-1777 | K. sacchari | ΔnifL::Prm1 ΔglnE::KO2 ΔamtB yjbE2_prm1 | PB401, PB402, pUC- | no |
| | | pehA-Prm9 | CI6-pehA-prm-gRNA | |
| 6-1778 | K. sacchari | ΔnifL::Prm1 ΔglnE::KO2 ΔamtB bcsII_prm2 | PB401, PB402, pUC- | no |
| | | pehA-Prm9 | CI6-pehA-prm-gRNA | |
| 6-1779 | K. sacchari | ΔnifL::Prm1 ΔglnE::KO2 ΔamtB bcsII_prm2 | PB401, PB402, pUC- | no |
| | | yjbE2_prm1 | CI6-yjbE 2-prm-gRNA | |
| 6-1848 | K. sacchari | ΔnifL::Prm5 ΔglnE::KO1 bcsIII_prm2 | PB401, PB402, pUC- | no |
| | | | CI6-bcsIII-prm-gRNA | |
| 6-1849 | K. sacchari | ΔnifL::Prm1 ΔglnE::KO1 pehA-Prm9 | PB401, PB402, pUC- | no |
| | | | CI6-pehA-prm-gRNA | |
| 6-1850 | K. sacchari | ΔnifL::Prm1 ΔglnE::KO1 bcsII_prm2 | PB401, PB402, pUC- | no |
| | | | CI6-bcsII-prm-gRNA | |
| 6-1851 | K. sacchari | ΔnifL::Prm1 ΔglnE::KO2 ΔamtB bcsIII_prm2 | PB401, PB402, pUC- | no |
| | | yjbE2_prm1 | CI6-yjbE_2-prm-gRNA | |
| 6-1852 | K. sacchari | ΔnifL::Prm1 ΔglnE::KO2 ΔamtB bcsIII_prm9 | PB401, PB402, pUC- | no |
| | | pehA-Prm9 | CI6-pehA-prm-gRNA | |
| 6-1853 | K. sacchari | ΔnifL::Prm1 ΔglnE::KO2 ΔamtB bcsIII_prm9 | PB401, PB402, pUC- | no |
| | | yjbE2_prm1 | CI6-yjbE_2-prm-gRNA | |
| 6-1867 | K. sacchari | ΔnifL::Prm5 ΔglnE::KO1 pehA-Prm9 | PB401, PB402, pUC- | no |
| | | | CI6-pehA-prm-gRNA | |
| 6-1880 | K. sacchari | ΔnifL::Prm5 ΔglnE::KO1 bcsII_prm2 | PB401, PB402, pUC- | no |
| | | | CI6-bcsII-prm-gRNA | |
| 6-2063 | K. sacchari | ΔnifL::Prm1 ΔglnE::KO1 ΔglgA | none | yes |
| 6-342 | K. sacchari | ΔnifL::Prm5 ΔglnE::KO1 | PB401, PB402, pUC- | no |
| | | | CI6-glnE-1-gRNA | |
| 6-346 | K. sacchari | ΔnifL::Prm1 ΔglnE::KO1 | PB401, PB402, pUC- | no |
| | | | CI6-glnE-1-gRNA | |
| 6-404 | K. sacchari | ΔnifL::Prm1 ΔglnE::KO1 | none | yes |
| 6-412 | K. sacchari | ΔnifL::Prm5 ΔglnE::KO1 | none | yes |
| 6-435 | K. sacchari | ΔnifL::Prm1 ΔglnE::KO1 ΔamtB | PB401, PB402, pUC- | no |
| | | | CI6-amtB-1-gRNA | |
| 6-848 | K. sacchari | ΔnifL::Prm1 ΔglnE::KO2 ΔamtB | none | yes |
| 6-881 | K. sacchari | ΔnifL::Prm1 ΔglnE::KO1 ΔamtB | none | yes |
| 6-915 | K. sacchari | ΔnifL::Prm1 ΔglnE::KO1 ΔamtB bcsI-Prm2 | PB401, PB402, pUC- | no |
| | | | CI6-bcsI-prm-gRNA | |
| 6-916 | K. sacchari | ΔnifL::Prm1 ΔglnE::KO1 ΔamtB cysZ-Prm1 | PB401, PB402, pUC- | no |
| | | | CI6-cysZ-prm-gRNA | |
| 6-921 | K. sacchari | ΔnifL::Prm1 ΔglnE::KO1 ΔamtB otsB-Prm1 | PB401, PB402, pUC- | no |
| | | | CI6-otsB-prm-gRNA | |
| 6-922 | K. sacchari | ΔnifL::Prm1 ΔglnE::KO1 ΔamtB otsB-Prm2 | PB401, PB402, pUC- | no |
| | | | CI6-otsB-prm-gRNA | |
| 6-923 | K. sacchari | ΔnifL::Prm1 ΔglnE::KO1 ΔamtB treZ-Prm2 | PB401, PB402, pUC- | no |
| | | | CI6-treZ-prm-gRNA | |
| CI006 | K. sacchari | wild type | none | N/A |
| PBC6.22 | K. sacchari | ΔnifL::Prm3 | PB401, PB402, pUC- | no |
| | | | CI6-nifL-2-gRNA | |
| 137- | Klebsiella | ΔglnE::KO2-v2 ΔnifL-Prm1.2 | none | yes |
| 1382 | variicola | | | |
| 137- | Klebsiella | ΔnifL::PinfC ΔglnE::KO2 | none | yes |
| 1586 | variicola | | | |
| 137- | Klebsiella | ΔnifL::PinfC ΔglnE::KO2 otsB_ATG_prm8.2 | none | yes |
| 1857 | variicola | | | |
| 137- | Klebsiella | ΔnifL::PinfC ΔglnE::KO2 otsB_ATG_prm16. | none | yes |
| 1858 | variicola | 1 | | |
| 137- | Klebsiella | ΔglnE::KO2-v2 ΔnifL-Prm1.2 ΔglgA | none | yes |
| 1862 | variicola | | | |

TABLE 11
Remodeled strains

| | Genotype | | |
|---|---|---|---|
| Species | Notation | | Notes |
| | | Prm(lowercase)-gene(uppercase) sequence | |
| Kosakonia | bcsII_ | Tcaccacggcgataaccataggttttcggcgtggccacatccatggtgaatcccacttttt | Gene |
| sacchari | prm2 | ccagcacgcgcgccacttcatcgggtcttaaatacatagattttcctcgtcatctttccaaa | sequence |
| | | gcctcgccaccttacatgactgagcatggaccgtgactcagaaaattccacaaacgaac | is of |
| | | ctgaaaggcgtgattgccgtctggccttaaaaattatggtctaaactaaaatttacatcga | first gene |
| | | aaacgagggaggatcctATGCATAACAATGAACCCAAAACGCAGTCAGA | in bcsII |
| | | CCCTACTCTGGGCTATACATTCCAAAATGATTTTCTGGCGTTAAGTA | operon |
| | | AGGCATTTTCATTGCCTGAAATTGATTACGCCGATATTTCCCAGCGA | |
| | | GAACAGTTGGCGGCGGCATTTAAGCGCTGGCCGCTGCTGGCAGAA | |
| | | TTAGCCCGTCAACAATAA (SEQ ID NO: 7) | |
| Kosakonia | bcsIII_ | cgtcctgtaataataaccggacaattcggactgattaaaaaagcgcccttgtggcgctttt | Gene |
| sacchari | prm1 | tttatattcccgcctccatttaaaataaaaaatccaatcggatttcactatttaaactggcc | sequence |
| | | attatctaagatgaatccgatggaagctcgctgttttaacacgcgttttttaaccttttattg | is of |
| | | aaagtcggtgcttctttgagcgaacgatcaaatttaagtggattcccatcaaaaaaatatt | first gene |
| | | ctcaacctaaaaaagtttgtgtaatacttgtaacgctacatggagattaactcaatctaga | in bcsIII |
| | | gggtattaataatgaatcgtactaaactggtactgggcATGCCTTTATTCTTTTCT | operon, |
| | | GTGCAAGATGATAACAACATGAAAAAAATCATCGGAATTATTTTCA | acsAB 2 |
| | | CGCTGATGCTGCTGTTATTATCGCTGCTGGTAGTGATTACTCCCATG | |
| | | AGCAGCGATAAACAATACCTCTTTGGCCTTAGCGTGATGGCAGCGG | |
| | | TATTTATCCTGGGAAGAGTGAAAAGCAAAAAAGCCGTACTGGCAAT | |
| | | GCTTTCCCTCTCCGTATTGATGTCGACACGTTATATTTGGTGGCGCG | |
| | | CCACGACAACGCTGCATTTTGATTCGACGGTAGAGATGCTGCTCGG | |
| | | CAGTTTACTTTTTGCTGCGGAAATTTACTCGTGGACGATTTTACTGC | |
| | | TGGGTTATATTCAAATGGCGTGGCCATTAGAACGCCCGATTGCGCC | |
| | | TTTACCGGCAGATAAATCAACCTGGCCGACCGTCGATATTTATGTCC | |
| | | CCTCTTATAATGAAAGCCTTGAGGTTGTTCGCGATACGGTTCTGGC | |
| | | GGCGCAGTGTATCGAATATCCGCAAGATAAAGTGAAGGTTTATATT | |
| | | CTTGACGACGGAAAGCGCGATGAATTCCGCGATTTCGCCGCTGAAG | |
| | | CGGGCGTTGGGTATATTACCCGCCCGGATAACAGCCACGCCAAAGC | |
| | | CGGTAATCTCAACCACGCGATGACCATCACCCATGGTGAGCTTATCT | |
| | | GCGTGTTCGACTGCGACCACGTGGCAACGCGCGTGTTCCTGCAAGC | |
| | | CACGATTGGCGAGTTTTTCCGCGACGATAAACTGGCGCTGATCCAG | |
| | | ACGCCGCACCACTTCTATTCGCTCGATCCATTTGAGCGCAACCTGAC | |
| | | AGCAGCAAAGCGCGTTCCCCACGAAGGGGCGCTGTTCTACGGCCC | |
| | | GGTGCAGCAAGGCAATGACAACTGGAACGCCACGTTCTTCTGCGG | |
| | | CTCCTGCGCGGTGATCCGTCGTAGCGCGCTCAATGAAGTCGGCGGC | |
| | | TTTGCCGTGGAAACCGTGACCGAAGATGCGCATACCGCGCTGAAAT | |
| | | TACAGCGTCGCGGCTGGAACACCGCGTTTCTGGATATTCCGCTGGC | |
| | | GGCGGGGCTGGCAACCGAGCGTCTGGCGCTGCATGTTAACCAGCG | |
| | | (SEQ ID NO: 8) | |
| Kosakonia | bcsIII_ | tcaccacggcgataaccataggttttcggcgtggccacatccatggtgaatcccacttttt | Gene |
| sacchari | prm2 | ccagcacgcgcgccacttcatcgggtcttaaatacatagattttcctcgtcatctttccaaa | sequence |
| | | gcctcgccaccttacatgactgagcatggaccgtgactcagaaaattccacaaacgaac | is of |
| | | ctgaaaggcgtgattgccgtctggccttaaaaattatggtctaaactaaaatttacatcga | first gene |
| | | aaacgagggaggatcctATGCCTTTATTCTTTTCTGTGCAAGATGATAAC | in bcsIII |
| | | AACATGAAAAAAATCATCGGAATTATTTTCACGCTGATGCTGCTGTT | operon, |
| | | ATTATCGCTGCTGGTAGTGATTACTCCCATGAGCAGCGATAAACAA | acsAB 2 |
| | | TACCTCTTTGGCCTTAGCGTGATGGCAGCGGTATTTATCCTGGGAA | |
| | | GAGTGAAAAGCAAAAAAGCCGTACTGGCAATGCTTTCCCTCTCCGT | |
| | | ATTGATGTCGACACGTTATATTTGGTGGCGCGCCACGACAACGCTG | |
| | | CATTTTGATTCGACGGTAGAGATGCTGCTCGGCAGTTTACTTTTTGC | |
| | | TGCGGAAATTTACTCGTGGACGATTTTACTGCTGGGTTATATTCAAA | |
| | | TGGCGTGGCCATTAGAACGCCCGATTGCGCCTTTACCGGCAGATAA | |
| | | ATCAACCTGGCCGACCGTCGATATTTATGTCCCCTCTTATAATGAAA | |
| | | GCCTTGAGGTTGTTCGCGATACGGTTCTGGCGGCGCAGTGTATCGA | |
| | | ATATCCGCAAGATAAAGTGAAGGTTTATATTCTTGACGACGGAAAG | |
| | | CGCGATGAATTCCGCGATTTCGCCGCTGAAGCGGGCGTTGGGTATA | |
| | | TTACCCGCCCGGATAACAGCCACGCCAAAGCCGGTAATCTCAACCA | |
| | | CGCGATGACCATCACCCATGGTGAGCTTATCTGCGTGTTCGACTGC | |
| | | GACCACGTGGCAACGCGCGTGTTCCTGCAAGCCACGATTGGCGAG | |
| | | TTTTTCCGCGACGATAAACTGGCGCTGATCCAGACGCCGCACCACTT | |
| | | CTATTCGCTCGATCCATTTGAGCGCAACCTGACAGCAGCAAAGCGC | |
| | | GTTCCCCACGAAGGGGCGCTGTTCTACGGCCCGGTGCAGCAAGGC | |
| | | AATGACAACTGGAACGCCACGTTCTTCTGCGGCTCCTGCGCGGTGA | |
| | | TCCGTCGTAGCGCGCTCAATGAAGTCGGCGGCTTTGCCGTGGAAAC | |
| | | CGTGACCGAAGATGCGCATACCGCGCTGAAATTACAGCGTCGCGG | |
| | | CTGGAACACCGCGTTTCTGGATATTCCGCTGGCGGCGGGGCTGGCA | |
| | | ACCGAGCGTCTGGCGCTGCATGTTAACCAGCG | |
| | | (SEQ ID NO: 9) | |
| Kosakonia | bcsIII_ | atattgacaccatgacgcgcgtaatgctgattggttctgtgacgctggtaatgattgtcga | Gene |
| sacchari | prm9 | aattctgaacagtgccatcgaagccgtagtagaccgtattggtgcagaattccatgaact | sequence |
| | | ttccgggcgggcgaaggatatggggtcggcggcggtgctgatgtccatcctgctggcgat | is of |
| | | gtttacctggatcgcattactctggtcacattttcgataacgcttccagaattcgataacgc | first gene |
| | | cctggttttttgcttaaatttggttccaaaatcgcctttagctgtatatactcacagcataac | in bcsIII |
| | | tgtatatacacccagggggcgggATGCCTTTATTCTTTTCTGTGCAAGATGA | operon, |
| | | TAACAACATGAAAAAAATCATCGGAATTATTTTCACGCTGATGCTGC | acsAB 2 |
| | | TGTTATTATCGCTGCTGGTAGTGATTACTCCCATGAGCAGCGATAAA | |
| | | CAATACCTCTTTGGCCTTAGCGTGATGGCAGCGGTATTTATCCTGGG | |
| | | AAGAGTGAAAAGCAAAAAAGCCGTACTGGCAATGCTTTCCCTCTCC | |
| | | GTATTGATGTCGACACGTTATATTTGGTGGCGCGCCACGACAACGC | |
| | | TGCATTTTGATTCGACGGTAGAGATGCTGCTCGGCAGTTTACTTTTT | |
| | | GCTGCGGAAATTTACTCGTGGACGATTTTACTGCTGGGTTATATTCA | |
| | | AATGGCGTGGCCATTAGAACGCCCGATTGCGCCTTTACCGGCAGAT | |
| | | AAATCAACCTGGCCGACCGTCGATATTTATGTCCCCTCTTATAATGA | |
| | | AAGCCTTGAGGTTGTTCGCGATACGGTTCTGGCGGCGCAGTGTATC | |
| | | GAATATCCGCAAGATAAAGTGAAGGTTTATATTCTTGACGACGGAA | |
| | | AGCGCGATGAATTCCGCGATTTCGCCGCTGAAGCGGGCGTTGGGT | |
| | | ATATTACCCGCCCGGATAACAGCCACGCCAAAGCCGGTAATCTCAA | |
| | | CCACGCGATGACCATCACCCATGGTGAGCTTATCTGCGTGTTCGACT | |
| | | GCGACCACGTGGCAACGCGCGTGTTCCTGCAAGCCACGATTGGCG | |
| | | AGTTTTTCCGCGACGATAAACTGGCGCTGATCCAGACGCCGCACCA | |
| | | CTTCTATTCGCTCGATCCATTTGAGCGCAACCTGACAGCAGCAAAGC | |
| | | GCGTTCCCCACGAAGGGGCGCTGTTCTACGGCCCGGTGCAGCAAG | |
| | | GCAATGACAACTGGAACGCCACGTTCTTCTGCGGCTCCTGCGCGGT | |
| | | GATCCGTCGTAGCGCGCTCAATGAAGTCGGCGGCTTTGCCGTGGAA | |
| | | ACCGTGACCGAAGATGCGCATACCGCGCTGAAATTACAGCGTCGC | |
| | | GGCTGGAACACCGCGTTTCTGGATATTCCGCTGGCGGCGGGGCTG | |
| | | GCAACCGAGCGTCTGGCGCTGCATGTTAACCAGCG | |
| | | (SEQ ID NO: 10) | |
| Kosakonia | bcsI- | Tcaccacggcgataaccataggttttcggcgtggccacatccatggtgaatcccacttttt | Gene |
| sacchari | Prm2 | ccagcacgcgcgccacttcatcgggtcttaaatacatagattttcctcgtcatctttccaaa | sequence |
| | | gcctcgccaccttacatgactgagcatggaccgtgactcagaaaattccacaaacgaac | is of |
| | | ctgaaaggcgtgattgccgtctggccttaaaaattatggtctaaactaaaatttacatcga | first gene |
| | | aaacgagggaggatcctATGGGTCATTACGATGATTTGCAGAGGTTTAA | in bcsI |
| | | GGACAAGACTCGCAATCAGAAGCACGATTTTAAGGATCTCTCCGCA | operon |
| | | CAGAACCTCGCACAAGACCAGAGCAATTGGGCAATCATTAACCAGC | |
| | | TTTCTCCTGCCACGGATGAATCAATGCTGGCAATGGGCGGCCATGT | |
| | | GTCACTGCCTGTCCCGCAATCCGTCGATCCTGCGCTTTTTGCCGCGC | |
| | | AAGAGCCGGCTGCGTTGGTGGATGATATTGCACCTGCGCCTGCGTC | |
| | | AACAACTTCTCTGCTAAAAGACGTCGCCAGCCAGCTTGATGCAGTG | |
| | | GCTCCGGCATTCAACGCCGCAGCCTACGCGCCTGCGCCTGCAAGCG | |
| | | TTGCACCGGTTATGCAGATGCCATCGCCTGCAAATCCTTCTTCGGCT | |
| | | CCTGTCAGCTATGCGCGCCTGTTCGCAGCGAAAGCGACGGAGGAG | |
| | | AAGCCCCGCGCGGAAAAAAATCAGCCCTTACACTCGTTGTTAGAAA | |
| | | GGATCGCTTCATGCCGTTGA (SEQ ID NO: 11) | |
| Kosakonia | cysZ- | Cgtcctgtaataataaccggacaattcggactgattaaaaaagcgcccttgtggcgctttt | |
| sacchari | Prm1 | tttatattcccgcctccatttaaaataaaaaatccaatcggatttcactatttaaactggcc | |
| | | attatctaagatgaatccgatggaagctcgctgttttaacacgcgttttttaaccttttattg | |
| | | aaagtcggtgcttctttgagcgaacgatcaaatttaagtggattcccatcaaaaaaatatt | |
| | | ctcaacctaaaaaagtttgtgtaatacttgtaacgctacatggagattaactcaatctaga | |
| | | gggtattaataatgaatcgtactaaactggtactgggcATGGTTTCATCATCCCCG | |
| | | TCTGTTGCTCGCAGCGGCATGTTTTACTTTTCCCAGGGCTGGAAACT | |
| | | GGTCACACTGCCAGGCATTCGTCGCTTTGTGATCTTGCCGTTAATGG | |
| | | TGAATATTTTGCTGATGGGCGGCGCGTTCTGGTGGCTGTTTACCCG | |
| | | CCTGGATAGTTGGATCCCTTCGCTGATGAGCCATGTGCCGGACTGG | |
| | | CTGCAATGGCTGAGCTATTTACTGTGGCCAATTGCCGTTGTCTCAAT | |
| | | TCTGTTGGTCTTCGGCTACTTCTTCTCAACCCTTGCGAACTGGATTGC | |
| | | CGCTCCCTTTAATGGCTTGCTGGCAGAACAGCTGGAAGCACGCCTT | |
| | | ACCGGCGCGACACCACCCGACGTCGGCGTTTTTGGGCTGCTCAAAG | |
| | | ATGTACCGCGCATCATGAAGCGCGAATGGCAAAAACTGGCCTGGT | |
| | | ATCTGCCCCGCGCCTTGCTGCTGCTGGTGCTTTATTTTATTCCCGGT | |
| | | ATTGGGCAAACCGTCGCCCCGGTGCTGTGGTTCCTGTTCAGTGCGT | |
| | | GGATGTTGGCTATCCAGTATTGCGACTATCCGTTTGATAACCATAAG | |
| | | GTGCCGTTTAAAGCGATGCGCGTTTCCTTGCGCGAACGCAAGGTTA | |
| | | TTAATATGCAATTCGGTGCGCTCACCAGCCTGTTTACGATGATTCCG | |
| | | GTGTTGAATTTGGTCGTGATGCCCGTAGCGGTGTGTGGCGCAACG | |
| | | GCGATGTGGGTCGATTGCTACCGGGAAAAACACGCGGTCTGGCGA | |
| | | TAA (SEQ ID NO: 12) | |
| Kosakonia | fhaB_ | cgtcctgtaataataaccggacaattcggactgattaaaaaagcgcccttgtggcgctttt | |
| sacchari | prm1 | tttatattcccgcctccatttaaaataaaaaatccaatcggatttcactatttaaactggcc | |
| | | attatctaagatgaatccgatggaagctcgctgttttaacacgcgttttttaaccttttattg | |
| | | aaagtcggtgcttctttgagcgaacgatcaaatttaagtggattcccatcaaaaaaatatt | |
| | | ctcaacctaaaaaagtttgtgtaatacttgtaacgctacatggagattaactcaatctaga | |
| | | gggtattaataatgaatcgtactaaactggtactgggcGTGATTAATTCGGAAAA | |
| | | GAAAGTCCAACGCCTTGTAAGAACTCAGCTGGCCCCGTTGACGGTA | |
| | | ACAATGATATTCGCATTACCTGCTGTTGCGCATTCCGCTGGTTTAAG | |
| | | TATGAAAAATGGCACGCTATATAATGCGAATGGCGTGCCGGTCGTG | |
| | | GATATTAATAAACCTAACAGCAATGGCTTGTCACATAACGTCTGGG | |
| | | ACAACCTGAACGTCGATAAAAACGGTGTTATTTTTAATAACAGCGG | |
| | | CTCAGCATCCGATACGGTATTGGGCGGCACCATCCAGGGTAACAGC | |
| | | AATCTGACATCCGGGTCGGCAAAAGTTATTCTCAACGAAGTTACCT | |
| | | CCAAAAATGCGTCGACGATCAACGGTATGATGGAAGTTGCGGGTG | |
| | | ATAAAGCCGCGTTGATTATCGCCAACCCCAATGGCATTGCGGTAAA | |
| | | TGGTGGCGGTACGATCAATACCGATAAACTCACTCTGACCACTGGC | |
| | | ACACCAGATATTCAGAACGACAAACTCGCTGGTTATTCCGTCAACG | |
| | | GCGGTACCATCACGATCAGCAAACTGGATAACGCCAGCCCAACTGA | |
| | | AATTTTGTCACGCAACGTAGTGCTCAGCGGGAAAGTTACCGCTGAC | |
| | | GAACTGAATATTGTTGCGGGTAATAACTACGTTAATACAGACGGCC | |
| | | AGGTGACCGGAACTGTCACTGCGTCAGGCTCGCGTAACGGCTATA | |
| | | GCGTTGACGTTGCGCAAGTTGGCGGCATGTACGCGAATAAAATTAA | |
| | | TCTTATCAGTACCGAAAGCGGCGTGGGCGTGCGTAATCAGGGCGT | |
| | | AATTGCCGGTGGCACTGGTGGGATCAGCATTGATGCTAATGGCCA | |
| | | GCTACTGAACAGTAATGCGCAGATTCAATCGACCGGTTCGACAAAG | |
| | | ATTAATACCAACGGCAAATTGGACAATACAACGGGTCTTATTACTTC | |
| | | TGCTGGTGCTATTTATCTCGATACTAATAAGAACGCTATTGTTAATG | |
| | | CCCGTTCAGGTACGATTTCGACGACGGCAGATATCTACGTTAATAG | |
| | | CGGCGCAGTTGATAATACCAACGGCAAAATTGCCGCCAGCGGCGT | |
| | | GCTGGCTGTCGATACCAATAACAATACTTTTACCAACGCCGGTAAA | |
| | | GGGAACGATGTCGGTATCGAAGCGGGTATTGTCACCTTAAAAACC | |
| | | GGTACGCTAAATAATAGTAACGGCCAGATCCACGGCGGCTATCTGG | |
| | | GGCTTGAATCCGGCGCGATTAACAATAACAGCGGTATTCTGGAAAC | |
| | | GTCAGGCGACATGGACATTGTCAGCCTTGGAAATGTCGACAATAAC | |
| | | AAAGGCCTGATCCGATCTGCCAGCGGCCATATCAACATCCTGTCTG | |
| | | GCGGCACTATCAACAACGGCAGTACCAAAACAGCGGATACCGTCA | |
| | | GTTCCGACTCCTTAGGCATCGCAGCCGATACCGGTGTAGTAATCAA | |
| | | CGCAAACAGCATCAATAATAGCGGTGGTCAGATCGCATCGAACGG | |
| | | CGATGTGTCGCTAAACAGTAAGGGCCTGGTCAATAACGATTCCGGT | |
| | | AATCTTCTTTCCAATAGCAAAGTGATTGTGAAGGGCGGTTCTTATCG | |
| | | TAATGACTACGGTGGCATCGGCGGCAAGAAGGGCGTGGAGGTGTC | |
| | | GGTTAACGGCGACCTGAGCAACTATATCGGCGTTGTGAGTGCGGA | |
| | | AGAAGGTGATATCTCACTGAGTGCAAATGCAGTGGATACCCACGG | |
| | | CGGTTTCATGATGGGGCAAAATATCTCCATCGACGCGAAAGCCGGC | |
| | | GTAAATACCACCCGATCGCTGGTATTAGCCAGTGAAAAGCTGGCTA | |
| | | TCCATGCAGGCGGAAATGTTGATAACCGTGATGGTAATAGCTTTGG | |
| | | CAATGACTTCGGCCTCTATTTCGGCATGCCGCAACAGACCGGTGGC | |
| | | TTGATCGGTAAAGGTGGCGTTGAGATTTCCGGGCAGAATATTTACA | |
| | | ACAACAACAGCCGCATCATTGCAGAAAATGGGCCAATGAGCCTGCA | |
| | | AGCGAAAGGGAACATTGATAACACGCGTGCGCTGATGGTCAGCGG | |
| | | GGCCGATACCGCAATCAAAACCGGCGGGGCTTTCTACAATAACTAC | |
| | | GCAACGATTTCCAGCGGAGGCAACCTCACACTCGATACCGTTTCCCT | |
| | | GGAAAACTCCAGCAGCGGCACGCTTATCGATAATAACGCCACAGGT | |
| | | TTCATCAACTCAGATAAAGATATCACGCTGAATGTGGACAACAATTT | |
| | | CACGAACTACGGCTGGATCAGTGGTAAAGGCGCAGTGGCGCTGAA | |
| | | TGTCGTGAAAGGGGCGTTTTATAACCGTAATACCATTGCGGCTGAT | |
| | | AAGTCACTTGCGATCAGTGCCCTGAACGGTATCGAGAACTTTAAAG | |
| | | ATATCTCTGCAGGCGGCAGCCTGACGCTGGATACGCAACGTCATGT | |
| | | GACAAATAACAGCAACAGCAATATGGTCGGTAACGGGATTACTATT | |
| | | AACGCGGTAAACGACATTAATAACCGCGGCAATATTGTCAGTGATG | |
| | | GCGATATGAGTGTTGTGACCAAAGGTAACCTGTATAACTACCTCTA | |
| | | TATGCTGGGCGCTGGTGATGTCGCGATTACCGCAAACAACGTCAAC | |
| | | AACAATAATTCACTTATTGAGGCTGGTGGTGATCTGACGATTACCTC | |
| | | TACTGGCAATGTGGGTAATAACCGTGGCACGCTGCGCTCCCTGACG | |
| | | GGCGATGTAAATGTGGCGGCAAAAGGAACGGTTGATAACTATAGC | |
| | | GGTAAGATTACCTCTGCTGGCGATGTCGGAGTCAGTGGAAACTGGT | |
| | | TGCTCAATGATTATGGTCAGATTGCTGGCTTAAGCAATATTAACCTG | |
| | | ATTCTGACGGGCAACTTCGACAGTTACAAAGGTTCTGTGACCTCTCA | |
| | | AATGGGCGATATCAAGCTGGTTGCCAACTCGGTGGATAATAACAAC | |
| | | GGGTTAATCGCAGGTGAAAATATCTCCATTGATTCCAAATCAGGTG | |
| | | TCTATAACAACACGGCGCTGATTGCCGCGAATAAAACACTTACCGT | |
| | | AAATGCTGTCGGCAATATTGAAAACAGAGACGGTAATAACTTCAGT | |
| | | TATTACAACGGCGAGTATTTTGGTGTCGCAGGCGATGTGGGTGGCA | |
| | | TGGTGGGTAACGCGGGCATCTCGCTTTCCGCACAGAATATCTACAG | |
| | | CACAAACAGCCGTATTCTGGCAGAAAATGGCCCACTTAACGTGCTG | |
| | | TCCCGGGGCGTATTCGATAACACCCGCAGCATGCTGATGAGCGGTG | |
| | | CCAACGCCATTATTAAAGCGGCCGGTGTTTTCTATAATAACTATGCG | |
| | | ATGACATATAGCGCAGGTAACCTCGACATCACTTCTGCTTACCTTGA | |
| | | GAACTACAGCAACGGTAAGCTGGAAGATGGAACGGCAACAGGCGT | |
| | | TATCGCTTCGGATAAGAACCTGAACCTGAGCGTGGATAATAGCACC | |
| | | ACCAACTACGGCTGGATCAGCGGTAAGGGCGATGTGAATTTCAGC | |
| | | GTGTTAAAAGGTGTGCTGTATAACCGCAACACCATCGCCGCTAATA | |
| | | ACGCGCTGGCGATTAATGCCCTGAACGGTATCGAGAACTTCAAAGA | |
| | | TATCCTGGCGGGAACCAGCCTTACCCTTACTACCCAACGGCACGTTA | |
| | | CCAACAACAGCAACAGTAATATGCTTGGGCAAAGCATTGGGATTAA | |
| | | TGCGGTAAACGATATTAATAACCGCGGCAATATTGTCAGCGATTAT | |
| | | GCATTAACCGTGAAAACAGACGGGAACGTGTATAACTACCTCAATA | |
| | | TGCTGAGTTATGGCACCGCAAAAGTAACCGCGAATAAAGTCACGAA | |
| | | CAGCGGCAAGGACGCGGTGCTGGGCGGTTTTTACGGCCTTGACCTT | |
| | | GAATCGAATGCGATCACCAATACCGGCACCATTGTAGGTCTGTAA | |
| | | (SEQ ID NO: 13) | |
| Kosakonia | fhaB_ | tcaccacggcgataaccataggttttcggcgtggccacatccatggtgaatcccacttttt | |
| sacchari | prm2 | ccagcacgcgcgccacttcatcgggtcttaaatacatagattttcctcgtcatctttccaaa | |
| | | gcctcgccaccttacatgactgagcatggaccgtgactcagaaaattccacaaacgaac | |
| | | ctgaaaggcgtgattgccgtctggccttaaaaattatggtctaaactaaaatttacatcga | |
| | | aaacgagggaggatcctGTGATTAATTCGGAAAAGAAAGTCCAACGCCT | |
| | | TGTAAGAACTCAGCTGGCCCCGTTGACGGTAACAATGATATTCGCA | |
| | | TTACCTGCTGTTGCGCATTCCGCTGGTTTAAGTATGAAAAATGGCAC | |
| | | GCTATATAATGCGAATGGCGTGCCGGTCGTGGATATTAATAAACCT | |
| | | AACAGCAATGGCTTGTCACATAACGTCTGGGACAACCTGAACGTCG | |
| | | ATAAAAACGGTGTTATTTTTAATAACAGCGGCTCAGCATCCGATAC | |
| | | GGTATTGGGCGGCACCATCCAGGGTAACAGCAATCTGACATCCGG | |
| | | GTCGGCAAAAGTTATTCTCAACGAAGTTACCTCCAAAAATGCGTCG | |
| | | ACGATCAACGGTATGATGGAAGTTGCGGGTGATAAAGCCGCGTTG | |
| | | ATTATCGCCAACCCCAATGGCATTGCGGTAAATGGTGGCGGTACGA | |
| | | TCAATACCGATAAACTCACTCTGACCACTGGCACACCAGATATTCAG | |
| | | AACGACAAACTCGCTGGTTATTCCGTCAACGGCGGTACCATCACGA | |
| | | TCAGCAAACTGGATAACGCCAGCCCAACTGAAATTTTGTCACGCAA | |
| | | CGTAGTGCTCAGCGGGAAAGTTACCGCTGACGAACTGAATATTGTT | |
| | | GCGGGTAATAACTACGTTAATACAGACGGCCAGGTGACCGGAACT | |
| | | GTCACTGCGTCAGGCTCGCGTAACGGCTATAGCGTTGACGTTGCGC | |
| | | AAGTTGGCGGCATGTACGCGAATAAAATTAATCTTATCAGTACCGA | |
| | | AAGCGGCGTGGGCGTGCGTAATCAGGGCGTAATTGCCGGTGGCAC | |
| | | TGGTGGGATCAGCATTGATGCTAATGGCCAGCTACTGAACAGTAAT | |
| | | GCGCAGATTCAATCGACCGGTTCGACAAAGATTAATACCAACGGCA | |
| | | AATTGGACAATACAACGGGTCTTATTACTTCTGCTGGTGCTATTTAT | |
| | | CTCGATACTAATAAGAACGCTATTGTTAATGCCCGTTCAGGTACGAT | |
| | | TTCGACGACGGCAGATATCTACGTTAATAGCGGCGCAGTTGATAAT | |
| | | ACCAACGGCAAAATTGCCGCCAGCGGCGTGCTGGCTGTCGATACCA | |
| | | ATAACAATACTTTTACCAACGCCGGTAAAGGGAACGATGTCGGTAT | |
| | | CGAAGCGGGTATTGTCACCTTAAAAACCGGTACGCTAAATAATAGT | |
| | | AACGGCCAGATCCACGGCGGCTATCTGGGGCTTGAATCCGGCGCG | |
| | | ATTAACAATAACAGCGGTATTCTGGAAACGTCAGGCGACATGGACA | |
| | | TTGTCAGCCTTGGAAATGTCGACAATAACAAAGGCCTGATCCGATC | |
| | | TGCCAGCGGCCATATCAACATCCTGTCTGGCGGCACTATCAACAAC | |
| | | GGCAGTACCAAAACAGCGGATACCGTCAGTTCCGACTCCTTAGGCA | |
| | | TCGCAGCCGATACCGGTGTAGTAATCAACGCAAACAGCATCAATAA | |
| | | TAGCGGTGGTCAGATCGCATCGAACGGCGATGTGTCGCTAAACAG | |
| | | TAAGGGCCTGGTCAATAACGATTCCGGTAATCTTCTTTCCAATAGCA | |
| | | AAGTGATTGTGAAGGGCGGTTCTTATCGTAATGACTACGGTGGCAT | |
| | | CGGCGGCAAGAAGGGCGTGGAGGTGTCGGTTAACGGCGACCTGA | |
| | | GCAACTATATCGGCGTTGTGAGTGCGGAAGAAGGTGATATCTCACT | |
| | | GAGTGCAAATGCAGTGGATACCCACGGCGGTTTCATGATGGGGCA | |
| | | AAATATCTCCATCGACGCGAAAGCCGGCGTAAATACCACCCGATCG | |
| | | CTGGTATTAGCCAGTGAAAAGCTGGCTATCCATGCAGGCGGAAAT | |
| | | GTTGATAACCGTGATGGTAATAGCTTTGGCAATGACTTCGGCCTCT | |
| | | ATTTCGGCATGCCGCAACAGACCGGTGGCTTGATCGGTAAAGGTG | |
| | | GCGTTGAGATTTCCGGGCAGAATATTTACAACAACAACAGCCGCAT | |
| | | CATTGCAGAAAATGGGCCAATGAGCCTGCAAGCGAAAGGGAACAT | |
| | | TGATAACACGCGTGCGCTGATGGTCAGCGGGGCCGATACCGCAAT | |
| | | CAAAACCGGCGGGGCTTTCTACAATAACTACGCAACGATTTCCAGC | |
| | | GGAGGCAACCTCACACTCGATACCGTTTCCCTGGAAAACTCCAGCA | |
| | | GCGGCACGCTTATCGATAATAACGCCACAGGTTTCATCAACTCAGA | |
| | | TAAAGATATCACGCTGAATGTGGACAACAATTTCACGAACTACGGC | |
| | | TGGATCAGTGGTAAAGGCGCAGTGGCGCTGAATGTCGTGAAAGGG | |
| | | GCGTTTTATAACCGTAATACCATTGCGGCTGATAAGTCACTTGCGAT | |
| | | CAGTGCCCTGAACGGTATCGAGAACTTTAAAGATATCTCTGCAGGC | |
| | | GGCAGCCTGACGCTGGATACGCAACGTCATGTGACAAATAACAGC | |
| | | AACAGCAATATGGTCGGTAACGGGATTACTATTAACGCGGTAAACG | |
| | | ACATTAATAACCGCGGCAATATTGTCAGTGATGGCGATATGAGTGT | |
| | | TGTGACCAAAGGTAACCTGTATAACTACCTCTATATGCTGGGCGCT | |
| | | GGTGATGTCGCGATTACCGCAAACAACGTCAACAACAATAATTCAC | |
| | | TTATTGAGGCTGGTGGTGATCTGACGATTACCTCTACTGGCAATGT | |
| | | GGGTAATAACCGTGGCACGCTGCGCTCCCTGACGGGCGATGTAAA | |
| | | TGTGGCGGCAAAAGGAACGGTTGATAACTATAGCGGTAAGATTAC | |
| | | CTCTGCTGGCGATGTCGGAGTCAGTGGAAACTGGTTGCTCAATGAT | |
| | | TATGGTCAGATTGCTGGCTTAAGCAATATTAACCTGATTCTGACGG | |
| | | GCAACTTCGACAGTTACAAAGGTTCTGTGACCTCTCAAATGGGCGA | |
| | | TATCAAGCTGGTTGCCAACTCGGTGGATAATAACAACGGGTTAATC | |
| | | GCAGGTGAAAATATCTCCATTGATTCCAAATCAGGTGTCTATAACAA | |
| | | CACGGCGCTGATTGCCGCGAATAAAACACTTACCGTAAATGCTGTC | |
| | | GGCAATATTGAAAACAGAGACGGTAATAACTTCAGTTATTACAACG | |
| | | GCGAGTATTTTGGTGTCGCAGGCGATGTGGGTGGCATGGTGGGTA | |
| | | ACGCGGGCATCTCGCTTTCCGCACAGAATATCTACAGCACAAACAG | |
| | | CCGTATTCTGGCAGAAAATGGCCCACTTAACGTGCTGTCCCGGGGC | |
| | | GTATTCGATAACACCCGCAGCATGCTGATGAGCGGTGCCAACGCCA | |
| | | TTATTAAAGCGGCCGGTGTTTTCTATAATAACTATGCGATGACATAT | |
| | | AGCGCAGGTAACCTCGACATCACTTCTGCTTACCTTGAGAACTACA | |
| | | GCAACGGTAAGCTGGAAGATGGAACGGCAACAGGCGTTATCGCTT | |
| | | CGGATAAGAACCTGAACCTGAGCGTGGATAATAGCACCACCAACTA | |
| | | CGGCTGGATCAGCGGTAAGGGCGATGTGAATTTCAGCGTGTTAAA | |
| | | AGGTGTGCTGTATAACCGCAACACCATCGCCGCTAATAACGCGCTG | |
| | | GCGATTAATGCCCTGAACGGTATCGAGAACTTCAAAGATATCCTGG | |
| | | CGGGAACCAGCCTTACCCTTACTACCCAACGGCACGTTACCAACAA | |
| | | CAGCAACAGTAATATGCTTGGGCAAAGCATTGGGATTAATGCGGTA | |
| | | AACGATATTAATAACCGCGGCAATATTGTCAGCGATTATGCATTAAC | |
| | | CGTGAAAACAGACGGGAACGTGTATAACTACCTCAATATGCTGAGT | |
| | | TATGGCACCGCAAAAGTAACCGCGAATAAAGTCACGAACAGCGGC | |
| | | AAGGACGCGGTGCTGGGCGGTTTTTACGGCCTTGACCTTGAATCGA | |
| | | ATGCGATCACCAATACCGGCACCATTGTAGGTCTGTAA | |
| | | (SEQ ID NO: 14) | |
| Kosakonia | fhaB_ | atattgacaccatgacgcgcgtaatgctgattggttctgtgacgctggtaatgattgtcga | |
| sacchari | prm9 | aattctgaacagtgccatcgaagccgtagtagaccgtattggtgcagaattccatgaact | |
| | | ttccgggcgggcgaaggatatggggtcggcggcggtgctgatgtccatcctgctggcgat | |
| | | gtttacctggatcgcattactctggtcacattttcgataacgcttccagaattcgataacgc | |
| | | cctggttttttgcttaaatttggttccaaaatcgcctttagctgtatatactcacagcataac | |
| | | tgtatatacacccagggggcgggGTGATTAATTCGGAAAAGAAAGTCCAAC | |
| | | GCCTTGTAAGAACTCAGCTGGCCCCGTTGACGGTAACAATGATATT | |
| | | CGCATTACCTGCTGTTGCGCATTCCGCTGGTTTAAGTATGAAAAATG | |
| | | GCACGCTATATAATGCGAATGGCGTGCCGGTCGTGGATATTAATAA | |
| | | ACCTAACAGCAATGGCTTGTCACATAACGTCTGGGACAACCTGAAC | |
| | | GTCGATAAAAACGGTGTTATTTTTAATAACAGCGGCTCAGCATCCG | |
| | | ATACGGTATTGGGCGGCACCATCCAGGGTAACAGCAATCTGACATC | |
| | | CGGGTCGGCAAAAGTTATTCTCAACGAAGTTACCTCCAAAAATGCG | |
| | | TCGACGATCAACGGTATGATGGAAGTTGCGGGTGATAAAGCCGCG | |
| | | TTGATTATCGCCAACCCCAATGGCATTGCGGTAAATGGTGGCGGTA | |
| | | CGATCAATACCGATAAACTCACTCTGACCACTGGCACACCAGATATT | |
| | | CAGAACGACAAACTCGCTGGTTATTCCGTCAACGGCGGTACCATCA | |
| | | CGATCAGCAAACTGGATAACGCCAGCCCAACTGAAATTTTGTCACG | |
| | | CAACGTAGTGCTCAGCGGGAAAGTTACCGCTGACGAACTGAATATT | |
| | | GTTGCGGGTAATAACTACGTTAATACAGACGGCCAGGTGACCGGA | |
| | | ACTGTCACTGCGTCAGGCTCGCGTAACGGCTATAGCGTTGACGTTG | |
| | | CGCAAGTTGGCGGCATGTACGCGAATAAAATTAATCTTATCAGTAC | |
| | | CGAAAGCGGCGTGGGCGTGCGTAATCAGGGCGTAATTGCCGGTGG | |
| | | CACTGGTGGGATCAGCATTGATGCTAATGGCCAGCTACTGAACAGT | |
| | | AATGCGCAGATTCAATCGACCGGTTCGACAAAGATTAATACCAACG | |
| | | GCAAATTGGACAATACAACGGGTCTTATTACTTCTGCTGGTGCTATT | |
| | | TATCTCGATACTAATAAGAACGCTATTGTTAATGCCCGTTCAGGTAC | |
| | | GATTTCGACGACGGCAGATATCTACGTTAATAGCGGCGCAGTTGAT | |
| | | AATACCAACGGCAAAATTGCCGCCAGCGGCGTGCTGGCTGTCGATA | |
| | | CCAATAACAATACTTTTACCAACGCCGGTAAAGGGAACGATGTCGG | |
| | | TATCGAAGCGGGTATTGTCACCTTAAAAACCGGTACGCTAAATAAT | |
| | | AGTAACGGCCAGATCCACGGCGGCTATCTGGGGCTTGAATCCGGC | |
| | | GCGATTAACAATAACAGCGGTATTCTGGAAACGTCAGGCGACATG | |
| | | GACATTGTCAGCCTTGGAAATGTCGACAATAACAAAGGCCTGATCC | |
| | | GATCTGCCAGCGGCCATATCAACATCCTGTCTGGCGGCACTATCAA | |
| | | CAACGGCAGTACCAAAACAGCGGATACCGTCAGTTCCGACTCCTTA | |
| | | GGCATCGCAGCCGATACCGGTGTAGTAATCAACGCAAACAGCATCA | |
| | | ATAATAGCGGTGGTCAGATCGCATCGAACGGCGATGTGTCGCTAA | |
| | | ACAGTAAGGGCCTGGTCAATAACGATTCCGGTAATCTTCTTTCCAAT | |
| | | AGCAAAGTGATTGTGAAGGGCGGTTCTTATCGTAATGACTACGGTG | |
| | | GCATCGGCGGCAAGAAGGGCGTGGAGGTGTCGGTTAACGGCGAC | |
| | | CTGAGCAACTATATCGGCGTTGTGAGTGCGGAAGAAGGTGATATCT | |
| | | CACTGAGTGCAAATGCAGTGGATACCCACGGCGGTTTCATGATGG | |
| | | GGCAAAATATCTCCATCGACGCGAAAGCCGGCGTAAATACCACCCG | |
| | | ATCGCTGGTATTAGCCAGTGAAAAGCTGGCTATCCATGCAGGCGGA | |
| | | AATGTTGATAACCGTGATGGTAATAGCTTTGGCAATGACTTCGGCC | |
| | | TCTATTTCGGCATGCCGCAACAGACCGGTGGCTTGATCGGTAAAGG | |
| | | TGGCGTTGAGATTTCCGGGCAGAATATTTACAACAACAACAGCCGC | |
| | | ATCATTGCAGAAAATGGGCCAATGAGCCTGCAAGCGAAAGGGAAC | |
| | | ATTGATAACACGCGTGCGCTGATGGTCAGCGGGGCCGATACCGCA | |
| | | ATCAAAACCGGCGGGGCTTTCTACAATAACTACGCAACGATTTCCA | |
| | | GCGGAGGCAACCTCACACTCGATACCGTTTCCCTGGAAAACTCCAG | |
| | | CAGCGGCACGCTTATCGATAATAACGCCACAGGTTTCATCAACTCA | |
| | | GATAAAGATATCACGCTGAATGTGGACAACAATTTCACGAACTACG | |
| | | GCTGGATCAGTGGTAAAGGCGCAGTGGCGCTGAATGTCGTGAAAG | |
| | | GGGCGTTTTATAACCGTAATACCATTGCGGCTGATAAGTCACTTGC | |
| | | GATCAGTGCCCTGAACGGTATCGAGAACTTTAAAGATATCTCTGCA | |
| | | GGCGGCAGCCTGACGCTGGATACGCAACGTCATGTGACAAATAAC | |
| | | AGCAACAGCAATATGGTCGGTAACGGGATTACTATTAACGCGGTAA | |
| | | ACGACATTAATAACCGCGGCAATATTGTCAGTGATGGCGATATGAG | |
| | | TGTTGTGACCAAAGGTAACCTGTATAACTACCTCTATATGCTGGGC | |
| | | GCTGGTGATGTCGCGATTACCGCAAACAACGTCAACAACAATAATT | |
| | | CACTTATTGAGGCTGGTGGTGATCTGACGATTACCTCTACTGGCAAT | |
| | | GTGGGTAATAACCGTGGCACGCTGCGCTCCCTGACGGGCGATGTA | |
| | | AATGTGGCGGCAAAAGGAACGGTTGATAACTATAGCGGTAAGATT | |
| | | ACCTCTGCTGGCGATGTCGGAGTCAGTGGAAACTGGTTGCTCAATG | |
| | | ATTATGGTCAGATTGCTGGCTTAAGCAATATTAACCTGATTCTGACG | |
| | | GGCAACTTCGACAGTTACAAAGGTTCTGTGACCTCTCAAATGGGCG | |
| | | ATATCAAGCTGGTTGCCAACTCGGTGGATAATAACAACGGGTTAAT | |
| | | CGCAGGTGAAAATATCTCCATTGATTCCAAATCAGGTGTCTATAACA | |
| | | ACACGGCGCTGATTGCCGCGAATAAAACACTTACCGTAAATGCTGT | |
| | | CGGCAATATTGAAAACAGAGACGGTAATAACTTCAGTTATTACAAC | |
| | | GGCGAGTATTTTGGTGTCGCAGGCGATGTGGGTGGCATGGTGGGT | |
| | | AACGCGGGCATCTCGCTTTCCGCACAGAATATCTACAGCACAAACA | |
| | | GCCGTATTCTGGCAGAAAATGGCCCACTTAACGTGCTGTCCCGGGG | |
| | | CGTATTCGATAACACCCGCAGCATGCTGATGAGCGGTGCCAACGCC | |
| | | ATTATTAAAGCGGCCGGTGTTTTCTATAATAACTATGCGATGACATA | |
| | | TAGCGCAGGTAACCTCGACATCACTTCTGCTTACCTTGAGAACTACA | |
| | | GCAACGGTAAGCTGGAAGATGGAACGGCAACAGGCGTTATCGCTT | |
| | | CGGATAAGAACCTGAACCTGAGCGTGGATAATAGCACCACCAACTA | |
| | | CGGCTGGATCAGCGGTAAGGGCGATGTGAATTTCAGCGTGTTAAA | |
| | | AGGTGTGCTGTATAACCGCAACACCATCGCCGCTAATAACGCGCTG | |
| | | GCGATTAATGCCCTGAACGGTATCGAGAACTTCAAAGATATCCTGG | |
| | | CGGGAACCAGCCTTACCCTTACTACCCAACGGCACGTTACCAACAA | |
| | | CAGCAACAGTAATATGCTTGGGCAAAGCATTGGGATTAATGCGGTA | |
| | | AACGATATTAATAACCGCGGCAATATTGTCAGCGATTATGCATTAAC | |
| | | CGTGAAAACAGACGGGAACGTGTATAACTACCTCAATATGCTGAGT | |
| | | TATGGCACCGCAAAAGTAACCGCGAATAAAGTCACGAACAGCGGC | |
| | | AAGGACGCGGTGCTGGGCGGTTTTTACGGCCTTGACCTTGAATCGA | |
| | | ATGCGATCACCAATACCGGCACCATTGTAGGTCTGTAA | |
| | | (SEQ ID NO: 15) | |
| Klebsiella | otsB_ATG | Gaatttacttacattaaggcggcgaggggcgcctatacttgatagttctgataccagaag | |
| variicola | _prm16.1 | aaggaagaactATGGATAACCAGATATCTGTACCGCCTGCGCTAACCG | |
| | | GAAACTACGCTTTTTTCTTTGACCTCGACGGTACTCTTGCCGATATCC | |
| | | AACCGCACCCCGATCAGGTGGTGATACCCGACAACACGTTGCAGGC | |
| | | GCTCAACGCGCTGGCCCAGCAGCAGGGTGGGGCGGTGGCATTGAT | |
| | | TTCAGGGCGCTCAATGGCTGAACTTGACGCGCTAACCCATCCCTGG | |
| | | CGGCTACCGCTGGCCGGCGTTCACGGGGCAGAACGCCGTGATATC | |
| | | AATGGTAAAACCTATATCGTTTCTCTGCCGTCGGCGCTGCGCGATG | |
| | | AAATCGCGACTGAGCTGACCTCGGCGCTGCAGGGGCTCTCCGGCT | |
| | | GCGAGCTGGAGAGTAAGGAGATGGCCTTCGCGCTGCACTACCGCC | |
| | | AGGCTCCGCAGCAGCAGAGCGCAGTACTGGAGCTGGCTCAGCGCA | |
| | | TCGTTCAGCGCTACCCGCTGCTGGCACTGCAGCTGGGTAAATGTGT | |
| | | GGTGGAGATTAAACCCCGCGGAGTGAACAAAGGGGAGGCGATCA | |
| | | GCGCCTTCATGCAGGAGGCGCCGTTTGCCGGCCGCGAACCGGTTTT | |
| | | TGTCGGCGATGATTTGACCGATGAGGCGGGATTTAGCGTGGTTAAT | |
| | | CAACTGCAGGGAATGTCGGTAAAAGTCGGCGCCGGGGAAACTCAG | |
| | | GCGCACTGGCGGTTGGCGGATGCCGCCGCTGTAAGGACGTGGTTG | |
| | | CAACATCTGGCGTATGACGCGCAAACCGAAAGGAGGGATGACCAT | |
| | | GAGTCGTTTAGTCGTAGTCTCTAA (SEQ ID NO: 16) | |
| Klebsiella | otsB_ATG | Cgccgtcctcgcagtaccattgcaaccgactttacagcaagaagtgattctggcacgcat | |
| variicola | _prm8.2 | ggaacaaattcttgccagtcgggctttatccgatgacgaacgcgcacagcttttatatgag | |
| | | cgcggagtgttgtatgatagtctcggtctgagggcattagcgcgaaatgatttttcacaag | |
| | | cgctggcaatccgacccgatatgcctgaagtattcaattacttaggcatttacttaacgca | |
| | | ggcaggcaattttgatgctgcctatgaagcgtttgattctgtacttgagcttgatcATGG | |
| | | ATAACCAGATATCTGTACCGCCTGCGCTAACCGGAAACTACGCTTTT | |
| | | TTCTTTGACCTCGACGGTACTCTTGCCGATATCCAACCGCACCCCGA | |
| | | TCAGGTGGTGATACCCGACAACACGTTGCAGGCGCTCAACGCGCTG | |
| | | GCCCAGCAGCAGGGTGGGGCGGTGGCATTGATTTCAGGGCGCTCA | |
| | | ATGGCTGAACTTGACGCGCTAACCCATCCCTGGCGGCTACCGCTGG | |
| | | CCGGCGTTCACGGGGCAGAACGCCGTGATATCAATGGTAAAACCT | |
| | | ATATCGTTTCTCTGCCGTCGGCGCTGCGCGATGAAATCGCGACTGA | |
| | | GCTGACCTCGGCGCTGCAGGGGCTCTCCGGCTGCGAGCTGGAGAG | |
| | | TAAGGAGATGGCCTTCGCGCTGCACTACCGCCAGGCTCCGCAGCAG | |
| | | CAGAGCGCAGTACTGGAGCTGGCTCAGCGCATCGTTCAGCGCTACC | |
| | | CGCTGCTGGCACTGCAGCTGGGTAAATGTGTGGTGGAGATTAAAC | |
| | | CCCGCGGAGTGAACAAAGGGGAGGCGATCAGCGCCTTCATGCAGG | |
| | | AGGCGCCGTTTGCCGGCCGCGAACCGGTTTTTGTCGGCGATGATTT | |
| | | GACCGATGAGGCGGGATTTAGCGTGGTTAATCAACTGCAGGGAAT | |
| | | GTCGGTAAAAGTCGGCGCCGGGGAAACTCAGGCGCACTGGCGGTT | |
| | | GGCGGATGCCGCCGCTGTAAGGACGTGGTTGCAACATCTGGCGTA | |
| | | TGACGCGCAAACCGAAAGGAGGGATGACCATGAGTCGTTTAGTCG | |
| | | TAGTCTCTAA (SEQ ID NO: 17) | |
| Kosakonia | otsB- | cgtcctgtaataataaccggacaattcggactgattaaaaaagcgcccttgtggcgctttt | |
| sacchari | Prm1 | tttatattcccgcctccatttaaaataaaaaatccaatcggatttcactatttaaactggcc | |
| | | attatctaagatgaatccgatggaagctcgctgttttaacacgcgttttttaaccttttattg | |
| | | aaagtcggtgcttctttgagcgaacgatcaaatttaagtggattcccatcaaaaaaatatt | |
| | | ctcaacctaaaaaagtttgtgtaatacttgtaacgctacatggagattaactcaatctaga | |
| | | gggtattaataatgaatcgtactaaactggtactgggcGTGGAAGACGCACTATC | |
| | | TATACCGCCTCTCATTTCCGGAAATTTTGCCTTTTTTTTCGATTTGGA | |
| | | TGGCACCCTTGCGGACATCAAACCGCACCCGGATGATGTTTTTATCC | |
| | | CTGAAGATGTGCTGACCAGACTTTCATTGCTTGCTGAGCTAAATGA | |
| | | CAGGGCGTTGGCATTGATTTCAGGGCGTTCAATTGCTGAGCTGGAA | |
| | | CAGCTCGCCAAACCGTATCGTTTCCCGCTGGCCGGGGTACATGGGG | |
| | | CGGAGCGCCGCGATATCAATGGTCGAACCGAACGAATGACCTTGC | |
| | | CGGAAGCGGTTTTACAACCGCTGGAACGTGAACTCCGGCATGGCAT | |
| | | TGCGCCGCTGACCGGCGTTGAACTTGAAACCAAAGGGATGGCATTT | |
| | | GCACTGCATTATCGCCAGGCGCCGCAGCATGAAGAGGCGGTCTTTG | |
| | | CCCTGGCAAAAACACTCATCGCGCACTACCCACAACTGGCAATGCA | |
| | | GCCGGGAAAATGTGTGGTCGAGCTGAAACCCTCTGGCATTCATAAA | |
| | | GGCGCCGCTATTGGCGCGTTTATGCAAACGCCGCCCTTTCTCGGCA | |
| | | AGAAACCGGTGTTTCTTGGCGACGATCTGACTGACGAACATGGATT | |
| | | TAAAACCGTTAATAAACTTGGCGGCATATCGATAAAAGTTGGCCCT | |
| | | GGCGCAACACAGGCAATGTGGCGTCTGGCTGGCGTAAATGATGTC | |
| | | TATCAATGGCTTGAAAAACTTGTCGACCATCAACAACAAGAACAAC | |
| | | GGGCGCTAACAAACAGGAGAGATGGCTATGAGTCGCTTAGTCGTA | |
| | | GTATCTAA (SEQ ID NO: 18) | |
| Kosakonia | otsB- | Tcaccacggcgataaccataggttttcggcgtggccacatccatggtgaatcccacttttt | |
| sacchari | Prm2 | ccagcacgcgcgccacttcatcgggtcttaaatacatagattttcctcgtcatctttccaaa | |
| | | gcctcgccaccttacatgactgagcatggaccgtgactcagaaaattccacaaacgaac | |
| | | ctgaaaggcgtgattgccgtctggccttaaaaattatggtctaaactaaaatttacatcga | |
| | | aaacgagggaggatcctGTGGAAGACGCACTATCTATACCGCCTCTCATT | |
| | | TCCGGAAATTTTGCCTTTTTTTTCGATTTGGATGGCACCCTTGCGGA | |
| | | CATCAAACCGCACCCGGATGATGTTTTTATCCCTGAAGATGTGCTGA | |
| | | CCAGACTTTCATTGCTTGCTGAGCTAAATGACAGGGCGTTGGCATT | |
| | | GATTTCAGGGCGTTCAATTGCTGAGCTGGAACAGCTCGCCAAACCG | |
| | | TATCGTTTCCCGCTGGCCGGGGTACATGGGGCGGAGCGCCGCGAT | |
| | | ATCAATGGTCGAACCGAACGAATGACCTTGCCGGAAGCGGTTTTAC | |
| | | AACCGCTGGAACGTGAACTCCGGCATGGCATTGCGCCGCTGACCG | |
| | | GCGTTGAACTTGAAACCAAAGGGATGGCATTTGCACTGCATTATCG | |
| | | CCAGGCGCCGCAGCATGAAGAGGCGGTCTTTGCCCTGGCAAAAAC | |
| | | ACTCATCGCGCACTACCCACAACTGGCAATGCAGCCGGGAAAATGT | |
| | | GTGGTCGAGCTGAAACCCTCTGGCATTCATAAAGGCGCCGCTATTG | |
| | | GCGCGTTTATGCAAACGCCGCCCTTTCTCGGCAAGAAACCGGTGTT | |
| | | TCTTGGCGACGATCTGACTGACGAACATGGATTTAAAACCGTTAAT | |
| | | AAACTTGGCGGCATATCGATAAAAGTTGGCCCTGGCGCAACACAG | |
| | | GCAATGTGGCGTCTGGCTGGCGTAAATGATGTCTATCAATGGCTTG | |
| | | AAAAACTTGTCGACCATCAACAACAAGAACAACGGGCGCTAACAAA | |
| | | CAGGAGAGATGGCTATGAGTCGCTTAGTCGTAGTATCTAA | |
| | | (SEQ ID NO: 19) | |
| Kosakonia | pehA- | Cgtcctgtaataataaccggacaattcggactgattaaaaaagcgcccttgtggcgctttt | Gene |
| sacchari | Prm1 | tttatattcccgcctccatttaaaataaaaaatccaatcggatttcactatttaaactggcc | sequence |
| | | attatctaagatgaatccgatggaagctcgctgttttaacacgcgttttttaaccttttattg | is of |
| | | aaagtcggtgcttctttgagcgaacgatcaaatttaagtggattcccatcaaaaaaatatt | first |
| | | ctcaacctaaaaaagtttgtgtaatacttgtaacgctacatggagattaactcaatctaga | gene in |
| | | gggtattaataatgaatcgtactaaactggtactgggcATGGGATATGACGCGTG | the pehA |
| | | TAATTTTATTAACTCGATCGTTAAAAATTTTAAAGAATCCATTGGAA | operon |
| | | TAATGAACGTGTTGTTACTGGAACCTGATGAGTCCGACGCTTTGCG | |
| | | CTTATTGTATCTAAAACTGGATGTTATCAGGGCTGGCATTGAGCAG | |
| | | GATGTCCGCATGTTAACCAAAGCCCGTCTGGACAGCAACGCCGCAC | |
| | | TTTGCCTTTACCAGGTACATAATGCGTTAAATGCTTTGAACTATTGC | |
| | | ATTGGGATTGTCAGAAATACGCCGTTAAATCAGCAGGTTTATTACG | |
| | | ATTTCGAAGATCTAAAGCGTATTGTGAACACTCTTGATGCAACATCC | |
| | | GGACGTGTTTTTAATAAACTGGATGGTAATGAGTACACATAA | |
| | | (SEQ ID NO: 20) | |
| Kosakonia | pehA- | Tcaccacggcgataaccataggttttcggcgtggccacatccatggtgaatcccacttttt | Gene |
| sacchari | Prm2 | ccagcacgcgcgccacttcatcgggtcttaaatacatagattttcctcgtcatctttccaaa | sequence |
| | | gcctcgccaccttacatgactgagcatggaccgtgactcagaaaattccacaaacgaac | is of |
| | | ctgaaaggcgtgattgccgtctggccttaaaaattatggtctaaactaaaatttacatcga | first |
| | | aaacgagggaggatcctATGGGATATGACGCGTGTAATTTTATTAACTCG | gene in |
| | | ATCGTTAAAAATTTTAAAGAATCCATTGGAATAATGAACGTGTTGTT | the pehA |
| | | ACTGGAACCTGATGAGTCCGACGCTTTGCGCTTATTGTATCTAAAAC | operon |
| | | TGGATGTTATCAGGGCTGGCATTGAGCAGGATGTCCGCATGTTAAC | |
| | | CAAAGCCCGTCTGGACAGCAACGCCGCACTTTGCCTTTACCAGGTA | |
| | | CATAATGCGTTAAATGCTTTGAACTATTGCATTGGGATTGTCAGAAA | |
| | | TACGCCGTTAAATCAGCAGGTTTATTACGATTTCGAAGATCTAAAGC | |
| | | GTATTGTGAACACTCTTGATGCAACATCCGGACGTGTTTTTAATAAA | |
| | | CTGGATGGTAATGAGTACACATAA (SEQ ID NO: 21) | |
| Kosakonia | pehA- | Atattgacaccatgacgcgcgtaatgctgattggttctgtgacgctggtaatgattgtcga | Gene |
| sacchari | Prm9 | aattctgaacagtgccatcgaagccgtagtagaccgtattggtgcagaattccatgaact | sequence |
| | | ttccgggcgggcgaaggatatggggtcggcggcggtgctgatgtccatcctgctggcgat | is of |
| | | gtttacctggatcgcattactctggtcacattttcgataacgcttccagaattcgataacgc | first |
| | | cctggttttttgcttaaatttggttccaaaatcgcctttagctgtatatactcacagcataac | gene in |
| | | tgtatatacacccagggggcgggATGGGATATGACGCGTGTAATTTTATTA | the pehA |
| | | ACTCGATCGTTAAAAATTTTAAAGAATCCATTGGAATAATGAACGT | operon |
| | | GTTGTTACTGGAACCTGATGAGTCCGACGCTTTGCGCTTATTGTATC | |
| | | TAAAACTGGATGTTATCAGGGCTGGCATTGAGCAGGATGTCCGCAT | |
| | | GTTAACCAAAGCCCGTCTGGACAGCAACGCCGCACTTTGCCTTTACC | |
| | | AGGTACATAATGCGTTAAATGCTTTGAACTATTGCATTGGGATTGTC | |
| | | AGAAATACGCCGTTAAATCAGCAGGTTTATTACGATTTCGAAGATC | |
| | | TAAAGCGTATTGTGAACACTCTTGATGCAACATCCGGACGTGTTTTT | |
| | | AATAAACTGGATGGTAATGAGTACACATAA (SEQ ID NO: 22) | |
| Kosakonia | treZ- | tcaccacggcgataaccataggttttcggcgtggccacatccatggtgaatcccacttttt | |
| sacchari | Prm2 | ccagcacgcgcgccacttcatcgggtcttaaatacatagattttcctcgtcatctttccaaa | |
| | | gcctcgccaccttacatgactgagcatggaccgtgactcagaaaattccacaaacgaac | |
| | | ctgaaaggcgtgattgccgtctggccttaaaaattatggtctaaactaaaatttacatcga | |
| | | aaacgagggaggatcctATGGCGTCGAAATCGTTTTGTAAAAGTTGGGG | |
| | | AGCCGAATACCTCGCGAATGGTCGCGTGCGTTTTCGCTTATGGGCC | |
| | | ACCGGGCAGCAGCAGGTTTCACTGCGTTTACCGGCTGGCGAATTGC | |
| | | CAATGCAACGCCTTGATGACGGCTGGTTTGAACTGACCGTCGATAA | |
| | | TATCGCCGCAGGTACGGCGTACCAGTTTGTCCTGAGCAACGGTTTG | |
| | | GCGGTTCCCGATCCCGCCTCGCGCGCGCAGGCCGCCGATGTTAACG | |
| | | GCCCTTCGCTGGTTGTCGACCCGGATCATTACGTCTGGCAACACCC | |
| | | GCAGTGGCAGGGTCGCCCGTGGCAAGAATCCGTGGTCTATGAACT | |
| | | GCATATTGGGACATTTACCCCGCGGGGCACTTTTCGCGCGGCAATG | |
| | | GAAAAACTGCCGTACCTCGCGCAACTCGGCATCACCATGATAGAGG | |
| | | TGATGCCCGTCTCCCAGTTTGGCGGCAATCGCGGCTGGGGCTATGA | |
| | | CGGCGTGCTGCTCTATGCGCCGCACTCGGCGTATGGCACGCCGGAT | |
| | | GATTTTAAAGCCTTTATCGACGCCGCTCACGGCTACGGCCTTTCAGT | |
| | | GGTGCTGGATATTGTGCTCAACCACTTTGGCCCGGAAGGTAACTAC | |
| | | TTACCCGCGCTGTCACCGGATTTTTTCCACGCCGAACGGCAGACGC | |
| | | CGTGGGGGCCGGGCATCGCGTATGACGTTGACGCGGTGCGGCGTT | |
| | | ACATCGTTGAAGCACCGCTCTACTGGCTCAACGAATACAACCTGGA | |
| | | CGGGCTGCGATTTGATGCCATCGATCAGATTGAAGACAGCGCCGA | |
| | | ACCGCCGGTGCTGGTGGAGATTGCCCGCCGTATTCGCGAGGAGAT | |
| | | CACCGACCGGCCGGTGCATCTGACCACCGAAGATTGCCGCAATATT | |
| | | ATTGCGCTGCATCCGCGCGAGCCGGATGGGCGTGCGCCGCTGTTTA | |
| | | CCGCCGAGTGGAATGACGATCTGCACAATGCCGCGCATGTGTTTGC | |
| | | CACCGGTGAAACCCATGCCTATTACCAGGATTTTGCGGATAAGCCC | |
| | | GAAGTGTGGCTGGCGCGCACGTTAACTGAAGGATTCGCGTTTCAG | |
| | | GGGGAGATTGCCCCGTCAACCGGTGAACCACGCGGAGTGGACAGC | |
| | | CGCAACCAGCCGCCGGTAGCGTTTGTCGATTTTATCCAGAACCACG | |
| | | ATCAGACCGGCAACCGCGCCCAGGGCGAGCGGCTTATCACGCTGG | |
| | | CGGGCGAGGAGAGAACGCGCGTGCTGCTCGGCGCGCTGCTGTTTT | |
| | | CCCCGCATATTCCGCTGCTGTTTATGGGCGAAGAGTATGGCGAAAC | |
| | | CAATCCTTTCCTGTTCTTTACCGATTTTCACGGCGATCTGGCGCGCG | |
| | | CCGTGCGTGAAGGGCGCGCGCGGGAATTCCAGGGCCATGCCGGAC | |
| | | ATGAAGGGGAAGCGGTTCCCGATCCGAATGCGCCTGACACTTTTAC | |
| | | CCGATCACAACTGGACTGGGCAAAAACGCAGCAGGAGAGCGGCCA | |
| | | GCGCTGGCTGACGCAAACCCGCGACTGGCTGACGCTGCGCCAGAA | |
| | | ATATGTGGTTCCGCTGCTGACAAATGCGCAGCGGCAAAATGGGGA | |
| | | AGTGATCGCCACCGCGCCAGGGTTTGTCGCGGTCCGCTGGCGCTTC | |
| | | CCGCAAGGGACATTATCGCTGGCGCTCAATATTGGCGAGGCGACG | |
| | | CAACCGTTGCCGGATCTTCCCGGCAAAACGCTGGCAGCCTGGCCGC | |
| | | GCGGACAAGATGACTTGCCGCCGAATGCGTTCATTGTTCGTCTGGC | |
| | | TTTGGGAGAGGGAATGTGA (SEQ ID NO: 23) | |
| Kosakonia | yjbE2_ | Cgtcctgtaataataaccggacaattcggactgattaaaaaagcgcccttgtggcgctttt | |
| sacchari | prm1 | tttatattcccgcctccatttaaaataaaaaatccaatcggatttcactatttaaactggcc | |
| | | attatctaagatgaatccgatggaagctcgctgttttaacacgcgttttttaaccttttattg | |
| | | aaagtcggtgcttctttgagcgaacgatcaaatttaagtggattcccatcaaaaaaatatt | |
| | | ctcaacctaaaaaagtttgtgtaatacttgtaacgctacatggagattaactcaatctaga | |
| | | gggtattaataatgaatcgtactaaactggtactgggcATGAAAAAAGTCCTGTA | |
| | | TGGCATTTTTGCCATATCCGCGCTTGCGGCGACTTCTGCTTTTGCTG | |
| | | CACCGGTGCAAATTGGTGAAGCGGCAGGGTCGGCAGCGACCTCTG | |
| | | TTTCTGCGGGTAGCTCCGCAGCAACGAGCGTTAGCACCGTAGGGTC | |
| | | CGCGGTGGGTGTCGCGCTGGCGGCAACCGGTGGCGGTGATGGCTC | |
| | | CAACACGGGAACCACGACCACCACGACCACAAGTACCCAGTAA | |
| | | (SEQ ID NO: 24) | |
| Kosakonia | yjbE2_ | Tcaccacggcgataaccataggttttcggcgtggccacatccatggtgaatcccacttttt | |
| sacchari | prm2 | ccagcacgcgcgccacttcatcgggtcttaaatacatagattttcctcgtcatctttccaaa | |
| | | gcctcgccaccttacatgactgagcatggaccgtgactcagaaaattccacaaacgaac | |
| | | ctgaaaggcgtgattgccgtctggccttaaaaattatggtctaaactaaaatttacatcga | |
| | | aaacgagggaggatcctATGAAAAAAGTCCTGTATGGCATTTTTGCCATA | |
| | | TCCGCGCTTGCGGCGACTTCTGCTTTTGCTGCACCGGTGCAAATTGG | |
| | | TGAAGCGGCAGGGTCGGCAGCGACCTCTGTTTCTGCGGGTAGCTCC | |
| | | GCAGCAACGAGCGTTAGCACCGTAGGGTCCGCGGTGGGTGTCGCG | |
| | | CTGGCGGCAACCGGTGGCGGTGATGGCTCCAACACGGGAACCACG | |
| | | ACCACCACGACCACAAGTACCCAGTAA (SEQ ID NO: 25) | |
| Kosakonia | yjbE2_ | Atattgacaccatgacgcgcgtaatgctgattggttctgtgacgctggtaatgattgtcga | |
| sacchari | prm9 | aattctgaacagtgccatcgaagccgtagtagaccgtattggtgcagaattccatgaact | |
| | | ttccgggcgggcgaaggatatggggtcggcggcggtgctgatgtccatcctgctggcgat | |
| | | gtttacctggatcgcattactctggtcacattttcgataacgcttccagaattcgataacgc | |
| | | cctggttttttgcttaaatttggttccaaaatcgcctttagctgtatatactcacagcataac | |
| | | tgtatatacacccagggggcgggATGAAAAAAGTCCTGTATGGCATTTTTG | |
| | | CCATATCCGCGCTTGCGGCGACTTCTGCTTTTGCTGCACCGGTGCAA | |
| | | ATTGGTGAAGCGGCAGGGTCGGCAGCGACCTCTGTTTCTGCGGGT | |
| | | AGCTCCGCAGCAACGAGCGTTAGCACCGTAGGGTCCGCGGTGGGT | |
| | | GTCGCGCTGGCGGCAACCGGTGGCGGTGATGGCTCCAACACGGGA | |
| | | ACCACGACCACCACGACCACAAGTACCCAGTAA | |
| | | (SEQ ID NO: 26) | |
| | | unique junction at deletion point (upstream of deletion | |
| | | is lowercase, downstream of deletion is uppercase) | |
| Kosakonia | ΔgIgA | Gataccgagtgagagcaaggcgttagaaagggtgcgaccaatcagaaactccatcgat | |
| sacchari | | aagtagtaaacctggcgcacttcctgagagagctgtgcacgggtagaacgcagccagcg | |
| | | ctccaccagccgatcgcgcacggcaaacagcgtggcgttcagccattcgtggcgattggc | |
| | | gacagccggatctttaccgatggtgaacatcagtttgtaggcaatcgaatgttttaacgcc | |
| | | tcaatacttaacgtaggtgaagcataagtaaaaggtgcattcatataatgattcctggaa | |
| | | aaTATCGCTCCTGTTCAAAACCCGTTGGCCTTGATGCCAGAAATGGG | |
| | | GAAACAGCGCGCGGACCCCTTCCGCGCAAGGTTATAATCAGATGCT | |
| | | TTCAGCTTTTAATTTGCGCAGCATTTCACGTGTGACCAACACAATGC | |
| | | CTTCTTCGGAACGGTAAAAGCGACGCGCATCTTCTTCCGCATTTTCA | |
| | | CCAATCACCGTTCCTTCCGGTATCACGCAGCCTCTGTCGATAATGCA | |
| | | GCGATGGAGGCGACAGGAGCGCCCCACCCACACTTCCGGCAGCAA | |
| | | GACCGAAGAGTCGATATTGCAGA (SEQ ID NO: 27) | |
| Klebsiella | ΔgIgA | Cggcggctgcattatctccgggtcggtggtggtgcagtcggtgctgttcccgcgcgtgcgg | |
| variicola | | gtgaactcattctgtaacattgattcggcagtcttgttgccagatgtctgggtggggcgctc | |
| | | atgccgcctgcgtcgctgcgtgattgaccgcgcctgcgtcatcccggaaggtatggtgatt | |
| | | ggtgagaacgcggaagaggatgcacgccgcttctatcgctcagaggaaggtatcgtgct | |
| | | ggtgacccgcgacatgctgcggaaattggggcacaaacaggagcgctaatgcaggtcT | |
| | | TGATGTAACCACTGGAGTCATTGTTATGAATGTACCCTTTAGCTATG | |
| | | CATCACCGACGCTGAGCGTTGAGGCGTTAAAGCACTCTATTGCCTA | |
| | | TAAGCTGATGTTTATCATCGGTAAAGATCCGGCCATCGCCAACAAG | |
| | | CATGAATGGCTTAACGCCACGCTGTTTGCCGTCCGCGATCGCATGG | |
| | | TGGAGCGCTGGCTGCGCTCCAACCGCGCGCAGCTCTCGCAGGAAG | |
| | | TGCGCCAGGTATACTATCTGTCGATGGAGTTCCTGATTGGCCGTAC | |
| | | CCTCTCTAACGCGCTGCTGTCGC (SEQ ID NO: 28) | |

TABLE 12
Promoter and gene sequences. Promoter sequences are
listed in lower case and gene sequences in upper case.
In the case of deletion mutations the sequence before the
deletion is listed in lower case and the sequence after the
deletion is listed in upper case.
Method: Ammonium excretion assay

1. Pick colonies and culture in 96-well deep well plate rich
media at 30C overnight
2. Use the overnight culture to inoculate a culture in
ARA media (N-free media) supplemented with
10 mM glutamine (gln) (also in 96-well plate)
3. Use culture in ARA + gln to inoculate a culture
in ARA without gln
4. Transfer to the anaerobic chamber and incubate
with shaking at room temp for 3 days
5. Spin down the 96-well plate with the cells and
remove a sample of the supernatant
6. Use the Megazyme Ammonium Assay kit to assay
ammonium concentration in the media

TABLE 13

| | | | | Fold increase over parent | | | | | | |
|---|---|---|---|---|---|---|---|---|---|---|
| | | | | | ATT | | ARA | | GOI transcript | |
| Strain | Cured | | ΔGenotype | | copies/ | % or fold | 0 mM | 5 mM | 0 mM | 5 mM |
| ID | ID | Parent | from Parent | AMM | well | ATT | gln | gln | gln | gln |
| 6-1098 | 6-1338 | 6-848 | ds786 [Ci006 | 0.60 | 2.3x | 2.8x | Not tested | | | |
| | | (CM093) | fhaB_prm2] | p = 0.04 | p = 0.013 | p = 0.006 | | | | |
| 6-1127 | Not | 6-848 | ds778 [CI006 | 0.93 | 2.5x | inoculum | 0.49x | 0.96x | 72x | 24x |
| | Cured | (CM093) | bcsIII_prm9] | p = 0.64 | p = 0.0005 | not | p = 0.06 | p = 0.84 | p = 0.11 | p = 0.40 |
| | | | | | | quantified | | | | |
| 6-1126 | 6-1337 | 6-848 | ds777 [CI006 | 1.03 | 1.7x | inoculum | 0.16x | 0.09x | 2.9x | 0.9x |
| | | (CM093) | bcsIII_prm2] | p = 0.90 | p = 0.06 | not | p = 0.03 | p = 0.01 | p = 0.12 | p = 0.86 |
| | | | | | | quantified | | | | |
| 6-1103 | 6-1286 | 6-848 | ds794 [CI006 | 0.71 | 1.9x | inoculum | 1.2x | 1.5x | 3.9x | 4.7x |
| | | (CM093) | yjbE2_prm1] | p = 0.18 | p = 0.07 | not | p = 0.37 | p = 0.09 | p = 0.61 | p = 0.55 |
| | | | | | | quantified | | | | |
| 6-1137 | Not | 6-881 | ds796 [CI006 | 0.78 | 3.3x | 1.7x | Not tested | | | |
| | Cured | (CM094) | yjbE2_prm9] | p = 0.11 | p = 0.03 | p = 0.32 | | | | |
| 6-1136 | 6-1331 | 6-848 | ds720 [CI006 | 1.46 | 1.9x | 0.7x | 0.46x | 0.76x | 212x | 106x |
| | | (CM093) | pehA-Prm9] | p = 0.34 | p = 0.13 | p = 0.68 | p =0.24 | p = 0.80 | p = 0.41 | p = 0.39 |
| 6-1142 | 6-1336 | 6-848 | ds774 [CI006 | 1.06 | 2.0x | 1.28 | 0.63x | 0.45x | 3.1x | 0.9x |
| | | (CM093) | bcsII_prm2] | p = 0.34 | p = 0.13 | p = 0.62 | p =0.55 | p = 0.45 | p = 0.61 | p = 0.89 |
| 6-1104 | Not | 6-848 | ds795 [CI006 | 1.07 | 0.72 | inoculum | Not tested | | | |
| | Cured | (CM93) | yjbE2_prm2] | p = 0.45 | p = 0.16 | not | | | | |
| | | | | | | quantified | | | | |
| 6-1077 | 6-1296 | 6-881 | ds786 [Ci006 | 0.72 | 1.06 | 0.94x | 0.99x | 0.19x | 3.5x | 3.0x |
| | | (CM94) | fhaB_prm2] | p = 0.39 | p = 0.97 | p = 0.94 | p = 0.97 | p = 0.09 | p = 0.05 | p = 0.33 |
| 6-1094 | 6-1285 | 6-881 | ds794 [CI006 | 1.43x | 0.90 | 0.58x | 1.0x | 1.1x | 4.5x | 16x |
| | | (CM094) | yjbE2_prm1] | p = 0.69 | p = 0.87 | p = 0.06 | p = 0.96 | p = 0.64 | p = 0.24 | p = 0.01 |

TABLE 14

| | | | sig. at 5 | | Greenhouse |
|---|---|---|---|---|---|
| Strain | Geno- | | mM gln only | | Colonization |
| ID | type | Target | ARA | transcript | Assay |
| 6-915 | bcsI- | cellulose | 3.2x | not tested | no sig. diff. |
| | Prm2 | synthesis | p = 0.314 | | |
| 6-916 | cysZ- | sulfate | 5.2x | 63.2x | not tested |
| | Prm1 | transport | p = 0.103 | p = 0.001 | |
| 6-921 | otsB- | trehalose | 37.5x | 173.0x | 16x sig decrease |
| | Prm1 | synthesis | p = 0.003 | p = 0.001 | in CFU/g FW |
| 6-922 | otsB- | trehalose | 19.2x | 4.3x | no sig. diff. |
| | Prm2 | synthesis | p = 0.053 | p = 0.021 | |
| 6-923 | treZ- | trehalose | 14.2x | 47.3x | no sig. diff. |
| | Prm2 | synthesis | p = 0.178 | p = 0.152 | |

### Example 10: Toxicology Study

To confirm the absence of human or mammalian toxicity for Klebsiella variicola and Kosakonia sacchari, toxicity study was conducted with these organisms. The contractor was Smithers Avanza Toxicology Services, a company that has extensive experience in toxicology studies.

The purpose of this study was to determine the toxicity of bacterial isolates in mice following a single subcutaneous injection. The CD-1 mice were used to maximize the number of bacterial isolates per kg of bodyweight. The subcutaneous route was selected as it is the appropriate route for the inoculation of bacterial isolates to animals, and it most simulates the most probable human exposure to the microbes.

Treatments with either Klebsiella variicola or Kosakonia sacchari bacterial isolates did not affect the mortality of the mice, all animals survived until the scheduled termination at the end of the study period. There were no abnormalities in clinical observations of any of the animals in the Klebsiella variicola and Kosakonia sacchari treatment groups throughout the course of the study. Also, no abnormalities were noted during cageside observations. Further, there were no abnormalities with dermal observations for the Klebsiella variicola and Kosakonia sacchari treatment groups. Mice body temperatures were normal for all groups, with mean temperatures ranging from 36.66° C. to 37.62° C. There was slight body weight loss for mice in the Klebsiella variicola and Kosakonia sacchari treatment groups, but this was within the normal range for mice in this study and from historical data for this type of study. Results of this study confirm Klebsiella variicola and Kosakonia sacchari bacterial isolates do not pose a risk of toxicity or pathogenicity to humans or other mammals.

The purpose of this study was to determine the toxicity of bacterial isolates in mice following a single subcutaneous injection. The CD-1 mice were used to maximize the number of bacterial isolates per kg of the bodyweight. The subcutaneous route was selected as it is the appropriate route for the inoculation bacterial isolates to animals.

Forty five naïve female CD-1 mice approximately 8 weeks old, were received from Charles River Breeding Labs on Jun. 8, 2017. Animals were group-housed (5 animals per cage) in polycarbonate cages containing hardwood bedding. Animals were fed Certified Global Teklad Laboratory Diet 2018 (pellets) ad libitum and provided drinking water via water bottles. Animal room environmental controls were set to maintain a temperature of 20-26° C., a relative humidity of 30-70%, a minimum of 10 air changes per hour, and a 12-hour light/12-hour dark cycle.

Forty mice were randomized into eight groups consisting of five mice each. Extra animals were moved to training/stock colony. Animals were treated as indicated in Table 15.

TABLE 15
Study Design
Table 15: strains used

| | | | | Test | | |
|---|---|---|---|---|---|---|
| | | | Test | Article | Females | |
| | | | Article Dosage | Volume | | Animal |
| Group | Treatment | Genus Species/ID | (isolates/animal) | (mL/animal) | N | Numbers |
| 1 | PB137 | Klebsiella variicola | ≥10⁸ | 0.1 | 5 | 21994-21998 |
| 2 | PB728 | Klebsiella variicola | ≥10⁸ | 0.1 | 5 | 21999-22003 |
| 3 | PB006 | Kosakonia sacchari | ≥10⁸ | 0.1 | 5 | 22004-22008 |
| 4 | PB063 | Rahrella aquatiiis | ≥10⁸ | 0.1 | 5 | 22009-22013 |
| 5 | PB019 | Rahnella aquatiiis | ≥10⁸ | 0.1 | 5 | 22014-22018 |
| 6 | PB747 | Rahnella aquiatiiis | ≥10⁸ | 0.1 | 5 | 22019-22023 |
| 7 | PB910 | Kluyvera intermedia | ≥10⁸ | 0.1 | 5 | 22024-22028 |
| 8 | PB1116 | Enterobacter tabaci | ≥10⁸ | 0.1 | 5 | 22029-22033 |

The test articles were supplied by the Sponsor in ready to dose form. All animals were dosed once via subcutaneous injection in the pre-scapular area on Study Day (SD) 1. Physical examinations were performed daily and cageside observations were performed twice daily. Dermal observations were also performed daily according to Table 16. Further observations are listed in Tables 17-27.

TABLE 16
dermal observations

| Score | Grade | Edema | Erythema |
|---|---|---|---|
| 0 | None | No Swelling | Normal color |
| 1 | Minimal | Slight swelling | Light pink |
| | | (barely perceptible) | (barely perceptible) |
| 2 | Mild | Defined swelling | Bright pink/ |
| | | (distinct) | Pale red |
| 3 | Moderate | Defined swelling | Bright red |
| | | (raised) | |
| 4 | Severe | Pronounced swelling | Dark red |

Body weights were recorded prior to dose administration (SD 1) and prior to termination (SD 8). Body temperature was taken prior to termination (SD 8). Following terminal data collection on SD 8, the animals were euthanized via CO₂ inhalation followed by cervical dislocation and discarded without necropsy.

Treatment with any of the bacterial isolates did not affect mortality. All animals survived until the scheduled termination.

Clinical observations were noted during physical exams. No abnormalities were noted in any of the animals throughout the course of the study.

No abnormalities were noted during cageside observations.

Dermal draize observations were performed. One animal from Group 6 exhibited minimal edema and erythema starting on SD 7 through the end of the observation period (SD 8). One animal from Group 7 exhibited mild erythema starting on SD 5 through the end of the observation period (SD 8). In Group 8, minimal or mild edema was observed in two animals from one to four days. Mild erythema was observed in Animal 22029 (8f) on SD 7 through SD 8, and moderate erythema was noted in Animal 22032 (8f) on SD 5 and 6, which subsided to minimal erythema on SD 7 and 8.

Body weights and body weight changes were noted. Groups 1, 2, 3, 5, 6, and 8 lost minimal weight throughout the study, with a mean percent change ranging from −1.91% to −6.87%. Groups 4 and 7 gained minimal weight throughout the study, with a mean percent change of 0.31% and 2.73%, respectively.

Body temperatures were tracked. Body temperatures were normal for all groups, with mean temperatures ranging from 36.66° C. to 37.62° C.

### Example 10 Field Trial

A field trial may be conducted to evaluate the efficacy of Pivot strains on corn growth and productivity under varying nitrogen regimes.

### Materials and Methods

Experiment design: Split Plot Design with N rates as Main plots and Treatments as Subplots.
Locations: Three geographically diverse locations in Argentina.
Data Provide within 1 week of planting geo referencing TAG coordinates to be provided, Field Plot, Treatment Locations and Decoder chart.
Treatments: 5 Main Plots—100%, 100%—25 lbs., 100%—50 lbs., 100%—75 lbs., 0%
   14 Subplot Seed Treatments (treated seed supplied by AGT)
Nitrogen Timing: Pre-plant Urea or other approved best management practices.
Replications: 6
Plots 420 per location
Hybrid: Supplied by AgriThority, one for northern locations & one for southern locations.
   NO biological applied to the seed.
Plot size minimum: 4 rows of 30′ length (˜3 acres total)
Observations: All observations (unless otherwise noted) taken from center 2 rows of plot. All destructive sampling to be taken from outside rows.
Maintaining samples: Keep treated seed samples refrigerated till use. Remove samples from refrigerator 1.5 to 2 hours before these are required for application.
Fertilizer Application Cooperator to provide recommended fertilizer rate and gain agreement from Pivot Bio prior to application.

### Local Agricultural: Practice

Seed: Commercial corn (provided) applied with commercial seed treatment with no biological. Seeding rate—local practice.
Planting date: local practice.
Local standard production practices—weed, insect management, etc.
Standard management practices should be followed with the exception of fungicide applications. Fungicide applications require consultation w/client.
Record all agricultural practices followed for raising crop

### Soil Characterization

Soil texture
Soil fertility analysis
   To determine nitrogen fertilizer level take a pre-plant soil samples from each location to insure 0-12″ and potentially 12″-24″ Nitrate Nitrogen is lower than 50 lbs,/Ac. Complete a standard soil test plus Nitrate Nitrogen, Ammonium Nitrogen, Total Nitrogen, Organic Matter and CEC. Send directly to lab,
   Acceptable nitrogen responsiveness. High delta yield between 0 & 100% nitrogen fertilization.
   pH, CEC, total K and P,
   Standard soil sampling procedure, e.g. soil cores 0 cm to 30 cm and 30 cm-60 cm
   Prior to planting and fertilization collect a Pivot Soil Sample—2 ml soil sample at 0 to 6-12 inches from the UTC. Collect one sample per rep per N regime using the middle of the row. (5 fertilizer regimes*6 reps=30 soil samples). Send to Pivot Bio.
   Post planting (V4-V6) collect a Pivot Soil Sample—2 ml soil sample at 0 to 6-12 inches from the UTC. Collect one sample per rep per N regime using. the middle of the row. (5 fertilizer regimes*6 reps=30 soil samples).
   Post Harvest collect a Pivot Soil Sample—2 ml soil sample at 0 to 6-12 inches from the UTCV. Collect one sample per rep per N regime using the middle of the row. (5 fertilizer regimes*6 reps=30 soil samples)
   Post Harvest soil samples from each location at 0-12″ and potentially 12″-24″. Complete a standard soil test plus Nitrate Nitrogen, Ammonium Nitrogen, Total Nitrogen, Organic Matter and CEC. Send directly to lab.
   A V6-V10 soil sample from each fertilizer regime (exclude the treatment of 100% and 100%+25 lbs. [in the 100% block] for all fertilizer regimens] at 0-12″ and 12″-24″. Complete a standard soil test plus Nitrate Nitrogen, Ammonium Nitrogen, Total Nitrogen, Organic Matter and CEC. Send directly to lab. (5 regimes times 2 depth=10 samples per location).
   A post harvest soil sample from each fertilizer regime (exclude the treatment of 100% and 100%+25 lbs [in the 100% block] for all fertilizer regimens) at 0-12″-24″. Complete a standard soil test plus Nitrate Nitrogen, Ammonium Nitrogen, Total Nitrogen, Organic Matter and CEC. Send directly to lab. {5 regimes times 2 depth=10 samples per location}.

### Assessments

Temperature probe (on going monitoring)
Initial plant population @-50% of UTC
Final plant population prior to harvest
Vigor (1 to 10 scale, w/10=excellent) V4 & V8-V10
Plant height V8-V10 & V14
Yield (Bu/acre), adjusted to standard moisture pct.
Test weight
Grain Moisture %
Stalk nitrate test @ black layer (420 plots×7 locations)
Colonization. 1 plant per plot into 1 zip lock bag at 0% and 100% fertilizer at V4-V6 (1 plant*14 treatments*6 reps*2 fertilizer Regimes=168 plants)
Transcriptomics. 1 plant per plot into one tube at 0% and 100% fertilizer at V4-V6 (1 plant*14 treatments*6 reps*2 fertilizer Regimes=168 plants)
Colonization. 1 plant per plot into I zip lock bag at 0% and 100% fertilizer at V10 V12 (1 plant*14 treatments*6 reps*2 fertilizer Regimes=168 plants)
NDVI or NDRE Determination using Greenseeker instrument at two time points (V4-V6, & VT)
   Assess each plot at all 7 locations (420 plots×2 time points×7 locations)
Stalk Characteristics measured at all 7 locations between R2 & R5
   Record stalk diameter of 10 plants/plot at 6 inch height
   Record length of first internode above 6 inch mark
   10 plants monitored, 5 consecutive plants from center of two inside rows
   Seven locations (420 plots×7 locations)
      a. Monitoring Schedule
AgriThority professionals to visit ail trials at V3-V4 stage to assess early-season response to treatments and during reproductive growth to monitor maturity
Joint visit with Pivot scheduled as conditions warrant and client desires.
   b. Weather Information
   Weather data from planting to harvest consisting of:
Daily maximum and minimum temperatures,
Soil temperature at seeding
Daily rainfall plus irrigation (if applied)
Unusual weather events e.g. excessive rain, wind, cold, heat
   c. Data Analyses and Reporting
AgriThority provides Pivot mid-season report by August 1, functional draft by November 30 and final report by December 31
Additional reports can be negotiated
Cooperator will provide replication data (raw data) to AgriThority upon completion of trial in Excel format (provided) by Nov. 15, 2016 including planting layout map.
The report should include all relevant information including that noted below:
Soil texture & test report
Row Spacing, Plot Size, Irrigation and records of water applied, Tillage, Previous Crop
Seeding Rate & Plant Population
Fertilizer Inputs for the Season—Source, Rate, Timing, Placement
Agronomic inputs (e.g. irrigation, herbicides used, etc.)
Harvest Area Dimensions
Method of Harvest—Hand, Machine, Measurement Tool (scales, yield monitor, etc).

TABLE 17
Screening of Bacterial Isolates for Toxicity in Mice
Following a Single Subcutaneous Dose
Dermal Draize Observations
Draize Grading-Scale

| Score | Grade | Edema | Erythema |
|---|---|---|---|
| 0 | None | No Swelling | Normal color |
| 1 | Minimal | Slight swelling | Light pink |
| | | (barely perceptible) | (barely perceptible) |
| 2 | Mild | Defined swelling | Bright pink/Pale |
| | | (distinct) | red |
| 3 | Moderate | Defined swelling | Bright red |
| | | (raised) | |
| 4 | Severe | Pronounced swelling | Dark red |

TABLE 18
Screening of Bacterial Isolates for Toxicity in Mice
Following a Single Subcutaneous Dose
Dermal Draize Observations
Day Numbers Relative to Start Date

| Group | Sex | Animal | Clinical Sign | 1 | 2 | 3 | 4 | 5 | 6 | 7 | 8 |
|---|---|---|---|---|---|---|---|---|---|---|---|
| 1 | f | 21494 | Edema (treated site-1) | 0 | 0 | 0 | 0 | 0 | 0 | 0 | 0 |
| | | | Erythema (treated site-1) | 0 | 0 | 0 | 0 | 0 | 0 | 0 | 0 |
| | | 21995 | Edema (treated site-1) | 0 | 0 | 0 | 0 | 0 | 0 | 0 | 0 |
| | | | Erythema (treated site-1) | 0 | 0 | 0 | 0 | 0 | 0 | 0 | 0 |
| | | 21996 | Edema (treated site-1) | 0 | 0 | 0 | 0 | 0 | 0 | 0 | 0 |
| | | | Erythema (treated site-1) | 0 | 0 | 0 | 0 | 0 | 0 | 0 | 0 |
| | | 21997 | Edema (treated site-1) | 0 | 0 | 0 | 0 | 0 | 0 | 0 | 0 |
| | | | Erythema (treated site-1) | 0 | 0 | 0 | 0 | 0 | 0 | 0 | 0 |
| | | 21998 | Edema (treated site-1) | 0 | 0 | 0 | 0 | 0 | 0 | 0 | 0 |
| | | | Erythema (treated site-1) | 0 | 0 | 0 | 0 | 0 | 0 | 0 | 0 |
| 2 | f | 21999 | Edema (treated site-1) | 0 | 0 | 0 | 0 | 0 | 0 | 0 | 0 |
| | | | Erythema (treated site-1) | 0 | 0 | 0 | 0 | 0 | 0 | 0 | 0 |
| | | 22000 | Edema (treated site-1) | 0 | 0 | 0 | 0 | 0 | 0 | 0 | 0 |
| | | | Erythema (treated site-1) | 0 | 0 | 0 | 0 | 0 | 0 | 0 | 0 |
| | | 22001 | Edema (treated site-1) | 0 | 0 | 0 | 0 | 0 | 0 | 0 | 0 |
| | | | Erythema (treated site-1) | 0 | 0 | 0 | 0 | 0 | 0 | 0 | 0 |
| | | 22002 | Edema (treated site-1) | 0 | 0 | 0 | 0 | 0 | 0 | 0 | 0 |
| | | | Erythema (treated site-1) | 0 | 0 | 0 | 0 | 0 | 0 | 0 | 0 |
| | | 22003 | Edema (treated site-1) | 0 | 0 | 0 | 0 | 0 | 0 | 0 | 0 |
| | | | Erythema (treated site-1) | 0 | 0 | 0 | 0 | 0 | 0 | 0 | 0 |

Nominal Dose: Animals were dosed according to Text Table 1

TABLE 19
Screening of Bacterial Isolates for Toxicity in Mice
Following a Single Subcutaneous Dose
Dermal Draize Observations
Day Numbers Relative to Start Date

| Group | Sex | Animal | Clinical Sign | 1 | 2 | 3 | 4 | 5 | 6 | 7 | 8 |
|---|---|---|---|---|---|---|---|---|---|---|---|
| 3 | f | 22004 | Edema (treated site-1) | 0 | 0 | 0 | 0 | 0 | 0 | 0 | 0 |
| | | | Erythema (treated site-1) | 0 | 0 | 0 | 0 | 0 | 0 | 0 | 0 |
| | | 22005 | Edema (treated site-1) | 0 | 0 | 0 | 0 | 0 | 0 | 0 | 0 |
| | | | Erythema (treated site-1) | 0 | 0 | 0 | 0 | 0 | 0 | 0 | 0 |
| | | 22006 | Edema (treated site-1) | 0 | 0 | 0 | 0 | 0 | 0 | 0 | 0 |
| | | | Erythema (treated site-1) | 0 | 0 | 0 | 0 | 0 | 0 | 0 | 0 |
| | | 22007 | Edema (treated site-1) | 0 | 0 | 0 | 0 | 0 | 0 | 0 | 0 |
| | | | Erythema (treated site-1) | 0 | 0 | 0 | 0 | 0 | 0 | 0 | 0 |
| | | 22008 | Edema (treated site-1) | 0 | 0 | 0 | 0 | 0 | 0 | 0 | 0 |
| | | | Erythema (treated site-1) | 0 | 0 | 0 | 0 | 0 | 0 | 0 | 0 |
| 4 | f | 22009 | Edema (treated site-1) | 0 | 0 | 0 | 0 | 0 | 0 | 0 | 0 |
| | | | Erythema (treated site-1) | 0 | 0 | 0 | 0 | 0 | 0 | 0 | 0 |
| | | 22010 | Edema (treated site-1) | 0 | 0 | 0 | 0 | 0 | 0 | 0 | 0 |
| | | | Erythema (treated site-1) | 0 | 0 | 0 | 0 | 0 | 0 | 0 | 0 |
| | | 22011 | Edema (treated site-1) | 0 | 0 | 0 | 0 | 0 | 0 | 0 | 0 |
| | | | Erythema (treated site-1) | 0 | 0 | 0 | 0 | 0 | 0 | 0 | 0 |
| | | 22012 | Edema (treated site-1) | 0 | 0 | 0 | 0 | 0 | 0 | 0 | 0 |
| | | | Erythema (treated site-1) | 0 | 0 | 0 | 0 | 0 | 0 | 0 | 0 |
| | | 22013 | Edema (treated site-1) | 0 | 0 | 0 | 0 | 0 | 0 | 0 | 0 |
| | | | Erythema (treated site-1) | 0 | 0 | 0 | 0 | 0 | 0 | 0 | 0 |

Nominal Dose: Animals were dosed according to Text Table 1

TABLE 20
Screening of Bacterial Isolates for Toxicity in Mice
Following a Single Subcutaneous Dose
Dermal Draize Observations
Day Numbers Relative to Start Date

| Group | Sex | Animal | Clinical Sign | 1 | 2 | 3 | 4 | 5 | 6 | 7 | 8 |
|---|---|---|---|---|---|---|---|---|---|---|---|
| 5 | f | 22014 | Edema (treated site-1) | 0 | 0 | 0 | 0 | 0 | 0 | 0 | 0 |
| | | | Erythema (treated site-1) | 0 | 0 | 0 | 0 | 0 | 0 | 0 | 0 |
| | | 22015 | Edema (treated site-1) | 0 | 0 | 0 | 0 | 0 | 0 | 0 | 0 |
| | | | Erythema (treated site-1) | 0 | 0 | 0 | 0 | 0 | 0 | 0 | 0 |
| | | 22016 | Edema (treated site-1) | 0 | 0 | 0 | 0 | 0 | 0 | 0 | 0 |
| | | | Erythema (treated site-1) | 0 | 0 | 0 | 0 | 0 | 0 | 0 | 0 |
| | | 22017 | Edema (treated site-1) | 0 | 0 | 0 | 0 | 0 | 0 | 0 | 0 |
| | | | Erythema (treated site-1) | 0 | 0 | 0 | 0 | 0 | 0 | 0 | 0 |
| | | 22018 | Edema (treated site-1) | 0 | 0 | 0 | 0 | 0 | 0 | 0 | 0 |
| | | | Erythema (treated site-1) | 0 | 0 | 0 | 0 | 0 | 0 | 0 | 0 |
| 6 | f | 22019 | Edema (treated site-1) | 0 | 0 | 0 | 0 | 0 | 0 | 0 | 0 |
| | | | Erythema (treated site-1) | 0 | 0 | 0 | 0 | 0 | 0 | 0 | 0 |
| | | 22020 | Edema (treated site-1) | 0 | 0 | 0 | 0 | 0 | 0 | 0 | 0 |
| | | | Erythema (treated site-1) | 0 | 0 | 0 | 0 | 0 | 0 | 0 | 0 |
| | | 22021 | Edema (treated site-1) | 0 | 0 | 0 | 0 | 0 | 0 | 0 | 0 |
| | | | Erythema (treated site-1) | 0 | 0 | 0 | 0 | 0 | 0 | 0 | 0 |
| | | 22022 | Edema (treated site-1) | 0 | 0 | 0 | 0 | 0 | 0 | 1 | 1 |
| | | | Erythema (treated site-1) | 0 | 0 | 0 | 0 | 0 | 0 | 1 | 1 |
| | | 22023 | Edema (treated site-1) | 0 | 0 | 0 | 0 | 0 | 0 | 0 | 0 |
| | | | Erythema (treated site-1) | 0 | 0 | 0 | 0 | 0 | 0 | 0 | 0 |

Nominal Dose: Animals were dosed according to Text Table 1

TABLE 21
Screening of Bactenal Isolates for Toxicity in Mice
Following a Single Subcutaneous Dose
Dermal Draize Observations
Day Numbers Relative to Start Date

| Group | Sex | Animal | Clinical Sign | 1 | 2 | 3 | 4 | 5 | 6 | 7 | 8 |
|---|---|---|---|---|---|---|---|---|---|---|---|
| 7 | f | 22024 | Edema (treated site-1) | 0 | 0 | 0 | 0 | 0 | 0 | 0 | 0 |
| | | | Erythema (treated site-1) | 0 | 0 | 0 | 0 | 0 | 0 | 0 | 0 |
| | | 22025 | Edema (treated site-1) | 0 | 0 | 0 | 0 | 0 | 0 | 0 | 0 |
| | | | Erythema (treated site-1) | 0 | 0 | 0 | 0 | 0 | 0 | 0 | 0 |
| | | 22026 | Edema (treated site-1) | 0 | 0 | 0 | 0 | 0 | 0 | 0 | 0 |
| | | | Erythema (treated site-1) | 0 | 0 | 0 | 0 | 0 | 0 | 0 | 0 |
| | | 22027 | Edema (treated site-1) | 0 | 0 | 0 | 0 | 0 | 0 | 0 | 0 |
| | | | Erythema (treated site-1) | 0 | 0 | 0 | 0 | 0 | 0 | 0 | 0 |
| | | 22028 | Edema (treated site-1) | 0 | 0 | 0 | 0 | 0 | 0 | 0 | 0 |
| | | | Erythema (treated site-1) | 0 | 0 | 0 | 0 | 2 | 2 | 2 | 2 |
| 8 | f | 22029 | Edema (treated site-1) | 0 | 0 | 0 | 0 | 0 | 0 | 0 | 0 |
| | | | Erythema (treated site-1) | 0 | 0 | 0 | 0 | 0 | 0 | 2 | 2 |
| | | 22030 | Edema (treated site-1) | 0 | 0 | 0 | 0 | 0 | 0 | 0 | 0 |
| | | | Erythema (treated site-1) | 0 | 0 | 0 | 0 | 0 | 0 | 0 | 0 |
| | | 22031 | Edema (treated site-1) | 0 | 0 | 0 | 0 | 0 | 0 | 0 | 0 |
| | | | Erythema (treated site-1) | 0 | 0 | 0 | 0 | 0 | 0 | 0 | 0 |
| | | 22032 | Edema (treated site-1) | 0 | 0 | 0 | 1 | 1 | 1 | 1 | 1 |
| | | | Erythema (treated site-1) | 0 | 0 | 0 | 0 | 3 | 3 | 1 | 1 |
| | | 22033 | Edema (treated site-1) | 0 | 0 | 0 | 0 | 0 | 0 | 0 | 2 |
| | | | Erythema (treated site-1) | 0 | 0 | 0 | 0 | 0 | 0 | 0 | 2 |

Nominal Dose: Animals were dosed according to Text Table 1

TABLE 22
Screening of Bacterial Isolates for Toxicity in Mice
Following a Single Subcutaneous Dose
Body Weights and Body Weight Changes (g)
Day Numbers Relative to Start Date

| | | | | | | Absolute | Percent |
|---|---|---|---|---|---|---|---|
| | | | | | | Change | Change |
| | | | | | 1 | 1 | 1 |
| Group | Sex | Animal | 1 | 8 | 8 | 8 | 8 |
| 1 | f | 21994 | 27.11 | 26.39 | −0.72 | −0.72 | −2.66 |
| | | 21995 | 26.62 | 27.37 | 0.75 | 0.75 | 2.87 |
| | | 21996 | 28.06 | 27.3 | −0.76 | −0.76 | −2.71 |
| | | 21997 | 25.01 | 25.49 | 0.48 | 0.48 | 1.92 |
| | | 21998 | 27.15 | 23.64 | −3.51 | −3.51 | −12.93 |
| | | Mean | 26.79 | 26.038 | −0.752 | −0.752 | −2.71 |
| | | S.D. | 1.123 | 1.544 | 1.687 | 1.687 | 6.25 |
| | | N | 5 | 5 | 5 | 5 | 5 |
| 2 | f | 21999 | 29.78 | 29.85 | 0.07 | 0.07 | 0.24 |
| | | 22000 | 29.01 | 28.58 | −0.43 | −0.43 | −1.48 |
| | | 22001 | 26.93 | 22.56 | −4.37 | −4.37 | −16.23 |
| | | 22002 | 25.44 | 22.25 | −3.19 | −3.19 | −12.54 |
| | | 22003 | 28.66 | 29.39 | 0.73 | 0.73 | 2.55 |
| | | Mean | 27.964 | 26.526 | −1.438 | −1.438 | −5.49 |
| | | S.D. | 1.755 | 3.791 | 2.217 | 2.217 | 8.34 |
| | | N | 5 | 5 | 5 | 5 | 5 |

Nominal Dose: Animals were dosed according to Text Table 1

TABLE 23
Screening of Bacterial Isolates for Toxicity in Mice
Following a Single Subcutaneous Dose
Body Weights and Body Weight Changes (g)
Day Numbers Relative to Start Date

| | | | | | | Absolute | Percent |
|---|---|---|---|---|---|---|---|
| | | | | | | Change | Change |
| | | | | | 1 | 1 | 1 |
| Group | Sex | Animal | 1 | 8 | 8 | 8 | 8 |
| 3 | f | 22004 | 26.1 | 27.98 | 1.88 | 1.88 | 7.2 |
| | | 22005 | 26.65 | 20.82 | −5.33 | −5.83 | −21.98 |
| | | 22006 | 26.07 | 25.58 | −0.49 | −0.49 | −1.88 |
| | | 22007 | 27.02 | 27.11 | 0.09 | 0.09 | 0.33 |
| | | 22008 | 26.31 | 26.88 | 0.57 | 0.57 | 2.17 |
| | | Mean | 26.43 | 25.674 | −0.756 | −0.756 | −2.81 |
| | | S.D. | 0.403 | 2.846 | 2.968 | 2.968 | 11.17 |
| | | N | 5 | 5 | 5 | 5 | 5 |
| 4 | f | 22009 | 26.59 | 28.37 | 1.78 | 1.78 | 6.69 |
| | | 22010 | 28.02 | 28.34 | 0.32 | 0.32 | 1.14 |
| | | 22011 | 27.21 | 26.25 | −0.96 | −0.96 | −3.53 |
| | | 20012 | 26.33 | 25.7 | -0.63 | −0.63 | −2.39 |
| | | 20013 | 27.06 | 26.96 | −0.1 | −0.1 | −0.37 |
| | | Mean | 27.042 | 27.124 | 0.082 | 0.082 | 0.31 |
| | | S.D. | 0.651 | 1.209 | 1.068 | 1.068 | 4 |
| | | N | 5 | 5 | 5 | 5 | 5 |

Nominal Dose: Animals were dosed according to Text Table 1

TABLE 24
Screening of Bacterial Isolates for Toxicity in Mice
Following a Single Subcutaeous Dose
Body Weights and Body Weight Changes (g)
Day Numbers Relative to Start Date

| | | | | | | Absolute | Percent |
|---|---|---|---|---|---|---|---|
| | | | | | | Change | Change |
| | | | | | 1 | 1 | 1 |
| Group | Sex | Animal | 1 | 8 | 8 | 8 | 8 |
| 5 | f | 22014 | 24.27 | 24.79 | 0.52 | 0.52 | 2.14 |
| | | 22015 | 27 | 26.56 | −0.44 | −0.44 | −1.63 |
| | | 27016 | 26.88 | 25.87 | −1.01 | −1.01 | −3.76 |
| | | 22017 | 26.12 | 23.67 | −2.45 | −2.45 | −9.38 |
| | | 22018 | 27.74 | 27.39 | −0.35 | −0.35 | −1.26 |
| | | Mean | 26.402 | 25.656 | −0.746 | −0.746 | −2.78 |
| | | S.D. | 1.322 | 1.463 | 1.098 | 1.098 | 4.25 |
| | | N | 5 | 5 | 5 | 5 | 5 |
| 6 | f | 22019 | 28.29 | 28.58 | 0.29 | 0.29 | 1.03 |
| | | 22020 | 26 | 27.64 | 1.64 | 1.64 | 6.31 |
| | | 22021 | 27.73 | 27.83 | 0.1 | 0.1 | 0.36 |
| | | 22022 | 27.2 | 21.62 | −5.58 | −5.58 | −20.51 |
| | | 22023 | 24 | 24.78 | 0.78 | 0.78 | 3.25 |
| | | Mean | 26.644 | 26.09 | −0.554 | −0.554 | −1.91 |
| | | S.D. | 1.703 | 2.886 | 2.872 | 2.872 | 10.65 |
| | | N | 5 | 5 | 5 | 5 | 5 |

Nominal Dose: Animals were dosed according to Text Table 1

TABLE 25
Screening of Bacterial Isolates for Toxicity in Mice
Following a Single Subcutaneous Dose
Body Weights and Body Weight Changes (g)
Day Numbers Relative to Start Date

| | | | | | | Absolute | Percent |
|---|---|---|---|---|---|---|---|
| | | | | | | Change | Change |
| | | | | | 1 | 1 | 1 |
| Group | Sex | Animal | 1 | 8 | 8 | 8 | 8 |
| 7 | f | 22024 | 26.81 | 27.35 | 0.54 | 0.54 | 2.01 |
| | | 22025 | 28.42 | 28.98 | 0.56 | 0.56 | 1.97 |
| | | 22026 | 26.73 | 27.06 | 0.33 | 0.33 | 1.23 |
| | | 22027 | 26.48 | 27.24 | 0.76 | 0.76 | 2.87 |
| | | 22028 | 27 | 28.5 | 1.5 | 1.5 | 5.56 |
| | | Mean | 27.088 | 27.826 | 0.738 | 0.738 | 2.73 |
| | | S.D. | 0.768 | 0.858 | 0.452 | 0.452 | 1.68 |
| | | N | 5 | 5 | 5 | 5 | 5 |
| 8 | f | 27029 | 30.52 | 30.39 | −0.13 | −0.13 | −0.43 |
| | | 22030 | 25.61 | 20.28 | −5.33 | −5.33 | −20.81 |
| | | 22031 | 25.35 | 26.14 | 0.79 | 0.79 | 3.12 |
| | | 22032 | 27.72 | 28.88 | 1.16 | 1.16 | 4.18 |
| | | 27033 | 26.99 | 21.48 | −5.51 | −5.51 | −20.41 |
| | | Mean | 27.238 | 25.434 | −1.804 | −1.804 | −6.87 |
| | | S.D. | 2.078 | 4.448 | 3.335 | 3.335 | 12.66 |
| | | N | 5 | 5 | 5 | 5 | 5 |

Nominal Dose: Animals were dosed according to Text Table 1

TABLE 26
Screening of Bacterial Isolates for Toxicity in Mice
Following a Single Subcutaneous Dose
Body Temperatures (° C.)
Day Numbers Relative to Start Date

| Group | Sex | Animal | 8 |
|---|---|---|---|
| 1 | f | 21994 | 35.8 |
| | | 21995 | 36.9 |
| | | 21996 | 36.7 |
| | | 21997 | 36.3 |
| | | 21998 | 37.6 |
| | | Mean | 36.66 |
| | | S.D. | 0.67 |
| | | N | 5 |
| 2 | f | 21999 | 36.4 |
| | | 22000 | 37.1 |
| | | 22001 | 37.4 |
| | | 22002 | 36.8 |
| | | 22003 | 36.4 |
| | | Mean | 36.82 |
| | | S.D. | 0.44 |
| | | N | 5 |
| 3 | f | 22004 | 37.2 |
| | | 22005 | 37 |
| | | 22006 | 36.8 |
| | | 22007 | 37.1 |
| | | 22008 | 37.1 |
| | | Mean | 37.16 |
| | | S.D. | 0.34 |
| | | N | 5 |
| 4 | f | 27009 | 37.3 |
| | | 22010 | 37.2 |
| | | 22011 | 37.6 |
| | | 20012 | 37.1 |
| | | 20013 | 37.8 |
| | | Mean | 37.4 |
| | | S.D. | 0.29 |
| | | N | 5 |

Nominal Dose: Animals were dosed according to Text Table 1

TABLE 27
Screening of Bacterial Isolates for Toxicity in Mice
Following a Single Subcutaneous Dose
Body Temperatures (° C.)
Day Numbers Relative to Start Date

| Group | Sex | Animal | 8 |
|---|---|---|---|
| 5 | f | 22014 | 36.4 |
| | | 22015 | 37.4 |
| | | 22016 | 37.3 |
| | | 22017 | 37.6 |
| | | 22018 | 37.8 |
| | | Mean | 37.3 |
| | | S.D. | 0.54 |
| | | N | 5 |
| 6 | f | 22019 | 36.4 |
| | | 22020 | 37.6 |
| | | 22021 | 37.8 |
| | | 22022 | 38.2 |
| | | 22023 | 38.1 |
| | | Mean | 37.62 |
| | | S.D. | 0.72 |
| | | N | 5 |
| 7 | f | 22024 | 37.3 |
| | | 22025 | 37.7 |
| | | 22026 | 37 |
| | | 22027 | 37.6 |
| | | 22028 | 37.8 |
| | | Mean | 37.48 |
| | | S.D. | 0.33 |
| | | N | 5 |
| 8 | f | 22029 | 38 |
| | | 22030 | 38.1 |
| | | 22031 | 36.8 |
| | | 22032 | 38 |
| | | 22033 | 37.2 |
| | | Mean | 37.62 |
| | | S.D. | 0.58 |
| | | N | 5 |

Nominal Dose: Animals were dosed according to Text Table 1

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. (canceled)

2. A bacterial composition, comprising:
a bacterial strain, wherein said strain has been remodeled to increase colonization of said bacterial strain on a plant.

3. The bacterial composition of claim 2, wherein said colonization of said bacterial strain occurs on a root of said plant.

4. The bacterial composition of claim 2, wherein said bacterial strain comprises a genetic modification in an enzyme or pathway involved in production of exopolysaccharides.

5. The bacterial composition of claim 0, wherein said genetic modification is in a gene selected from the group consisting of bcsII, bcsIII, and yjbE.

6. The bacterial composition of claim 2, wherein said bacterial strain comprises a genetic modification in an enzyme or pathway involved in production of a filamentous hemagglutinin.

7. The bacterial composition of claim 0, wherein said genetic modification is in a fhaB gene.

8. The bacterial composition of claim 2, wherein said bacterial strain comprises a genetic modification in an enzyme or pathway involved in production of an endo-polygalaturonase.

9. The bacterial composition of claim 0, wherein said genetic modification is in a pehA gene.

10. The bacterial composition of claim 2, wherein said bacterial strain comprises a genetic modification in an enzyme or pathway involved in production of trehalose.

11. The bacterial composition of claim 10, wherein said genetic modification is in a gene selected from the group consisting of: otsB and treZ.

12. The bacterial composition of claim 2, wherein said bacterial composition is formulated for application to a field.

13. The bacterial composition of claim 2, wherein said bacterial strain provides a nutrient to said plant.

14. The bacterial composition of claim 2, wherein said bacterial strain provides fixed nitrogen to said plant.

15. The bacterial composition of claim 2, wherein said plant is selected from the group consisting of corn, barley, wheat, sorghum, soy, and rice.

16. A method of increasing the colonization of a bacterial strain on a plant, said method comprising:
introducing into said bacterial strain a genetic modification in a gene involved in a pathway selected from the group consisting of: exopolysaccharide production, endo-polygalaturonase production, and trehalose production.

17-18. (canceled)

19. A method of increasing nitrogen available to a plant, said method comprising:
applying a plurality of remodeled bacteria onto a plant, said plurality of remodeled bacteria having an increased amount of at least one nitrogenase cofactor within said remodeled bacteria.

20. The method of claim 19, wherein said cofactor is sulfur.

21. The method of claim 19, wherein said remodeled bacteria have increased expression of cysZ.

22. The method of claim 19, wherein said remodeled bacteria have increased expression of a sulfur transporter.
